SEQUENCE LISTING (155 sequences)

SEQ ID NO: 1 - DNA, 1515 bp, Cricetulus griseus
atgttaatatgcctcaccagaaacttgggtggtgtctctgggttcttccaagtggtcttcgtggtcagtctatttctggtgcttttcaaggcagtccgattctacttaagaaggcagtggctgctcaagaccctgcagaagtttccatccacgccttcccattggctttgggggcaccaactgaaggacaaggaactcgagcagattcttatctgggtagaggaattcccaagtgcctgcttacactggctctcagggagcaaggcacgagtcgtgctctatgacgctgactatgtgaagatgattctggggagatcagatccaaaggcttctggaatttatggactctttgctccctggattggttacggtttgcttctgttgaatgggaagaagtggttccagcaccggcggatgttgaccccagctttccattatgacatcctcaaaccctatgtcagaatcatggcggactctgtcaatacaatgctagacagatgggagaagcttgatggtgagaactgccctctggagatctttcactatatctcttcaatgacactggacactgttatgaagtgtgctttcagtcaccagggcagtgtccaactggatgaaaattccaggtcctacactaaggctgttgaggatctgaacagccttgctttctttcgaatgaggagtgccttctatgagaataacaccatctataaaatgtcctctgatggccgttggttctaccgtgcctgccagcttgctcatgagcacacagatggagtgatcaagatgaggaaggctcagctgcagaatgaggaagagctggagagggtcaagaagaagaggcgcttggatttcctggacatcctcttgtttgccaaaatggaggatgggagcagcttgtctgatgaggacctgcgtgcagaggtggatacattcatgtttgaaggtcacgataccacagccagtggaatatcctggattttctatgctctggccacacaccctgaataccagcagagatgcagggaagaggtgcagagcatcctgggagatggaacctctgtcacctgggatcacctggaccagatgccctacactaccatgtgcatcaaggaggccctgaggctctacccaccagtaccaattgttagtcgagagctcaacacacctatcaccttcccagatggacgctccttacctaaaggtatcacagttgcaatctccatttatggtcttcaccataatccaagtttatggccaaatcctaaggagtttgacccatctagatttgcaccagattcttctcggcacagctatgctttcctgcccttctcaggaggagcaaggaattgcattgggaaacagtttgctatgaatgagctgaaggtggccgtggccctgactctgctccgctttgagctgttgcctgatcccaccagggtcccagtactcatacaaagacttgttttgaagtccaaggatgggatttatcttcgtctcaagaagctaagataa

SEQ ID NO: 2 - DNA, 1368 bp, Cricetulus griseus
atggtggcctcgcctctcgctgtcctgcgaacaagcaggttgtgccaatggggctggaagagccgggcgcggttgtcagggtctctgcccttcggtcggggaggatggaccacttgggcccctgcgcggacaaatgcgacgctgagcctggagcccgcgggccgcagctgttgggacgagccgctgggtatctccgtgcacggcctggcccccgagcagcccgtcacgctgcgcgcggccctgcgagacgagaaaggcgcgctcttccgcgcccacgcgcgctaccgcgccgacgaccacggcggcctggacctggcgcgcgcgcccgcgctgggcggcagcttcgccgggatcgagcccatggggctgctctgggctctggagcccgaacggccattctggcgcctgatcaagcgcgacgtgcagacgcccttcgtggtggagctggaggtgctggacggccacgagcccgacggcgggcggctgctggcgcgcgcggtgcacgagcgtcacttcatggctcccggggtgcggcgcgtgcccgtgcgcgagggccgtgtgcgcgccacgctcttcctgcctccagaacctgggccctatcctgggatcctggacctttttggagttggaggtggccttctggagtaccgagctagtctgctggctgggaagggctttgctgtcatggctctggcttattataactacgatgacctccccaagggcatggacaccatgcgcatggagtactttgaagaagcagtgaactacctgctcagccaccctgaggtgaagggtccaggaattggactgcttgggatttccaaagggggtgaacttggccttgctatggcctccttcctgaagggcatcaaagctgctgttgtgatcaatggctctgtggctgctgttgggaacaccatatactacaaggatgagactataccccctgtttctcttctgagaaatcgagttaaaatgaccaaagatggcctcttggatgttgtggaaggtctgcaaagccctttggtggaagagaagagcttcatccccgtggaaaggtctgacacggccttcctgttgcttgtaggtcaggatgaccacaactggaagagcgagttctatgccaatgagatctccaaacgcttagaggcccatgggaaggggaagccccagatcatctgctacccagaagcagggcactatattgagcccccttacttcccactgtacaaggctggcatgcacctcctggtgggtgctaatatcacctttggaggggagcctaagcctcatgctgtggcccaggtggatgcatggcagcagctccagactttcttacacaaacacttgggtggtaagagttag

SEQ ID NO: 3 - DNA, 1527 bp, Cricetulus griseus
atgcagcggcttttggctccagcaaggagggtcttgcaagtgaaaagagtgatgcaggatgcttggatcacacctgctcacccgctctcagcagcccagcagaggttttttacaatccctcctgctcccctggccaaaacagacacttggccaaaagatgttggcatccttgccctggaggtctactttccagcccaatatgtggaccaaactgacctggagaagttcaacaatgtggaagccgggaagtatacagtgggcttgggccagacccgtatgggcttctgttcagtccaggaggacatcaactcactgtgtctaacagtggtgcagaggctgatggagcgcacaaagctgccatgggacgctgtgggccgcctagaagtgggcaccgagaccatcattgacaaatccaaggctgtcaagacagtgctcatggaactctttcaggattcaggcaacactgacattgaaggcatagataccaccaacgcctgctacggtggcaccgcctccctcttcaatgctgccaactggatggaatccagctactgggatggtcgctatgccctggtggtctgtggtgatattgcagtctacccagtaggcaacgccaggcccacaggtggcgccggggctgtggcaatgctgattggacccaaggctcctctagtcctggagcaagggctgaggggaacccacatggagaacgcatatgacttctacaaaccaaacttgtcctcggagtatccactagtggacgggaagctctcaatccagtgctacatgcgggccttggatcggtgctatgcagcctaccgcaagaaaatccagaatcagtggaagcgagagggaaacaacaagcctttcaccctcgatgatgtgcaatacatgatcttccacacacccttttgcaagatggtccagaaatccctggctcgactgatgttcaatgacttcctgtcgtccagcagtgacgcacggaatgatttatacaaggggctggaggccttcaggggtctaaagctggaagaaacctacaccaacaaagatgtagacaaggctattttgaaggcctccctggacatgttcaacaagaaaaccaaggcttccctttacctgtccacgaacaatgggaacatgtacacctcatccctctatgggtgcctggcctcacttctctcccaccactctgcccaagaattggctggctccaggattggtgctttctcgtatggctcaggcttagcagcgagtttcttttcatttcgagtgtccaaggatgcctctccaggttcccctctggaggagctggtgtctagtgtgtcagatctgcccaagcgtctagactcccggaggcgcatgacccctgaggaattcacagaaataatgaaccaaagagagcagttctaccataaggtgaacttctcaccacctggtgacacaagaaacctcttcccaggtacttggtatctggaacgagtggatgagatgcatcgccgaaagtatgcccggcgtcccgtctag

SEQ ID NO: 4 - DNA, 1434 bp, Cricetulus griseus
atgtttttctgccagggattttgggagagccaattggaactcagggagagatacgggcctctgtgtgggtactatcttggccgtcggatgtatattgtcatttctgagccagccatgatcaagcaggtgttggttgagaacttcactaacttttccaacagaatggcctcaggtctggaacccaagcctgtagcagacagcatcctgtttttacgagacaaaaggtgggaagaagtcagaggtgccttgatgtctgcattcagtcctgaaaagttgaatgagatgacgcctctcatcagccaagcctgtgaacatctcctgagtcacttaaagctctatgcagcatccagagacacattcgacatccagaggtgttactgctgttacaccacaaatgtggtggccagtgtggcctttggcacccaggtggactcccagaacactccggaagatccctttgtgcaacactgccgtcaattctttgccttgcgtatccccaggcctctcctggctttaatcttatcatttccatccataatggtcccattggcccggattctgcccaataagaaccgagatgaactgaatggcttttttaacaaactcattaggaacgtgattgccttacgggaccagcaagcagcagaagagaggcggagagacttcttgcagatggtgctggatgcccggcactccacaagctctgtgggtgtggaagactttgacatggtcaaagaagccctgtcctctagcgagtgtccagtgaaccctccccaaagaggccagcctgcatccatgcctgagcctttaaccacggatgaaattgtgggccaggccttcctcttcctcatcgctggccaagagatcatcactaacacactctccttcatcacgtacctgctggccacccaccctgactgccaggagaggcttctggaggaggtggacctcttcgtggagaaacacccagcccctgggtactgcagcctgcaggaaggcctgccctatctggacatggtgattgcagagaccctgaggatgtacccaccagctttcagattcacacgggaggcagcccaagactgtgaggtgctgggacagtacatccccgcaggtgcagtgctggaggtagccgtgggtgccctacaccatgacccagagcactggccacaccctgagacctttgaccctgaaaggttcacagcagaggcccggcttcagcgggggcccttcacatacctgcccttcggagctggccccaggagctgcctcggggtgcggctggggctgatggtggtcaaactgacactactccaggtcctacacaaattccgctttgaagccagctctgagactcaggttccacttcagctagaatccaaatctgccctaagtcccaaaaatggagtctacatcaagattgtgtctcgttga

SEQ ID NO: 5 - DNA, 1125 bp, Cricetulus griseus
atgggcactgctggaaaagttatcaagtgcaaagcagctgtactatggggaatgaaccagcccttctccattgaggaaatagaagtggccccaccaaaagctaaggaagttcgtgttaaggttttggccacaggaatctgtcgcacagatgaccatgtgataaaaggagcaatggtgtctaagtttccagtgattttgggacatgaagcagttggggttgtagagagtgttggggaaggagtcactacagtaagaccaggtgacaaggtcatccccctctttctaccacagtgtagagaatgcaatgcctgctgtaacccagagggcaatctctgcattaggagcgatctgacgggtcgtggagtactggccgatggcactaccagattcatgtgcaagggcaaaccggtccagcatttcatgaacaccagcaccttcaccgagtacacggtgctagatgagtcatccgtagctaagatagatggcgcagctgctcctgagaaagcctgcctcattggctgtgggttttccacaggctatggggctgctgttaaaaccgccaaggtcacccctggctccacttgtgtagtgttcggcctgggaggtgttggcctatcggtcatcatgggctgtaaggcagctggagcctccaggatcattggaattgacatcaacaaggacaaattccagaaggccctggctgtaggtgccacagagtgtatcaatcccaaggacttcaccaagcccatcagtgaggtgctgtcagacatgacagggaacaccgtgcagtacacatttgaagttattgggcgtcttgaaaccatggtcgacgccctctcgtcttgtcatatgaactatgggaccagtgtggtggttggcgctcctccatcagccaagacgctcacttacgacccaatgctgctcttcactgggcggacgtggaagggatgcgtctttggaggttggaagagcagagatgacgtaccaaaactggtgactgagctcctggaagagaaatttgacctggaccagttgatcacccacgtcttgccttttgaaagcatcaaagaagggtttgaattgctttattcagggcaaagcattcggactgtcctgacattttga

SEQ ID NO: 6 - DNA, 1038 bp, Cricetulus griseus
atgccccgcatagatgcggatcttaaactcgacttcaaggatgttctgctgcgacctaagcggagcagcctcaaaagccgagccgaggtggatctcgagcgaacctttacatttcgaaactcaaagcagacctactcagggattcccatcattgtagccaacatggacactgtggggacatttgagatggctgcggtaatgtcacagcacgccatgtttacagccgttcacaagcattactccttggatgactggaaacgttttgcggaaaaccaccctgagtgcctgcagcatgtagccgtgagttcgggcagtgggcagaatgatctggagaagatgagccacatcttggaagccgtgccacaggtgaagttcatctgcctggatgtggccaatgggtattcggaacattttgtggaattcgtgaaactggtccgatccaaatttcctgaacacaccatcatggcaggaaatgtggtgacaggagagatggtggaagagctcatcctctctggagcagatatcatcaaagtgggagttggaccaggttctgtgtgtaccacccgaaccaagacaggagtgggctacccgcagctgagtgccgtaatagagtgtgctgactctgcccatggcctcaaggggcacatcatctctgatggaggctgtacatgtccaggagatgtcgccaaagcctttggagccggtgcagacttcgtcatgctgggaggcatgttctcaggccacactgagtgtgcaggggaggtgatcgaacggaacggacagaagctgaaactcttctatggcatgagctcggacacagccatgaagaaacactcgggaggagtcgccgagtacagggcctctgagggcaagactgtggaagtgccttacaaaggggatgtggagaacacaattctggatatcctgggaggacttcggtctacctgtacctacgtaggggctgccaagctcaaagagctcagcaggagagcaacattcatccgggtgacccaacagcacaacacagtgtttggttag

SEQ ID NO: 7 - DNA, 876 bp, Cricetulus griseus
atggacactgtggggacatttgagatggctgcggtaatgtcacagcacgccatgtttacagccgttcacaagcattactccttggatgactggaaacgttttgcggaaaaccaccctgagtgcctgcagcatgtagccgtgagttcgggcagtgggcagaatgatctggagaagatgagccacatcttggaagccgtgccacaggtgaagttcatctgcctggatgtggccaatgggtattcggaacattttgtggaattcgtgaaactggtccgatccaaatttcctgaacacaccatcatggcaggaaatgtggtgacaggagagatggtggaagagctcatcctctctggagcagatatcatcaaagtgggagttggaccaggttctgtgtgtaccacccgaaccaagacaggagtgggctacccgcagctgagtgccgtaatagagtgtgctgactctgcccatggcctcaaggggcacatcatctctgatggaggctgtacatgtccaggagatgtcgccaaagcctttggagccggtgcagacttcgtcatgctgggaggcatgttctcaggccacactgagtgtgcaggggaggtgatcgaacggaacggacagaagctgaaactcttctatggcatgagctcggacacagccatgaagaaacactcgggaggagtcgccgagtacagggcctctgagggcaagactgtggaagtgccttacaaaggggatgtggagaacacaattctggatatcctgggaggacttcggtctacctgtacctacgtaggggctgccaagctcaaagagctcagcaggagagcaacattcatccgggtgacccaacagcacaacacagtgtttggttag

SEQ ID NO: 8 - DNA, 1587 bp, Cricetulus griseus
atgtccatgtcagaacaaggcaagaatgttcagcccagtggcggggaggtgaggctggaactgaggccaacagcccagtggaaccagggctgcagcaacgcagccttccaggccttggggcatcagagggcaatgcgagtcttcaatgggggttggcggcacctggtgatgcggtctccatctggacgaagttgggatgaagaccttgaagagcaggactcaccaggaaacagcacagtcaggagcagagttttacagagcggggaacaaggaaatgccaaacagggtgacaggcagatcacgattgaggaggagtcgtctccaggaaacaaagaagacgtggagggtgatgaccaggatgaccatcaaaaagggtgtttggaaagaaagtatgatatgatttgtgagttttgtagaaagcacaggatcattctccgacgcatcatcggaggcattttattgactggtttcctggctctggtgattgcagcctgtgtactaaacttccacagagcccttcctctatttgtgatcaccgtggttaccatcttctttgttatctgggatcacttgatggccaaatatgagcatcgaatcgatgagttcctatctccatgcagaaggcttctcaacaggcattggttctggctgaaatgggtgatgtggagctcactggtcctggtggttattttctggttgatccttgacaccgccaggctgggcaagcagcagctggtgtcctttggaggactcatactgtacatcgtactgttgtttctcttttccaaacatccaaccagagtttactggaggcctgtcttttggggaattggattacagtttcttcttgggctcttaattctgaggactaatcctggatttattgcttttgattggctgggaagacaagttcagacttttctggagtacactgatactggagctatgtttgtcttcggtgagaagtacacagaccacttcttcgcatttaagatcctgccgatcgtggttttcttcagcactgtgatgtccgtgctctactacgtgggactgatgcaatgggtgatccgaaaggttggatggttaatgcttgtcactatggggtcatctcccattgagtctgtagttgctgctggcaacatatttgttgggcaaacagagtctccactactggtccaaccatatttaccacatgtcaccaggtcagaactccatgccattatgacagctggctttgccacaattgctggaagcgtattgggtgcctatatctcctttggggtgtcatccacacacttgctaactgcctcagtcatgtcagcaccggcatccctggctgtggccaaactcttttggcctgaaacagaaaaacccaaaatcaccctcaagaatgccatgaagattgagagtggtgattcaagaaatctcctggaggcagcaacacagggtgcatcctcgtccatccccctggtagcaaacattgcggtgaatctgatcgccttcctggccttattgtcttttgtgaactcagccctgtcctggttcggaaacatgcttgactacccacagttgagttttgaggtatga

SEQ ID NO: 9 - DNA, 684 bp, Cricetulus griseus
atggcttcttttcacattcgccagttccaggagagagactatgagcaggtcgtggatgtgttctccaggggcctggaggagcacatccccaatgccttccgccacttgctgctgctgccccggaccctcctgctcttagttgtggtgcctcttgccgtagtcctggtgtctggatcctggctcctggctgttgtatgtaacttcttcctgcttggattcttgcggttccttgctagttatccctggaagaattatgtgtctacatgtttgcacactgacatggctgacatcaccaagtcctacctgagtgtgtgtggctcaggtttctgggtggcagaggctggaggacaagtggtgggcctagtggggggtctgccagtcaagaatcctccattagggaggaagcagctacagctctttcgcctgtctgtgtcctcacagcatcgaggacaggggatagcgaaagcactggtcagaactgtcctccagtttgcacgggaccagggctacagcgatgttgtccttgagaccagccttttgcagcaaggtgctgtaaccctctattacagcatgggcttccagaagacaggagaatccttcatgaacagactcacatggcttgtggatctctctctaattcatttcatatactcactcccctctgctcaggaacatgagctgtga

SEQ ID NO: 10 - DNA, 1284 bp, Cricetulus griseus
atggcagaagctgtcttggttgatctctttggtttaaaattgaactcccagaaaatctgccatcagacattacttaagacattgaatggcattcacggccaccatgcccccaaggccaagttcctttgcataatgtgttgcagtaatgaaagggatggtgaacatgatctttgtgactgggaaacaggaaatggattttcacttttcatgagagactttgagactgttagcaatcccagtatggctgccactttgtatgcaattaaacaaaaaattgatgaaaaaaatctgagccacattaaggtgattgtccctgagcacaggaaaacgtttgtgaaggcttttattgatcagctcttcactggggtttatgattttgagtttgaagatttgcaggtagcttggagggagagcctgttgaaaccagccactgaaataaacgaacccacaactcatcaactagaagaaatccagaatgaaatagaaacatacttgagaagcctacccgctctgaaaggggccttaactattatcagatctcctttgatcccagatctcttcatacatggatttactacacgaacaggtggcatatcttacataccaactcttagctcactcaatcttttcagtagttccaaacggagagatcccaaagtagttgtccaagaaaatcttcgaaggttggcaaacgctgcaggattcaatgcagagaaattttaccgaataaagactgatcacgccaatgacgtgtgggtgatggggaagaaggagcctgaatcttacgatggaatagtcacaaaccagagaggagtcaccatcacagctctcggtgctgactgtatacctatagtctttgcagatcccgtgaaaacagcatgtggggttgcccactctggctggaaaggtactttgttgggtgtagctatggctactgtgaatgctatgatagcagaatatggctgtaatttggaagacatcattgttgttctgggaccttccgtggggccttgctgttttactcttcccagagaatcggcaacctcatttcataatcttcatccttcatgtgtgcggcagttggactcactgaatccctatgttgacatccggaaagccactaggattcttctagaacgaggaggaattcttccacagaatattcaagaccagaagggagatcttgacctctgcacatcctgccatcctgataagtttttctcccatgttcgagatggccttaacttcggtacacagattggcttcatatccattagagaatga

SEQ ID NO: 11 - DNA, 1827 bp, Cricetulus griseus
atggaagatcaccattccaggccagcattcggataccagaatgccaggaccgcaaaccggaatgaggtctccttcccctcccattcctggtccttggagatgttgctacgcagactgaaggccatcgatgccaagagggaagacaagctggccagtgttctgagcgccgtgggggagtttggcgccttccagtggaggctggtggtcctcacgttcatccccagcatgctgtcatcctacttcttgctctctgatcacttcatgctcacagcccagaggccttactgtaacaccagctggatcctggcagtgggccccaatctctccttggatgaacagctgaacttgactctgccccgagcacccaatggcagcttcctgacatgcctcatgtacatacctgtgccgtgggatctagattccatcatccattttggtctcaattatacagaaacttgcaaatttgggtggatctacccttatgctaagacacgctctctgatcaacgagtttgacctggtgtgtggcaatgaaccaaacatggagaacatgcagaccgtgttcctcgcgggcatcttgacggggtctctcctctttgggttcttaagcgacaagatgggccgctatccaaccatcctggtgtcactgctggggctcctcgtctttggctttggggctgcctttgtgaaaagcttttaccagtacctgttcttccgctttggtatggcccaggtagccgtgggctatgccatcagcagtcactgcttcatttctgtgggcatcatgctcctgtcggggtgcgcctacaaaatcttccactggcggctgctgttcctgatgggcggtgtgcccatgttacctctcatctccaacatctgggtcctccgagaatccccacggtggctgatggtgaaagggaaggtgcaagaggccaggaaggtgctgtgctatgcagcagaggtgaacaagaagaccatacctttcaatctactcaatgagctgcagctgcagggaaagaaggttaataaagcctccatcctggacttctacaccaatcagcaccttttcaaggtggtcttatccatggcctgcgtgtggtataccgtcagttacatctatttaacactgagcctcaagatgaaggatttggggatagacaactacttcagacaagtgatcccaggcattatggaagtgccagcccggctgagttgcgttgttctcctggagtactttggaaggaagtgtagtctctacctgagcctcaccctagctaccgtcttctgtttgtttctccttttcctccctcaagagctgaggtcaacaataatcttgatgattgtgctcggagagtttagcctggctgccactgtgactgtgttcttcatctacaccgctgagctccttcccaccgtcctcaggtctacaggtctgggcatggtgtctctcgcctgggcggctggagccatcacagctctagcagtcttcaaccaggtcaaaacacaactgcccatcttcttctgctgcctgtcttgcgttgtggccttgtgctttgcctccttggtgccagaaacaggagaccaccctctccgtgatagcatcgagtacagtccaagcacccaaaaagattctactgaatccaagaggaaaaaccaggacattgcttcaatggtaatagatgaggagtcaatgtctgatgttgtgaatgatgaagtgacaaaaaataccattctcaatgccctgcctctaaaatcagaagcaggcaggttcttcaacacagccttggagaaactcaccaacaaagaagtactcagtctagatccctga

SEQ ID NO: 12 - DNA, 2283 bp, Cricetulus griseus
  misc_feature (2267)..(2267): n is a, c, g, or t
ggtgtcgtgaggattagcttggtactaatacgactcacaagcttgccgccaccatgagggctggatgagggctggactggagacagcagacattgccatagtggccctgtattttatcctggtcatgtgcattggtttttttgccatgtggaaatctaatagaagcacagtgagtggatacttcctggctgggcgctctatgacctgggtggcaattggtgcctctctgtttgtgagcaatattgggagtgaacacttcattgggctggcaggatctggagcagcaagtggatttgcagtgggcgcatgggaattcaatgccttactgctcttgcaacttctgggatgggttttcatccctatttacatccggtcaggggtatacactatgcctgaatacttgtccaagagattcggtggccataggattcaggtctattttgcggccttgtctctgctcctctatatcttcaccaagctctcagtggatctgtattcaggtgccctctttatccaggagtccttgggttggaacctctatgtgtctgtcatcctgctcattggcatgaccgccctgctgactgtcaccggaggccttgttgcagtgatctacacagacacgctacaggctctgctcatgattatcggggcactcacacttatggtcattagcatgatggaggttggagggtttgaggaagtaaagaagaggtacatgttggcctcacccaatgttgcttccattttgttgacacacaacctttccaacacaaattcatgtaacgtccaccctaaggatgatgccctaaaaatgttgcgagatccaacagatgaagatgttccatggcctggattcattcttgggcagaccccagcctctgtatggtactggtgtgctgaccaagtcatcgtgcagagggttctagcagccaaaaacattgctcatgccaaaggctctactctaatggctggcttcttgaagctgctaccgatgtttatcatagttgtaccagggatgatttccaggatactgtttgttgatgacatagcttgtatcaacccagagcactgcatgcaagtgtgtgggagcagagctgggtgctccaatatcgcctatccacgcctggtgatgaagttggtccctgtgggcctccggggcttgatgatggccgtgatgattgcagctctgatgagtgacctggactccatctttaacagtgccagtaccatattcacccttgatgtgtacaaacttatccgcaagagtgcaagctcccgggaactaatgatagtgggcaggatatttgtggctttcatggttgtcgtcagcatagcctgggtgccgattattgtagagatgcaaggaggccagatgtacctgtatatccaggaggtggcagactatctgacccctcccgtggcagccctcttcctgctggcaattttctggaaacgctgcaatgagcaaggagctttctatggtggaatggcaggctttgttcttggagccgtccgtttgatactggcttttacctaccgtgctcctgagtgtgaccaacctgataacaggccaggcttcatcaaagacatccattatatgtacgtggccacagcactgttctggatcacaggactcatcactgtgatcgttagtcttctcacaccgcctcccacaaaggaccagattcgcaccaccacattttggtcgaagaagactctggtaacaaaggagagctgctctcagaaagacgagccatacagaatgcaagagaagagcattctgaggtgcagtgacaatagtgaggttgtcaaccatgtcattcccaacgggaaatcggaagacagcattaagggactgcagcctgaagacgtcaatctgttggtggcatgcagagaagagggcaacccggtcgcttccctgggccactcagaggcagaaacaccagtagacgcctattccaatgggcaggcagctctcatgggtgagaaagagagagagaaggaaacagaaaacaggagcaggtattgggagttcgtggactggttctgtggctttaaaagtaaaagccttagcaagaggagtctcagagacttgatggacgaggaggccgtttgttcacagatgttagaagagtctccacaagtaaaagtggtgttaaacattggactttttgctgtgtgttcacttggaattttcatgtttgtttatttctccttatgatctagagtcggggcggccggccgctcgctgatcagcctcgactgtnccttctagtgccagcc

SEQ ID NO: 13 - DNA, 1686 bp, Cricetulus griseus
atgtcctcggaggtgatcagggcgaccgcagggatggtactagcagagctgtatgtctctgatcgagaaggaaatgatgcaacaggagatggaactaaggagaaaccatttaaaacaggcctaaaggctttgatgacagttggaaaagagccatttcccaccatttatgtggattcacaaaaggaaaatgagaggtgggatgtcatttctaagtcccagatgaagaacattaaaaagatgtggcacagagagcagatgaagaatgattctcgggagaagaaagaggcagaagataacttacggagagaaaagaacctggaggaagcaaagaaaattatcattaaaaacgacccaagcctcccagagccagcatgtgtaaagatttgtgcattagaaggatatagaggtcaaagagtgaaggtgtttggctgggtccacaggttactcagggagggaaagaatttgatgtttctggtgttgcgagatggtacaggctatcttcagtgtgtcttgtcagatgacttgtgtcagtgttacaatggagtagtcttgtccaccgagagcagtgtggcagtatatggaacactaaacctaactccaaaaggcaaacaggctccgggaggccatgagctgagctgtgacttctgggagctggtggggctggccccagctgggggggctgataacctgatcaatgaggagtctgacgtagacgtccagctcaacaaccggcacatgatgatccgaggagagaacatgtccaaaatcttgaaagcacgctccatgatcaccaggtgcttcagggatcacttcttcgacagaggctactgtgaagttactactccaacactggtgcagacgcaggtggaaggtggggccacactcttcaagcttgactattttggagaagaagcgtttttaacccagtcctcgcagctgtacctggagacctgccttccggccctgggggatgttttctgtattgcccagtcatacagggctgaacagtccagaacacgaaggcacctggctgaattcactcatgtggaaactgagtctgagtgtcccttcctgacctttgaggacctcctgaaccgtttagaggacctagtgtgtgatgtggtggacagagtcttgaagtcaccagtggcaagcatagtgtatgacctcaacccgaactttaaaccccctaaacggcctttcaggcggatgaactattcagatgctatcgagtggctgagggagcacgatgtaaagaaagaagacgggacactctatgaatttggagatgatattcccgaagctcctgaaagactgatgacagacaccattaatgaaccaatcctgctgtgtcggtttcctgtggagatcaagtccttctatatgcagcgatgtcctgaggatcctcgacttactgaatctgttgacgtgttgatgcccaatgttggtgagattgtgggtggctctatgcgctcttgggatagtgaagaaattctagaaggctataaaagggaaggaattgaccctgctccttattattggtatacagatcagagaaaatatggtacatgtcctcatggaggctatggcttgggcttggaaagattcctaagttggattctgaacaggtatcatatccgagatgtgtgcttgtaccctcgattcgtccagcgctgcaggccataa

SEQ ID NO: 14 - DNA, 873 bp, Cricetulus griseus
atgagcttcgtggcctacgaggagctgatcaaggaaggcgatacagccatcctgtcgctgggccatggctcgatgatggcagtgcgtgtgcagcgtggggcacagacccagacccggcatggcgtcctgcggcactcggtggacctcattggccgcccatttggctccaaggtgatctgcagcagaggcggctgggtgtacgtgcttcaccccactcctgagctctggactgtgaacctgccccaccgcacacagatcctctactccaccgacattgccttaatcaccatgatgctggagctgcgacccggttctgtggtctgtgagtcaggcacgggcagtggttctgtctcccacgccatcatccgcagcatcgcccccactggccacctacacactgtggagttccaccaacagcgggcagacaaggcccgggaggagttccaggagcatcgtgtgagccagtgggtgacggtgcacacccaggatgtttgccgcagtggcttcggtgtcacccacgtggctgatgctgtcttcttggatattccatcaccctgggaaactgtgggccatgcctgggatgccctcaaggttgaaggtgggcgcttctgttccttctctccatgcattgagcaagtgcagcgcacatgccaggcactggcagcccatggtttcacagagctcagtaccctggaggtgctgccacaggtctacaatgtacgcaccgtcagtctgcccttgcctgacctgggggcaagtgacttagaggtcagcatgggctctgatgccagccctttccgtagtggcacacccatgaaggagactgtgggccacactggctacctgacttttgccacgaagactccaggatag

SEQ ID NO: 15 - DNA, 1134 bp, Cricetulus griseus
atgtgtgacgacgaggagaccactgctttggtttgcgacaatggctctgggctggtgaaggccggcttcgcgggtgatgatgcccccagagctgttttcccttccatcgtgggtcgcccacgccaccagggtgtcatggtgggtatggggcagaaggactcctatgtaggtgatgaagcccagagcaagcgaggtatcctgactctgaagtaccccattgagcatggcatcatcaccaactgggacgatatggagaagatctggcaccatacattctacaatgagctccgtgtggcccctgaggagcacccgaccctgctcactgaagccccactgaaccccaaggccaaccgtgagaagatgacccagatcatgtttgagaccttcaatgtccctgccatgtatgtggccatccaagcagtgctgtctctttatgcttctggaagaaccacaggcattgttctggactctggtgatggtgtaactcacaatgtccccatctatgagggttatgccttaccccatgccatcatgcgtctggatctagctggtcgggatctcactgactacctcatgaagatcctcactgagcgcgggtactcctttgtcaccactgctgaacgtgaaatcgtgcgtgacattaaggaaaaactgtgttacgttgccctggactttgagaatgagatggctacagctgcctcttcctcctctctggagaagagctatgaactgcctgatggccaggtcatcaccattggcaacgagcgtttccgctgtccagagacactcttccagccttctttcattggtatggaatctgctggcatccatgaaactacttacaatagcatcatgaagtgtgacatcgatatccgcaaggacctatatgccaacaatgtcctatctggaggcaccactatgtaccctggtattgctgatcgtatgcaaaaggaaatcactgcactggctcccagcaccatgaagattaagattattgctccccctgagcgtaaatactctgtgtggattgggggctctattctggcctctctgtccaccttccaacaaatgtggatcagcaagcaagagtatgacgaggcaggcccatccattgtccatcgcaagtgcttctaa

SEQ ID NO: 16 - DNA, 930 bp, Cricetulus griseus
atggagacgctgctgaagcgtgagctgggctgcagctcggtcaaagctacgggccactcgggaggcgggtgcattagccagggccagagttacgacacggacaaaggacgagtgtttgtgaaagtgaactccaaggcagaggcaagaagaatgtttgaaggtgagatggcaagtttaatggccattatgaagacagacacagtgaaagtacccaagcccatcaaggttctggatgccccaggaggtgggagcgtgctagtgatggagcatttggacatgcggtatctgagcagtcatgccgccaagcttgggtcccagcttgcagatctacacctcgagaacaagaagcttggagaagggctactcaaggaggctggcaccgtggggaaaggaagtggacaggcagagcggctctttgtggaccagtttggatttgatgtggtgacatgttgtggataccttccccaggtaaatgactggcagaatgattgggtcacattctatgccaggcagcgcattcagccccagatggatatggtagagaagaggtctggagaccgggaagctcttgagctgtggtctgctctgcagttgaagattcctgacctattccatgatctggaaattgtgccctctttacttcacggagacctctggggagggaacgtagcagaagattcctctgggcccatcatttttgatccagcttccttctacggccactcagaatatgagctggcaatagccggcatgtttgggggcttcagtagctccttttactctgcctatcatagtaaaatccctaaaaccccaggatttgagaaacgcttgcagttgtatcagctctttcactatctgaaccactggaatcattttggatctgggtacagagggtcctctctaaacataatgcggaatctcagcaaatga

SEQ ID NO: 17 - DNA, 1941 bp, Cricetulus griseus
atgaggaagctctctccttcatctgacttctctggatcattagaaccagagctcaaagtgtcactgtttgatcaacccttgtcaatcatctgtggagagaatgacacactccccaaacccatccaggacatcctcgccatcctctgccttaaaggcccctcaactgaagggatattcaggaaagctgccagtgagaaagcccgcaaggagctgaaggaggaacttaactgtgggagccccgtgaacctgaaccagctcccagtacacctcctggctgtggtcttcaaggacttcctccgaggaatccccctgaagctgctgtcctgtgacctctttgaggagtggatggacgccctggagaagccaagtgaggaggacagaatcgaggccctaaagcaggttgcagactgcctcccccggcccaaccttctactgctcaggcacttggtctatgtgctgcacctgatcagcaagaatgccgaggtcaacaagatggactctagcaacctagccatctgcatcgggcccaacgtgctcactctgaagaatgaccagagcctgtcattccaggtccagaaggacctgaacagtaaggttaagacattggtggaattcctcattgacaactgctttgaaatatttggggagaacattcccacacattcccgtatcacttctgacgactccctggaacacactgacagttcagatgtgtcaactctgcagaatgactcagcttatgacagcaatgacccggatgtggagcccacaagcgccatcacttctcccagcaggcagctggaggggcccactgccacagtgactggcccagataaccgggcttcacaggacacctgtgagtcaaattcagaacctgctgatagcatggtagccaggttgaaaagctccattggccagccagacagacggttctctgaacccaacatgtcaccctcgcaagagtgcctcatggaccaaatgacgaaacaaaagctaaccaagagcgaggataacttcaccatgccccaggaagtctcttgttttgagggtcctgaggctgaagatccatttacagaggaagtcttcccagcagttgaaggcaagcccaagagaccagtggatttaaagataaagaacttgacccacggtttagcatcaccacagggatgggtacccaaaactttgtccagagtctcctcaggtaaatctttggacagctcacccataccttctccttcctttcccaaaagaagcttcttcactaggcaccagagtttcaccacaaagacagacaagatcaaaccacaaagagaaattagaaagcactccatgtcgttttccttcacctctcacaaaaaagtgctgccccggacctccagcactgggtctgagaaatccaaagacttttctagagaccaaatcaagaagggtttgaggaaagacagccagcttgctggcagaatcatccaggaaaatgagtcagaaatccaaaacttcagcttgtctagaacctgggccctctcggttgacaatgtgttccagctaattgatgtgaggaagccaggaagcccaccatcctacgaagaggccattcattaccaggcatctggattcacagcctacagtggccagacggtcagcagtatgagagcaagaatgctcaaatggaacacggctctgcctcctctgccttctcgccttggaggtgaccacagtgaagggacacccggtggacacagactgtctcccctgactgagtgctggaaacagagtcagactgtcagtgtctctgtggaaactcaggggagatctgagcagcacggattgaggatggtgtctgaaacgatgcagaaggctaagttagactgtctcaggcgacaacacagccacatggtctttgaggtcgaccaactacagggtgctaaagaatcctacatttag

SEQ ID NO: 18 - DNA, 186 bp, Cricetulus griseus
atggaccccaactgctcctgctccaccggcagcacctgcacctgctccagttcctgtggctgcaaagactgcaagtgcacctcctgcaagaagagctgctgctcctgctgcccagtgggctgctccaagtgtgcccagggctgcgtctgcaaaggggcatcggacaagtgcacgtgctgtgcctaa

SEQ ID NO: 19 - DNA, 1407 bp, Mus musculus
atggtggacacagagagccccatctgtcctctctccccactggaggcagatgacctggaaagtcccttatctgaagaattcttacaagaaatgggaaacattcaagagatttctcagtccatcggtgaggagagctctggaagctttggttttgcagactaccagtacttaggaagctgtccgggctccgagggctctgtcatcacagacaccctctctccagcttccagcccttcctcagtcagctgccccgtgatccccgccagcacggacgagtcccccggcagtgccctgaacatcgagtgtcgaatatgtggggacaaggcctcagggtaccactacggagttcacgcatgtgaaggctgtaagggcttctttcggcgaactattcggctgaagctggtgtacgacaagtgtgatcggagctgcaagattcagaagaagaaccggaacaaatgccagtactgccgttttcacaagtgcctgtctgtcgggatgtcacacaatgcaattcgctttggaagaatgccaagatctgaaaaagcaaaactgaaagcagaaattcttacctgtgaacacgacctgaaagattcggaaactgcagacctcaaatctctgggcaagagaatccacgaagcctacctgaagaacttcaacatgaacaaggtcaaggcccgggtcatactcgcgggaaagaccagcaacaacccgccttttgtcatacatgacatggagaccttgtgtatggccgagaagacgcttgtggccaagatggtggccaacggcgtcgaagacaaagaggcagaggtccgattcttccactgctgccagtgcatgtccgtggagaccgtcacggagctcacagaatttgccaaggctatcccaggctttgcaaacttggacttgaacgaccaagtcaccttgctaaagtacggtgtgtatgaagccatcttcacgatgctgtcctccttgatgaacaaagacgggatgctgatcgcgtacggcaatggctttatcacacgcgagttccttaagaacctgaggaagccgttctgtgacatcatggaacccaagtttgacttcgctatgaagttcaatgccttagaactggatgacagtgacatttccctgtttgtggctgctataatttgctgtggagatcggcctggccttctaaacataggctacattgagaagttgcaggaggggattgtgcacgtgcttaagctccacctgcagagcaaccatccagatgacaccttcctcttcccaaagctccttcaaaaaatggtggaccttcggcagctggtcacggagcatgcgcagctcgtacaggtcatcaagaagaccgagtccgacgcagcgctgcacccactgttgcaagagatctacagagacatgtactga

SEQ ID NO: 20 - DNA, 1308 bp, Cricetulus griseus
atggctccctttggaagaaatttgttgaagacgcgacataagaacagatctccaactaaagacatggattctgaggagaaggaaattgtggtttgggtttgccaggaggaaaagattgtctgtggattaactaaacgcaccacctccaccgatgtcatccaggctttgctggaggaacatgaggctacatttggagagaagagatttctactgggcaaggccagtgactactgcatcgtagagaagtggaggggctcagagcgggcccttcctccactgacgaggatcctgaagctatggaaggcttggggagatgagcaagccaacatgcagtttgttttggttaaaacagatgcctttctcccagttccactgtggaggacagcggaaaccaaactagtgcaaaatagtgaaaagccttgggagctcagtccggcgaactacatgaaaactttgccaccagataaacaaaaacgaatcgtcaggaaaaccttccgaaaactggctaaaattaagcaggacatgttttctcatgatcgggacagtatggagactttggttcatctaattatttctcaggaccacactattcaccagcaagtacaaagaatgaaagagttagatatggaaattgagaaatgtgaggctaagatccacttggaccgggtagggaatgatggggacaattacgttcaggaggcatatttaatgcccagggttagtgaaaaggagcaaatgctagactttcaacctgaggacagccaggctctggtggacgtgcacgatggtgaggggatggcacagctagaagaacggctgcagtactaccgagtgctcatcgaaaagctctcggctgaaattgagagagagttgaagagcttgggcatcgacggaagtgaagattcagagggggcagctgcctgtgaactcgaaaactctgatttagaaaatgtgaagtgcgatttggagaagagtatgaaagctggtttgaaaatctactctcacttgagtggcatccagaaagagattaaatacagtgactcattgcttcagatgaaagcaagagaatacgaactcctggccaaggagttcagttcgtttcaccttagcaacaaagatggatgccaattaaaagaaaacaaaggaaaggaacacgaggctgccagcggcagtgcggagatccctccattaactcaaagggcattcaacacatacacaaatgacacagattcggacactggcattagctccaaccacagccaggactctgagacaactctgggagatgtgctgctgttgtcaacgtaa

SEQ ID NO: 21 - DNA, 825 bp, Cricetulus griseus
atggaaatctccaggtccgcatgcctagtcctttcatttatcctcatgtgtgggctggtcccagaagtcatggcagatgctgaagaaacctcaggcggtctcggcactacccaggaacccatatggctcccagacatcccagctacggaggaattcatcagtgctgctgaagtggccatcataggtttcttccaggatttagaaataccgatagtgcccatattccgcagcacggctcaacagttccgagacataccattcggaatcagcaacagttctgcggttctgagccactacaacgtcaccagaaacagcatctgcctcttccgactggttgacaatgaacaactgcacttggatgctgaagacattgaaaacttagatgcctccaagttgagccgtttcattcagatgcacagcctccgctgggtgacagagtacagccctctgattgcagctggcttatttaacaccatggttcagactcaccttctcctgataatgaacaagacttccccggagtatgaaaagagcctgcagcaataccgggaggcagctaagctcttccagggacagatcctctttgttctggtggacagcggcaagggggggaaccgaaaggtgatatcctatttccaactaaaggagtctcagctgccggctctggccatttatgaaagtgtgaatgacaagtgggacacactgcccatcacagaagtcacagtggagaaagtccaaggcttctgtgatggcttcctaaaggggacactgctgagagatcacgatgtggaaggagatgcagaggcgcggaaggaggagctctga

SEQ ID NO: 22 - DNA, 1518 bp, Cricetulus griseus
atgcgcttcagctgcctagcgctgctcccgggcgtggcgctgctgctcgcctcggcccgcctcgccgccgcctctgacgtgctggaactcacggacgaaaacttcgagagtcgcgtctccgacacgggctctgcgggcctcatgctagtcgagttcttcgccccttggtgtggacattgcaagaggcttgcccctgagtatgaagctgcagcaactagattgaaaggaatagtcccattagcaaaggttgattgcactgccaacacaaacacctgtaataagtatggagtcagtggctacccaacccttaaaatatttagagatggtgaagaagcaggtgcttatgatgggcctaggactgccgatggaattgtcagccacttgaagaaacaagcaggaccagcttcggttcctctcaggtctgaggaagaatttaagaagttcattagtgataaagatgcttcggtggtgggctttttcagggatttattcagtgacggtcactctgaattcctaaaagcagccagcaacttgagagataactacagatttgcacacaccaacgttgagtctctggtgaaggagtatgatgataatggagaggggatcactttatttcgtccttcacatcttgctaacaagtttgaagacaaaactgtggtatatactgaacagaaaatgaccagtggcaagataaaaaggtttatccaggaaagcatttttggtatctgtcctcatatgacagaagacaataaagatttgatacagagcaaggacttacttacagcctactatgatgttgactatgaaaagaatgctaaaggttccaactattggagaaacagagtgatgatggtggcaaaaaaattcctggatgctggacacaaactcaactttgctgtagctagccgtaaaacctttagccatgagctgtccgactttggcttagaaagcactactggagaggttcctgttgtagcaatcagaactgctaaaggagagaagtttgtcatgcaggaagagttctcgagggacgggaaggctctggagcggttcctgcaggattactttgatggcaacctgaagagatacctcaagtctgaacctatcccagagaccaacgatgggcctgtcaaggttgtggtagcagaaaattttgatgacatagtgaataatgaagataaggatgtgctgattgaattttatgccccttggtgtggtcactgtaagaatctggagcccaaatataaagaactgggagagaaactcagcaaagacccaaatattgttatagccaagatggatgccacagctaatgatgtgccttctccatatgaagtcaaaggttttcctaccatctacttttcaccagccaacaagaagctaaatccaaagaagtatgaaggtggccgtgaattaaatgattttattaactatctacaacgagaagctacaaccccccctataattcaagaagaaaaacccaagaagaagaagaaggcacaagaggacctctaa

SEQ ID NO: 23 - DNA, 2874 bp, Cricetulus griseus
atgactctcctgctgctgtccctcttgctggcctccctgctccagctcagctctggcaacaaagccaacaagcacaagccctggatcgaggcagagtaccagggcattgtcatggagaatgacaacactgtcctgttgaatccaccactctttgccttggataaagatgccccgctgcgctatgcaggtgagatctgtggcttccggctgcatgggtctggggtaccctttgaggctgtgatcctggacaaggcaacaggtgaagggctgatccgggccaaggagccggtggactgtgaggcccagaaggaacacacctttaccatccaggcttatgactgtggcgaggggcccgatggcaccaacaccaagaagtctcacaaggcaaccgtgcatgtccgcgtcaacgatgtgaatgagtttgccccggtctttgtggagcgtctgtaccgtgctgcagtgacagaggggaagctgtatgaccgcatcttacgcgtggaagccatcgatggcgactgctcccctcagtacagccagatctgctactatgagattcttacacccaacattcctttcctcatcgacaatgacggcaacattgagaacacagagaagctgcagtacagtggtgagaaactctacaagttcacggtgacagcgtacgactgtgggaagaagcgggctgcggatgacgcggaggtggagatacaggtgaagcccacctgcaagcccagctggcaaggctggaacaaaaggattgagtacgccccaggcgccgggagtttggctttgttccctggcatccgcctggagacctgtgatgaaccattgtggaacatccaggccaccatagagctgcagaccagccatgtggccaagggctgtgaccgagacaactactccgaaagggcactgcggaagctctgtggcgctgctactggggaggtagacctgttgcccatgcctggccccaatgccaactggacagcgggactctcagtgcactacagccaggacagcagcctcatctactggttcaatggcacccaagctgtgcaagttcccctgggtggcccagcagggctgggctctgggccgcaggatggccttagtgaccactttaccctgtccttctggatgaaacatggcgtgactcccaacaaggggaagaaggaggaagagaccatcgtgtgtaacacagtccagaatgaggacggcttctctcactactcactgactgtacatggctgcagaatctccttcctctactggccactgcttgagagtgcccgtccggtcaagttcctctggaagctggagcaggtatgtgacgacgagtggcaccactatgccctgaacctcgagttccccacggtcacactctacactgatggcatctcctttgaccctgctctcatccatgacaatggtctcatccacccacccaggagggagcctgcactcatgattggggcctgctggactgaagagaagaacagagaaaaggaaaaaggaggagacaacagtacagacacaaccccaggagaccccttgctgatccaccactacttccacggctacctggctggtttcagcgtgcgctcaggtcgcctggagagccgtgaggtcatcgagtgcctctatgcatgccgggaggggctggactatagggatttcgagagcctgggcaaaggcatgaaggtccacgtgaacccctcgcagtccctgctcaccctggagggggatgatgtggagaccttcaaccatgccctgcagcatgtggcttacatgaacactctgcgctttgccacacccggcgtcaggccgctgcgcctcaccaccgctgtcaagtgcttcagtgaagagtcctgcgtctccatccctgaagtggaaggctatgtggtggtccttcagcctgatgccccccagatccttctgagtggcacagctcattttgcccgcccagcggtggactttgagggacccgacggggtccctttgttccctgaccttcagatcacctgctccatttctcaccaggtggaggccaaagcagatgagagctggcagggcacagtgacagacacacgcatgtcagatgagattgtacacaacctggacggctgtgaaatttctctggtgggagatgacctagaccctgagcgagaaagcctgctcttggacatggcatccttgcagcagcgaggcctggaactcaccaacacatctgcctacctcaccattgctggggtggaaaccatcactgtgtatgaagagatcctgaggcacgctcgttatcagctccggcatggagctgccctgtatgctaggaaattcaggctttcctgttcggagatgaatggccgctactccagcaacgaattcattgtggaggtcaacgtccttcatagcatgaaccgggtggcccaccccagccatgtgctcagctcacagcagttcttgcaccgaggtcaccagcctcctcctgagatggctggacacagcctggccagctcccaccggaactccatggtccccagtgctgcgactctcatcattgtggtgtgtgtgggcttcctggtgcttatggtcatcctgggcctagtgcgaatccactcccttcaccgccgcgtctcaggaactggcggaccctcaggggcttccactgacccgaaagaccctgacctcttctgggatgactctgctctcaccattatcgtgaaccccatggagtcctaccagaaccagcaggccagtgtggcaggggttgctggtggccagcaagaggaagaggacagcagtgactcagaggcagccgacttccccagcagcgacgagagacgcatcgttgaaagccccccacaccgctactga

SEQ ID NO: 24 - DNA, 1308 bp, Cricetulus griseus
atgggtcaggagctgtgtgcaaagagacagccaggatgcagttgctaccaccgttcggagagaggggaggcgcacagctgtcagagaagccagcctgggagcaccgaggctgctgcgttcgagctaacagacataaaaggagcaacgtgttccttgtcttcatttcaccccaggggactccagggccctggtgaccagaagctggagagcaaaaggccccggagtaacagtgactcttttcaggaagagaatctgaggcaagggttgccatggaagaagagcctcccttttggggcagcctcatcttatttgaacttggagaagctgggtgaaggctcttatgccacagtttacaaggggattagcaggataaatggacaactagtagctctaaaggtgatcagcatgaacgccgaggagggtgtgcctttcacagccatccgggaagcctccctcctgaagggtctgaagcatgccaacattgtcctcttgcatgacattgtgcacaccaaagagactctgacattcgtatttgaatacatgcacacagatctggcccagtacatgtctcagcatcccggaggccttcatcctcacaatgtcaggcttttcatgtttcaacttctgcgaggcctggcatacatccaccatcagcatgttcttcacagggacctgaaacctcagaacttgctcatcagtcacctgggagagctcaaattggctgactttggtcttgctcgagccaagtccattcccagccagacgtactcttcagaagtggtgacactgtggtaccggccacctgacgccttgctgggagccactgagtactcctctgagctagacatatggggcgcaggctgcatctttattgaaatgttccagggtcaacctttgtttcctggggtttccaacatcctggaacagctggagaagatctgggaggtactgggagtccctacagaggacacctggcctggagtctccaagctgcctaactacaatccagaatggttcccgctgcctaagcctcaaagcctgcagattgtctggagcaggttgggtggagttcctgaagctgaagacctggcctcccagatgctaaaaggtttccctagggaccgagtatcagcacaggaagcactcgttcatgattatttcagtgtccttccatcccagttgtaccagcttcctgatgaagagtctttgtttgcagtttcaggagtgaagctaaagccagaaatgtgtgacctttcagcctcctacaggaagggtcactacctagccgggtttaacaaatgttggtga

SEQ ID NO: 25 - DNA, 783 bp, Cricetulus griseus
atggctaaggagtggggttatgccagccacaatggtcctgaacactggcatgaactttatccaattgccaaaggggacaatcagtcacccattgagctgcatactaaagacatcaagcatgacccctctttgcaaccctggtcagcatcttacgatcctggctctgctaagaccatcctgaataatgggaagacctgcagagttgtgtttgatgacacgtatgacaggtctatgctgaggggtggtcctctctctggaccctatcgacttcgccagtttcatcttcactggggctcctcggatgaccatggctctgagcacacagtggatggagtgaagtatgctgctgagcttcacctggttcactggaacccaaagtacaacaccttcggagaagctctaaagcaacccgatggaatcgctgttgttggcatttttctgaagataggacgggagaaaggagagttccagattttccttgatgccctggacaaaattaagactaagggcaaggaggctcctttcacccacttcgacccatcctgcctgtttcctgcttgccgcgactactggacctatcatggctccttcaccacgccaccctgcgaggagtgcatcgtgtggctgctgttgaaggagcccatgaccgtgagctcagaccagatggccaagctgcgtagtctattcgccagcacggagaatgagcccccggtgcctctggtggggaattggcgccccccgcagccggtcaagggtagggtggtgagggcctctttcaagtga

SEQ ID NO: 26 - DNA, 1305 bp, Cricetulus griseus
atgaactccggcctgggttccatgaactccatgaacagctacatgaccatgaacaccatgaccacgagcggcaatatgacaccggcttccttcaacatgtcctacgccaacccgggcctaggagccggcctgagtccgggtgctgtggccggcatgcccggaggctctgccggcgccatgaacagcatgaccgcggcaggcgtcacggccatgggtgcagcgctgagcccgggaggcatgggctccatgggcgcgcagcccgcagcctctatgaacggcctgggtccctatgcggcagccatgaacccgtgcatgagccccatggcgtacgcgccgtccaacttgggccgcagccgcgctgggggcggcggcgactccaagacattcaagcgcagctacccgcacgccaagccgccctactcctacatctcgctcatcaccatggccatccagcaggcgcccagcaagatgctcacactgagcgagatctaccagtggatcatggacctcttcccctattaccgtcaaaaccagcagcgctggcagaactccatccgccactcgctgtctttcaacgactgtttcgtcaaggtggcgcgatccccggacaaaccgggcaagggctcctactggacgctgcacccggactccggcaacatgttcgagaacggctgctacttgcgccgtcaaaagcgcttcaagtgtgagaagcagccgggggccggaggcgggagcgggggcgccggctccaagggtggccccgaaagccgcaaggacccctcaggcgcaggtaaccacgccgagtcgccccttcatcggggtgtgcacggaaaggctggccagctagagggcgcgccggcccccgggcccgccgccagcccccagactctggaccacagcggggccacagcgacagggggcgcttcggagttgaagtctccagcctcttcctctgcgccccccataagctccgggcctggggcgctggcatctgtacccccctctcacccggcgcacggcctggcaccccacgaatcgcagctgcacctgaaaggggacccccactactcctttaatcacccgttctctatcaacaacctcatgtcctcttcggagcagcagcacaagctggacttcaaggcatatgagcaggcactccagtactctccttatggtgccaccttgcccgccagtctgccccttggcagcgcctcagtggccacaaggagccccatcgagccctcagcactggagccggcctactaccaaggtgtgtattccagacccgtgctaaatacttcctag

SEQ ID NO: 27 - DNA, 879 bp, Cricetulus griseus
atggaagactcgatggatatggacatgagccctcttaggccacagaactaccttttcggttgtgaactaaaagctgacaaagattatcactttaaggtggataatgatgagaatgaacaccagttgtcactaagaacggttagtttaggggcaggggcaaaagatgaattacacatcgtagaggcagaagcaatgaattatgaaggcagtccaattaaagtaacgctggcaacattgaaaatgtccgtacaaccaacagtttcccttggtggctttgaaattacaccacctgtggtgttacggttgaggtgtggttcagggcctgtgcacattagtggacagcatctagtagctgtagaggaagatgcagagtcagaggatgaagatgaggaggatgtgaaactcttaagtatgtctggcaagcgatctgctcctggaggtggtagcaaagttccacagaaaaaagtaaaaatcgatgaagatgaggatgatgatgatgaagatgatgaagacgatgaagatgatgatgatgatgattttgatgatgaagaaactgaagaaaaagttccagtgaagaaatctgtacgagataccccagccaaaaatgcacaaaaatcaaaccaaaatggaaaagacttaaaaccatcaacaccaagatcaaaaggtcaagagtccttcaaaaaacaggaaaaaactcctaaaaccccaaaaggacctagttctgtagaagacattaaggcaaaaatgcaagcaagtatagaaaaaggtggttctcttcccaaagtggaagccaagttcattaattatgtgaagaattgcttccggatgactgaccaggaggctattcaagatctctggcagtggaggaaatctctttaa

SEQ ID NO: 28 - DNA, 2106 bp, Cricetulus griseus
atggcggaccggcgtcggcagcgcgcttcccaggacactgaggacgaagaatctggtgcttcgggttctgacagcggctctccggcgcggggcggcggcagctgcagcgggagcgccggaggcggcggcagtggctcgctgccgtctcagcgcggcggccgcggcgggggccttcatctgcggcgggtggagagcgggggcgccaagagcgccgaggagtcggagtgtgagagtgaagatggcatggaaggcgatgctgttctttccgattatgaaagtgcagaagattcagaaggtgaagaaggagaatacagtgaggaggaaaactccaaggtggagctgaaatcagaaggtaatgatgctgctgattcctcagcaaaagagaagggagaagaaaagcctgatactaaaggcactgtgactggagagaggcaaagtggggatggacaggagagcacagagcctgtagagaataaagtgggtaaaaaaggccctaagcatttggatgatgatgaagatcggaaaaatccagcgtatataccaaggaaagggctcttctttgagcatgatcttcgagggcaaactcaggaagaggaagtccgacccaagggacgtcagcgaaagctatggaaggatgagggtcgctgggagcatgataagttccgtgaagatgaacaggctccaaagtcccgacaggaactcattgccctttatggctatgatattcgatctgctcacaatcctgatgacatcaaaccccggagaatccggaagcctcgatttggaagtcccccacaaagagatccaaattggattggtgatagatcaaacaagtcccatcgccaccagggtcctgggggtaccctaccaccaaggacatttattaataggaatgcatcaggtactggccgcatgtctgcatccaggaattattcacgatctgggggcttcaaggaaggtcgtactagtttcaggcctgtggaggctggtgggcagcatggtggccggtctggtgagactcataaacatgaagctaattaccggtcacgacgtctggagcaaacccctatgagggatccatctccagaaccagatgctccgttgcttggcagtcctgagaaagaagaggcggcctcggagacaccagctgccgctcctgacactgtaccaccagctcctgacaggcccattgagaagaaatcctattcccgggcaagaaggacccggaccaaagtgggggatgcaatgaaagctgctgaggaggctcctcctccagctgaagggctcacttcagcagccacaatcccagaaacagctcctcctccagctgctaagactgggaactgggaggctccagtagattctaccacaggtggactggagcaagatgtggcacagctaaatatagcagaacaaaactggagtccaggacagccttcgttcctgcaaccacgggagcttcgaggtatgcccaaccacatacatatgggagcaggacctccacctcagtttaaccggatggaagaaatgggtgtccagggtagtcgagctaaacgttattcatcccagcggcagagacctgtgccagagccccctgctcctcctgtccatatcagtatcatggagggacattactatgatccattgcagttccagggaccaatctatacccatggcgacagccctgccccactgcctccacagggcatgattgtacagccagaaatgcaccttcctcatccaggtttacatccccatcagtcaccagcacctctgcccaatccgggtctctacccaccaccagtgtccatgtctccaggacaaccaccaccccagcagttgctcgctcctacttacttttctgctccaggtgtcatgaattttggtaaccccagttacccttatgctccaggagcattacctcctcctccacctcctcatctgtatcctaacacacaggctccatcacaggtgtatgggggagtgacctactataaccctgcccagcagcaggtgcagccaaaaccctccccaccccggaggactccccagccagtgtccatcaagccccctccacctgaggttgtaagcaggggttccagttaa

SEQ ID NO: 29 - DNA, 1101 bp, Cricetulus griseus
atggctaagccccaggtggttgtggctcctgtgctaatgtctaagctgtctgcgaacgcccccgaattttacccatcaggttattctaattataccgaatcctatgaggatgtttgtgaggattatcccactctaccagaatatgttcaggatttcctgaatcatctcacagagcagcctggcagttttgaaacagaaattgaacagtttgcagaaaccctgaatggatgggtcacaacagacgatgctttgcaagagcttgtggaactcatctaccagcaggccacgtctatcccaaatttctcttacatgggagctcgcctgtgtaattacctgtcccatcatctgacaattagcccacagagtggcaacttccgccaattgctacttcaaaggtgccggacggaatatgaagctaaagacgaagctgcaaaaggagatgaagtgactcgaaaacgattccatgcctttgttctgtttctgggagaactgtatcttaacctggagatcaaaggaacaaatggacaggttacaagagcagatattcttcaggttggcctacgggaactgctgaatgctctgttctccaatcctatggatgacaacttaatttgtgcagtaaagttgctaaagttgacagggtcagttttagaagatacttggaaggaaaaggggaaaactgatatggaagaagtaattcagagaattgaaaatgttgtcctagatgcaaactgcagcagagatgtaaaacagatgctcttgaagcttgtagaacttcgatcaagtaactggggcagagtccatgcaacttcaacatacagagaagctacccctgaaaatgaccctaactactttatgaatgaaccaacattttatacatctgatggtgttcctttcactgcagctgacccagattaccaagagaagtatcaggagttacttgaaagagaggacttttttccagattatgaagaaaatggaacagatttatccggggctggtgacccatacttggatgatattgatgatgagatggacccagagattgaagaagcttatgaaaagttttgtttggaatcagagcgtaagcgaaaacagtaa

SEQ ID NO: 30 - DNA, 1440 bp, Cricetulus griseus
atgtcggacagtttcgatcgggctccaggtgctggtcggggccggagccggggcctgggccgcggagggggcgggcctgagggcggcgggttcccgaacggagcggggcctgcggagcggtcgcggcaccagccgccgccgcagcccaaagccccgggcttcctgcagccgccgccgctgcgccagcccaggaccgccccgccgccaggggcccagagcgaggcccccgccggctccgcgcggcctccccgacctggggcgctcccagaacaaacgcgtccccagagagctccacctagttcacaggataaaatcccacagcagaactcggagtcagcaatggctaagccccaggtggttgtggctcctgtgctaatgtctaagctgtctgcgaacgcccccgaattttacccatcaggttattctaattataccgaatcctatgaggatgtttgtgaggattatcccactctaccagaatatgttcaggatttcctgaatcatctcacagagcagcctggcagttttgaaacagaaattgaacagtttgcagaaaccctgaatggatgggtcacaacagacgatgctttgcaagagcttgtggaactcatctaccagcaggccacgtctatcccaaatttctcttacatgggagctcgcctgtgtaattacctgtcccatcatctgacaattagcccacagagtggcaacttccgccaattgctacttcaaaggtgccggacggaatatgaagctaaagacgaagctgcaaaaggagatgaagtgactcgaaaacgattccatgcctttgttctgtttctgggagaactgtatcttaacctggagatcaaaggaacaaatggacaggttacaagagcagatattcttcaggttggcctacgggaactgctgaatgctctgttctccaatcctatggatgacaacttaatttgtgcagtaaagttgctaaagttgacagggtcagttttagaagatacttggaaggaaaaggggaaaactgatatggaagaagtaattcagagaattgaaaatgttgtcctagatgcaaactgcagcagagatgtaaaacagatgctcttgaagcttgtagaacttcgatcaagtaactggggcagagtccatgcaacttcaacatacagagaagctacccctgaaaatgaccctaactactttatgaatgaaccaacattttatacatctgatggtgttcctttcactgcagctgacccagattaccaagagaagtatcaggagttacttgaaagagaggacttttttccagattatgaagaaaatggaacagatttatccggggctggtgacccatacttggatgatattgatgatgagatggacccagagattgaagaagcttatgaaaagttttgtttggaatcagagcgtaagcgaaaacagtaa

SEQ ID NO: 31 - DNA, 1440 bp, Homo sapiens
atgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacaca

SEQ ID NO: 32 - DNA, 822 bp, Homo sapiens
atgggcaccgacagccgcgcggccaaggcgctcctggcgcgggcccgcaccctgcacctgcagacggggaacctgctgaactggggccgcctgcggaagaagtgcccgtccacgcacagcgaggagcttcatgattgtatccaaaaaaccttgaatgaatggagttcccaaatcaacccagatttggtcagggagtttccagatgtcttggaatgcactgtatctcatgcagtagaaaagataaatcctgatgaaagagaagaaatgaaagtttctgcaaaactgttcattgtagaatcaaactcttcatcatcaactagaagtgcagttgacatggcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtaaaaccaaatagtaaccaagttaatcttgcctcctgctgtgtgatgccaccagatttgactgcatttgctaaacaatttgacatacagctgttgactcacaatgatccaaaagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttct

SEQ ID NO: 33 - DNA, 1131 bp, Homo sapiens
atgtgtgacgacgaggagaccaccgccctggtgtgcgacaacggctctgggctggtgaaggccggctttgcgggcgatgacgcgccccgcgctgtcttcccgtccatcgtgggccgcccgcggcaccagggagttatggtgggtatgggtcagaaggactcctacgtaggtgatgaagcccagagcaagagaggcatcctgaccctgaagtatcccatcgagcatggtatcatcaccaactgggacgacatggagaagatctggcaccacaccttctacaatgagctccgtgtggctcccgaggagcaccccaccctgctcacagaggccccgctgaaccccaaggccaaccgggagaagatgactcagatcatgtttgagaccttcaatgtccctgccatgtacgtggccatccaggcagtgctatccctgtatgcttctggccgtaccacaggcattgttctggactctggggatggtgtaactcacaatgtccccatctatgagggctacgctttgccccatgccatcatgcgtctggatctggctggtcgggacctcactgactacctcatgaagatcctcactgagcgtggctactcctttgtcaccactgctgaacgtgaaattgtccgtgacattaaagagaagctgtgctatgtcgccctggattttgagaatgagatggccacagctgcctcttcctcctccctggagaagagctatgaactgcctgatggccaagtcatcactattggcaatgagcgcttccgctgtcctgagacactcttccagccctccttcattggtatggaatctgctggcatccatgaaacaacttacaatagcatcatgaagtgtgacattgatatccgcaaggacctgtatgccaacaatgtcttatctggaggcaccactatgtaccctggtattgctgatcgtatgcagaaggaaatcactgctctggctcctagcaccatgaagattaagattattgctccccctgagcgtaaatactctgtctggattgggggctccatcctggcctctctgtccaccttccagcaaatgtggattagcaagcaagagtacgatgaggcaggcccatccattgtccaccgcaaatgcttc

SEQ ID NO: 34 - DNA, 564 bp, Homo sapiens
atggccaactcgggctgcaaggacgtcacgggtccagatgaggagagttttctgtactttgcctacggcagcaacctgctgacagagaggatccacctccgaaacccctcggcggcgttcttctgtgtggcccgcctgcaggattttaagcttgactttggcaattcccaaggcaaaacaagtcaaacttggcatggagggatagccaccatttttcagagtcctggcgatgaagtgtggggagtagtatggaaaatgaacaaaagcaatttaaattctctggatgagcaagaaggggttaaaagtggaatgtatgttgtaatagaagttaaagttgcaactcaagaaggaaaagaaataacctgtcgaagttatctgatgacaaattacgaaagtgctcccccatccccacagtataaaaagattatttgcatgggtgcaaaagaaaatggtttgccgctggagtatcaagagaagttaaaagcaatagaaccaaatgactatacaggaaaggtctcagaagaaattgaagacatcatcaaaaagggggaaacacaaactctt

SEQ ID NO: 35 - DNA, 462 bp, Homo sapiens
atggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaa

SEQ ID NO: 36 - DNA, 822 bp, Cricetulus griseus
atggaaatctccaggtccgcatgcctagtcctttcatttatcctcatgtgtgggctggtcccagaagtcatggcagatgctgaagaaacctcaggcggtctcggcactacccaggaacccatatggctcccagacatcccagctacggaggaattcatcagtgctgctgaagtggccatcataggtttcttccaggatttagaaataccgatagtgcccatattccgcagcacggctcaacagttccgagacataccattcggaatcagcaacagttctgcggttctgagccactacaacgtcaccagaaacagcatctgcctcttccgactggttgacaatgaacaactgcacttggatgctgaagacattgaaaacttagatgcctccaagttgagccgtttcattcagatgcacagcctccgctgggtgacagagtacagccctctgattgcagctggcttatttaacaccatggttcagactcaccttctcctgataatgaacaagacttccccggagtatgaaaagagcctgcagcaataccgggaggcagctaagctcttccagggacagatcctctttgttctggtggacagcggcaagggggggaaccgaaaggtgatatcctatttccaactaaaggagtctcagctgccggctctggccatttatgaaagtgtgaatgacaagtgggacacactgcccatcacagaagtcacagtggagaaagtccaaggcttctgtgatggcttcctaaaggggacactgctgagagatcacgatgtggaaggagatgcagaggcgcggaaggaggagctc

SEQ ID NO: 37 - DNA, 1515 bp, Cricetulus griseus
atgcgcttcagctgcctagcgctgctcccgggcgtggcgctgctgctcgcctcggcccgcctcgccgccgcctctgacgtgctggaactcacggacgaaaacttcgagagtcgcgtctccgacacgggctctgcgggcctcatgctagtcgagttcttcgccccttggtgtggacattgcaagaggcttgcccctgagtatgaagctgcagcaactagattgaaaggaatagtcccattagcaaaggttgattgcactgccaacacaaacacctgtaataagtatggagtcagtggctacccaacccttaaaatatttagagatggtgaagaagcaggtgcttatgatgggcctaggactgccgatggaattgtcagccacttgaagaaacaagcaggaccagcttcggttcctctcaggtctgaggaagaatttaagaagttcattagtgataaagatgcttcggtggtgggctttttcagggatttattcagtgacggtcactctgaattcctaaaagcagccagcaacttgagagataactacagatttgcacacaccaacgttgagtctctggtgaaggagtatgatgataatggagaggggatcactttatttcgtccttcacatcttgctaacaagtttgaagacaaaactgtggtatatactgaacagaaaatgaccagtggcaagataaaaaggtttatccaggaaagcatttttggtatctgtcctcatatgacagaagacaataaagatttgatacagagcaaggacttacttacagcctactatgatgttgactatgaaaagaatgctaaaggttccaactattggagaaacagagtgatgatggtggcaaaaaaattcctggatgctggacacaaactcaactttgctgtagctagccgtaaaacctttagccatgagctgtccgactttggcttagaaagcactactggagaggttcctgttgtagcaatcagaactgctaaaggagagaagtttgtcatgcaggaagagttctcgagggacgggaaggctctggagcggttcctgcaggattactttgatggcaacctgaagagatacctcaagtctgaacctatcccagagaccaacgatgggcctgtcaaggttgtggtagcagaaaattttgatgacatagtgaataatgaagataaggatgtgctgattgaattttatgccccttggtgtggtcactgtaagaatctggagcccaaatataaagaactgggagagaaactcagcaaagacccaaatattgttatagccaagatggatgccacagctaatgatgtgccttctccatatgaagtcaaaggttttcctaccatctacttttcaccagccaacaagaagctaaatccaaagaagtatgaaggtggccgtgaattaaatgattttattaactatctacaacgagaagctacaaccccccctataattcaagaagaaaaacccaagaagaagaagaaggcacaagaggacctc

SEQ ID NO: 38 - DNA, 615 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaag

SEQ ID NO: 39 - DNA, 708 bp, Escherichia coli
atgaagaaaggttttatgttgtttactttgttagcggcgttttcaggctttgctcaggctgatgacgcggcaattcaacaaacgttagccaaaatgggcatcaaaagcagcgatattcagcccgcgcctgtagctggcatgaagacagttctgactaacagcggcgtgttgtacatcaccgatgatggtaaacatatcattcaggggccaatgtatgacgttagtggcacggctccggtcaatgtcaccaataagatgctgttaaagcagttgaatgcgcttgaaaaagagatgatcgtttataaagcgccgcaggaaaaacacgtcatcaccgtgtttactgatattacctgtggttactgccacaaactgcatgagcaaatggcagactacaacgcgctggggatcaccgtgcgttatcttgctttcccgcgccaggggctggacagcgatgcagagaaagaaatgaaagctatctggtgtgcgaaagataaaaacaaagcgtttgatgatgtgatggcaggtaaaagcgtcgcaccagccagttgcgacgtggatattgccgaccattacgcacttggcgtccagcttggcgttagcggtactccggcagttgtgctgagcaatggcacacttgttccgggttaccagccgccgaaagagatgaaagaattcctcgacgaacaccaaaaaatgaccagcggtaaa

SEQ ID NO: 40 - DNA, 810 bp, Escherichia coli
atgaaatcactgtttaaagtaacgctgctggcgaccacaatggccgttgccctgcatgcaccaatcacttttgctgctgaagctgcaaaacctgctacagctgctgacagcaaagcagcgttcaaaaatgacgatcagaaatcagcttatgcactgggtgcctcgctgggtcgttacatggaaaactctctaaaagaacaagaaaaactgggcatcaaactggataaagatcagctgatcgctggtgttcaggatgcatttgctgataagagcaaactctccgaccaagagatcgaacagactctacaagcattcgaagctcgcgtgaagtcttctgctcaggcgaagatggaaaaagacgcggctgataacgaagcaaaaggtaaagagtaccgcgagaaatttgccaaagagaaaggtgtgaaaacctcttcaactggtctggtttatcaggtagtagaagccggtaaaggcgaagcaccgaaagacagcgatactgttgtagtgaactacaaaggtacgctgatcgacggtaaagagttcgacaactcttacacccgtggtgaaccgctttctttccgtctggacggtgttatcccgggttggacagaaggtctgaagaacatcaagaaaggcggtaagatcaaactggttattccaccagaactggcttacggcaaagcgggtgttccggggatcccaccgaattctaccctggtgtttgacgtagagctgctggatgtgaaaccagcgccgaaggctgatgcaaagccggaagctgatgcgaaagccgcagattctgctaaaaaa

SEQ ID NO: 41 - DNA, 483 bp, Escherichia coli
atgaaaaagtggttattagctgcaggtctcggtttagcactggcaacttctgctcaggcggctgacaaaattgcaatcgtcaacatgggcagcctgttccagcaggtagcgcagaaaaccggtgtttctaacacgctggaaaatgagttcaaaggccgtgccagcgaactgcagcgtatggaaaccgatctgcaggctaaaatgaaaaagctgcagtccatgaaagcgggcagcgatcgcactaagctggaaaaagacgtgatggctcagcgccagacttttgctcagaaagcgcaggcttttgagcaggatcgcgcacgtcgttccaacgaagaacgcggcaaactggttactcgtatccagactgctgtgaaatccgttgccaacagccaggatatcgatctggttgttgatgcaaacgccgttgcttacaacagcagcgatgtaaaagacatcactgccgacgtactgaaacaggttaaa

SEQ ID NO: 42 - DNA, 1350 bp, Homo sapiens
atggaagggtctaagacgtccaacaacagcaccatgcaggtgagcttcgtgtgccagcgctgcagccagcccctgaaactggacacgagtttcaagatcctggaccgtgtcaccatccaggaactcacagctccattacttaccacagcccaggcgaaaccaggagagacccaggaggaagagactaactcaggagaggagccatttattgaaactcctcgccaggatggtgtctctcgcagattcatccccccagccaggatgatgtccacagaaagtgccaacagcttcactctgattggggaggcatctgatggcggcaccatggagaacctcagccgaagactgaaggtcactggggacctttttgacatcatgtcgggccagacagatgtggatcacccactctgtgaggaatgcacagatactcttttagaccagctggacactcagctcaacgtcactgaaaatgagtgtcagaactacaaacgctgtttggagatcttagagcaaatgaatgaggatgacagtgaacagttacagatggagctaaaggagctggcactagaggaggagaggctgatccaggagctggaagacgtggaaaagaaccgcaagatagtggcagaaaatctcgagaaggtccaggctgaggctgagagactggatcaggaggaagctcagtatcagagagaatacagtgaatttaaacgacagcagctggagctggatgatgagctgaagagtgttgaaaaccagatgcgttatgcccagacgcagctggataagctgaagaaaaccaacgtctttaatgcaaccttccacatctggcacagtggacagtttggcacaatcaataacttcaggctgggtcgcctgcccagtgttcccgtggaatggaatgagattaatgctgcttggggccagactgtgttgctgctccatgctctggccaataagatgggtctgaaatttcagagataccgacttgttccttacggaaaccattcatatctggagtctctgacagacaaatctaaggagctgccgttatactgttctggggggttgcggtttttctgggacaacaagtttgaccatgcaatggtggctttcctggactgtgtgcagcagttcaaagaagaggttgagaaaggcgagacacgtttttgtcttccctacaggatggatgtggagaaaggcaagattgaagacacaggaggcagtggcggctcctattccatcaaaacccagtttaactctgaggagcagtggacaaaagctctcaagttcatgctgacgaatcttaagtggggtcttgcttgggtgtcctcacaattttataacaaa

SEQ ID NO: 43 - DNA, 1815 bp, Homo sapiens
atggcggggctgtactcgctgggagtgagcgtcttctccgaccagggcgggaggaagtacatggaggacgttactcaaatcgttgtggagcccgaaccgacggctgaagaaaagccctcgccgcggcggtcgctgtctcagccgttgcctccgcggccgtcgccggccgcccttcccggcggcgaagtctcggggaaaggcccagcggtggcagcccgagaggctcgcgaccctctcccggacgccggggcctcgccggcacctagccgctgctgccgccgccgttcctccgtggcctttttcgccgtgtgcgacgggcacggcgggcgggaggcggcacagtttgcccgggagcacttgtggggtttcatcaagaagcagaagggtttcacctcgtccgagccggctaaggtttgcgctgccatccgcaaaggctttctcgcttgtcaccttgccatgtggaagaaactggcggaatggccaaagactatgacgggtcttcctagcacatcagggacaactgccagtgtggtcatcattcggggcatgaagatgtatgtagctcacgtaggtgactcaggggtggttcttggaattcaggatgacccgaaggatgactttgtcagagctgtggaggtgacacaggaccataagccagaacttcccaaggaaagagaacgaatcgaaggacttggtgggagtgtaatgaacaagtctggggtgaatcgtgtagtttggaaacgacctcgactcactcacaatggacctgttagaaggagcacagttattgaccagattccttttctggcagtagcaagagcacttggtgatttgtggagctatgatttcttcagtggtgaatttgtggtgtcacctgaaccagacacaagtgtccacactcttgaccctcagaagcacaagtatattatattggggagtgatggactttggaatatgattccaccacaagatgccatctcaatgtgccaggaccaagaggagaaaaaatacctgatgggtgagcatggacaatcttgtgccaaaatgcttgtgaatcgagcattgggccgctggaggcagcgtatgctccgagcagataacactagtgccatagtaatctgcatctctccagaagtggacaatcagggaaactttaccaatgaagatgagttatacctgaacctgactgacagcccttcctataatagtcaagaaacctgtgtgatgactccttccccatgttctacaccaccagtcaagtcactggaggaggatccatggccaagggtgaattctaaggaccatatacctgccctggttcgtagcaatgccttctcagagaattttttagaggtttcagctgagatagctcgagagaatgtccaaggtgtagtcataccctcaaaagatccagaaccacttgaagaaaattgcgctaaagccctgactttaaggatacatgattctttgaataatagccttccaattggccttgtgcctactaattcaacaaacactgtcatggaccaaaaaaatttgaagatgtcaactcctggccaaatgaaagcccaagaaattgaaagaacccctccaacaaactttaaaaggacattagaagagtccaattctggccccctgatgaagaagcatagacgaaatggcttaagtcgaagtagtggtgctcagcctgcaagtctccccacaacctcacagcgaaagaactctgttaaactcaccatgcgacgcagacttaggggccagaagaaaattggaaatcctttacttcatcaacacaggaaaactgtttgtgtttgc

SEQ ID NO: 44 - DNA, 468 bp, Homo sapiens
atgggcgcctccaggctctataccctggtgctggtcctgcagcctcagcgagttctcctgggcatgaaaaagcgaggcttcggggccggccggtggaatggctttgggggcaaagtgcaagaaggagagaccatcgaggatggggctaggagggagctgcaggaggagagcggtctgacagtggacgccctgcacaaggtgggccagatcgtgtttgagttcgtgggcgagcctgagctcatggacgtgcatgtcttctgcacagacagcatccaggggacccccgtggagagcgacgaaatgcgcccatgctggttccagctggatcagatccccttcaaggacatgtggcccgacgacagctactggtttccactcctgcttcagaagaagaaattccacgggtacttcaagttccagggtcaggacaccatcctggactacacactccgcgaggtggacacggtc

SEQ ID NO: 45 - DNA, 492 bp, Homo sapiens
atgacggccagcgcacagccgcgcgggcggcggccaggagtcggagtcggagtcgtggtgaccagctgcaagcatccgcgttgcgtcctcctggggaagaggaaaggctcggttggagctggcagtttccaactccctggaggtcatctggagttcggtgaaacctgggaagaatgtgctcaaagggaaacctgggaagaagcagctcttcacctgaaaaatgttcactttgcctcagttgtgaattctttcattgagaaggagaattaccattatgttactatattaatgaaaggagaagtggatgtgactcatgattcagaaccaaagaatgtagagcctgaaaaaaatgaaagttgggagtgggttccttgggaagaactacctcccctggaccagcttttctggggactgcgttgtttaaaagaacaaggctatgatccatttaaagaagatctgaaccatctggtgggatacaaaggaaatcatctc

SEQ ID NO: 46 - DNA, 969 bp, Homo sapiens
atggcctcggagggcctggcgggggcgctggcttccgtgctggctggccaggggtccagcgtgcacagctgcgactcggcgccggccggggagccgccggcgcccgtgcggctgcggaagaacgtgtgctacgtggtgctggccgtgttcctcagcgagcaggatgaggtgctactgatccaggaggccaagagggagtgccgggggtcgtggtacctgcctgcggggagaatggagccaggggagaccatcgtggaggcgctgcagcgggaggtgaaggaggaggcggggctgcactgtgagcccgagacactgctgtccgtggaggagcggggcccctcctgggtccgcttcgtgttcctcgctcgccccacaggtggaattctcaagacttccaaggaggccgatgcggagtccctgcaggctgcctggtacccacggacctccctgcccactccgctgcgagcccatgacatcctgcacctggttgaactagccgcccagtatcgccagcaagccaggcaccctctcattctgccccaagagctaccctgtgatctggtctgccagcggctcgtggctacctttaccagcgcccagacagtgtgggtgttagtgggcacagtggggatgcctcacttgcctgtcactgcctgtggcctcgaccctatggagcagaggggtggcatgaagatggccgtcctgcggctgctgcaggagtgtctgaccctgcaccacttggtggtggagatcaaggggttgcttggactgcagcacctgggccgagatcacagtgatggcatctgtttgaatgtgctggtgaccgtggcttttcggagcccagggatccaggatgaacccccaaaagttcggggtgagaacttctcttggtggaaggtgatggaggaagacctgcaaagccagctcctccagcggcttcagggatcctctgttgtcccagtgaacaga

SEQ ID NO: 47 - DNA, 1404 bp, Homo sapiens
atggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtac

SEQ ID NO: 48 - DNA, 1431 bp, Homo sapiens
atgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtac

SEQ ID NO: 49 - DNA, 1515 bp, Homo sapiens
atgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtac

SEQ ID NO: 50 - DNA, 2241 bp, Homo sapiens
atggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatca

SEQ ID NO: 51 - DNA, 4323 bp, Homo sapiens
atggcgacggcggcggcagcggcggcggtgatggctcctccgggctgcccgggttcgtgccccaacttcgccgtagtctgctccttcttggagcgctacgggccgctgctagacctgcctgagttgccgttccctgagctggagcgggtgctgcaggcgccgccgccggacgtcggcaacggagaagtaccaaaagaattggtggagctccatttgaagctgatgaggaaaattggcaaatctgttactgcagacagatgggaaaaatatttgatcaagatatgccaagagtttaacagtacctgggcatgggagatggagaagaagggctatcttgaaatgagtgttgaatgcaaactagcactcttaaagtacctctgtgagtgtcagtttgatgacaatctcaaattcaagaatattattaatgaggaggatgccgatactatgcgtctccagccaattggtcgagacaaagatggcctcatgtactggtaccaattggatcaagatcacaatgtcagaatgtacatagaagaacaagatgatcaagatggctcttcatggaaatgcattgtcagaaatcgaaacgagttggctgagactcttgcactcctgaaagcacaaattgatcctgtactattgaaaaactctagccaacaagacaactcttctcgggaaagtcccagcttagaggatgaggagactaaaaaagaggaagaaacacctaaacaagaggaacagaaagaaagtgaaaagatgaaaagtgaggagcagcctatggatttagaaaaccgttctacagccaatgttctagaagagactactgtgaaaaaagaaaaagaagatgaaaaggaacttgtgaaactgccagtcatagtgaagctagaaaaacctttgccagaaaatgaagaaaaaaagattatcaaagaagaaagtgattccttcaaggaaaatgtcaaacccattaaagttgaggtgaaggaatgtagagcagatcctaaagataccaaaagtagcatggagaagccagtggcacaggagcctgaaaggatcgaatttggtggcaatattaaatcttctcacgaaattactgagaaatctactgaagaaactgagaaacttaaaaatgaccagcaggccaagataccactaaaaaaacgagaaattaaactgagtgatgattttgacagtccagtcaagggacctttgtgtaaatcagttactccaacaaaagagtttttgaaagatgaaataaaacaagaggaagagacttgtaaaaggatctctacaatcactgctttgggtcatgaagggaaacagctggtaaatggagaagttagtgatgaaagggtagctccaaattttaagacagaaccaatagagacaaagttttatgagacaaaggaagagagctatagcccctctaaggacagaaatatcatcacggagggaaatggaacagagtccttaaattctgtcataacaagtatgaaaacaggtgagcttgagaaagaaacagcccctttgaggaaagatgcagatagttcaatatcagtcttagagatccatagtcaaaaagcacaaatagaggaacccgatcctccagaaatggaaacttctcttgattcttctgagatggcaaaagatctctcttcaaaaactgctttatcttccaccgagtcgtgtaccatgaaaggtgaagagaagtctcccaaaactaagaaggataagcgcccaccaatcctagaatgtcttgaaaagttagagaagtccaaaaagacttttcttgataaggacgcacaaagattgagtccaataccagaagaagttccaaagagtactctagagtcagaaaagcctggctctcctgaggcagctgaaacttctccaccatctaatatcattgaccactgtgagaaactagcctcagaaaaagaagtggtagaatgccagagtacaagtactgttggtggccagtctgtgaaaaaagtagacctagaaaccctaaaagaggattctgagttcacaaaggtagaaatggataatctggacaatgcccagacctctggcatagaggagccttctgagacaaagggttctatgcaaaaaagcaaattcaaatataagttggttcctgaagaagaaaccactgcctcagaaaatacagagataacctctgaaaggcagaaagagggcatcaaattaacaatcaggatatcaagtcggaaaaagaagcccgattctccccccaaagttctagaaccagaaaacaagcaagagaagacagaaaaggaagaggagaaaacaaatgtgggtcgtactttaagaagatctccaagaatatctagacccactgcaaaagtggctgagatcagagatcagaaagctgataaaaaaagaggggaaggagaagatgaggtggaagaagagtcaacagctttgcaaaaaactgacaaaaaggaaattttgaaaaaatcagagaaagatacaaattctaaagtaagcaaggtaaaacccaaaggcaaagttcgatggactggttctcggacacgtggcagatggaaatattccagcaatgatgaaagtgaagggtctggcagtgaaaaatcatctgcagcttcagaagaggaggaagaaaaggaaagtgaagaagccatcctagcagatgatgatgaaccatgcaaaaaatgtggccttccaaaccatcctgagctaattcttctgtgtgactcttgcgatagtggataccatactgcctgccttcgccctcctctgatgatcatcccagatggagaatggttctgcccaccttgccaacataaactgctctgtgaaaaattagaggaacagttgcaggatttggatgttgccttaaagaagaaagagcgtgccgaacgaagaaaagaacgcttggtgtatgttggtatcagtattgaaaacatcattcctccacaagagccagacttttctgaagatcaagaagaaaagaaaaaagattcaaaaaaatccaaagcaaacttgcttgaaaggaggtcaacaagaacaaggaaatgtataagctacagatttgatgagtttgatgaagcaattgatgaagctattgaagatgacatcaaagaagccgatggaggaggagttggccgaggaaaagatatctccaccatcacaggtcatcgtgggaaagacatctctactattttggatgaagaaagaaaagaaaataaacgaccccagagggcagctgctgctcgaaggaagaaacgccggcgattaaatgatctggacagtgatagcaacctggatgaagaagagagcgaggatgaattcaagatcagtgatggatctcaagatgagtttgttgtgtctgatgaaaacccagatgaaagtgaagaagatccgccatctaatgatgacagtgacactgacttttgtagccgtagactgaggcgacacccctctcggccaatgaggcagagcaggcgtttgcgaagaaagaccccaaagaaaaaatattccgatgatgatgaagaggaggaatctgaggagaatagtagagactctgaaagtgacttcagtgatgattttagtgatgattttgtagaaactcggcgaaggcggtcaaggagaaatcagaaaagacaaattaactacaaagaagactcagaaagtgacggttcccagaagagtttgcgacgtggtaaagaaataaggcgagtacacaagcgaagactttccagctcagagagtgaagagagctatttgtccaagaactctgaagatgatgagctagctaaagaatcaaagcggtcagttcgaaagcggggccgaagcacagacgagtattcagaagcagatgaggaggaggaggaagaggaaggcaaaccatcccgcaaacggctacaccggattgagacggatgaggaggagagttgtgacaatgctcatggagatgcaaatcagcctgcccgtgacagccagcctagggtcctgccctcagaacaagagagcaccaagaagccctaccggatagaaagtgatgaggaagaggactttgaaaatgtaggcaaagtggggagcccattggactatagcttagtggacttaccttcaaccaatggacagagccctggcaaagccattgagaacttgattggcaagcctactgagaagtctcagacccccaaggacaacagcacagccagtgcaagcctagcctccaatgggacaagtggtgggcaggaggcaggagcaccagaagaggaggaagatgagcttttgagagtgactgaccttgttgattatgtctgtaacagtgaacagtta

SEQ ID NO: 52 - DNA, 1122 bp, Homo sapiens
atgtcaagggttccaagtcctccacctccggcagaaatgtcgagtggccccgtagctgagagttggtgctacacacagatcaaggtagtgaaattctcctacatgtggaccatcaataactttagcttttgccgggaggaaatgggtgaagtcattaaaagttctacattttcatcaggagcaaatgataaactgaaatggtgtttgcgagtaaaccccaaagggttagatgaagaaagcaaagattacctgtcactttacctgttactggtcagctgtccaaagagtgaagttcgggcaaaattcaaattctccatcctgaatgccaagggagaagaaaccaaagctatggagagtcaacgggcatataggtttgtgcaaggcaaagactggggattcaagaaattcatccgtagagattttcttttggatgaggccaacgggcttctccctgatgacaagcttaccctcttctgcgaggtgagtgttgtgcaagattctgtcaacatttctggccagaataccatgaacatggtaaaggttcctgagtgccggctggcagatgagttaggaggactgtgggagaattcccggttcacagactgctgcttgtgtgttgccggccaggaattccaggctcacaaggctatcttagcagctcgttctccggtttttagtgccatgtttgaacatgaaatggaggagagcaaaaagaatcgagttgaaatcaatgatgtggagcctgaagtttttaaggaaatgatgtgcttcatttacacggggaaggctccaaacctcgacaaaatggctgatgatttgctggcagctgctgacaagtatgccctggagcgcttaaaggtcatgtgtgaggatgccctctgcagtaacctgtccgtggagaacgctgcagaaattctcatcctggccgacctccacagtgcagatcagttgaaaactcaggcagtggatttcatcaactatcatgcttcggatgtcttggagacctctgggtggaagtcaatggtggtgtcacatccccacttggtggctgaggcataccgctctctggcttcagcacagtgcccttttctgggacccccacgcaaacgcctgaagcaatcc

SEQ ID NO: 53 - DNA, 738 bp, Homo sapiens
atgggaccccagcatttgagacttgtgcagctgttctgccttctaggggccatctccactctgcctcgggctggagctcttttgtgctatgaagcaacagcctcaagattcagagctgttgctttccataactggaagtggcttctgatgaggaacatggtgtgtaagctgcaagagggctgcgaggagacgctagtgttcattgagacagggactgcaaggggagtcgtgggctttaaaggctgcagctcgtcttcgtcttaccctgcgcaaatctcctaccttgtttccccacccggagtgtccattgcctcctacagtcgcgtctgccggtcttatctctgcaacaacctcaccaatttggagccttttgtgaaactcaaggccagcactcctaagtctatcacatctgcgtcctgtagctgcccgacctgtgtgggcgagcacatgaaggattgcctcccaaattttgtcaccactaattcttgccccttggctgcttctacgtgttacagttccaccttaaaatttcaggcagggtttctcaataccaccttcctcctcatggggtgtgctcgtgaacataaccagcttttagcagattttcatcatattgggagcatcaaagtgactgaggtcctcaacatcttagagaagtctcagattgttggtgcagcatcctccaggcaagatcctgcttggggtgtcgtcttaggcctcctgtttgccttcagggac

SEQ ID NO: 54 - DNA, 1503 bp, Homo sapiens
atggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgccttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccctctcctgctttggctccatgaacggtggtgtgtttgctgtctccaggttattctatgttgcgtctcgagagggtcaccttccagaaatcctctccatgattcatgtccgcaagcacactcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcagagaaaataaccagaacattacaaataatactggaagttgtaccagaagaagataagtta

SEQ ID NO: 55 - DNA, 1218 bp, Homo sapiens
atgcatcctgccgtcttcctatccttacccgacctcagatgctcccttctgctcctggtaacttgggtttttactcctgtaacaactgaaataacaagtcttgatacagagaatatagatgaaattttaaacaatgctgatgttgctttagtaaatttttatgctgactggtgtcgtttcagtcagatgttgcatccaatttttgaggaagcttccgatgtcattaaggaagaatttccaaatgaaaatcaagtagtgtttgccagagttgattgtgatcagcactctgacatagcccagagatacaggataagcaaatacccaaccctcaaattgtttcgtaatgggatgatgatgaagagagaatacaggggtcagcgatcagtgaaagcattggcagattacatcaggcaacaaaaaagtgaccccattcaagaaattcgggacttagcagaaatcaccactcttgatcgcagcaaaagaaatatcattggatattttgagcaaaaggactcggacaactatagagtttttgaacgagtagcgaatattttgcatgatgactgtgcctttctttctgcatttggggatgtttcaaaaccggaaagatatagtggcgacaacataatctacaaaccaccagggcattctgctccggatatggtgtacttgggagctatgacaaattttgatgtgacttacaattggattcaagataaatgtgttcctcttgtccgagaaataacatttgaaaatggagaggaattgacagaagaaggactgccttttctcatactctttcacatgaaagaagatacagaaagtttagaaatattccagaatgaagtagctcggcaattaataagtgaaaaaggtacaataaactttttacatgccgattgtgacaaatttagacatcctcttctgcacatacagaaaactccagcagattgtcctgtaatcgctattgacagctttaggcatatgtatgtgtttggagacttcaaagatgtattaattcctggaaaactcaagcaattcgtatttgacttacattctggaaaactgcacagagaattccatcatggacctgacccaactgatacagccccaggagagcaagcccaagatgtagcaagcagtccacctgagagctccttccagaaactagcacccagtgaatataggtatactctattgagggatcgagatgagctt

SEQ ID NO: 56 - DNA, 1515 bp, Homo sapiens
atgcgcctccgccgcctagcgctgttcccgggtgtggcgctgcttcttgccgcggcccgcctcgccgctgcctccgacgtgctagaactcacggacgacaacttcgagagtcgcatctccgacacgggctctgcgggcctcatgctcgtcgagttcttcgccccctggtgtggacactgcaagagacttgcacctgagtatgaagctgcagctaccagattaaaaggaatagtcccattagcaaaggttgattgcactgccaacactaacacctgtaataaatatggagtcagtggatatccaaccctgaagatatttagagatggtgaagaagcaggtgcttatgatggacctaggactgctgatggaattgtcagccacttgaagaagcaggcaggaccagcttcagtgcctctcaggactgaggaagaatttaagaaattcattagtgataaagatgcctctatagtaggttttttcgatgattcattcagtgaggctcactccgagttcctaaaagcagccagcaacttgagggataactaccgatttgcacatacgaatgttgagtctctggtgaacgagtatgatgataatggagagggtatcatcttatttcgtccttcacatctcactaacaagtttgaggacaagactgtggcatatacagagcaaaaaatgaccagtggcaaaattaaaaagtttatccaggaaaacatttttggtatctgccctcacatgacagaagacaataaagatttgatacagggcaaggacttacttattgcttactatgatgtggactatgaaaagaacgctaaaggttccaactactggagaaacagggtaatgatggtggcaaagaaattcctggatgctgggcacaaactcaactttgctgtagctagccgcaaaacctttagccatgaactttctgattttggcttggagagcactgctggagagattcctgttgttgctatcagaactgctaaaggagagaagtttgtcatgcaggaggagttctcgcgtgatgggaaggctctggagaggttcctgcaggattactttgatggcaatctgaagagatacctgaagtctgaacctatcccagagagcaatgatgggcctgtgaaggtagtggtagcagagaattttgatgaaatagtgaataatgaaaataaagatgtgctgattgaattttatgccccttggtgtggtcactgtaagaacctggagcccaagtataaagaacttggcgagaagctcagcaaagacccaaatatcgtcatagccaagatggatgccacagccaatgatgtgccttctccatatgaagtcagaggttttcctaccatatacttctctccagccaacaagaagctaaatccaaagaaatatgaaggtggccgtgaattaagtgattttattagctatctacaaagagaagctacaaacccccctgtaattcaagaagaaaaacccaagaagaagaagaaggcacaggaggatctc

SEQ ID NO: 57 - DNA, 822 bp, Homo sapiens
atggaagctgccccgtccaggttcatgttcctcttatttctcctcacgtgtgagctggctgcagaagttgctgcagaagttgagaaatcctcagatggtcctggtgctgcccaggaacccacgtggctcacagatgtcccagctgccatggaattcattgctgccactgaggtggctgtcataggcttcttccaggatttagaaataccagcagtgcccatactccatagcatggtgcaaaaattcccaggcgtgtcatttgggatcagcactgattctgaggttctgacacactacaacatcactgggaacaccatctgcctctttcgcctggtagacaatgaacaactgaatttagaggacgaagacattgaaagcattgatgccaccaaattgagccgtttcattgagatcaacagcctccacatggtgacagagtacaaccctgtgactgtgattgggttattcaacagcgtaattcagattcatctcctcctgataatgaacaaggcctccccagagtatgaagagaacatgcacagataccagaaggcagccaagctcttccaggggaagattctctttattctggtggacagtggtatgaaagaaaatgggaaggtgatatcatttttcaaactaaaggagtctcaactgccagctttggcaatttaccagactctagatgacgagtgggatacactgcccacagcagaagtttccgtagagcatgtgcaaaacttttgtgatggattcctaagtggaaaattgttgaaagaaaatcgtgaatcagaaggaaagactccaaaggtggaactctga

SEQ ID NO: 58 - DNA, 1872 bp, Homo sapiens
atgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgt

SEQ ID NO: 59 - DNA, 2274 bp, Homo sapiens
atggcggtgtctgccgggtccgcgcggacctcgcccagctcagataaagtacagaaagacaaggctgaactgatctcagggcccaggcaggacagccgaatagggaaactcttgggttttgagtggacagatttgtccagttggcggaggctggtgaccctgctgaatcgaccaacggaccctgcaagcttagctgtctttcgttttctttttgggttcttgatggtgctagacattccccaggagcgggggctcagctctctggaccggaaataccttgatgggctggatgtgtgccgcttccccttgctggatgccctacgcccactgccacttgactggatgtatcttgtctacaccatcatgtttctgggggcactgggcatgatgctgggcctgtgctaccggataagctgtgtgttattcctgctgccatactggtatgtgtttctcctggacaagacatcatggaacaaccactcctatctgtatgggttgttggcctttcagctaacattcatggatgcaaaccactactggtctgtggacggtctgctgaatgcccataggaggaatgcccacgtgcccctttggaactatgcagtgctccgtggccagatcttcattgtgtacttcattgcgggtgtgaaaaagctggatgcagactgggttgaaggctattccatggaatatttgtcccggcactggctcttcagtcccttcaaactgctgttgtctgaggagctgactagcctgctggtcgtgcactggggtgggctgctgcttgacctctcagctggtttcctgctcttttttgatgtctcaagatccattggcctgttctttgtgtcctacttccactgcatgaattcccagcttttcagcattggtatgttctcctacgtcatgctggccagcagccctctcttctgctcccctgagtggcctcggaagctggtgtcctactgcccccgaaggttgcaacaactgttgcccctcaaggcagcccctcagcccagtgtttcctgtgtgtataagaggagccggggcaaaagtggccagaagccagggctgcgccatcagctgggagctgccttcaccctgctctacctcctggagcagctattcctgccctattctcattttctcacccagggctataacaactggacaaatgggctgtatggctattcctgggacatgatggtgcactcccgctcccaccagcacgtgaagatcacctaccgtgatggccgcactggcgaactgggctaccttaaccctggggtatttacacagagtcggcgatggaaggatcatgcagacatgctgaagcaatatgccacttgcctgagccgcctgcttcccaagtataatgtcactgagccccagatctactttgatatttgggtctccatcaatgaccgcttccagcagaggatttttgaccctcgtgtggacatcgtgcaggccgcttggtcaccctttcagcgcacatcctgggtgcaaccactcttgatggacctgtctccctggagggccaagttacaggaaatcaagagcagcctagacaaccacactgaggtggtcttcattgcagatttccctggactgcacttggagaattttgtgagtgaagacctgggcaacactagcatccagctgctgcagggggaagtgactgtggagcttgtggcagaacagaagaaccagactcttcgagagggagaaaaaatgcagttgcctgctggtgagtaccataaggtgtatacgacatcacctagcccttcttgctacatgtacgtctatgtcaacactacagagcttgcactggagcaagacctggcatatctgcaagaattaaaggaaaaggtggagaatggaagtgaaacagggcctctacccccagagctgcagcctctgttggaaggggaagtaaaagggggccctgagccaacacctctggttcagacctttcttagacgccaacaaaggctccaggagattgaacgccggcgaaatactcctttccatgagcgattcttccgcttcttgttgcgaaagctctatgtctttcgccgcagcttcctgatgacttgtatctcacttcgaaatctgatattaggccgtccttccctggagcagctggcccaggaggtgacttatgcaaacttgagaccctttgaggcagttggagaactgaatccctcaaacacggattcttcacattctaatcctcctgagtcaaatcctgatcctgtccactcagagttc

SEQ ID NO: 60 - DNA, 2103 bp, Homo sapiens
atggcggtgtctgccgggtccgcgcggacctcgcccagctcagggttcttgatggtgctagacattccccaggagcgggggctcagctctctggaccggaaataccttgatgggctggatgtgtgccgcttccccttgctggatgccctacgcccactgccacttgactggatgtatcttgtctacaccatcatgtttctgggggcactgggcatgatgctgggcctgtgctaccggataagctgtgtgttattcctgctgccatactggtatgtgtttctcctggacaagacatcatggaacaaccactcctatctgtatgggttgttggcctttcagctaacattcatggatgcaaaccactactggtctgtggacggtctgctgaatgcccataggaggaatgcccacgtgcccctttggaactatgcagtgctccgtggccagatcttcattgtgtacttcattgcgggtgtgaaaaagctggatgcagactgggttgaaggctattccatggaatatttgtcccggcactggctcttcagtcccttcaaactgctgttgtctgaggagctgactagcctgctggtcgtgcactggggtgggctgctgcttgacctctcagctggtttcctgctcttttttgatgtctcaagatccattggcctgttctttgtgtcctacttccactgcatgaattcccagcttttcagcattggtatgttctcctacgtcatgctggccagcagccctctcttctgctcccctgagtggcctcggaagctggtgtcctactgcccccgaaggttgcaacaactgttgcccctcaaggcagcccctcagcccagtgtttcctgtgtgtataagaggagccggggcaaaagtggccagaagccagggctgcgccatcagctgggagctgccttcaccctgctctacctcctggagcagctattcctgccctattctcattttctcacccagggctataacaactggacaaatgggctgtatggctattcctgggacatgatggtgcactcccgctcccaccagcacgtgaagatcacctaccgtgatggccgcactggcgaactgggctaccttaaccctggggtatttacacagagtcggcgatggaaggatcatgcagacatgctgaagcaatatgccacttgcctgagccgcctgcttcccaagtataatgtcactgagccccagatctactttgatatttgggtctccatcaatgaccgcttccagcagaggatttttgaccctcgtgtggacatcgtgcaggccgcttggtcaccctttcagcgcacatcctgggtgcaaccactcttgatggacctgtctccctggagggccaagttacaggaaatcaagagcagcctagacaaccacactgaggtggtcttcattgcagatttccctggactgcacttggagaattttgtgagtgaagacctgggcaacactagcatccagctgctgcagggggaagtgactgtggagcttgtggcagaacagaagaaccagactcttcgagagggagaaaaaatgcagttgcctgctggtgagtaccataaggtgtatacgacatcacctagcccttcttgctacatgtacgtctatgtcaacactacagagcttgcactggagcaagacctggcatatctgcaagaattaaaggaaaaggtggagaatggaagtgaaacagggcctctacccccagagctgcagcctctgttggaaggggaagtaaaagggggccctgagccaacacctctggttcagacctttcttagacgccaacaaaggctccaggagattgaacgccggcgaaatactcctttccatgagcgattcttccgcttcttgttgcgaaagctctatgtctttcgccgcagcttcctgatgacttgtatctcacttcgaaatctgatattaggccgtccttccctggagcagctggcccaggaggtgacttatgcaaacttgagaccctttgaggcagttggagaactgaatccctcaaacacggattcttcacattctaatcctcctgagtcaaatcctgatcctgtccactcagagttc

SEQ ID NO: 61 - DNA, 528 bp, Homo sapiens
atggcggctcccgtcctgctaagagtgtcggtgccgcggtgggagcgggtggcccggtatgcagtgtgcgctgccggaatcctgctctccatctacgcctaccacgtggagcgggagaaggagcgggaccccgagcaccgggccctctgcgacctggggccctgggtgaagtgctccgccgcccttgcctccagatggggtcgaggatttggtcttttgggttccatttttggaaaggatggtgtattaaaccagccaaacagtgtctttggacttatattttatatactacagttattacttggcatgacagcaagcgctgtggcggctttgatcctcatgacgtcctccatcatgtcggtcgtggggtccctgtacctggcctacattctgtactttgtgctgaaggagttctgcatcatctgcatcgtcacgtacgtgctgaacttccttcttctcattatcaactacaaacgactagtttacttgaacgaggcctggaagcggcagctgcaacccaagcaggac

SEQ ID NO: 62 - DNA, 240 bp, Homo sapiens
atgaaactgtcaggcatgtttctgctcctctctctggctcttttctgctttttaacaggtgtcttcagtcagggaggacaggttgactgtggtgagttccaggaccccaaggtctactgcactcgggaatctaacccacactgtggctctgatggccagacatatggcaataaatgtgccttctgtaaggccatagtgaaaagtggtggaaagattagcctaaagcatcctggaaaatgc

SEQ ID NO: 63 - DNA, 1296 bp, Escherichia coli
atgcaagtttcagttgaaaccactcaaggccttggccgccgtgtaacgattactatcgctgctgacagcatcgagaccgctgttaaaagcgagctggtcaacgttgcgaaaaaagtacgtattgacggcttccgcaaaggcaaagtgccaatgaatatcgttgctcagcgttatggcgcgtctgtacgccaggacgttctgggtgacctgatgagccgtaacttcattgacgccatcattaaagaaaaaatcaatccggctggcgcaccgacttatgttccgggcgaatacaagctgggtgaagacttcacttactctgtagagtttgaagtttatccggaagttgaactgcagggtctggaagcgatcgaagttgaaaaaccgatcgttgaagtgaccgacgctgacgttgacggcatgctggatactctgcgtaaacagcaggcgacctggaaagaaaaagacggcgctgttgaagcagaagaccgcgtaaccatcgacttcaccggttctgtagacggcgaagagttcgaaggcggtaaagcgtctgatttcgtactggcgatgggccagggtcgtatgatcccgggctttgaagacggtatcaaaggccacaaagctggcgaagagttcaccatcgacgtgaccttcccggaagaataccacgcagaaaacctgaaaggtaaagcagcgaaattcgctatcaacctgaagaaagttgaagagcgtgaactgccggaactgactgcagaattcatcaaacgtttcggcgttgaagatggttccgtagaaggtctgcgcgctgaagtgcgtaaaaacatggagcgcgagctgaagagcgccatccgtaaccgcgttaagtctcaggcgatcgaaggtctggtaaaagctaacgacatcgacgtaccggctgcgctgatcgacagcgaaatcgacgttctgcgtcgccaggctgcacagcgtttcggtggcaacgaaaaacaagctctggaactgccgcgcgaactgttcgaagaacaggctaaacgccgcgtagttgttggcctgctgctgggcgaagttatccgcaccaacgagctgaaagctgacgaagagcgcgtgaaaggcctgatcgaagagatggcttctgcgtacgaagatccgaaagaagttatcgagttctacagcaaaaacaaagaactgatggacaacatgcgcaatgttgctctggaagaacaggctgttgaagctgtactggcgaaagcgaaagtgactgaaaaagaaaccactttcaacgagctgatgaaccagcaggcg

SEQ ID NO: 64 - DNA, 453 bp, Homo sapiens
atgatggcagaagagcacacagatctcgaggcccagatcgtcaaggatatccactgcaaggagattgacctggtgaaccgagaccccaagaacattaacgaggacatagtcaaggtggattttgaagacgtgatcgcagagcctgtgggcacctacagctttgacggcgtgtggaaggtgagctacaccaccttcactgtctccaagtactggtgctaccgtctgttgtccacgctgctgggcgtcccactggccctgctctggggcttcctgttcgcctgcatctccttctgccacatctgggcggtggtgccatgcattaagagctacctgatcgagatccagtgcatcagccacatctactcactctgcatccgcaccttctgcaacccactcttcgcggccctgggccaggtctgcagcagcatcaaggtggtgctgcggaaggaggtc

SEQ ID NO: 65 - DNA, 2238 bp, Homo sapiens
atgctggtgctcgggctgctggtagccggagcggcggacggatgcgagcttgtgccccggcacctccgcgggcggcgggcgactggctctgccgcaactgccgcctcctctcccgccgcggcggccggcgatagcccggcgctcatgacagatccctgcatgtcactgagtccaccatgctttacagaagaagacagatttagtctggaagctcttcaaacaatacataaacaaatggatgatgacaaagatggtggaattgaagtagaggaaagtgatgaattcatcagagaagatatgaaatataaagatgctactaataaacacagccatctgcacagagaagataaacatataacgattgaggatttatggaaacgatggaaaacatcagaagttcataattggacccttgaagacactcttcagtggttgatagagtttgttgaactaccccaatatgagaagaattttagagacaacaatgtcaaaggaacgacacttcccaggatagcagtgcacgaaccttcatttatgatctcccagttgaaaatcagtgaccggagtcacagacaaaaacttcagctcaaggcattggatgtggttttgtttggacctctaacacgcccacctcataactggatgaaagattttatcctcacagtttctatagtaattggtgttggaggctgctggtttgcttatacgcagaataagacatcaaaagaacatgttgcaaaaatgatgaaagatttagagagcttacaaactgcagagcaaagtctaatggacttacaagagaggcttgaaaaggcacaggaagaaaacagaaatgttgctgtagaaaagcaaaatttagagcgcaaaatgatggatgaaatcaattatgcaaaggaggaggcttgtcggctgagagagctaagggagggagctgaatgtgaattgagtagacgtcagtatgcagaacaggaattggaacaggttcgcatggctctgaaaaaggccgaaaaagaatttgaactgagaagcagttggtctgttccagatgcacttcagaaatggcttcagttaacacatgaagtagaagtgcaatactacaatattaaaagacaaaacgctgaaatgcagctagctattgctaaagatgaggcagaaaaaattaaaaagaagagaagcacagtctttgggactctgcacgttgcacacagctcctccctagatgaggtagaccacaaaattctggaagcaaagaaagctctctctgagttgacaacttgtttacgagaacgactttttcgctggcaacaaattgagaagatctgtggctttcagatagcccataactcaggactccccagcctgacctcttccctttattctgatcacagctgggtggtgatgcccagagtctccattccaccctatccaattgctggaggagttgatgacttagatgaagacacacccccaatagtgtcacaatttcccgggaccatggctaaacctcctggatcattagccagaagcagcagcctgtgccgttcacgccgcagcattgtgccgtcctcgcctcagcctcagcgagctcagcttgctccacacgccccccacccgtcacaccctcggcaccctcaccacccgcaacacacaccacactccttgccttcccctgatccagatatcctctcagtgtcaagttgccctgcgctttatcgaaatgaagaggaggaagaggccatttacttctctgctgaaaagcaatgggaagtgccagacacagcttcagaatgtgactccttaaattcttccattggaaggaaacagtctcctcctttaagcctcgagatataccaaacattatctccgcgaaagatatcaagagatgaggtgtccctagaggattcctcccgaggggattcgcctgtaactgtggatgtgtcttggggttctcccgactgtgtaggtctgacagaaactaagagtatgatcttcagtcctgcaagcaaagtgtacaatggcattttggagaaatcctgtagcatgaaccagctttccagtggcatcccggtgcctaaacctcgccacacatcatgttcctcagctggcaacgacagtaaaccagttcaggaagccccaagtgttgccagaataagcagcatcccacatgacctttgtcataatggagagaaaagcaaaaagccatcaaaaatcaaaagcctttttaagaagaaatctaag

SEQ ID NO: 66 - DNA, 2262 bp, Homo sapiens
atgctggtgctcgggctgctggtagccggagcggcggacggatgcgagcttgtgccccggcacctccgcgggcggcgggcgactggctctgccgcaactgccgcctcctctcccgccgcggcggccggcgatagcccggcgctcatgacagatccctgcatgtcactgagtccaccatgctttacagaagaagacagatttagtctggaagctcttcaaacaatacataaacaaatggatgatgacaaagatggtggaattgaagtagaggaaagtgatgaattcatcagagaagatatgaaatataaagatgctactaataaacacagccatctgcacagagaagataaacatataacgattgaggatttatggaaacgatggaaaacatcagaagttcataattggacccttgaagacactcttcagtggttgatagagtttgttgaactaccccaatatgagaagaattttagagacaacaatgtcaaaggaacgacacttcccaggatagcagtgcacgaaccttcatttatgatctcccagttgaaaatcagtgaccggagtcacagacaaaaacttcagctcaaggcattggatgtggttttgtttggacctctaacacgcccacctcataactggatgaaagattttatcctcacagtttctatagtaattggtgttggaggctgctggtttgcttatacgcagaataagacatcaaaagaacatgttgcaaaaatgatgaaagatttagagagcttacaaactgcagagcaaagtctaatggacttacaagagaggcttgaaaaggcacaggaagaaaacagaaatgttgctgtagaaaagcaaaatttagagcgcaaaatgatggatgaaatcaattatgcaaaggaggaggcttgtcggctgagagagctaagggagggagctgaatgtgaattgagtagacgtcagtatgcagaacaggaattggaacaggttcgcatggctctgaaaaaggccgaaaaagaatttgaactgagaagcagttggtctgttccagatgcacttcagaaatggcttcagttaacacatgaagtagaagtgcaatactacaatattaaaagacaaaacgctgaaatgcagctagctattgctaaagatgaggttgctgcttcatatctgattcaggcagaaaaaattaaaaagaagagaagcacagtctttgggactctgcacgttgcacacagctcctccctagatgaggtagaccacaaaattctggaagcaaagaaagctctctctgagttgacaacttgtttacgagaacgactttttcgctggcaacaaattgagaagatctgtggctttcagatagcccataactcaggactccccagcctgacctcttccctttattctgatcacagctgggtggtgatgcccagagtctccattccaccctatccaattgctggaggagttgatgacttagatgaagacacacccccaatagtgtcacaatttcccgggaccatggctaaacctcctggatcattagccagaagcagcagcctgtgccgttcacgccgcagcattgtgccgtcctcgcctcagcctcagcgagctcagcttgctccacacgccccccacccgtcacaccctcggcaccctcaccacccgcaacacacaccacactccttgccttcccctgatccagatatcctctcagtgtcaagttgccctgcgctttatcgaaatgaagaggaggaagaggccatttacttctctgctgaaaagcaatgggaagtgccagacacagcttcagaatgtgactccttaaattcttccattggaaggaaacagtctcctcctttaagcctcgagatataccaaacattatctccgcgaaagatatcaagagatgaggtgtccctagaggattcctcccgaggggattcgcctgtaactgtggatgtgtcttggggttctcccgactgtgtaggtctgacagaaactaagagtatgatcttcagtcctgcaagcaaagtgtacaatggcattttggagaaatcctgtagcatgaaccagctttccagtggcatcccggtgcctaaacctcgccacacatcatgttcctcagctggcaacgacagtaaaccagttcaggaagccccaagtgttgccagaataagcagcatcccacatgacctttgtcataatggagagaaaagcaaaaagccatcaaaaatcaaaagcctttttaagaagaaatctaag

SEQ ID NO: 67 - DNA, 2055 bp, Homo sapiens
atggatgtatgcgtccgtcttgccctgtggctcctctggggactcctcctgcaccagggccagagcctcagccatagtcacagtgagaaggcgacaggaaccagctcgggggccaactctgaggagtccactgcagcagagttttgccgaattgacaagcccctgtgtcacagtgaggatgagaaactcagcttcgaggcagtccgtaacatccacaaactgatggacgatgatgccaatggtgatgtggatgtggaagaaagtgatgagttcctgagggaagacctcaattaccatgacccaacagtgaaacacagcaccttccatggtgaggataagctcatcagcgtggaggacctgtggaaggcatggaagtcatcagaagtatacaattggaccgtggatgaggtggtacagtggctgatcacatatgtggagctgcctcagtatgaggagaccttccggaagctgcagctcagtggccatgccatgccaaggctggctgtcaccaacaccaccatgacagggactgtgctgaagatgacagaccggagtcatcggcagaagctgcagctgaaggctctggatacagtgctctttgggcctcctctcttgactcgccataatcacctcaaggacttcatgctggtggtgtctatcgttattggtgtgggcggctgctggtttgcctatatccagaaccgttactccaaggagcacatgaagaagatgatgaaggacttggaggggttacaccgagctgagcagagtctgcatgaccttcaggaaaggctgcacaaggcccaggaggagcaccgcacagtggaggtggagaaggtccatctggaaaagaagctgcgcgatgagatcaaccttgctaagcaggaagcccagcggctgaaggagctgcgggagggtactgagaatgagcggagccgccaaaaatatgctgaggaggagttggagcaggttcgggaggccttgaggaaagcagagaaggagctagaatctcacagctcatggtatgctccagaggcccttcagaagtggctgcagctgacacatgaggtggaggtgcaatattacaacatcaagaagcaaaatgctgagaagcagctgctggtggccaaggagggggctgagaagataaaaaagaagagaaacacactctttggcaccttccacgtggcccacagctcttccctggatgatgtagatcataaaattctaacagctaagcaagcactgagcgaggtgacagcagcattgcgggagcgcctgcaccgctggcaacagatcgagatcctctgtggcttccagattgtcaacaaccctggcatccactcactggtggctgccctcaacatagaccccagctggatgggcagtacacgccccaaccctgctcacttcatcatgactgacgacgtggatgacatggatgaggagattgtgtctcccttgtccatgcagtcccctagcctgcagagcagtgttcggcagcgcctgacggagccacagcatggcctgggatctcagagggatttgacccattccgattcggagtcctccctccacatgagtgaccgccagcgtgtggcccccaaacctcctcagatgagccgtgctgcagacgaggctctcaatgccatgacttccaatggcagccaccggctgatcgagggggtccacccagggtctctggtggagaaactgcctgacagccctgccctggccaagaaggcattactggcgctgaaccatgggctggacaaggcccacagcctgatggagctgagcccctcagccccacctggtggctctccacatttggattcttcccgttctcacagccccagctccccagacccagacacaccatctccagttggggacagccgagccctgcaagccagccgaaacacacgcattccccacctggctggcaagaaggctgtggctgaggaggataatggctctattggcgaggaaacagactccagcccaggccggaagaagtttcccctcaaaatctttaagaagcctcttaagaag

SEQ ID NO: 68 - DNA, 903 bp, Homo sapiens
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcc

SEQ ID NO: 69 - DNA, 762 bp, Homo sapiens
atgagtgctgagcttaacgtgcctatcgacccctctgctcctgcctgccctgagcccggccataagggcatggattaccgggactgggtccgccgcagctacctggaactggtcacctctaaccaccactcggtacaggccctgtcgtggcggaagctctacctgagcagggccaagctgaaggcctccagcaggacctccgccctcctctccggctttgccatggtggccatggtggaggtgcagctggagacgcagtaccagtacccgcggccgctgctgattgccttcagcgcctgcaccacggtgctggtggccgtgcacctgttcgccctcctcatcagcacctgcatcctgcccaatgtggaggccgtgagcaacatccacaacctgaactccatcagcgagtccccgcatgagcgcatgcacccctacatcgagctggcctggggcttctccaccgtgcttggcatcctactcttcctggccgaggtggtgctgctctgctggatcaagttcctccccgtggatgcccggcgccagcctggccccccacctggccctgggagtcacacgggctggcaggccgccctggtgtccaccatcatcatggtgcccgtgggcctcatcttcgtggtcttcaccatccacttctaccgctccctggtgcgccacaaaacggagcgccacaaccgcgagatcgaggagctccacaagctcaaggtccagctggacgggcatgagcgcagcctgcaggtcttg

SEQ ID NO: 70 - DNA, 885 bp, Homo sapiens
atgaagggcggcgagggggacgcgggcgagcaggccccgctgaaccctgagggcgagagccctgcaggctcggccacgtaccgggagttcgtgcaccgcggctacctggacctcatgggggccagtcagcactcgctgcgggcgctcagctggcgccgcctctacctcagccgggccaagctcaaagcttccagccgcacgtctgccttgctctcgggcttcgccatggtggccatggtggaggtgcagctggagagtgaccacgagtacccaccaggcctgctggtggccttcagtgcctgcaccaccgtgctggtggctgtgcacctctttgcactcatggtctccacgtgtctgctgccccacattgaagctgtgagcaacatccacaacctcaactctgtccaccagtcgccacaccagagactgcaccgctacgtggagctggcctggggcttctccactgccctgggcacctttctcttccttgctgaagttgtcctggttggttgggtcaagtttgtgcccattggggctcccttggacacaccgacccccatggtgcccacatcccgggtgcccgggactctggcaccagtggctacctcccttagtccagcttccaatctcccacggtcctctgcgtctgcagcaccgtcccaggctgagccagcctgcccaccccggcaagcctgtggtggtggtggggcccatgggccaggctggcaagcagccatggcctccacagccatcatggtacccgtggggctcgtgtttgtggcctttgccctgcatttctaccgctccttggtggcacacaagacagaccgctacaagcaggaactagaggaactgaatcgcctgcagggggagctgcaggctgtg

SEQ ID NO: 71 - DNA, 1752 bp, Homo sapiens
atgaagatgatgctggtgcgccggttccgcgtgctcatcctgatggtgttcctggtggcctgcgcgctgcacatcgccctggacctgctgcccaggctggagcgacgcggcgcgcggccctcgggggagcccggctgttcgtgcgcgcagcccgccgccgaggtggccgcgcccggctgggcccaggttcggggccgccccggggagcccccggccgcctcctccgccgccggcgacgcgggctggcccaacaagcacacgctccgcatcctgcaggacttcagctccgacccctcctccaacctctcgtcccactcgctggagaaactgccgcccgcggccgagccggccgagcgcgccttgcgggggcgggatcccggcgccctaagaccccacgaccccgcgcaccggccgctgctgcgagaccccggcccgcgtcggtccgagtcgccccccggccccggcggagacgcctccctcctggccaggctgttcgagcacccgctttaccgggtggcggttccgccgctcacggaggaggacgtcctgttcaatgtgaacagcgacaccaggctcagccccaaagcggcggagaacccggactggccgcatgcgggtgctgaaggtgcagaattcctctcccccggggaggcggccgtggactcctatcccaactggctcaagttccacattggtatcaaccggtacgagctgtactccagacacaacccggccatcgaggccctgctgcacgacctcagctcccagaggatcaccagcgtggccatgaagtcggggggcacgcagctgaagctcatcatgaccttccagaattacgggcaagcgctgttcaaacccatgaaacaaacgagggagcaggagacaccccctgactttttttatttctctgactacgagaggcacaatgcggagattgctgccttccacctggacaggatcctggacttccgccgggtccctcccgtggccggcaggatggtcaacatgaccaaggagatccgggacgtcacacgggacaagaagctctggaggaccttcttcatctctccagccaacaacatctgcttctacggcgagtgttcctactactgctccacggagcacgccctgtgcgggaagccagaccagatcgagggctcgctggcggccttcctgcccgacctgtccctggccaagaggaagacctggcggaacccttggcggcgttcctaccacaagcgcaagaaggccgagtgggaggtggaccctgactactgcgaggaggtgaagcagacaccgccctacgacagcagccaccgcatcctggacgtcatggacatgacgatcttcgacttcctcatgggaaacatggaccgtcaccactacgagacttttgagaagtttgggaatgaaacgttcatcatccacttagacaatggaagagggtttgggaagtattcgcacgacgagctctccatcctggtgccgctacagcagtgctgcaggatccggaagtccacctacctgcgtctgcagctcctggccaaggaggagtacaagctgagcctgctgatggccgagtctctgcggggggaccaggtggcacccgtgctgtaccagccgcacctggaggccctggaccggcggctccgcgtcgtgctaaaggccgtccgggactgcgtggagaggaacgggctccacagcgtggtggatgacgacctggacactgagcacagagccgcctcggcgagg

SEQ ID NO: 72 - DNA, 1095 bp, Homo sapiens
atgcggcctcaggacagcaccggggtcgcggagctccaggagcccgggctgccgctaacggacgatgcacccccgggcgccactgaggagccggcggccgccgaggcagctggggcgccagaccgcggtaggtgctggctctgcctttcctcgccgtgttgctcccgcaccgagccggaagccaagaagaaagcaccctgtcctggacttggcttgttttacacattattgtctgccttccttttctcagtgggctctttatttgttaaaaaagtgcaagacgtccatgctgtagagattagtgcgtttcgatgtgtgttccaaatgctagttgttatcccttgcttaatatacagaaaaactgggtttataggcccaaaaggtcaacgaattttcctcattctcagaggagtccttggttctaccgccatgatgcttatatactatgcttaccagacaatgtccctcgctgatgccacagttatcacgtttagcagtccagtgtttacgtccatatttgcttggatatgtctcaaggaaaaatatagcccttgggatgctcttttcaccgtgttcacaatcactggagtgatccttatcgtgagaccaccatttttgtttggttccgacacttcggggatggaagaaagctattcaggccaccttaagggaacattcgcagcaattggaagtgccgtatttgctgcatcgactctagttatcctaagaaaaatgggaaaatctgtggactactttctgagcatttggtattatgtagtacttggcctcgttgaaagtgtcatcatcctctctgtattaggagagtggagtctgccttactgtgggttggacaggctatttctcatattcattgggctctttggtttggggggtcagatatttatcacaaaagcacttcaaatagaaaaagcagggccagtagcaataatgaagacaatggatgtggtctttgcttttatctttcagattattttctttaataatgtgccaacgtggtggacagtgggtggtgctctctgcgtagtagccagtaatgttggagcggccattcgtaaatggtaccaaagttccaaa

SEQ ID NO: 73 - DNA, 2295 bp, Homo sapiens
atgagtggggaccacctccacaacgattcccagatcgaagcggatttccgattgaatgattctcataaacacaaagataaacacaaagatcgagaacaccggcacaaagaacacaagaaggagaaggaccgggaaaagtccaagcatagcaacagtgaacataaagattctgaaaagaaacacaaagagaaggagaagaccaaacacaaagatggaagctcagaaaagcataaagacaaacataaagacagagacaaggaaaaacgaaaagaggaaaaggttcgagcctctggggatgcaaaaataaagaaggagaaggaaaatggcttctctagtccaccacaaattaaagatgaacctgaagatgatggctattttgttcctcctaaagaggatataaagccattaaagagacctcgagatgaggatgatgctgattataaacctaagaaaattaaaacagaagataccaagaaggagaagaaaagaaaactagaagaagaagaggatggtaaattgaaaaaacccaagaataaagataaagataaaaaagttcctgagccagataacaagaaaaagaagccgaagaaagaagaggaacagaagtggaaatggtgggaagaagagcgctatcctgaaggcatcaagtggaaattcctagaacataaaggtccagtatttgccccaccatatgagcctcttccagagaatgtcaagttttattatgatggtaaagtcatgaagctgagccccaaagcagaggaagtagctacgttctttgcaaaaatgctcgaccatgaatatactaccaaggaaatatttaggaaaaatttctttaaagactggagaaaggaaatgactaatgaagagaagaatattatcaccaacctaagcaaatgtgattttacccagatgagccagtatttcaaagcccagacggaagctcggaaacagatgagcaaggaagagaaactgaaaatcaaagaggagaatgaaaaattactgaaagaatatggattctgtattatggataaccacaaagagaggattgctaacttcaagatagagcctcctggacttttccgtggccgcggcaaccaccccaagatgggcatgctgaagagacgaatcatgcccgaggatataatcatcaactgtagcaaagatgccaaggttccttctcctcctccaggacataagtggaaagaagtccggcatgataacaaggttacttggctggtttcctggacagagaacatccaaggttccattaaatacatcatgcttaaccctagttcacgaatcaagggtgagaaggactggcagaaatacgagactgctcggcggctgaaaaaatgtgtggacaagatccggaaccagtatcgagaagactggaagtccaaagagatgaaagtccggcagagagctgtagccctgtacttcatcgacaagcttgctctgagagcaggcaatgaaaaggaggaaggagaaacagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtttttaagaacctacaactatttatggagaacaagcagcccgaggatgatctttttgatagactcaatactggtattctgaataagcatcttcaggatctcatggagggcttgacagccaaggtattccgtacatacaatgcctccatcacgctacagcagcagctaaaagaactgacagccccggatgagaacatcccagcgaagatcctttcttataaccgtgccaatcgagctgttgcaattctttgtaaccatcagagggcaccaccaaaaacttttgagaagtctatgatgaacttgcaaactaagattgatgccaagaaggaacagctagcagatgcccggagagacctgaaaagtgctaaggctgatgccaaggtcatgaaggatgcaaagacgaagaaggtagtagagtcaaagaagaaggctgttcagagactggaggaacagttgatgaagctggaagttcaagccacagaccgagaggaaaataaacagattgccctgggaacctccaaactcaattatctggaccctaggatcacagtggcttggtgcaagaagtggggtgtcccaattgagaagatttacaacaaaacccagcgggagaagtttgcctgggccattgacatggctgatgaagactatgagttt

SEQ ID NO: 74 - DNA, 1374 bp, Homo sapiens
atggcggcacagggagctgctgcggcggttgcggcggggacttcaggggtcgcgggggagggcgagcccgggcccggggagaatgcggccgctgaggggaccgccccatccccgggccgcgtctctccgccgaccccggcgcgcggcgagccggaagtcacggtggagatcggagaaacgtacctgtgccggcgaccggatagcacctggcattctgctgaagtgatccagtctcgagtgaacgaccaggagggccgagaggaattctatgtacactacgtgggctttaaccggcggctggacgagtgggtagacaagaaccggctggcgctgaccaagacagtgaaggatgctgtacagaagaactcagagaagtacctgagcgagctcgcagagcagcctgagcgcaagatcactcgcaaccaaaagcgcaagcatgatgagatcaaccatgtgcagaagacttatgcagagatggaccccaccacagcagccttggagaaggagcatgaggcgatcaccaaggtgaagtatgtggacaagatccacatcgggaactacgaaattgatgcctggtatttctcaccattccccgaagactatgggaaacagcccaagctctggctctgcgagtactgcctcaagtacatgaaatatgagaagagctaccgcttccacttgggtcagtgccagtggcggcagccccccgggaaagagatctaccgcaagagcaacatctccgtgtacgaagttgatggcaaagaccataagatttactgtcagaacctgtgtctgctggccaagcttttcctggaccataagacactgtactttgacgtggagccgttcgtcttttacatcctgactgaggtggaccggcagggggcccacattgttggctacttctccaaggagaaggagtccccggatggaaacaatgtggcctgcatcctgaccttgcccccctaccaacgccgcggctacgggaagttcctcatcgctttcagttatgagctctccaagctggagagcacagtcggctccccggagaagccactgtctgacctgggcaagctcagctaccgcagctactggtcctgggtgctgctagagatcctgcgggacttccggggcacactgtccatcaaggacctcagccagatgaccagtatcacccaaaatgacatcatcagtaccctgcaatccctcaatatggtcaagtactggaagggccagcacgtgatctgtgtcacacccaagctggtggaggagcacctcaaaagtgcccagtataagaaaccacccatcacagtggactccgtctgcctcaagtgggcaccccccaagcacaagcaagtcaagctctccaagaag

SEQ ID NO: 75 - DNA, 1053 bp, Homo sapiens
atggaaaggcggatgcagctggtaaagaaggataacgagaaagaaaggcacaagctgtttcagggctatgaaactgaagagagagaggaaacagagctatctgagaaaattaaactggagtgccagccggagctttccgagacatcccagactctgcctcccaagcccttctcatgtgggcggagtggaaagggacataaaaggaaatccccatttggaagtacagaaagaaagactcctgttaaaaagctggctcctgaattttcaaaagtcaaaacaaaaactcctaagcactctcctattaaagaggaaccctgtggttccttatctgaaactgtttgtaaacgtgaattgaggagccaagaaaccccagaaaagccccggtcttcagtggacaccccaccaagactctccactccccaaaagggacccagcacccatcccaaggagaaagccttctcaagtgagatagaagatttgccgtacctttccaccacagaaatgtatttgtgtcgttggcaccagcctcccccatcaccgttaccattacgggaatcctctccaaagaaggaggagactgtagcaaggtgtctgatgccatcaagtgttgcaggagaaacttcagtcttggctgttccttcttggagggaccactcagtagagcctctaagggacccaaatccttcagaccttttggagaacctggatgacagtgtgttttcgaagcggcatgcaaaactggagctggatgagaagagaaggaaaagatgggatattcagaggatcagggaacaaagaattttacagcgactgcagctcagaatgtataaaaagaaaggaattcaggaatctgagcctgaggttacctcatttttccctgagccagatgatgttgaaagtttgatgattacccccttcttgcctgttgtagcatttggacgaccattaccaaaattaactccacagaattttgagctaccctggttggatgagcgtagccgatgcagattggagatccagaagaagcaaacacctcaccggacgtgtaggaaa

SEQ ID NO: 76 - DNA, 1122 bp, Homo sapiens
atggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggac

SEQ ID NO: 77 - DNA, 1350 bp, Homo sapiens
atggccacctttagccgccaggaatttttccagcagctactgcaaggctgtctcctgcctactgcccagcagggccttgaccagatctggctgctccttgccatctgcctcgcctgccgcctcctctggaggctcgggttgccatcctacctgaagcatgcaagcaccgtggcaggcgggttcttcagcctctaccacttcttccagctgcacatggtttgggtcgtgctgctcagcctcctgtgctacctcgtgctgttcctctgccgacattcctcccatcgaggcgtcttcctatccgtcaccatcctcatctacctactcatgggtgagatgcacatggtagacaccgtgacatggcacaagatgcgaggggcacagatgattgtggccatgaaggcagtgtctctgggcttcgacctggaccggggcgaggtgggtacggtgccctcgccagtggagttcatgggctacctctacttcgtgggcaccatcgtcttcgggccctggatatccttccacagctacctacaagctgtccaaggccgcccactgagctgccggtggctgcagaaggtggcccggagcctggcactggccctgctgtgccttgtgctgtccacttgcgtgggcccctacctcttcccgtacttcatccccctcaacggtgaccgcctccttcgcaagtggctgcgagcctacgagagtgctgtctccttccacttcagcaactattttgtgggctttctttccgaggccacggccacgttggcgggggctggctttaccgaggagaaggatcacctggaatgggacctgacggtgtccaagccactgaatgtggagctgcctcggtcaatggtggaagttgtcacaagctggaacctgcccatgtcttattggctaaataactatgttttcaagaatgctctccgcctggggaccttctcggctgtgctggtcacctatgcagccagcgccctcctacatggcttcagtttccacctggctgcggtcctgctgtccctggcttttatcacttacgtggagcatgtcctccggaagcgcctggctcggatcctcagtgcctgtgtcttgtcaaagcggtgcccgccagactgttcgcaccagcatcgcttgggcctgggggtgcgagccttaaacttgctctttggagctctggccatcttccacctggcctacctgggctccctgtttgatgtcgatgtggatgacaccacagaggagcagggctacggcatggcatacactgtccacaagtggtcagagctcagctgggccagtcactgggtcacttttggatgctggatcttctaccgtctcataggc

SEQ ID NO: 78 - DNA, 1845 bp, Escherichia coli
atgccaattcaggtcttaccgccacaactggcgaaccagattgccgcaggtgaggtggtcgagcgacctgcgtcggtagtcaaagaactagtggaaaacagcctcgatgcaggtgcgacgcgtatcgatattgatatcgaacgcggtggggcgaaacttatccgcattcgtgataacggctgcggtatcaaaaaagatgagctggcgctggcgctggctcgtcatgccaccagtaaaatcgcctctctggacgatctcgaagccattatcagcctgggctttcgcggtgaggcgctggcgagtatcagttcggtttcccgcctgacgctcacttcacgcaccgcagaacagcaggaagcctggcaggcctatgccgaagggcgcgatatgaacgtgacggtaaaaccggcggcgcatcctgtggggacgacgctggaggtgctggatctgttctacaacaccccggcgcggcgcaaattcctgcgcaccgagaaaaccgaatttaaccacattgatgagatcatccgccgcattgcgctggcgcgtttcgacgtcacgatcaacctgtcgcataacggtaaaattgtgcgtcagtaccgcgcagtgccggaaggcgggcaaaaagaacggcgcttaggcgcgatttgcggcaccgcttttcttgaacaagcgctggcgattgaatggcaacacggcgatctcacgctacgcggctgggtggccgatccaaatcacaccacgcccgcactggcagaaattcagtattgctacgtgaacggtcgcatgatgcgcgatcgcctgatcaatcacgcgatccgccaggcctgcgaagacaaactgggggccgatcagcaaccggcatttgtgttgtatctggagatcgacccacatcaggtggacgtcaacgtgcaccccgccaaacacgaagtgcgtttccatcagtcgcgtctggtgcatgattttatctatcagggcgtgctgagcgtgctacaacagcaactggaaacgccgctaccgctggacgatgaaccccaacctgcaccgcgttccattccggaaaaccgcgtggcggcggggcgcaatcactttgcagaaccggcagctcgtgagccggtagctccgcgctacactcctgcgccagcatcaggcagtcgtccggctgccccctggccgaatgcgcagccaggctaccagaaacagcaaggtgaagtgtatcgccagcttttgcaaacgcccgcgccgatgcaaaaattaaaagcgccggaaccgcaggaacctgcacttgcggcgaacagtcagagttttggtcgggtactgactatcgtccattccgactgtgcgttgctggagcgcgacggcaacatttcacttttatccttgccagtggcagaacgttggctgcgtcaggcacaattgacgccgggtgaagcgcccgtttgcgcccagccgctgctgattccgttgcggctaaaagtttctgccgaagaaaaatcggcattagaaaaagcgcagtctgccctggcggaattgggtattgatttccagtcagatgcacagcatgtgaccatcagggcagtgcctttacccttacgccaacaaaatttacaaatcttgattcctgaactgataggctacctggcgaagcagtccgtattcgaacctggcaatattgcgcagtggattgcacgaaatctgatgagcgaacatgcgcagtggtcaatggcacaggccataaccctgctggcggacgtggaacggttatgtccgcaacttgtgaaaacgccgccgggtggtctgttacaatctgttgatttacatccggcgataaaagccctgaaagatgag

SEQ ID NO: 79 - DNA, 2022 bp, Homo sapiens
atggccccaggccaagcaccccatcaggctaccccgtggagggatgcccaccctttcttcctcctgtccccagtgatgggcctcctcagccgcgcctggagccgcctgaggggcctgggacctctagagccctggctggtggaagcagtaaaaggagcagctctggtagaagctggcctggagggagaagctaggactcctctggcaatcccccataccccttggggcagacgccctgaagaggaggctgaagacagtggaggccctggagaggacagagaaacactggggctgaaaaccagcagttcccttcctgaagcctggggacttttggatgatgatgatggcatgtatggtgagcgagaggcaaccagtgtccctagagggcagggaagtcaatttgcagatggccagcgtgctcccctgtctcccagccttctgataaggacactgcaaggttctgataagaacccaggggaggagaaagccgaggaagagggagttgctgaagaggagggagttaacaagttctcttatccaccatcacaccgggagtgttgtccagccgtggaggaggaggacgatgaagaagctgtaaagaaagaagctcacagaacctctacttctgccttgtctccaggatccaagcccagcacttgggtgtcttgcccaggggaggaagagaatcaagccacggaggataaaagaacagaaagaagtaaaggagccaggaagacctccgtgtccccccgatcttcaggctccgaccccaggtcctgggagtatcgttcaggagaggcgtccgaggagaaggaggaaaaggcacacaaagaaactgggaaaggagaagctgccccagggccgcaatcctcagccccagcccagaggccccagctcaagtcctggtggtgccaacccagtgatgaagaggagggtgaggtcaaggctttgggggcagctgagaaggatggagaagctgagtgtcctccctgcatccccccaccaagtgccttcctgaaggcctgggtgtattggccaggagaggacacagaggaagaggaagatgaggaagaagatgaggacagtgactctggatcagatgaggaagagggagaagctgaggcttcctcttccactcctgctacaggtgtcttcttgaagtcctgggtctatcagccaggagaggacacagaggaggaggaagatgaggacagtgatacaggatcagccgaggatgaaagagaagctgagacttctgcttccacaccccctgcaagtgctttcttgaaggcctgggtgtatcggccaggagaggacacggaggaggaggaagatgaggatgtggatagtgaggataaggaagatgattcagaagcagccttgggagaagctgagtcagacccacatccctcccacccggaccagagggcccacttcaggggctggggatatcgacctggaaaagagacagaggaagaggaagctgctgaggactggggagaagctgagccctgccccttccgagtggccatctatgtacctggagagaagccaccgcctccctgggctcctcctaggctgcccctccgactgcaaaggcggctcaagcgcccagaaacccctactcatgatccggaccctgagactcccctaaaggccagaaaggtgcgcttctccgagaaggtcactgtccatttcctggctgtctgggcagggccggcccaggccgcccgccagggcccctgggagcagcttgctcgggatcgcagccgcttcgcacgccgcatcacccaggcccaggaggagctgagcccctgcctcacccctgctgcccgggccagagcctgggcacgcctcaggaacccacctttagcccccatccctgccctcacccagaccttgccttcctcctctgtcccttcgtccccagtccagaccacgcccttgagccaagctgtggccacaccttcccgctcgtctgctgctgcagcggctgccctggacctcagtgggaggcgtggc

SEQ ID NO: 80 - DNA, 2370 bp, Homo sapiens
atggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggc

SEQ ID NO: 81 - DNA, 2931 bp, Homo sapiens
atgccggcccggcggctgctgctgctgctgacgctgctgctgcccggcctcgggatttttggaagtaccagcacagtgacgcttcctgaaaccttgttgtttgtgtcaacgctggatggaagtttgcatgctgtcagcaagaggacaggctcaatcaaatggactttaaaagaagatccagtcctgcaggtcccaacacatgtggaagagcctgcctttctcccagatcctaatgatggcagcctgtatacgcttggaagcaagaataatgaaggcctgacgaaacttccttttaccatcccagaattggtgcaggcatccccatgccgaagttcagatggaatcctctacatgggtaaaaagcaggacatctggtatgttattgacctcctgaccggagagaagcagcagactttgtcatcggcctttgcagatagtctctgcccatcaacctctcttctgtatcttgggcgaacagaatacaccatcaccatgtacgacaccaaaacccgagagctccggtggaatgccacctactttgactatgcggcctcactgcctgaggacgacgtggactacaagatgtcccactttgtgtccaatggtgatgggctggtggtgactgtggacagtgaatctggggacgtcctgtggatccaaaactacgcctcccctgtggtggccttttatgtctggcagcgggagggtctgaggaaggtgatgcacatcaatgtcgctgtggagaccctgcgctatctgaccttcatgtctggggaggtggggcgcatcacaaagtggaagtacccgttccccaaggagacagaggccaagagcaagctgacgcccactctgtatgttgggaaatactctaccagcctctatgcctctccctcaatggtacacgagggggttgctgtcgtgccccgcggcagcacacttcctttgctggaagggccccagactgatggcgtcaccattggggacaagggggagtgtgtgatcacgcccagcacggacgtcaagtttgatcccggactcaaaagcaagaacaagctcaactacttgaggaattactggcttctgataggacaccatgaaaccccactgtctgcgtctaccaagatgctggagagatttcccaacaatctacccaaacatcgggaaaatgtgattcctgctgattcagagaaaaagagctttgaggaagttatcaacctggttgaccagacttcagaaaacgcacctaccaccgtgtctcgggatgtggaggagaagcccgcccatgcccctgcccggcccgaggcccccgtggactccatgcttaaggacatggctaccatcatcctgagcaccttcctgctgattggctgggtggccttcatcatcacctatcccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctc

SEQ ID NO: 82 - DNA, 2196 bp, Homo sapiens
atgacggagggcacgtgtctgcggcgccgagggggcccctacaagaccgagcccgccaccgacctcggccgctggcgactcaactgcgagaggggccggcagacgtggacctacctgcaggacgagcgcgccggccgcgagcagaccggcctggaagcctacgccctggggctggacaccaagaattactttaaggacttgcccaaagcccacaccgcctttgagggggctctgaacgggatgacattttacgtggggctgcaggctgaggatgggcactggacgggtgattatggtggcccacttttcctcctgccaggcctcctgatcacttgccacgtggcacgcatccctctgccagccggatacagagaagagattgtgcggtacctgcggtcagtgcagctccctgacggtggctggggcctgcacattgaggataagtccaccgtgtttgggactgcgctcaactatgtgtctctcagaattctgggtgttgggcctgacgatcctgacctggtacgagcccggaacattcttcacaagaaaggtggtgctgtggccatcccctcctgggggaagttctggctggctgtcctgaatgtttacagctgggaaggcctcaataccctgttcccagagatgtggctgtttcctgactgggcaccggcacacccctccacactctggtgccactgccggcaggtgtacctgcccatgagctactgctacgccgttcggctgagtgccgcggaagacccgctggtccagagcctccgccaggagctctatgtggaggacttcgccagcattgactggctggcgcagaggaacaacgtggcccccgacgagctgtacacgccgcacagctggctgctccgcgtggtatatgcgctcctcaacctgtatgagcaccaccacagtgcccacctgcggcagcgggccgtgcagaagctgtatgaacacattgtggccgacgaccgattcaccaagagcatcagcatcggcccgatctcgaaaaccatcaacatgcttgtgcgctggtatgtggacgggcccgcctccactgccttccaggagcatgtctccagaatcccggactatctctggatgggccttgacggcatgaaaatgcagggcaccaacggctcacagatctgggacaccgcattcgccatccaggctctgcttgaggcgggcgggcaccacaggcccgagttttcgtcctgcctgcagaaggctcatgagttcctgaggctctcacaggtcccagataaccctcccgactaccagaagtactaccgccagatgcgcaagggtggcttctccttcagtacgctggactgcggctggatcgtttctgactgcacggctgaggccttgaaggctgtgctgctcctgcaggagaagtgtccccatgtcaccgagcacatccccagagaacggctctgcgatgctgtggctgtgctgctgaacatgagaaatccagatggagggttcgccacctatgagaccaagcgtggggggcacttgctggagctgctgaacccctcggaggtcttcggggacatcatgattgactacacctatgtggagtgcacctcagccgtgatgcaggcgcttaagtatttccacaagcgtttcccggagcacagggcagcggagatccgggagaccctcacgcagggcttagagttctgtcggcggcagcagagggccgatggctcctgggaaggctcctggggagtttgcttcacctacggcacctggtttggcctggaggccttcgcctgtatggggcagacctaccgagatgggactgcctgtgcagaggtctcccgggcctgtgacttcctgctgtcccggcagatggcagacggaggctggggggaggactttgagtcctgcgaggagcggcgttatttgcagagtgcccagtcccagatccataacacatgctgggccatgatggggctgatggccgttcggcatcctgacatcgaggcccaggagagaggagtccggtgtctacttgagaaacagctccccaatggcgactggccgcaggaaaacattgctggggtcttcaacaagtcctgtgccatctcctacacgagctacaggaacatcttccccatctgggccctcggccgcttctcccagctgtaccctgagagagcccttgctggccacccc

SEQ ID NO: 83 - DNA, 1179 bp, Homo sapiens
atgcgcgggcgcgggcgagcagggtctccgggtgggcggcggcgacgccccgcgcaggctggaggccgccgaggctcgccatgccgggagaactctaactcccccatggagtcggccgacttctacgaggcggagccgcggcccccgatgagcagccacctgcagagccccccgcacgcgcccagcagcgccgccttcggctttccccggggcgcgggccccgcgcagcctcccgccccacctgccgccccggagccgctgggcggcatctgcgagcacgagacgtccatcgacatcagcgcctacatcgacccggccgccttcaacgacgagttcctggccgacctgttccagcacagccggcagcaggagaaggccaaggcggccgtgggccccacgggcggcggcggcggcggcgactttgactacccgggcgcgcccgcgggccccggcggcgccgtcatgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcg

SEQ ID NO: 84 - DNA, 1128 bp, Homo sapiens
atggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtc

SEQ ID NO: 85 - DNA, 561 bp, Homo sapiens
atggtggcggcgacggtggcagcggcgtggctgctcctgtgggctgcggcctgcgcgcagcaggagcaggacttctacgacttcaaggcggtcaacatccggggcaaactggtgtcgctggagaagtaccgcggatcggtgtccctggtggtgaatgtggccagcgagtgcggcttcacagaccagcactaccgagccctgcagcagctgcagcgagacctgggcccccaccactttaacgtgctcgccttcccctgcaaccagtttggccaacaggagcctgacagcaacaaggagattgagagctttgcccgccgcacctacagtgtctcattccccatgtttagcaagattgcagtcaccggtactggtgcccatcctgccttcaagtacctggcccagacttctgggaaggagcccacctggaacttctggaagtacctagtagccccagatggaaaggtggtaggggcttgggacccaactgtgtcagtggaggaggtcagaccccagatcacagcgctcgtgaggaagctcatcctactgaagcgagaagactta

SEQ ID NO: 86 - DNA, 1215 bp, Homo sapiens
atgcaggaaaaagacgcctcctcacaaggtttcctgccacacttccaacatttcgccacgcaggcgatccatgtgggccaggatccagagcaatggacctccagggctgtagtgccccccatctcactgtccaccacgttcaagcaaggggcgcctggccagcactcgggttttgaatatagccgttctggaaatcccactaggaattgccttgaaaaagcagtggcagcactggatggggctaagtactgtttggcctttgcttcaggtttagcagccactgtaactattacccatcttttaaaagcaggagaccaaattatttgtatggatgatgtgtatggaggtacaaacaggtacttcaggcaagtggcatctgaatttggattaaagatttcttttgttgattgttccaaaatcaaattactagaggcagcaattacaccagaaaccaagcttgtttggatcgaaacccccacaaaccccacccagaaggtgattgacattgaaggctgtgcacatattgtccataagcatggagacattattttggtcgtggataacacttttatgtcaccatatttccagcgccctttggctctgggagctgatatttctatgtattctgcaacaaaatacatgaatggccacagtgatgttgtaatgggcctggtgtctgttaattgtgaaagccttcataatagacttcgtttcttgcaaaactctcttggagcagttccatctcctattgattgttacctctgcaatcgaggtctgaagactctacatgtccgaatggaaaagcatttcaaaaacggaatggcagttgcccagttcctggaatctaatccttgggtagaaaaggttatttatcctgggctgccctctcatccacagcatgagttggtgaagcgtcagtgtacaggttgtacagggatggtcaccttttatattaagggcactcttcagcatgctgagattttcctcaagaacctaaagctatttactctggccgagagcttgggaggattcgaaagccttgctgagcttccggcaatcatgactcatgcatcagttcttaagaatgacagagatgtccttggaattagtgacacactgattcgactttctgtgggcttagaggatgaggaagacctactggaagatctagatcaagctttgaaggcagcacaccctccaagtggaagtcacagc

SEQ ID NO: 87 - DNA, 1653 bp, Homo sapiens
atgccttctgagaccccccaggcagaagtggggcccacaggctgcccccaccgctcagggccacactcggcgaaggggagcctggagaaggggtccccagaggataaggaagccaaggagcccctgtggatccggcccgatgctccgagcaggtgcacctggcagctgggccggcctgcctccgagtccccacatcaccacactgccccggcaaaatctccaaaaatcttgccagatattctgaagaaaatcggggacacccctatggtcagaatcaacaagattgggaagaagttcggcctgaagtgtgagctcttggccaagtgtgagttcttcaacgcgggcgggagcgtgaaggaccgcatcagcctgcggatgattgaggatgctgagcgcgacgggacgctgaagcccggggacacgattatcgagccgacatccgggaacaccgggatcgggctggccctggctgcggcagtgaggggctatcgctgcatcatcgtgatgccagagaagatgagctccgagaaggtggacgtgctgcgggcactgggggctgagattgtgaggacgcccaccaatgccaggttcgactccccggagtcacacgtgggggtggcctggcggctgaagaacgaaatccccaattctcacatcctagaccagtaccgcaacgccagcaaccccctggctcactacgacaccaccgctgatgagatcctgcagcagtgtgatgggaagctggacatgctggtggcttcagtgggcacgggcggcaccatcacgggcattgccaggaagctgaaggagaagtgtcctggatgcaggatcattggggtggatcccgaagggtccatcctcgcagagccggaggagctgaaccagacggagcagacaacctacgaggtggaagggatcggctacgacttcatccccacggtgctggacaggacggtggtggacaagtggttcaagagcaacgatgaggaggcgttcacctttgcccgcatgctgatcgcgcaagaggggctgctgtgcggtggcagtgctggcagcacggtggcggtggccgtgaaggccgcgcaggagctgcaggagggccagcgctgcgtggtcattctgcccgactcagtgcggaactacatgaccaagttcctgagcgacaggtggatgctgcagaagggctttctgaaggaggaggacctcacggagaagaagccctggtggtggcacctccgtgttcaggagctgggcctgtcagccccgctgaccgtgctcccgaccatcacctgtgggcacaccatcgagatcctccgggagaagggcttcgaccaggcgcccgtggtggatgaggcgggggtaatcctgggaatggtgacgcttgggaacatgctctcgtccctgcttgccgggaaggtgcagccgtcagaccaagttggcaaagtcatctacaagcagttcaaacagatccgcctcacggacacgctgggcaggctctcgcacatcctggagatggaccacttcgccctggtggtgcacgagcagatccagtaccacagcaccgggaagtccagtcagcggcagatggtgttcggggtggtcaccgccattgacttgctgaacttcgtggccgcccaggagcgggaccagaag

SEQ ID NO: 88 - DNA, 618 bp, Homo sapiens
atgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctg

SEQ ID NO: 89 - DNA, 1335 bp, Homo sapiens
atgctgaagaagcagtctgcagggcttgtgctgtggggcgctatcctctttgtggcctggaatgccctgctgctcctcttcttctggacgcgcccagcacctggcaggccaccctcagtcagcgctctcgatggcgaccccgccagcctcacccgggaagtgattcgcctggcccaagacgccgaggtggagctggagcggcagcgtgggctgctgcagcagatcggggatgccctgtcgagccagcgggggagggtgcccaccgcggcccctcccgcccagccgcgtgtgcctgtgacccccgcgccggcggtgattcccatcctggtcatcgcctgtgaccgcagcactgttcggcgctgcctggacaagctgctgcattatcggccctcggctgagctcttccccatcatcgttagccaggactgcgggcacgaggagacggcccaggccatcgcctcctacggcagcgcggtcacgcacatccggcagcccgacctgagcagcattgcggtgccgccggaccaccgcaagttccagggctactacaagatcgcgcgccactaccgctgggcgctgggccaggtcttccggcagtttcgcttccccgcggccgtggtggtggaggatgacctggaggtggccccggacttcttcgagtactttcgggccacctatccgctgctgaaggccgacccctccctgtggtgcgtctcggcctggaatgacaacggcaaggagcagatggtggacgccagcaggcctgagctgctctaccgcaccgactttttccctggcctgggctggctgctgttggccgagctctgggctgagctggagcccaagtggccaaaggccttctgggacgactggatgcggcggccggagcagcggcaggggcgggcctgcatacgccctgagatctcaagaacgatgacctttggccgcaagggtgtgagccacgggcagttctttgaccagcacctcaagtttatcaagctgaaccagcagtttgtgcacttcacccagctggacctgtcttacctgcagcgggaggcctatgaccgagatttcctcgcccgcgtctacggtgctccccagctgcaggtggagaaagtgaggaccaatgaccggaaggagctgggggaggtgcgggtgcagtatacgggcagggacagcttcaaggctttcgccaaggctctgggtgtcatggatgaccttaagtcgggggttccgagagctggctaccggggtattgtcaccttccagttccggggccgccgtgtccacctggcgcccccaccgacgtgggagggctatgatcctagctggaat

SEQ ID NO: 90 - DNA, 1341 bp, Homo sapiens
atgaggttccgcatctacaaacggaaggtgctaatcctgacgctcgtggtggccgcctgcggcttcgtcctctggagcagcaatgggcgacaaaggaagaacgaggccctcgccccaccgttgctggacgccgaacccgcgcggggtgccggcggccgcggtggggaccacccctctgtggctgtgggcatccgcagggtctccaacgtgtcggcggcttccctggtcccggcggtcccccagcccgaggcggacaacctgacgctgcggtaccggtccctggtgtaccagctgaactttgatcagaccctgaggaatgtagataaggctggcacctgggccccccgggagctggtgctggtggtccaggtgcataaccggcccgaatacctcagactgctgctggactcacttcgaaaagcccagggaattgacaacgtcctcgtcatctttagccatgacttctggtcgaccgagatcaatcagctgatcgccggggtgaatttctgtccggttctgcaggtgttctttcctttcagcattcagttgtaccctaacgagtttccaggtagtgaccctagagattgtcccagagacctgccgaagaatgccgctttgaaattggggtgcatcaatgctgagtatcccgactccttcggccattatagagaggccaaattctcccagaccaaacatcactggtggtggaagctgcattttgtgtgggaaagagtgaaaattcttcgagattatgctggccttatacttttcctagaagaggatcactacttagccccagacttttaccatgtcttcaaaaagatgtggaaactgaagcagcaagagtgccctgaatgtgatgttctctccctggggacctatagtgccagtcgcagtttctatggcatggctgacaaggtagatgtgaaaacttggaaatccacagagcacaatatgggtctagccttgacccggaatgcctatcagaagctgatcgagtgcacagacactttctgtacttatgatgattataactgggactggactcttcaatacttgactgtatcttgtcttccaaaattctggaaagtgctggttcctcaaattcctaggatctttcatgctggagactgtggtatgcatcacaagaaaacctgtagaccatccactcagagtgcccaaattgagtcactcttaaataataacaaacaatacatgtttccagaaactctaactatcagtgaaaagtttactgtggtagccatttccccacctagaaaaaatggagggtggggagatattagggaccatgaactctgtaaaagttatagaagactgcag

SEQ ID NO: 91 - DNA, 954 bp, Homo sapiens
atgccgaagcgtgggaaaaagggagcggtggcggaagacggggatgagctcaggacagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagattgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactg

SEQ ID NO: 92 - DNA, 387 bp, Escherichia coli
atgctggtttcttgcgcaatgcgacttcactcgggcgtttttccagattatgctgaaaaattacctcaggaagaaaagatggaaaaagaagattcatttccccaacgcgtctggcaaatcgtcgccgctattcccgaaggctatgtcaccacttacggtgatgtggcgaaactggcgggatcgccccgcgccgcgcgccaggtgggcggtgtgttaaagcgtctccctgaaggcagcaccttaccctggcaccgggtggttaatcgccacggcacaatttcgctaaccggaccggatttacagcgtcagcgacaggcattactggcagaaggtgtgatggtatcgggaagcgggcaaatcgacttgcagcgttatcgctggaactac

SEQ ID NO: 93 - DNA, 1851 bp, Homo sapiens
atgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggagactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccac

SEQ ID NO: 94 - DNA, 1449 bp, Homo sapiens
atggaggaaggaggcggcggcgtacggagtctggtcccgggcgggccggtgttactggtcctctgcggcctcctggaggcgtccggcggcggccgagcccttcctcaactcagcgatgacatccctttccgagtcaactggcccggcaccgagttctctctgcccacaactggagttttatataaagaagataattatgtcatcatgacaactgcacataaagaaaaatataaatgcatacttccccttgtgacaagtggggatgaggaagaagaaaaggattataaaggccctaatccaagagagcttttggagccactatttaaacaaagcagttgttcctacagaattgagtcttattggacttacgaagtatgtcatggaaaacacattcggcagtaccatgaagagaaagaaactggtcagaaaataaatattcacgagtactaccttgggaatatgttggccaagaaccttctatttgaaaaagaacgagaagcagaagaaaaggaaaaatcaaatgagattcccactaaaaatatcgaaggtcagatgacaccatactatcctgtgggaatgggaaatggtacaccttgtagtttgaaacagaaccggcccagatcaagtactgtgatgtacatatgtcatcctgaatctaagcatgaaattctttcagtagctgaagttacaacttgtgaatatgaagttgtcattttgacaccactcttgtgcagtcatcctaaatataggttcagagcatctcctgtgaatgacatattttgtcaatcactgccaggatctccatttaagcccctcaccctgaggcagctggagcagcaggaagaaatactaagggtgccttttaggagaaataaagaggaagatttgcaatcaactaaagaagagagatttccagcgatccacaagtcgattgctattggctctcagccagtgctcactgttgggacaacccacatatccaaattgacagatgaccaactcataaaagagtttcttagtggttcttactgctttcgtgggggtgtcggttggtggaaatatgaattctgctatggcaaacatgtacatcaataccatgaggacaaggatagtgggaaaacctctgtggttgtcgggacatggaaccaagaagagcatattgaatgggctaagaagaatactgctagagcttatcatcttcaagacgatggtacccagacagtcaggatggtgtcacatttttatggaaatggagatatttgtgatataactgacaaaccaagacaggtgactgtaaaactaaagtgcaaagaatcagattcacctcatgctgttactgtatatatgctagagcctcactcctgtcaatatattcttggggttgaatctccagtgatctgtaaaatcttagatacagcagatgaaaatggacttctttctctccccaac

SEQ ID NO: 95 - DNA, 2184 bp, Homo sapiens
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtca

SEQ ID NO: 96 - DNA, 312 bp, Homo sapiens
atggctggcaaagcacacaggctgagcgctgaggagagggaccagctgctgccaaacctgagggctgtggggtggaatgagctggaaggccgtgatgccatcttcaagcagtttcatttcaaagacttcaacagggcctttgggttcatgacaagagtggccctgcaggctgagaaactggaccaccatcctgaatggtttaacgtgtacaacaaggtccacatcacgctgagcacccatgagtgtgccggcctttcagaacgggacataaacctggccagcttcatcgaacaagtagcagtgtccatgaca

SEQ ID NO: 97 - DNA, 444 bp, Homo sapiens
atggcccgggccctgtgccgcctcccgcggcgcggcctctggctgctcctggcccatcacctcttcatgaccactgcctgccaggaggctaactacggtgccctcctccgggagctctgcctcacccagttccaggtagacatggaggccgtcggggagacgctgtggtgtgactggggcaggaccatcaggagctacagggagctggccgactgcacctggcacatggcggagaagctgggctgcttctggcccaatgcagaggtggacaggttcttcctggcagtgcatggccgctacttcaggagctgccccatctcaggcagggccgtgcgggacccgcccggcagcatcctctaccccttcatcgtggtccccatcacggtgaccctgctggtgacggcactggtggtctggcagagcaagcgcactgagggcattgtg

SEQ ID NO: 98 - DNA, 525 bp, Homo sapiens
atggcctcgctccgggtggagcgcgccggcggcccgcgtctccctaggacccgagtcgggcggccggcagcgctccgcctcctcctcctgctgggcgctgtcctgaatccccacgaggccctggctcagcctcttcccaccacaggcacaccagggtcagaaggggggacggtgaagaactatgagacagctgtccaattttgctggaatcattataaggatcaaatggatcctatcgaaaaggattggtgcgactgggccatgattagcaggccttatagcaccctgcgagattgcctggagcactttgcagagttgtttgacctgggcttccccaatcccttggcagagaggatcatctttgagactcaccagatccactttgccaactgctccctggtgcagcccaccttctctgaccccccagaggatgtactcctggccatgatcatagcccccatctgcctcatccccttcctcatcactcttgtagtatggaggagtaaagacagtgaggcccaggcc

SEQ ID NO: 99 - DNA, 444 bp, Homo sapiens
atggagactggagcgctgcggcgcccgcaacttctcccgttgctgctgctgctctgcggtgggtgtcccagagcaggcggctgcaacgagacaggcatgttggagaggctgcccctgtgtgggaaggctttcgcagacatgatgggcaaggtggacgtctggaagtggtgcaacctgtccgagttcatcgtgtactatgagagtttcaccaactgcaccgagatggaggccaatgtcgtgggctgctactggcccaaccccctggcccagggcttcatcaccggcatccacaggcagttcttctccaactgcaccgtggacagggtccacttggaggaccccccagacgaggttctcatcccgctgatcgttatacccgtcgttctgactgtcgccatggctggcctggtggtgtggcgcagcaaacgcaccgacacgctgctg

SEQ ID NO: 100 - DNA, 1644 bp, Escherichia coli
atggattcgcaacgcaatcttttagtcatcgctttgctgttcgtgtctttcatgatctggcaagcctgggagcaggataaaaacccgcaacctcaggcccaacagaccacgcagacaacgaccaccgcagcgggtagcgccgccgaccagggcgtaccggccagtggccaggggaaactgatctcggttaagaccgacgtgcttgatctgaccatcaacacccgtggtggtgatgttgagcaagctctgctgcctgcttacccgaaagagctgaactctacccagccgttccagctgttggaaacttcaccgcagtttatttatcaggcacagagcggtctgaccggtcgtgatggcccggataacccggctaacggcccgcgtccgctgtataacgttgaaaaagacgcttatgtgctggctgaaggtcaaaacgaactgcaggtgccgatgacgtataccgacgcggcaggcaacacgtttaccaaaacgtttgtcctgaaacgtggtgattacgctgtcaacgtcaactacaacgtgcagaacgctggcgagaaaccgctggaaatctcctcgtttggtcagttgaagcaatccatcactctgccaccgcatctcgataccggaagcagcaacttcgcactgcacaccttccgtggcgcggcgtactccacgcctgacgagaagtatgagaaatacaagttcgataccattgccgataacgaaaacctgaacatctcttcgaaaggtggttgggtggcgatgctgcaacagtatttcgcgacggcgtggatcccgcataacgacggtaccaacaacttctataccgctaatctgggtaacggcatcgccgctatcggctataaatctcagccggtactggttcagcctggtcagactggcgcgatgaacagcaccctgtgggttggcccggaaatccaggacaaaatggcagctgttgctccgcacctggatctgaccgttgattacggttggttgtggttcatctctcagccgctgttcaaactgctgaaatggatccatagctttgtgggtaactggggcttctccattatcatcatcacctttatcgttcgtggcatcatgtacccgctgaccaaagcgcagtacacctccatggcgaagatgcgtatgttgcagccgaagattcaggcaatgcgtgagcgtctgggcgatgacaaacagcgtatcagccaggaaatgatggcgctgtacaaagctgagaaggttaacccgctgggcggctgcttcccgctgctgatccagatgccaatcttcctggcgttgtactacatgctgatgggttccgttgaactgcgtcaggcaccgtttgcactgtggatccacgacctgtcggcacaggacccgtactacatcctgccgatcctgatgggcgtaacgatgttcttcattcagaagatgtcgccgaccacagtgaccgacccgatgcagcagaagatcatgacctttatgccggtcatcttcaccgtgttcttcctgtggttcccgtcaggtctggtgctgtactatatcgtcagcaacctggtaaccattattcagcagcagctgatttaccgtggtctggaaaaacgtggcctgcatagccgcgagaagaaaaaatcc

SEQ ID NO: 101 - DNA, 1194 bp, Homo sapiens
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagc

SEQ ID NO: 102 - DNA, 1500 bp, Homo sapiens
atgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaagcagcagaatctccggaccagaaagacacagatggagggcccaaggaggaggaaagtccagtc

SEQ ID NO: 103 - DNA, 1119 bp, Homo sapiens
atgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctctagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaat

SEQ ID NO: 104 - DNA, 1725 bp, Homo sapiens
atgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccg

SEQ ID NO: 105 - DNA, 420 bp, Homo sapiens
atggccgtagtgttgccggcggttgtggaggagctcctgagcgagatggcggcggcggtgcaggagagcgcgcgaattcctgatgaatatctgttatcgctgaagtttctctttggctcatcagccacccaggccttggacctagttgatcgacagtccatcaccttaatctcatcacccagtggaaggcgtgtttaccaggtccttggaagttccagtaaaacatacacatgtttggcttcttgtcattactgttcatgtcctgcatttgcattctcagtgctacggaagagtgacagcatcctgtgcaagcatctcttggcagtttacctgagtcaggttatgaggacctgtcagcagctaagtgtctctgacaagcagttgactgacatattattgatggagaagaaacaagaagca

SEQ ID NO: 106 - DNA, 1011 bp, Homo sapiens
atggctgccccgagagacaatgtcactttattattcaagttatactgcttggcagtgatgaccctgatggctgcagtctataccatagctttaagatacacaaggacatcagacaaagaactctacttttcaaccacagccgtgtgtatcacagaagttataaagttattgctaagtgtgggaattttagctaaagaaactggtagtctgggtagattcaaagcatctttaagagaaaatgtcttggggagccccaaggaactgttgaagttaagtgtgccatcgttagtgtatgctgttcagaacaacatggctttcctagctcttagcaatctggatgcagcagtgtaccaggtgacctaccagttgaagattccgtgtactgctttatgcactgttttaatgttaaaccggacactcagcaaattacagtgggtttcagtttttatgctgtgtgctggagttacgcttgtacagtggaaaccagcccaagctacaaaagtggtggtggaacaaaatccattattagggtttggcgctatagctattgctgtattgtgctcaggatttgcaggagtatattttgaaaaagttttaaagagttcagatacttctctttgggtgagaaacattcaaatgtatctatcagggattattgtgacattagctggcgtctacttgtcagatggagctgaaattaaagaaaaaggatttttctatggttacacatattatgtctggtttgtcatctttcttgcaagtgttggtggcctctacacttctgttgtggttaagtacacagacaacatcatgaaaggcttttctgcagcagcggccattgtcctttccaccattgcttcagtaatgctgtttggattacagataacactcacctttgccctgggtactcttcttgtatgtgtttccatatatctctatggattacccagacaagacactacatccatccaacaaggagaaacagcttcaaaggagagagttattggtgtg

SEQ ID NO: 107 - DNA, 2196 bp, Cricetulus griseus
atgtctttctcatgctctccaggccagcaggtggccatcaaaaagatacccaatgcttttgatgtggtgaccaatgccaaacggaccctcagggaactgaagatcctcaaacacttcaaacatgacaatatcatcgccatcaaggacatcctgaggcccactgtgccctatggagagttcaaatctgtctatgtagtactggacctcatggagagtgacctacaccagatcatccactcctcacagccactcacactggagcatgtgcgatacttcttgtaccagctgcttcggggcctcaagtatatgcactctgctcaggtcatccaccgtgaccttaaaccctctaaccttttggtgaatgagaactgtgagctcaagattggtgactttggaatggcccgtgggctgtgtacctcccctgctgaacaccagtacttcatgactgagtacgtggccactcgctggtaccgtgcccctgaactcatgctttccctgcatgagtacacacaggctattgacctctggtctgtgggctgcatctttggtgagatgctggctcggcgacaactcttcccaggcaaaaactatgtgcaccagttacagctgatcatgatggtgttgggaaccccatcaccagctgtgattcaggctgtgggggctgaaagggtgcgggcatatatccagagcctgccaccacggcagcctgtaccctgggagacagtatacccaggtgctgaccgccaggccctctcactgctgggccgcaagaagccatttgaacccagtgcccggatctcagctgctgctgcccttcgtcaccccttcctggctaagtaccatgaccctgatgatgagcctgattgtgccccaccttttgactttgcttttgaccgtgaagcccttactagggagcgcattaaggaagccattgtggctgagattgaggacttccatgcacgacgggagggcatccgccaacaaatccgcttccagccttctctgcagcctgtggctagtgagcctgtgtgtccagatgttgagatgcccagtccctgggctccaagtggagactgtgccatggagtcacctcctccagcaccaccaccatgctctggccctgcacctgacactgttgatctgactctgaagcctgcgcccccagccagtgagcttgccccaccaaaaagagagggtgccatctcagacaacaccaaagcagccctcaaagctgccctgctcaagtccctgaggagccgactcagagatggccccagtgcacccttggaggcacctgaacctcgaaagcctgtgacagctcaggaacgccagcgagagcgagaagagaagcgtaggaggcgacaagagagagccaaggagagggagaagcgacgacaagagagggaacgcaaggagagaggagctggtgccttgggggggccctctactgaccctctggctggactggtgcttagtgacaatgaccgaagcctgctagagcgtcgccctcctgccccaactccagccccagccccagctccatcccctgcccaacctgctagtcctcccactggccctgtatctcagtctgctggtcctcctctacagcctgcaggctctattcctggtcctgcctcccagcctgtttgcccacccactggccctgctccccagccttctggccctcctggacctatccctgccccactccaaactgccccttccactagccttctggcctctcagtcacttgtgccaactagtgggttgcctggctccagtgccccagaagttctctcatacttcccatctggcccaccacctccagatcctgggggcccccctcagccttctacatcagagtcaccagatgtcaacctggtgacccagcaactgtctaagtctcaggtggaggaccccctgcctcctgtgttttcaggcactccaaagggcagtggggctggctacggtgttggctttgacctagaggaattcttaaaccaatctttcgatatgggtgtggctgatgggccacaggatggccaggcagactcagcttcactctcagcctccctcctggctgactggcttgagggccacggcatgaacccggctgacatcgagtctctgcagcgtgaaatccagatggactccccaatgctgctggctgacctgcctgacctccaggagccctga

SEQ ID NO: 108 - DNA, 1683 bp, Cricetulus griseus
atgcccatccagatcttttgctctgtgtcattctcctctggagaagaggccccaggatccatgggagatatctggggtcctcatcaccaccggcagcagcaggacagctcagaatcagaagaggaagaggaagagaaggaaaaagaggcactgaagagggagcttgatgacggagactcacctagggacttggtggccttcgccaacagttgtaccctccatggtgccagccatgtctttgtggaagggggcccagggccaaggcaggccctgtgggcagtggcttttgtcatagcactgggtgccttcctgtgccaggtaggggaccgagttgcttattacctcagctatccacatgtgactttgctagatgaagtcgccaccacagagctggtcttcccggcggtcacattctgtaacaccaatgccgtgcggttgtcccagctcagctaccctgatctgctctacctggcccccatgctagggctggatgaaagtgatgatcctggggtgccccttgctccaccggggccagaggccttctccggggagccttttaatctccatcgtttctacaatcgctcctgccaccggctggaggacatgctgctctattgttcctactgtgggggcccctgtggtccccacaacttctcagtggtcttcacacggtatgggaagtgttacacattcaactcgggccaagatgggcggccacggctgaagacaatgaaaggtgggactggcaatggcctggagatcatgctggacatccagcaagatgaatacctgcctgtgtggggagagaccgacgagacatccttcgaagcaggtatcaaagtgcagatccacagccaggatgaacctcctttcatcgaccagctgggctttggtgtggccccaggcttccagacctttgtgtcttgccaggagcagaggctcatctacctgccctcaccctggggcacctgcaatgctgttaccatggactcggatttcttcgactcctacagtatcaccgcctgccgcattgattgtgagacacgttacctggtggaaaactgcaactgccgtatggtacacatgccaggggatgctccatactgcactccagagcagtacaaggagtgtgcagatcctgccctggacttcctagtggagaaagaccaggaatactgcgtgtgtgaaatgccctgcaacctgacacgctatggcaaggagctgtccatggtcaagatacccagcaaagcctcagccaagtacctggccaagaagttcaacaaatctgagcagtacataggggagaacatcctggtgctggatattttctttgaagtcctcaactatgagaccatcgagcagaaaaaggcctatgagatcgcagggctcttgggtgacatcgggggccagatggggttgttcatcggggccagcatcctcacagtgctggaactctttgactatgcctatgaggtcattaagcaccgtttgtgtagacgagggaaatgccagaaggaggccaagaggagcagcgcagataagggcgtagcgctcagcctggacgacgtcaaaagacacaatccctgcgagagcctccggggacaccctgccgggatgacatacgctgccaacatcctacctcaccatcctgctcgaggcacctttgaggactttacctgctaa

SEQ ID NO: 109 - DNA, 1974 bp, Cricetulus griseus
atggcgctgctgaatggacttgcctccggggctgtgccgtcagtgcggctgcgcgcgagccgggtgccgcggtacttaagttggctgcacacactgtcagaggtagccatgaggagagcagatcccgagcagcccgctaagcggcctcgctgcgacagcagcccaagaacgccacagagaacccctcctgcagcggccagcgcgtctcccggctccgagctccacatcgacctccagacctgggaagtggaggacgtgtgctcctacctaaagggatgcggcttccaagacaagaaagtgctggacagcttcaaagaaaatgaaatcactggtccatttttgccctttctcgatgagtcacgtcttgaaaaacttggagtaagttccttggaggagaggaagaacatgattgcatgtatccaagaactgagtcaatctcaagtagagctaatgaaggtatttaatgatcctattcatggccatattgagttccaccctctccttatccgaatcattgacacacctcagtttcagcggcttcgatacatcaaacagctgggaggcggctactatgttttcccaggagcttcacacaatcgctttgaacacagtctaggggttgggtacctagcaggctacctcgttcgaacacttgcagaaaaacagccagagctaaagatcagtgagcgagatatgctctgtgttcagattgccggactctgccatgaccttggtcatgggccattttctcatatgtttgatggaagatttatcccatctgctcgccgaggtgaaacgtggaagcatgaacagggatcagctaagatgtttgaacatctggttaattctaatgaactcggacctatcatgaaaaactatgggctcaaccctgaagaagatattgtcttcattaaggaacaaattgagggaccaacttcaactccaatcaaggactgctcatggccatataaaggacgccctgccgagaagagcttcctttatgagatagtgtctaacaaaagaaatggcattgatgtggacaaatgggattattttgccagggactgtcaccatcttggaatccaaaacaattttgattacaagcgctatgttaagtttgtccgtgtctgtgaggtggaagaatcttgtggctgcaagatgaagcacatttgtaccagagaaaaggaggttgggaatctgtatgacatgttccacacacgcaactgcttacaccgcagagcttaccaacataaaatcagcaacctcatcgatataatgatcacagaggccttctgcaaagcagacccctacatggagattctgggggccaaggggaagaagttcagtatttctacagccattgatgacatggaagccttcaccaagctgacggataacattttcctggaaattttatactctactgatccaaatttgtctgaggcaagaaatattttaaggaacatcgaatgccgtaatctatacaagtatttgggtgagacccagccaaacggtcaagacaagattacaggggaagaatatgatcagcttccccgagacattgttaacaccaaacctgatgtgcccctgtatgcccaactgaaggctgaagatttcatagtggatgttatcaacatggattatgggatgaaagacaagaatccaattgaccatgttcacttctattgtaaaaataacacgaagcacgcagtcaagatctctaaagatcaggtgtcaaagcttctgccacagaaattcacagatcagctcattcgagtgtactgtaagaagaaagacaagaagagcctgcatgctgctaggcaacatttcactcaatggtgtgcagacagaaacttcaccaagccacaggatggtgacatcacagccccacttataacacctctgaaatggcaaaaagaagctacaggtcactgccaagaagcatccaaagtcaaaacatgcctaaaattttaa

SEQ ID NO: 110 - DNA, 4893 bp, Cricetulus griseus
atgaggaagagacacttgagggtgggtcagcaaacgtgggctcttttctgcaagaactggctcaagaaatgcaggatgagaagagaaacagtgatggaatggtttttctcacttcttctagtattttttgcatacctattatccttcaatttacatcaagttcatgatgctcctcaaattcctgaaatggatctcggacgtgttgataattttaatgattctaactatattattgcctttgctcctgagtctaaaactatctatgagataatgagcatagtggcatcaaccccattcatgaaaggaagaagaatcgtggcatgtcctgatgaaacaagtatgaacgaactagatttaaattactcaatggatgcagtgagagtcatctttaaggatacattctcatatcatttgaaatttccttggggtcacaggatccctccagtaaaagaacacaaagaccattcagctccttgtgaaacagtgaacaacacaataacttgtagaaattcagtcttctgggagaaaggttttgtagcctttcaagctgccatcaatgctggcatcatagagatgacaacacatcattcagtgatggaagagctgatgtcagttattggcgtaaatatgaagatcctaccttttattgcccaaggaggagttacaactgattttttaattttcttctgtgttatatccttttctccatttatatactatttatcagtcaacattacccaagaaaggcaatacattacaatactgatggcaatgatgggacttcgagagtctgctttctggctctcctggggtttgatgtatgctggctttatccttgtcgtggctactttgatgtctcttattgtgaaatctgcccaaattgttgtcttaactggtttcatggtggtgttccttctttttctcctgtacggcctgtctttgatcactttggctttcctcatgagtgtgttgctgaagaaaccctttctcactggtttggctgtcttcctccttaccatcttttggggaagtctaggattcacagcgttatacaaacatcttcctgcctttatggaatggaccttgtgttttcttagcccctttgcctttactgctggaatggcccagcttatacatttggactacgatgtgaattctaatgttaatttggactctccaaacaactcgtacctaatcatagccaccctttttatgttggttttggatgctcttctctatttggttctggcattgtatttcgacagagttttactcagtaagtatggacatcgacgttctccattgtttttcctgaagtcgtcttattggtttcaacacagaggagctaatcatgtggtccttgagaatgaaatagattctgatccatcactgaatgactcatttgaaccagtgcctccagaattccaggagaaagaagccatcaggataaaaaatcttaaaaaggaatatgcaggaaagcatggaaaagttgaagctttaagaggtctgggatttgacatatatgaaggccagatcactgccctccttggccacagtggagctgggaaaaccacactgataaatatactctgtggactatcacccccaactacaggttccatcaccatctataatcagacactttcagaagtggatgactttgatgctgtcatcaagctcactggagtttgtccacagtccaatatacaatttggttttctcactgtgagagaaaatctcaggctttttgctaaaataaagggaattctgccacatgaagtggagcaggaggtacaacgagtgttacaggacttagaaatggaaaatattcaagacattcttgctcagaatttaagtggtggacaaaaaaggaaactgacttttgggattgccattttaggagaccctcaggttttgctgctggatgaaccaactgctgggttggatccattttcaaggcattgtatatggaacctactgaaggagaggaaactagacagagtaattgtgtttagtacacagttcatggatgaggctgatatcctggctgacaggaaggtgtttatatccaatgggagactgaggtgtgcaggctcatctctgttcctgaagaagaaatggggcattggctatcatctaagtttacatctgaatgaaatgtgtgatccagagagtataacttcactggtcaaaaagcacatccctgatgccagactgaccacacaaagtgaagaaagactcatgtatatcttgcccctggaaagcacgaacaaatttccagacctttacagagatcttgataggtgttctaaccaaggcattgagaattatggtgtttccatgacaactctgaatgaggtgttcctgaaattagaagggaagtcaatgactgacgaatcaggtgaccggtcttatagttcaggtgttggaatctctgggcacttacaaagcgaaggggcaaaagacatgggaagccttgttgagctggagcaagtgctattatctctagattcatcaggaaacacagtgagtggcatggcgctgtggaggcagcagctctgtgcagttgcaaaggttcgcttcctcaagctcaagaatgaaagaaaaagcctgatgaccatgttattgctttttggtattagctttcttccccaacttttggaacatctgttctatgagctataccacaaaagttactcatggggactgtctccaagtatgtatttcctttcaccgggacagccaccacaggatcctctgacccacttactggtcatcaataaaacaggttcaagcatcgataaatttgtacactcactaagacagcagagcatagcattagacgtggatgtcctgggaaccagaaatggcagagaagaggcactatacaatggcgctatcactgtattgggtgatgaaaaggatctcagattttcaatagcatgcaataccaaaagactaaactgctttccagtcctcatggatattattagcaatgggctccttggaatctttaattcttcacaacgcatccagactgagagaagcacgttttttgaagagcagatgctttatgagagcggatacgtgagcaatgcgctctgggtcccattggctgccagccttgctccttacgtcgcgatgagcagcatcagtgactacaaaaaaaaaattctttcccagctgtgggtctcaggcctatacccttctgcttactggtgtggacaggctctggtggacatccctctgtactttctgattctccttctcatgcaaataatggagtgtgtttttagccaggaggagattatatttataatggaaaacctgttaattcaggtcctgtgtagcattggatatgcatcgtctcttgttttcctgacatacataatttcattcattttccgtaacggtagaaaaaacagtggcatttggtcatttttcttcttgattgtcaccatcttctccttagttgctactgacataaatgagcgtgggcttctaggactcctactgtgcacctttttgattccacccttcacactgattggctctctattaattttttctgagatttcttatgactccgtggattcctcaggagcctcagactctcaagtcgaatttctggcactgctgataccttacttccattttctccttttcttttttgttctgcgatgtctggaaaggtacttcaggaagaagtcactgagaatggaccctgtgttcagaatctctccaagaagctgtaatgctttccctaacccagaagaccccggagaagaggatgaagatgttcagatggaaagaataagaacagcagattctgtgactgcccttcagatagatgagaaaccagttatcattgctagctgtttgcggaaggaatatacaggcaaaaagaaaaaatgcttttccaaagcaaagaaaaagatcgccacaaggaacatctccttctgtgttaaaaaaggtgaagttctaggacttttaggacacaatggagctggtaaaagtacaacgattagtatgataactggagacacaaaaccaactgccggacaggtatttttgaaagggtgcggtgaagacgctgccctcgggttcttggggtactgtccgcaggagaacgtgctgtggcccatcctgacagtgaaggaccacctggaggcgtatgccgctgtgaaggggctgaagaaaaaggatgccttggtcactgttacacggttggtgaatgcactcaaactgcaggaccagttgaaggctcctgtaaagaccctatcagagggaataaagagaaagctgtgctttgcactgagcatcctggggaacccgtcggtggtgcttctggatgagccctcaaccgggatggaccctgaagggcagcagcagatgtggcaggcgatccgggcctccttcacaaacacagagaggggcgcccttctgaccacccattacatggcagaggcggaggctgtgtgtgaccgcgtggccatcatggtgtctgggaggctgagatgcattggttccattcaacatctcaagagcaaatttggcaaagattacctgctggagatgaaggtgaagaccctcgcacaggtggagcccctcaacatggagatcatgaagctttttccccaggctgctcgacaggagagatactcctccctgatggtctataagttgcctgttgaggatgtatggcctttatcacaggctttcttcaaattggagagattgaaagagaactttgacttggaagaatacagcctctcacagtccactctggagcaggtcttcctggagctctccaaggagcaggagctggatgattttgatgaggaactagattcctcagtgaagtggaagctcctcccacaggaagagctgtaa

SEQ ID NO: 111 - DNA, 1155 bp, Cricetulus griseus
atgtttcacagtcttgaccggaaccaggatggtcacatcgatgtctccgagattcagcagagcttccgagcactgggcatctccatctctctggagcaggcagagaaaatcctacacagcatggaccgcgatggcaccatgaccattgattggcaggaatggcgagaccattttctgctgcactctctggagaatgtggaggatgtcctgtatttctggaagcattctacggtcctggacattggcgaatgcctgacggtgccggatgagttctccaagcaggaaaaacttacaggcatgtggtggaagcagctggtagctggcgcagtggcaggtgctgtgtcacggacaggcacggctcctctggaccgtctcaaggtcttcatgcaggtccatgcctccaagtccaaccggctcaacatcctagggggcctgcggaacatggttcaagaagggggcatcttgtccctctggcgaggcaatggcatcaatgtgctcaagatcgcccctgagtcggccatcaagttcatggcttacgaacagatcaaacgggccatccgggggcagcaagaaacactgcacgttcaggagcgcttcgtggctggctccctggctggggccacagctcaaaccatcatataccccatggaggtactgaagacaaggctgactctgcgcagaaccggccagtacaaggggctcctggactgtgcaaggcggatcctggagcgtgaagggcctcgcgccttctaccgtggctacctgcctaacgtgctgggcatcatcccctatgctggaattgacctagccgtctacgagaccctgaagaatcactggctccagcagtacagccgcgaatcagcaaatccaggcattcttgtgctcctggcctgcggcaccatctccagcacctgtggccagattgccagttaccctctggcactggtccggacccgaatgcaggcccaagcctccatcgaaggtggcccacaggtctccatggtgggtctgcttcgacacatcttatcccaggagggtgtatggggcctctaccggggcattgcccccaacttcatgaaggtcattccagccgtgagcatctcctacgtggtctacgagaacatgaagcaggctctgggggtcacatccaggtga

SEQ ID NO: 112 - DNA, 981 bp, Cricetulus griseus
atgtatagagagtgggcagtggtgaatattcttatgatgtcctacttgtacctggtgcagggcttcaatagtgaacatggaccagtgaaggatttttcttttgagcaatcatcccagtcaatgttggaacgatctgaacaacagatcagagctgcttctagtttggaggagctactgcaaatcgcacactctgaggactggaagctgtggagatgccggctgaagctcaaaagtcttgctagtatggattctcgctcaacatcccatcgatccaccagatttgcagcaactttctatgatactgaaacactaagagttatagatgaggaatggcagagaacccagtgcatccctagagagacatgcgtagaagtcgccagtgagctggggaagacaaccaacacgttcttcaagcccccctgtgtgaacgtgttccggtgtggaggctgctgcaatgaagaaagtgtgatgtgtatgaacacaagcacctcctacatctccaaacagctctttgagatatcggtgcctctgacatcagtgcctgagttagttcctgttaaaattgccaaccatacaggctgtaagtgcttgcctactgccccccgccatccctactcaattatcagaagatccattcagataccagaaggagatcaatgttctcattccaagaaactctgtcctattgacatgctgtgggataacaccaaatgtacttgtgttttacaagatgagaatccactccctggaacagaagaccattcttaccttcaggaaccagctctctgtggaccacacatgaagtttgatgaagagcgttgtgagtgtgtctgcaaaacatcatgtcctggagatctcattcagcacccagaaaactgcagttgctttgaatgcaaagaaagtctggagagctgctgccagaagcataagatttttcacccagacacctgcaggtcaatggtcttttcacttaccccttaa

SEQ ID NO: 113 - DNA, 1536 bp, Cricetulus griseus
atgggcagtgtctgtatgaggctctggacgtacttgcggccccttctcccctgctggtcccaagaggcagacaagtcagtggtgattgagaatcctggggcctgctgtccccctgagtctccccggtcacctgagcctgagagaagccatgggaactacttcgtggctctttttgattaccaagctcgtaccgcagaggacctgagcttccgtgcaggcgacaaactccaagtcttggacacttcgcacgagggctggtggttggccaggcatttggagaagagagctggcttgggccagcagctgcagggctacattccttctaattacgtggcagaggaccggagtctccaggctgagccgtggttctttggagcaatcaaaagagcagatgcagaaaaacagctaaagtattcggaaaaccggacaggcgcctttctcatcagagagagtgaaagccagaagggggacttttccctctcagttttagatggaggtgacgtaaaacactacagaatcagaaggctggatgaaggtggttttttccttaccaggaggaaaaccttttcaaccttgaatgaatttgtgagttactacagcacaacaagtgatggcctctgtgtgaagctgggaaagccatgcttaaagatacaggtcccagctccttttgatttgtcatataaaactgtggaccagtgggagatagaccgcaactccatccagcttctgaagcgactgggatctggccagtttggagaagtttgggaaggcctgtggaataataccactccagtggctgtaaaaacattaaaaccaggttcaatggatccaaatgacttcctgagggaggcacagataatgaagagcctaagacatccaaagcttatccagctttatgctgtttgcactttagaagatccaatttatattattacagagttgatgagacatggaagtctgcaagaatatctccaaaatgatggtggaacaaaaatccatctgactcaacaggtagacatggcagcacaggtggcctctggaatggcctacctggagtcccagaactacatccacagggatctggctgcaagaaatgttcttgttggtgaacataatatctacaaagtagcagattttggacttgcaagagtttttaaggtagataatgaagacatctatgaatctaaacacgaaataaagctgccagtgaagtggactgcgcccgaagccattcgtactaataaattcagcattaagtcagatgtgtggtcttttggaatccttctctatgaaatcattacttacggcaaaatgccttacagtggtatgacaggtgctcaagtaattcaggtgttgagtcaaaactacaggctcccacaaccatctaactgtccacagcaattttacagcatcatgctggagtgctggaatgcagagccgaagctacggccaccatttgagaccctgcactgtaaacttgaagactactttgaaacagactcctcatattcagatacaaataatttcataaattga

SEQ ID NO: 114 - DNA, 609 bp, Cricetulus griseus
atggattattttcccgtgatcttctctctgctgtttgtggctttccaaggagctccagaaacagccgtcttgggagcagaactcagcacccgggctgagaatggagggtacagccctactcctagcacatcctggagaccccgtaggtccaagcgttgttcctgttcctccttgatggacaaggagtgtgtctacttctgccacttggacatcatctgggtcaacactcccgagcgtgttgttccatatggactgggaagcccttccaggtccaagcgttccctgaaggacttactttctccaaagaccacagaccacgggaataggtgtcagtgtgctcagcaaaaagacaggaagtgctggaatttctgccaaggaggaaaagaactcagagcccaaaacaccatgcagaaaggcttagaagacttcaagaaaagaaaagcctgttccaagctgggcaagaagtgtatcaaccagcaactggtagaagaaagaaaaataagaaggttggaggccatcaccaatagcatcaagacatcttttcgtgttgcaaagttgaaagctgatctctatagagaccagaagttgacacacaaccgagcacactga

SEQ ID NO: 115 - DNA, 2631 bp, Cricetulus griseus
atggggctgcccactctggagttcagcgactcgtacttggacagcccggatttcagggagagacttcagtgtcacgagatcgagctagagagaaccaacaagttcatcaaagagctgttgaaggatggctccctgctcatcggggcgctgaggaatctatccatggctgtacagaagttttcccagtctttgcaagacttccagtttgaatgtatcggagatgcggaaactgatgacgaaatcagtattgcccaatcactgaaagaattcgcaaggctgcttattgccgtagaggaagaaaggcgtaggctgatccaaaatgctaatgatgtattaattgcaccacttgagaaatttcgaaaagaacagatcggtgcagctaaagatggaaaaaagaagtttgacaaggagagtgaaaaatactattctattctggacaagcatttgaacttgtcggcaaagaaaaaggagtctcacttgcaagaggcagattcacagattggccgagaacatcagaacttctatgaagcatcattagaatatgtctttaaaatccaagaggttcaagagaagaagaagtttgagtttgtggagccgcttttgtccttcctgcaaggactatttacgttttaccacgagggatatgaacttgcccaggaattcgcaccatataagcaacagctgcagttcaacttgcagaatgtaagaatgaataactttgaaagtactcgacaagaggtagaaagactgatgcagcggatgaaatctgccaaccaggattacagaccacccagccagtggaccatggagggttatctctatgtccaggagaaaagaccattgggttttacatggattaaacattactgcacttacgacaaaggaagcaaaatgttcacaatgagtgtctcagaagtgaaagccagtgggaagatgaatggcctccttactggctcacctgaaatgtttaaactaaagtcatgtatccgaagaaagacagattcaattgacaaacgcttctgctttgacatagaagtggtcgaaaggcatggcatcattacactacaggcattctctgaagctaaccggaaactctggctggaggccatggatggaaaagaaccgatttacacactgcctgccataattagcaagaaagaagaaatgtacttaaatgaagcggggttcaactttgtgagaaaatgcatccaagctgtggaaacaagaggtatcaccattttaggcctgtaccgaataggaggtgtgaactccaaggtccagaaacttatgaacaccacattttctcccaagtcccctcctgatatggacattgatattgaactgtgggacaataagactataacaagtgggctgaaaaactacctcaggtgccttgcagaaccactgatgacttacagattacacaaggatttcatcattgctgtcaagtcagatgaccaggactacagggtcgaggccgtccatgcactggtgcacaaattgccggagaaaaacagagagatgctggacatcttgataaaacacctgctcaaagtctcattacacagccaacagaatctcatgacaatctcaaatcttggcgtcatatttggcccaaccctgatgagagcgcaggaggaaactgtggctgccatgatgaacatcaagtttcagaacatagtggttgaaatcctgatcgaacactatgaaaagatttttcacaccgccccagacccaaacatccctcttcctcagcctcagtctcggtctagttcccgaaggacccgtgccatctgcctgtccacaggttctcggaagcccagagggaggtacacgccatgcctggcagagccggatagtgattcctacagcagcagcccagacagtacacccatgggcagcatagagtctctctcttcccactcctctgaacaaaacagcaccacaaagtcgacttcctgccagcccagggagaagtcaggtggtattccctggatcgcatccccatcatcttccaatggacagaagagtctgggtctgtggacaactagcccagagtcaagttctcgagaagatgcaaccaagacagacgtggagtcagattgccaaagcgtggcctcggtcaccagcccaggggatgtctctccccccatagacctagtcaagaaagggccttatggaccctcaggactgaaaagaacctctgcctcctcctctctcagatccatctctgcagttgaaggaaacaagagctacagtggatccattcaaagcttaacttctataggttccaaagagtcacccaaagccacaccaaacccagagctgcctccaaaaatgtgcaggaggttaaggctagacactgcctccagcaatggctaccagcgaccgggctcagtagtggcagcaaaggctcaaatatttgaaaatgctggttcactcaagccagtttcttcaggacgccaagccaaagccatgtactcctgcaaagcagagcacagccatgaactgtccttcccgcagggggcgatcttttctaacgtgcacccatcagtggagccagggtggctgaaggcgacttatgaaggcagaacaggactagtccctgaaaactatgttgtcttcctctaa

SEQ ID NO: 116 - DNA, 957 bp, Cricetulus griseus
atggcgcgcccccaggccctcgccttcgggctcctgctcgcggtggccacggcgacgctggccgcggctcagcaaagtgaagcctgtgtctgtgaaaactacaagctggggacaagttgctctctgaatgacagaggcgaatgccagtgtacttcccttggtacatcgaatactgtggtgtgctccaaactggcatccaaatgtttggtgatgaaggcggaactgacttacagcaacaagtctgggaggaggctgaaacccgagggggccatacagaacaacgatggcatgtacaaccctgaatgtgacgagctgggcctcttcaaagccaagcagtgcaatggcaccgccacctgctggtgtgtgaacactgccggtgtccggagaaccgacaaagacacggaaataacctgctccgagcgagtgaggacctactggatcatcatcgaaataaaacacaaagaaagagaaagcccttacaaccttcagaatttgaagactgcacttgaggaggtgttcatgtctcgatacaaactgaatcagaaatttatcaaaaatattctgtatgagaacaatgttatcactattgatctgatgcaaaactcttctcagaaaactcaagatgatgtggacatagctgatgtggcttactattttgaaaaagatgtgaaaggggaatccttgttccatcattctaagaaaatggacctgagagtaaatggagaacaacttgatctggaccctggccagaccctgatttactatgttgatgaaaaagccccggagttctcaatgcagggactcaaagccggcatcattgctgttattgtggtagtggtgttagcaatcatcgcgggggtagttgtgctggttatgtctacaaggaagagatcagcgaagtatgagaaggctgagataaaggagatgggtgagatacacagagaactcaatgcctaa

SEQ ID NO: 117 - DNA, 7827 bp, Cricetulus griseus
atggtgtgggttcctgaaggggggatgctgaagatcaccaaccggatcttgcaggctcaagctcctggtgtccgagccgaggacatcatctacaagatcacacacggccacccccagtttggggaggtggtcctcctgatggatctgcctgcagacagccctgccggccctgctggtcctgctgctggtcctaccagccctgccgaggaagggcagcacctgcctgatggaagaatggccacacccaccagcaccttcacccagcaggacatcgatgatggcatagtgtggtacagacactcaggagccctgacccagagtgatgccttccacttccaggtgtctagtgccgctgatgcccacacccacctggaaagtcacatgttcaacattgcaatcttacctcaggcacctgaggcacctaaactctccttggggacatctctccatatgaccgctcgggaagatggcctaactgtcattcagcctcactccttgtcctttgtgaactcaaagagtcccatcggaaagattatatacaacatcaccgtacctctgcatccaaatcaaggtgccattgagcacagagaccagcctcactctcccatccagtatttctcccaggaagatattaaccagggcaaaatcgtgtatcgccctcccgctgcggcctcccacctccaggagatcatggcctactcctttgttggtctcccagaatcagtgagagtttacttcacagtttctgacggacatcacacgagtccagagatggccctcaccattcacttacttcactctgatcggcagccacccgcatttcagataagggcgcccctgttggaggttagccctggaggccgcacctctctaggcctgcagttggtagtgagagatgctcagatggtaccagaagaactcttcttccagcttcagaagccgccacagcatggctctctcctgaagtccacagcagagtcctcagggcccatggcagcaggtgacactttcacatacggtgaagtcgagaggaatgctctgcagtacgtacacgatgggtcctctgcctgggaggacagcatggtgatctcagtctcagatggcctgacagtgacaacatcagaagtaaaagtggacgtgtccctgtcagagaacagagggcctcggctggccccaggttcctccctcagcatgactgttgctagtcaacggacagccataatcaccaggtcaaaccttgcctatgtggacgattcttcccctgacccagagatctggattcagttaagttctctgcccatgtatggtgtgctcttagtggatgagggaggcagggcaccactctccctccaccatttctttgctacggatgatcacgacaaccttcagggtgatgctgtcattaaactaagcgctctacccaagtatggctgcattgagaacacaggaacaggagatcgctttggcccgggagctaccagtgacccagaggcatcattccctattcaagatgtcctagaaaactatatttactactttcagagtgttcatgaaagcatcgaaccaacccatgatgtgttcagtttttatgtgagtgacggaagcagtcgctctgaaattcacagcatcaatgtcaccattgagaggaagaatgatgagcctcccaggatgaccttgtggcccctcagagtccggctgagctcgggagtggtgataagcaattcttccttgagcctgcaagacctggacaccccagataatgagctggtttttgtgttgatgaaaaagcctgaccacggccgcctacttcagaggcccactgcttctgaccctctagagaatgggactgttttagaccaaggctcctccttcacctaccaggatgtcctggctgggctggttgggttcttacctaatgacaatggtatgaccgtagatgagtttcggttctctctcactgatggccttcatgtagacatgggaaggatggagatctacatagaactgcctacaggcgacagcccccacttggtgattaaccaaggacttcagctccccgcagggtctgtagcacgcatcacagagcagcacttgagagtgacagatgctgactcagaagatggacagattctgtacatcatgaaggaggaccctggtgcaggacgcctgcagatggtgaagcctgactacccagagcagatctctgttagaggccccatccggagcttcacccaggcagacattaaccaagggcagatagaatacagccatggaccaggagaacctctcgggagctttgcttttaaatttgatgtgattgatagagaaggcaataagttggctgaccagtcatttgctatcagtgttttagaagccgaattcccaccagtcattatcaccaataaagggcttgtcttggatgagaactcagggaagaaaatcacccctgcacagctcttggccactgaccaggacagcaaggctgcagagctgatctacagaatcactgcccagccccagctaggccgcctggaacatgtggcatcaccaggggtccagattagttcctttactcaagctgacctgacttcaggaaatgttcagtatgtccattccagtgagactaggaagtactccgacgccttcagctttttgttgtctgatggcgtgcatgaggtgacacagaccttccacatcactattcaccctgtggatgactcactgccccttgtgcagaaccagggcatgcgtgtgcaggagggcgtgaggaagaccatcacagagtttgagctgaaggcagtggatttggacacggaggctgagtctatcaccttcaccattgtgcagcccccacgccatggcgccattgagagagcagccagggggcagcatttccacctcacctcctccttcactatggaagacatctaccagaaccgggtcagctatagccatgatggcagcaactccctcaaagaccgatttaccttcaccgtttctgatgggaccaaccccttctttatgatagaggaaggaggaaaagagattatgaccgcagcacctcagcagttccatgtggacatcctcccggtagatgatggcacccctagaattgtcaccaacctgggtctgcagtggctggagtatatggatggcaaggcaaccaacctgatcaccaagaaggaactgctgaccatggacccagacacagaggactcacagcttgtctatgaggtcaccgtgggccccatgcacggctatgtggagaacaagctgcaaccaggaagagctgcagccaccttcacccaggaggatgtgaacttggggttgattcgttatgtgttgcatgaggagaagatccagaaagtaatggatagttttcagtttctggtgaaagacagtaaacccaacgtggtcagcaacaatatcttccatatccagtggtctctcatcagcttcaaatataccagctataacgtcagtgagaaggcagggtctgtcagtgtcacagtgcagaggactgggaacctgaaccagtatgccatcgtcctgtgtcgcaccgaacaaggcacagccagctccagctctagagtcagctctcatcctggacagcaagactacatggagtacgctggccaggtgcagtttgatgaacgagaggacacaaagtcctgcaccattgtcatcaatgatgatgatgtatttgagagtgttgaaagtttcaccgtggagctcagcatgccagcctacgccctgctcggagagttcacccaggcaaaggttgtcatcagtgacactgaggatgagcccacactggagtttgataagaagacctaccgggtcaatgagagcgctggttttctgtttgcacccattgaaagaaaaggagactcaagcagcattgtgtctgccatttgctacacagtccctaaatcggctatgggaagtagtctgtatgctctggaatcaggttctgattttaagtccagagggaggtctcctgagagtcgtgtgatatttggacctggtatcaccatgtctacgtgtgatgtcatgatcattgatgacagcgagtatgaagaggaagaggagtttgagattgccctggctgattcttctgataatgcccgaattggaaggctggcagtggccaaggtgctcattagtggtcccaatgatgcctcgactgtctccctgggcaacacagctttcacgatcagtgaggatgcaggcacagtgaaaattccagttatccgccatgggactgatcttactaccttcacatctgtctggtgtgcaacacgaccctcagatccagcttctgccactccaggagttgactatgtccctagctcaaggaaggtggaatttggaccaggtgtcaccgaacagtactgtaccttgactatcttggatgatacgcaatacccagtaattgaaggactggagacatttgtggttttcctcagctcagcacaaggagctgaactgaccaaaccctttcaggctgtcattgcgataaatgacacattccaagacgttcccagcatgcagtttgccaaggatttgcttctagtgaaggagaaagagggtgtcctgcacgttcctatcatccggagtggggacctgagctatgagtcatcagtgaggtgctacactcagagccactcagctcaggtcatggaggactttgaggagagaagaaatgcagactcttcacggattacgtttctgaaaggggagaaggcgaagaactgcactgtctacatccatgatgactccatgtttgagcctgaggagcagttcagggtctaccttggccatccccttgggaaccactggagtggagccagagtcgggaagaacagtgtggccactgtgaccatatccaatgacgaagatgctcctaccattgagtttgaagaagctgcctaccaagtgcgggaacctgcagggccagatgccattgcagttctgagcatcaaggtcatccgcagaggcgatcaaaataggacttctaagatccgctgcagcactcgagacgggtctgcccagtctggtgtggattattaccccaagagcagagtcctgaaattcagtcctggtgtggatcacatcttcttcaaagttgagatcctgtccaatgaagatcgggaatggcacgaatccttttctctagtccttggcccagatgacccagtggaagcagtgcttggggacgtgaccacagcgactgtgacaattctagaccaagaggcagcaggaagtctcatattgccagcaccacccattgtcgtcacccttgctgactatgatcatgtggaagaggttacaaaggaaggaatcaagaaagccccatctccaggctacccactggtctgtgtcactccctgtgacccccacttccctaggtatgctgtcatgaaggagcgctgtagcgaggctggcatcaaccaaacatccgtgcaattcagctgggaagtggctgcccccactgatggcaatggggctcggtctccctttgagaccattactgataacacaccattcaccagtgtcaaccacaaggtcctagacagcatttacttcagccggcggttccatgtgcgttgtgtggcaaaggctgtggacaaggtgggccatgtagggacccctttgaggagcaatgttgttaccattggaacagacagtgctatctgccataccccagtggtggccgggacagctcgaggcttccaggcacagtccttcattgcaaccttgaaatacctggacgtcaaacacaaagagcatccaaacagaatccacatctcggtgcagattccccaccaggacggaatgctgcctctcatctccacaatgcccttacacaacttgcatttcctgttgtccgagtccatctacagacaccagcacgtttgctccaacttagtcactgcccgtgacctgcgaggactctcagaggcagggttcctagatgattcaggcttccatagtactgctctgggccctggctatgaccgccccttccagtttgacccaagtgtccgggagcctaagaccatccagctgtacagacacctgaacctgaagagctgtgtatggacctttgatgcgtactatgacatgacagagctgattgacgtgtgtgggggctccgtgactgctgacttccaggtgagggactctgctcagtcattcctaacagtacatgtgcctctctatgtgtcctacatctatgtgacagcgcccaggggctgggcctccttggagcaccacacagagatggaattttctttcttctatgacacggttctttggagaacagggatccagacagacagtgtgctctctgccaggcttcagatcataagaatctacattagagaggatggccgtctcgtcattgagttcaagacccatgccaaattcagaggacagttcgtgatggagcatcacactctcccagatgtgaaatctttcatactgactccagaccacctaggaggaattgaatttgaccttcaactcctatggagtgctcagacgtttgactctccacatcaactctggagagccacaagctcttacaacagaaaggactattcgggggaatacaccatctacctgatcccttgcactgtgcagcccacacagccgtgggtggacccaggagagaagtctttggcttgcactgcacatgctccagaaaggttcctgatacccattgcattccagcagaccaaccgtcccgtgccagttgtgtattcgctcaataccgaatttcagctctgcaataatgagaaggtattcttgatggatcccaacacgtctgatatgtcactggcagaaatggattacaaaggagccttttccaaagggcagatcctttacggccgagtcctttggaatccggaacagaaccttcattctgcttacaaactgcagctggagaaagtctatctttgtactggcaaggatggctacgtgcctttctttgatcctactgggacaatctacaatgaggggccacagtacggatgcatccaaccaaacaaacacctgaagcacagattcctgctgctggaccgcagccagccagaggtcaccgacaagtacttccacgatgtgcctttcgaggcccacttcgcttctgagttgcctgacttccatgtggtcagtagcatgccaggtgtggatggattcaccctcaaagtagacgccctctataaggtggaagcagggcaccagtggtatctccaggtgatctatgtcattggccccgacagcatctcgaggccccgggtccagcgctctctcatagcctctctgagacgccaccaaagggacctggtggacccccgtggctggttgtctcttgatgactccctcatctatgacaatgaaggagaccaagtcaagaatggcaccaacatgaagtccctgaacctggagatgcaggagcccattgtagccgcttccctgtctcaaactggagcatctattggcagtgccctggctgctattatgcttctccttctgctgtttctggtggcctgtttcatcaccagaaaatgccagaagcagaagaagaaacagcccccagaggacactctggaggaataccccttgaataccaaggtggatgtagccaagagaaatgcagacaaggtagagaagaatgtcaacagacagtactgcactgtgcgcaatgtcaacatactgagtgacagtgagggctcttacacattcaaaggtgctaaagttaaaaggatgaatctggaagtcagagtccacaacaatttacaagatggaacagaagtttaa

SEQ ID NO: 118 - DNA, 2394 bp, Cricetulus griseus
atggaggccagagaggagtgctttcttaaacaaaggcaagtcttgcttctttttgtttttctaggtgggtctctggctgggtctgggtctaggcgctattctgtggtcgaggaaaaagagaagggattcttaatagccaatctagcgaaggatttggggctcagggtagaagaactggctgaaaggagagctcaggctgtctccaaagggaacatacagtattttcacctcagccatcagactggagatttgattttggttgagaaaatggaccgggaggagctgtgcggttccacagagccttgtgtgctgcattttcagatactgctgcatgatcctttgcagattattacacatgagcttgagatcgtagatgtaaatgacaatgccccagagttctttgaaaatgcaatgcagctcaaagtcctggaaagctccccagccgggacaataattcccttgggaaatgctgtggacctggacgtgggaagaaacggactccaaaactacacgatctctcccaagtcccatttccatgttcgcacccgccgccgtagggatggaaggaagcatccagaactagtgctagatagagctctggatcgggaggagcagccagagctcagtttaactctcacagccctggatggaggatccccacctaggtctggcactgcccaggtccacatcctgatcttagacataaatgataatgccccagaatttacacagtcactctatgaggttcaaattctagagaagagccttgttggctctgtcattatcacagtctcagcttctgacttagatacagggaattttggtgcaatatcatatgtgttcttccatgcatctgaagaaattctcaaaacttttcaactaaatcccattactggtgatatacagttagttaagagtttggattatgaaaaaattaatacttacgaagttgacatagaggctaaggatggtggaggcctttcaggaaaatgtacagtcatagtccaggtagttgacgtgaacgataacccaccggaactgactttgacatcagttaacagtcctatccctgagaattcggcagagattgtagtggctgttttcagtgttgctgatttagactctggagataatggaaaagtgacgtgttccatccagaacaatctccctttcatcctgaaacgttctgtagagaacttctacactatagtgtctgaaggagctctggacagagagagcagagctgagtacaacatcaccatcacagtcaccgacctgggcacacccaggctcacaacccagcacaccataacagtgcaggtgtccgatgtcaacgacaacgcccccgccttcacacaaacctcctacaccctgtttgtccaggagaacaacagccccgccctgcacataggcaccatcagcgccacagattcagactcaggctccaatgcccacatcacctactcgctgctgcccacccacgaccctcagctggccctctcgtcgctcatctccatcaatgcagacaacggacagctgttcgcgctcagggcgctggactatgaggctctgcagaccttcgagttccacgtgggagccacagaccaaggctcgcctgcgctcagcagtcaggctctggtgcgcgtgctcgtgctggacgccaacgacaatgctcccttcgtgctctacccactgcagaacgcgtctgctccctgcacagagctgctgcccagggcggcagagccaggctacctggtcaccaaggtggtggcagtggatcgcgactctggccagaatgcctggctgtccttccagctgctcaaagccacggagcctgggctgttcaccgtgtgggctcacaatggcgaggtgcgcacctccaggctgctgagtgagcgcgatgctcccaagcacaggctgctgctgctggtcaaggacaatggcgaccctcccaggtctgccagtgtcactctgcaagtgctggtggtggatggcttctctcagccctacctgcctctgccggaggtggcgcacgaccccgcgcaggaggatgacttgctcactctctatttggtcatcgccttgacgtcagtgtcttcgctcttcctcttgtctgtgttgctgtttgtggctgtgaggctgtgcaggaggggcagggcggcctctctgggtggctgctctgtacctgagggacactttcctggtcacctggtggatgtcagcggcgcaggaaccctctcccagagctaccagtatgaggtgtgtctaacgggagactctgggactggtgagttcaaattcctgaatccgctgattccgaacctgtttatggaagaatctgagagagaaattaaggaaagtccccactgcaggagtagctttgtagtccgctaa

SEQ ID NO: 119 - DNA, 1665 bp, Cricetulus griseus
atgagctgcttgggggacagcctgagcagtcggaacatcatcatggggtctactgatgcggacatcgaagaactggaaaacgccacttacaagtatctcattggagaacagacggagaagatgtggcaacgcctgaaaggaatactaagatgtctagtgaagcagctggagaaaggtgatgtcaatgtggtcgacctaaagaagaacattgaatatgcagcatcagtgctggaagcagtttacattgatgagacaagaaaacttctggacactgaagatgagctcagtgacatacagactgactcagttccatcagaagtccgggattggttggcttctaccttcacacggaaaatggggctgatgaaaaagaaaacggaggaaaaaccaaagtttcgaagcattgtccatgctgttcaggctgggatttttgtggaaaggatgtacaggaagaattatcacatggttggtttgacataccctgcagctgtcataataccactcaaggaggttgataaatggtcttttgacgtatttgccttaaatgaagcaagcggggagcatagtctcaagtttatgatttatgaactgtttaccagatatgatcttatcaaccgattcaagattcctgtttcttgcctaattgcctttgcagaagctttagaagttgggtacaccaagcacaaaaatccatatcataatttggttcatgcagctgatgtcactcaaactgtgcattacataatgcttcatacaggtatcatgcactggctcactgaactggaaattttagcaatggtttttgcagctgccattcatgactttgagcatacaggaacaacaaacaacttccatattcaaacaaggtcagacgttgccattctgtacaatgaccgctcggtgcttgagaaccaccacgtgagtgcagcctatcgacttatgcaagaggaagaaatgaatatctttgtaaatttatctaaagatgactggagggatcttcggaacttagtgattgaaatggtgttatctaccgacatgtcaggccatttccagcaaattaaaaacataagaaacagtttgcagcagcctgaagggattgacagagccaaaaccatgtccctgatactccatgcagccgatatcagtcacccagccaaaacttgggagctgcactacaggtggaccatggccctaatggaggagttcttccggcagggagacaaagaagccgaattaggacttccattttctccactctgtgatcggaagtccacgatggttgcccagtctcaaataggtttcattgatttcatagtggagccaacattttctctcctgacagacacaacagagaaaattgttattcctcttatagaggaatcagcaaaatcagagtcttctaactataggccaagcagcacgttccatgttgctgactccctgaggcggtcaaatgtgaaatgccccatgagtgatgggagctatactccagactactctctgtcagctgtggacctgaagagtttcaaaaacaacctggtggacatcattcagcagaacaaagagaggtggaaagagttagctacccaaggtgaacttgatcttcataagaactcagaagacttagggaatgctgaagaaaagcatgctgacacacatccatag

SEQ ID NO: 120 - DNA, 22 bp, Artificial Sequence
  Synthetic primer
tgcagctggcttatttaacacc

SEQ ID NO: 121 - DNA, 21 bp, Artificial Sequence
  Synthetic primer
tggaactcacggacgaaaact

SEQ ID NO: 122 - DNA, 20 bp, Artificial Sequence
  Synthetic primer
tggcgtggatgtcactaagg

SEQ ID NO: 123 - DNA, 20 bp, Artificial Sequence
  Synthetic primer
aaaggggacccccactactc

SEQ ID NO: 124 - DNA, 20 bp, Artificial Sequence
  Synthetic primer
ggaatcgctgttgttggcat

SEQ ID NO: 125 - DNA, 20 bp, Artificial Sequence
  Synthetic primer
tggcacagctagaagaacgg

SEQ ID NO: 126 - DNA, 20 bp, Artificial Sequence
  Synthetic primer
tgtttggtgatgaaggcgga

SEQ ID NO: 127 - DNA, 21 bp, Artificial Sequence
  Synthetic primer
ctgggtctaggcgctattctg

SEQ ID NO: 128 - DNA, 21 bp, Artificial Sequence
  Synthetic primer
tccgagattcagcagagcttc

SEQ ID NO: 129 - DNA, 20 bp, Artificial Sequence
  Synthetic primer
tgggtgtttcttggggtagg

SEQ ID NO: 130 - DNA, 20 bp, Artificial Sequence
  Synthetic primer
agaaggttggaggccatcac

SEQ ID NO: 131 - DNA, 20 bp, Artificial Sequence
  Synthetic primer
cacacccacctggaaagtca

SEQ ID NO: 132 - DNA, 20 bp, Artificial Sequence
  Synthetic primer
gcccagtcccctcttgattt

SEQ ID NO: 133 - DNA, 24 bp, Artificial Sequence
  Synthetic primer
cagttcaacttgcagaatacaagg

SEQ ID NO: 134 - DNA, 20 bp, Artificial Sequence
  Synthetic primer
gcccaccatcctacgaagag

SEQ ID NO: 135 - DNA, 20 bp, Artificial Sequence
  Synthetic primer
gactccgatgggtcgttctt

SEQ ID NO: 136 - DNA, 20 bp, Artificial Sequence
  Synthetic primer
gcggactacgagaagcacaa

SEQ ID NO: 137 - DNA, 20 bp, Artificial Sequence
  Synthetic primer
cctggaagagcttagctgcc

SEQ ID NO: 138 - DNA, 20 bp, Artificial Sequence
  Synthetic primer
aggggcgaagaactcgacta

SEQ ID NO: 139 - DNA, 20 bp, Artificial Sequence
  Synthetic primer
cagcacctgccctttgtagt

SEQ ID NO: 140 - DNA, 20 bp, Artificial Sequence
  Synthetic primer
tgccttgaagtccagcttgt

SEQ ID NO: 141 - DNA, 20 bp, Artificial Sequence
  Synthetic primer
gagcctccttgcccttagtc

SEQ ID NO: 142 - DNA, 20 bp, Artificial Sequence
  Synthetic primer
tcttcacttccgtcgatgcc

SEQ ID NO: 143 - DNA, 20 bp, Artificial Sequence
  Synthetic primer
tcgttgttctgtatggcccc

SEQ ID NO: 144 - DNA, 20 bp, Artificial Sequence
  Synthetic primer
ctaccctgagccccaaatcc

SEQ ID NO: 145 - DNA, 21 bp, Artificial Sequence
  Synthetic primer
cgccattcctgccaatcaatg

SEQ ID NO: 146 - DNA, 20 bp, Artificial Sequence
  Synthetic primer
acccaccaagagtcacgttg

SEQ ID NO: 147 - DNA, 20 bp, Artificial Sequence
  Synthetic primer
tgctcggttgtgtgtcaact

SEQ ID NO: 148 - DNA, 20 bp, Artificial Sequence
  Synthetic primer
gttaggccatcttcccgagc

SEQ ID NO: 149 - DNA, 20 bp, Artificial Sequence
  Synthetic primer
gcagagctgagagagttccc

SEQ ID NO: 150 - DNA, 20 bp, Artificial Sequence
  Synthetic primer
tggctgggtggtctgtaatc

SEQ ID NO: 151 - DNA, 20 bp, Artificial Sequence
  Synthetic primer
gagccgtgttccatttgagc

SEQ ID NO: 152 - DNA, 20 bp, Artificial Sequence
  Synthetic primer
catgacggagcaggagaaca

SEQ ID NO: 153 - DNA, 20 bp, Artificial Sequence
  Synthetic primer
cggttgaacgacttggtcac

SEQ ID NO: 154 - DNA, 18 bp, Artificial Sequence
  Synthetic oligonucleotide
  modified_base (2)..(2): a, c, t, g, unknown or other
  misc_feature (2)..(2): n is a, c, g, or t
  modified_base (4)..(5): a, c, t, g, unknown or other
  misc_feature (4)..(5): n is a, c, g, or t
  modified_base (7)..(8): a, c, t, g, unknown or other
  misc_feature (7)..(8): n is a, c, g, or t
  modified_base (11)..(11): a, c, t, g, unknown or other
  misc_feature (11)..(11): n is a, c, g, or t
  modified_base (15)..(15): a, c, t, g, unknown or other
  misc_feature (15)..(15): n is a, c, g, or t
  modified_base (17)..(17): a, c, t, g, unknown or other
  misc_feature (17)..(17): n is a, c, g, or t
rnynncnngyngktnyny

SEQ ID NO: 155 - DNA, 15 bp, Artificial Sequence
  Synthetic primer
  modified_base (3)..(3): a, c, t, g, unknown or other
  misc_feature (3)..(3): n is a, c, g, or t
  modified_base (10)..(10): a, c, t, g, unknown or other
  misc_feature (10)..(10): n is a, c, g, or t
  modified_base (13)..(14): a, c, t, g, unknown or other
  misc_feature (13)..(14): n is a, c, g, or t
gtnwayattnatnnr

(19) United States
(12) Patent Application Publication
(10) Pub. No.: US 2023/0193341 A1
(43) Pub. Date: Jun. 22, 2023

(54) EXPRESSION SYSTEMS, RECOMBINANT CELLS AND USES THEREOF

(71) Applicant: Selexis SA, Plan-les-Ouates (CH)

(72) Inventors: Lucille Pourcel, Plan-les-Ouates (CH); Audrey Berger, Plan-les-Ouates (CH); Valerie Le Fourn, Plan-Les-Ouates (CH); Severine Fagete, Plan-les-Ouates (CH); David Calabrese, Plan-les-Ouates (CH); Alexandre Regamey, Plan-les-Ouates (CH); Nicolas Mermod, Plan-Les-Ouates (CH); Fabien Palazzoli, Plan-les-Ouates (CH); Pierre-Alain Girod, Plan-les-Ouates (CH)

(73) Assignee: Selexis SA, Plan-les-Ouates (CH)

(21) Appl. No.: 17/287,366

(22) PCT Filed: Oct. 23, 2019
(86) PCT No.: PCT/IB2019/059076

§ 371 Date: Apr. 21, 2021

Related U.S. Application Data
(60) Provisional application No. 62/749,789, filed on Oct. 24, 2018.

(51) Int. Cl. C12P 21/02; C12N 15/10; C12N 15/85; C12N 9/10; C12N 9/88; C07K 14/47; C12N 9/90; C07K 14/705; A61K 48/00

(52) U.S. Cl. CPC C12P 21/02; C12N 15/1082; C12N 15/85; C12N 9/104; C12Y 203/02004; C12N 9/88; C12Y 402/01001; C07K 14/4716; C07K 14/4702; C12Y 503/04001; C12N 9/90; C07K 14/70567; A61K 48/0066; C12N 2800/107

(57) ABSTRACT

A transcriptomic analysis of genes consistently upregulated in high producer clones were each evaluated for their ability to increase the production of a protein of interest. The products of these genes (metabolism influencing products (MIP)), such as actin, Erp27, Erp57, Foxa1, PPAR, Ca3, and Tagap, could be sub-categorized into different functional categories such as signaling, protein folding, cytoskeleton organization and cell survival.

### CROSS-REFERENCE TO RELATED APPLICATIONS

This is the U.S. national stage of International application PCT/IB2019/059076, filed Oct. 23, 2019 designating the United States and claiming priority to U.S. 62/749,789, filed Oct. 24, 2018, which is incorporated herein by reference in its entirety.

### REFERENCE TO SEQUENCE LISTING SUBMITTED VIA EFS-WEB

This application includes an electronically submitted sequence listing in .txt format. The .txt file contains a sequence listing entitled “3024-276NS_ST25.txt” created on Nov. 9, 2021 and is 241,016 bytes in size. The sequence listing contained in this .txt file is part of the specification and is hereby incorporated by reference herein in its entirety.

### BACKGROUND AND FIELD OF THE INVENTION

Considerable improvements in the expression of recombinant transgenes have steadily enhanced productivity of cells expressing recombinant proteins, in particular recombinant therapeutic proteins (Farrell, McLoughlin, Milne, Marison, & Bones, 2014; Kim, Kim, & Lee, 2012; Wurm, 2004).

Chinese hamster ovary (CHO) cells are a widely used host cell factory for the production of recombinant therapeutic proteins. They provide several advantages including their capacity to produce human-like post-translational modifications and to grow at high density in suspension in chemically-defined culture media. Moreover, CHO cells are considered to be a safe host for the production of recombinant therapeutic proteins (Hansen, Pristovsek, Kildegaard, & Lee, 2017).

Cell engineering has so far mainly focused on improving the time integral of viable cell concentration, by increasing the maximum viable cell density and extending culture duration, as well as on increasing the specific productivity of CHO cells, since both parameters are determinant for the volumetric productivity of recombinant therapeutic proteins (Farrell et al., 2014; Kim et al., 2012). This was notably achieved by modulating the expression of genes involved in various cellular functions such as apoptosis, metabolism, cell cycle and secretion (Fischer, Handrick, & Otte, 2015).

However, the fact that some cellular processes are not optimal in CHO cells or remain limiting for therapeutic protein production, and thus could be improved by the overexpression, downregulation or knock-out of specific genes has not yet been the subject of such intensive investigation (Baek, Kim, Park, & Lee, 2015; Hansen et al., 2017).

Protein folding in the endoplasmic reticulum (ER) is notably a critical step for therapeutic protein production, and it has therefore been widely investigated (Hansen et al., 2017). The protein disulfide isomerase (PDI) is an enzyme that catalyzes native disulfide bond formation, thus promoting protein folding. PDI is also involved in the rearrangement of erroneously formed disulfide bonds (Wang, Wang, & Wang, 2015). While some studies reported an increase in the specific productivity of several therapeutic proteins upon PDI overexpression, other studies observed no influence, or even a decrease in specific productivity or protein titer (Borth, Mattanovich, Kunert, & Katinger, 2005; Davis, Schooley, Rasmussen, Thomas, & Reddy, 2000; Hayes, Smales, & Klappa, 2010; Johari, Estes, Alves, Sinacore, & James, 2015; Mohan, Park, Chung, & Lee, 2007; Pybus et al., 2014). Another member of the PDI family, Erp57, was also investigated for its potential in improving therapeutic protein production. Erp57 triggers disulfide bond formation of glycosylated proteins via interaction with the two ER lectin chaperones calreticulin (CRT) and calnexin (CNX) (Tannous, Pisoni, Hebert, & Molinari, 2015). Upregulation of CHO cell derived-Erp57 or of both CNX and CRT was found to increase thrombopoietin specific productivity in CHO cells (Chung, Lim, Hong, Hwang, & Lee, 2004; Hwang, Chung, & Lee, 2003). However, expression of the mouse version of Erp57 decreased specific productivity of the CC-Antitrypsin and of the C1 esterase inhibitor (Hansen et al., 2015). These contradictory effects might result from distinct enzyme expression level, origin, as well as on the expressed therapeutic protein (Hansen et al., 2017).

Given the plethora of genes whose expression may be modulated to possibly improve therapeutic protein production, more global engineering strategies have focused on the expression of transcription factors that can act as master regulators of gene expression (Gutierrez-Gonzalez et al., 2019). Notably, overexpression of the ER stress-related transcription factors sXBP1, sATF6, ATF4 and CHOP successfully increased the specific productivity and/or titer of various therapeutic proteins (Becker, Florin, Pfizenmaier, & Kaufmann, 2008; Cain et al., 2013; Gulis, Simi, de Toledo, Maranhao, & Brigido, 2014; Haredy et al., 2013; Ku, Ng, Yap, & Chao, 2008; Nishimiya, Mano, Miyadai, Yoshida, & Takahashi, 2013; Ohya et al., 2008; Pybus et al., 2014; Tigges & Fussenegger, 2006), although contradictory results were obtained upon sXBPI overexpression (Ku et al., 2008; Rahimpour et al., 2013). Furthermore, overexpression of YY1, a zinc finger transcription factor with pleiotropic effects on many cellular processes, led to an increase in antibody titer in CHO cells (Tastanova et al., 2016).

Despite these progresses, it has been so far difficult to identify CHO cell activities whose up or downregulation may consistently yield favorable effects, irrespective of the therapeutic protein. Moreover, few of the Chinese hamster genes have been investigated for their potential in improving therapeutic protein production. At the same time, there is a growing number of chimerical or engineered therapeutic proteins that remain difficult to express at sufficient titers for clinical and therapeutic use (Hansen et al., 2017).

It was previously described how the deprivation of vitamin B5 in CHO (Chinese Hamster Ovary) cell culture medium combined with the transfection of an expression vector for a vitamin B5 transport protein, allowed the identification of cell variants that are capable of expressing recombinant proteins at very high levels, including the “easy-to-express” (ETE) Trastuzumab antibody, as well as “difficult-to-express” (DTE) proteins that otherwise cannot be expressed at levels that are sufficient for therapeutic protein production, such as Infliximab (International patent publication WO2016/156574, US Patent publication no. 20180066268).

However, these highly productive and efficient cells still constitute a very small proportion of all stably transfected and selected cells, and therefore remain hard to identify and to isolate.

The publications, including patents and patent publications, referenced in the text and/or in the appended bibliography are incorporated herein by reference in their entirety.

There is a need in the art to identify the specific alterations of these high producer cells that confer such desired production properties to construct cell lines, in particular CHO cell lines, that are permanently more efficient in the production of a molecule of interest. There is also a need in the art to use the knowledge of the specific alterations found in high producer cells to produce expression systems and cells that have such properties. This and other needs are addressed by the invention described herein.

### SUMMARY OF THE INVENTION

Disclosed herein is the use of expression vectors/systems expressing MIPs (i.e. metabolism influencing products), in particular their use to express a MIP or combination of MIPs, preferably to improve the metabolism of mammalian cells such as CHO cells, more specifically to improve the metabolism of mammalian cells that causes an increase of the production of, e.g., a protein of interest, preferably a therapeutic protein. Disclosed herein are also cell engineered to express the MIP(s). The MIP candidates are listed in Table 1, and preferably pertain to the cellular functions listed in FIG. 1D.

MIPs preferably comprise the mPPARα and/or Foxa1 transcription factors, m(mouse)PPARα- and/or Foxa1-activated CHO cell genes or homologs such as human homologs, structural proteins such as actin, proteins involved in the cell basal metabolism such as mRNA translation, signaling and trafficking activities such as Tagap, Rassf9, Erp27, Erp57, Clstn3, cell survival proteins CDK15 and Ca3, apoptosis such as CFLAR or SOD1, glutathione catabolism such as GCLM or GGCT, or specific combinations thereof. The cells of the present invention overexpress said MIP or MIP human homolog, and/or are treated with a chemical that increases the activity of said MIP, such as the bezafibrate PPAR agonist and other chemical or biological agonists.

In one embodiment, the invention is directed at a eukaryotic expression system comprising:

at least one metabolism influencing product (MIP) expression vector comprising at least one nucleic acid encoding the at least one MIP under the control of at least one regulatory sequence, wherein the MIP is preferably one of Table 1, in particular:
at least one transcription factor, more preferably a pioneer transcription factor such as Foxa1 (Forkhead box protein A1) or at least one transcription factor involved in fatty acid metabolism such as at least one PPAR (Peroxisome proliferator-activated receptors),
at least one factor that regulates RNA translation, such as Casc3 and/or
at least one structural protein such as actin and/or protein folding proteins such as Erp27 (Endoplasmic Reticulum Protein 27), or a protein interacting with the respective protein folding protein such as Erp57 (Endoplasmic Reticulum Protein 57),
at least one protein involved in signal transduction, vesicular trafficking and or cell adhesion activities such as Tagap (T cell activation GTPase activating protein), Rassf9 (Ras Association Domain Family Member 9), and/or Clstn3 (Calsyntenin 3),
at least one protein involved in cell survival and/or proliferation such as CDK15 (Cyclin Dependent Kinase 15) or Ca3 (Carbonic Anhydrase 3),
at least one protein involved in apoptosis such as CFLAR (CASP8 And FADD Like Apoptosis Regulator) or SOD1 (Superoxide Dismutase 1) and/or
at least one protein involved in glutathione catabolism such as GCLM (Glutamate-
Cysteine Ligase Modifier Subunit) or GGCT (Gamma-glutamylcyclotransferase).

The at least one MIP may comprise at least one PPAR, in particular PPARα, PPARβ/δ or PPARγ and/or Foxa1, actin, Erp27 optionally combined with Erp57. The at least one regulatory sequence maybe a promoter selected from the group of CMV, EF1alpha, CMV/EF1alpha, SV40, RSV, PGK, a promoter having an expression level of CMV, EF1alpha, CMV/EF1alpha, SV40, RSV, PGK and combinations thereof.

The at least one MIP may comprise at least one (including, e.g., two or three) primary MIP and at least one, or two or three further MIPs which is/are neither a primary nor a secondary MIP. There may be at least 2, 3, 4, 5 or more MIPs in one eukaryotic expression system. The MIP expression vector may further comprise a first ITR (inverted terminal repeat) upstream and a second ITR downstream of the nucleic acid encoding the MIP. The at least one regulatory sequence may comprise a MAR element or MAR construct, such as MAR 1-68 and/or MAR X-29, including a singular MAR element or MAR construct, optionally between the first and second ITR. The MIP expression vector may be a transposon donor vector. The expression system may further comprise a transposase-expressing helper vector or mRNA. The transposase expressing helper vector may comprise the PB (piggybac) transposase coding sequence, optionally flanked, upstream and downstream by untranslated terminal regions (UTR).

The eukaryotic expression system may further comprise a carrier vector comprising at least one restriction enzyme cleavage site adapted for insertion of a nucleic acid encoding a protein of interest. The carrier vector may further comprise an antibiotic resistance gene and/or a vitamin transport protein such as sodium-multivitamin transporter SLC5A6. The elements of the carrier vector may also be part of another vector of the expression system.

In another embodiment, the invention is directed at a method comprising:

(a) transfecting a cell with any one of the expression vectors of the expression system disclosed herein and/or

adding to the eukaryotic cell at least one activator of a protein product of a gene expressing a MIP, and

(b) optionally, transfecting the cells with a carrier vector comprising a protein of interest.

The at least one activator added to the eukaryotic cell may be an activator of at least one, two or all PPARs in particular PPARα, PPARβ/δ or PPARγ, such as bezafibrate. The MA/EL of the protein of interest may be more than 1.5× the ML, more than 2× the ML or even more than 2.5× or 3× the ML.

In certain embodiments, the invention is directed at a kit comprising in one container, said eukaryotic expression system of any one of the preceding claims and, in a second container, instructions of how to use said system. The kit may further comprise at least one activator of the at least one MIP, wherein the MIP is preferably at least one PPAR, in particular PPARα, PPARβ/δ or PPARγ, and the activator may be an activator of at least one, two or all PPARs such as bezafibrate.

The invention is also, in certain embodiments, directed at a recombinant eukaryotic cell, such as a Chinese Hamster Ovary (CHO) cell, comprising any of the eukaryotic expression systems disclosed herein. The cell may be stably transfected with the MIP expression vector or a part thereof comprising the at least one, at least two, three or four MIPs.

The invention is also directed to an eukaryotic cell comprising at least one endogenous or exogenous MIP under the control of at least one exogenous promoter, which might be part of a promoter ladder, selected from the group of CMV, EF1alpha, CMV/EF1alpha, SV40, RSV, PGK, a exogenous or recombinant endogenous promoter having an expression level of CMV, EF1alpha, CMV/EF1alpha, SV40, RSV, PGK and combinations thereof.

### BRIEF DESCRIPTION OF THE FIGURES

FIG. 1: Identification of MIP through transcriptomic analyses.

(A) Transcriptomic analyses outline by RNASeq, comparing non-selected, B5-selected and antibiotic-selected cells producing an easy-to-express (ETE) or difficult-to-express (DTE) protein of interest. (B) Graphs representing the two main expression patterns of the selected B5 target genes, here in ETE cells. Gene expression correspond to gene read counts from RNA-Seq analyses. (C) Identification of transcripts upregulated in Trastuzumab high producing clones compared to CHO-M wild type (WT) cells and compared to cells polyclonal for Trastuzumab production. 51 mRNAs encoded by 32 genes were identified. (D) Functional classes of the candidate genes identified through transcriptomic analyses and literature screening (see Table 1).

FIG. 2: Effect of candidate MIPs on easy-to-express (ETE) proteins of interest: Trastuzumab production.

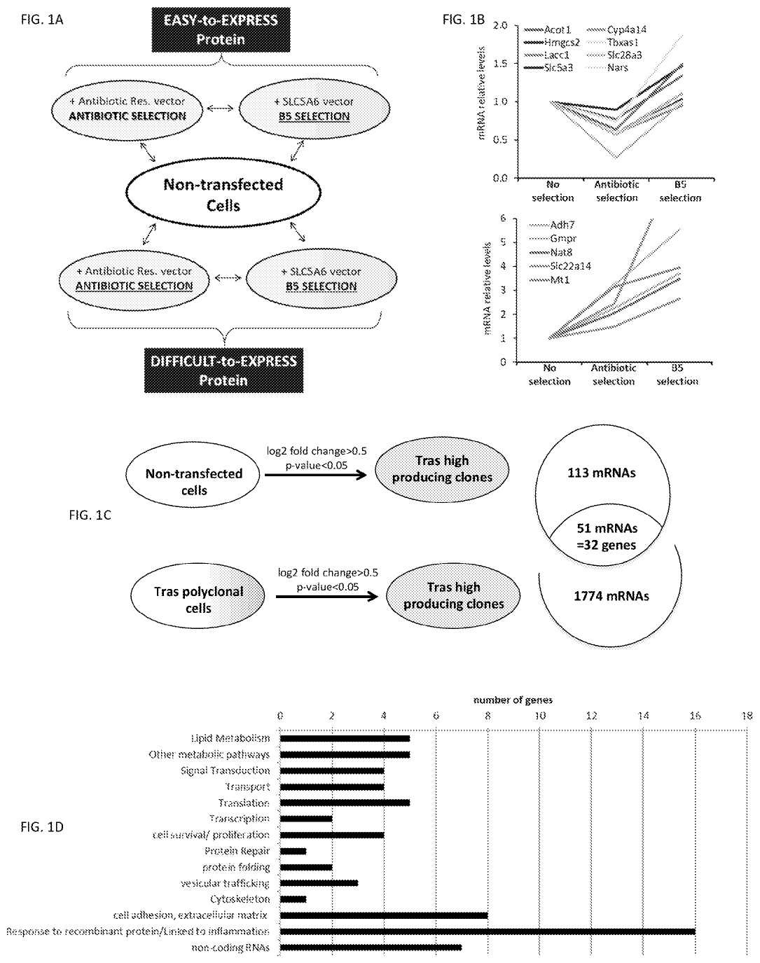

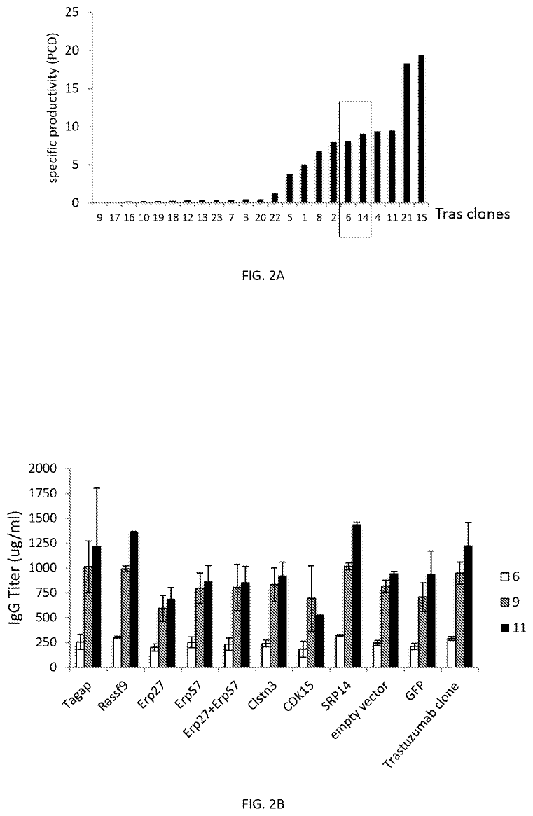

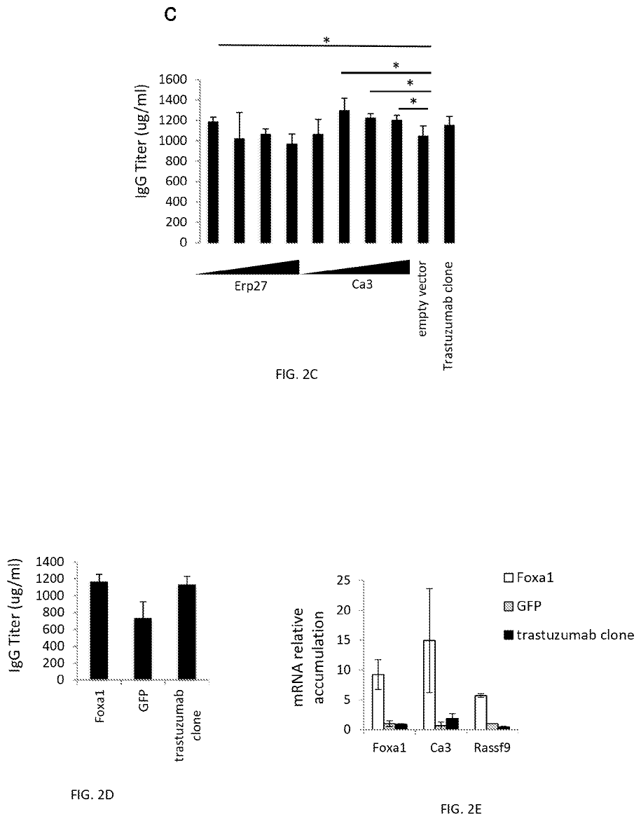

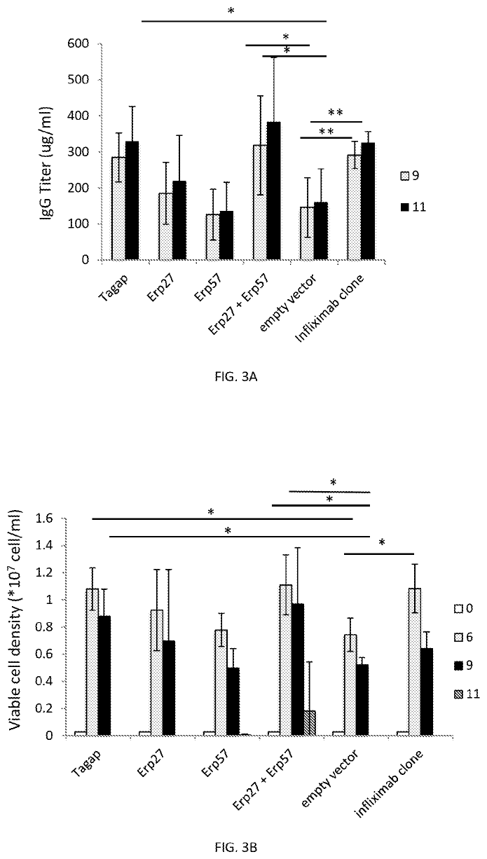

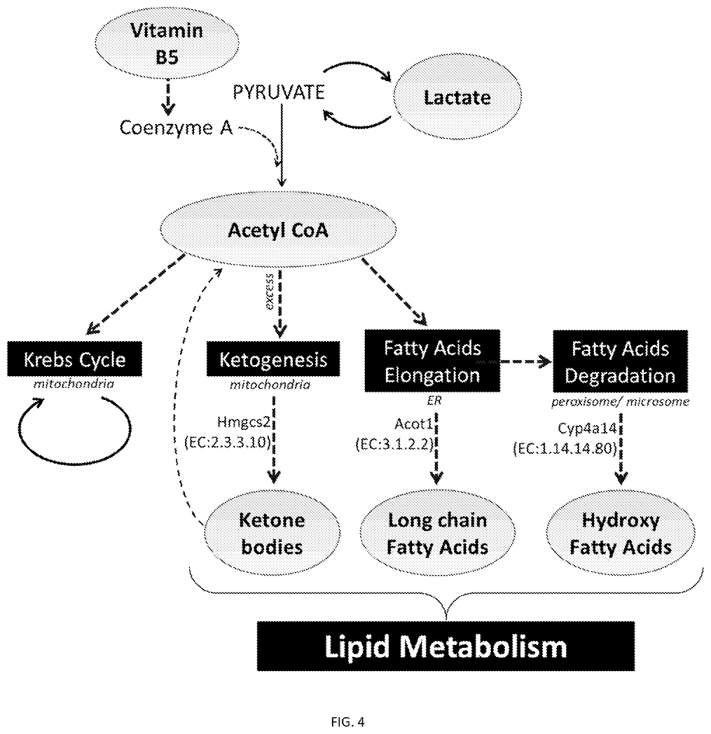

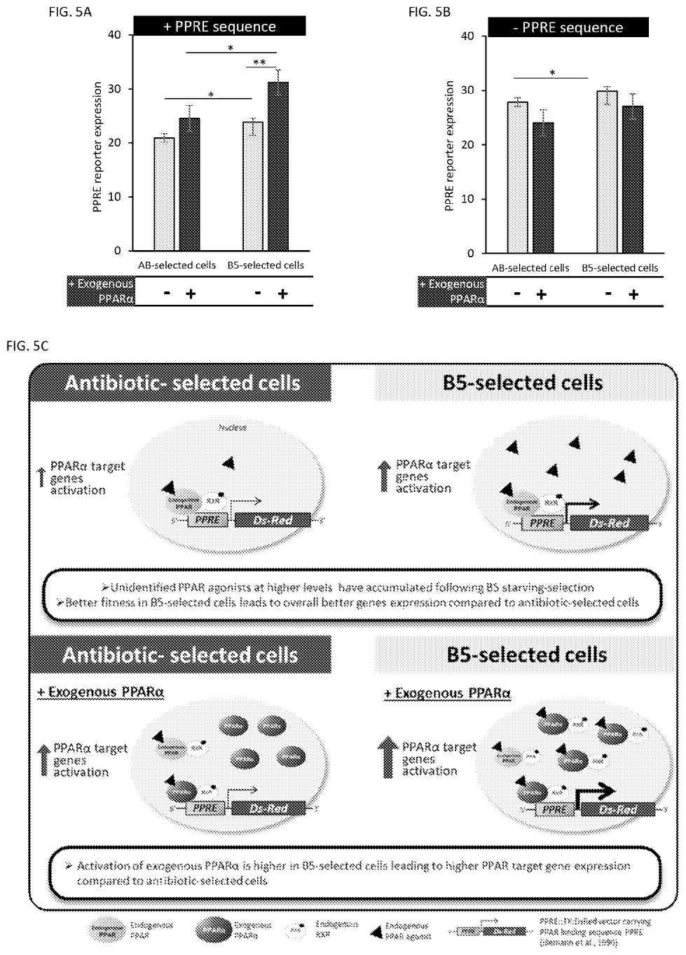

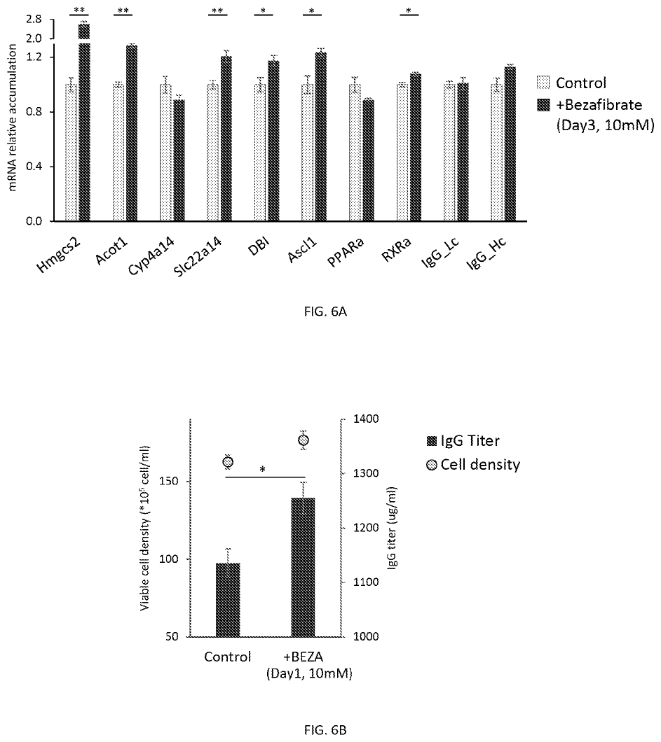

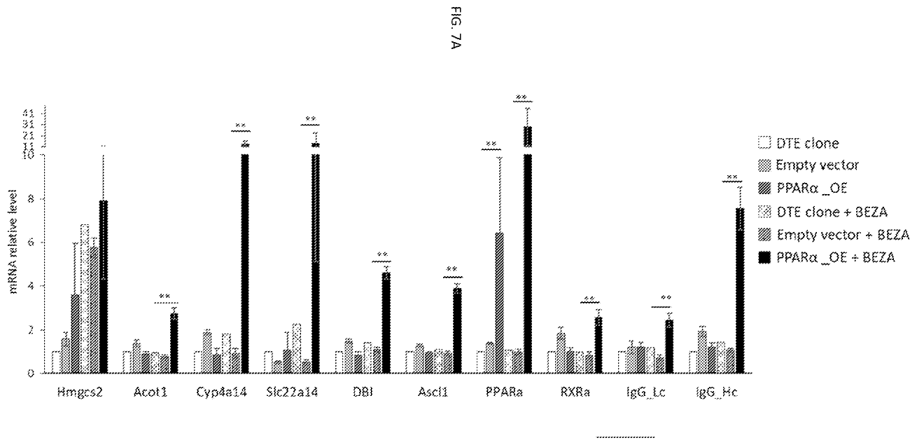

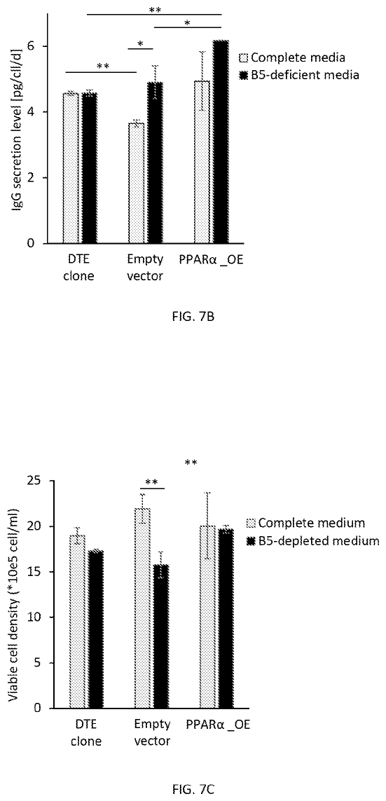

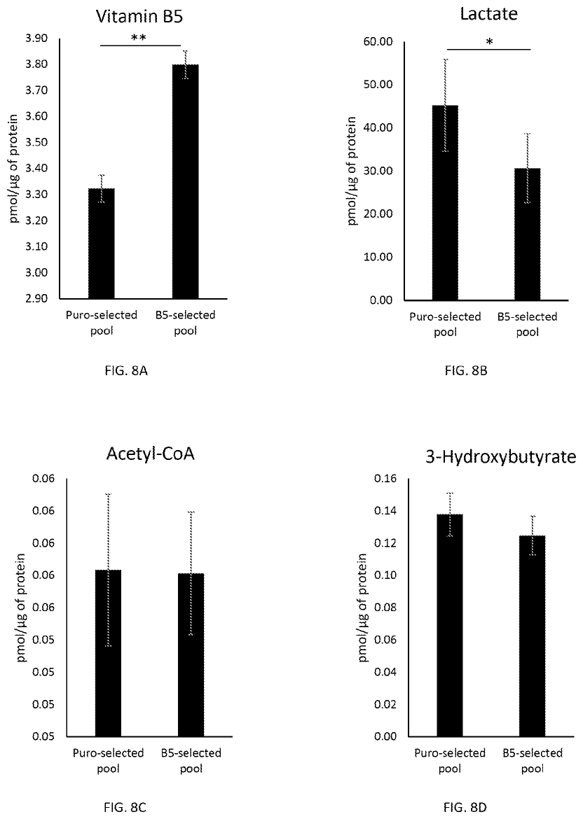

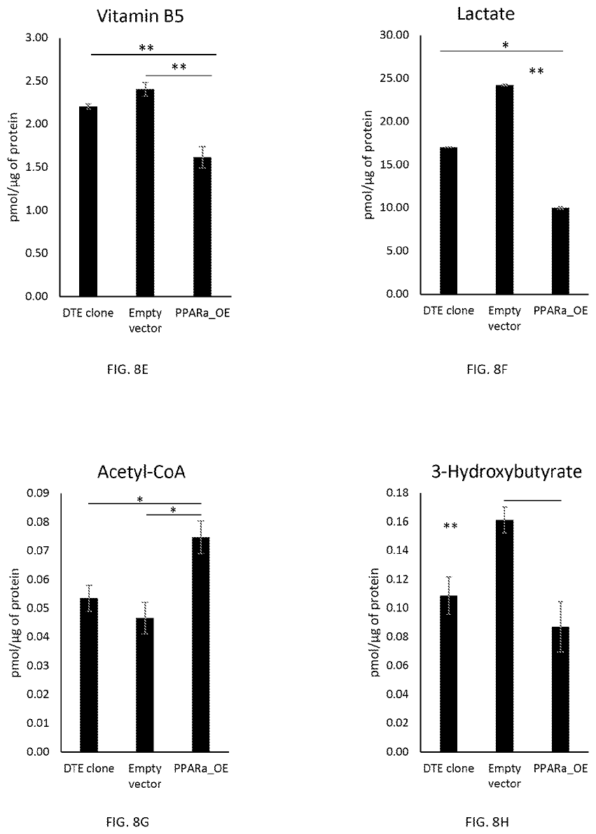

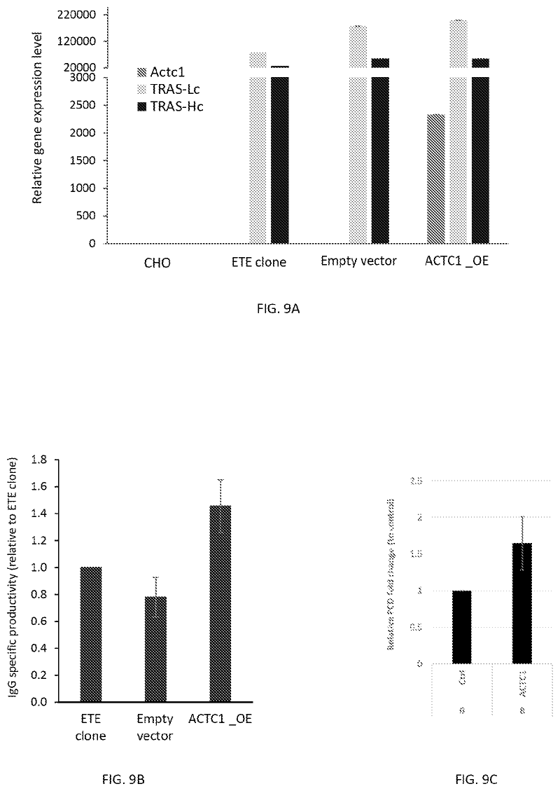

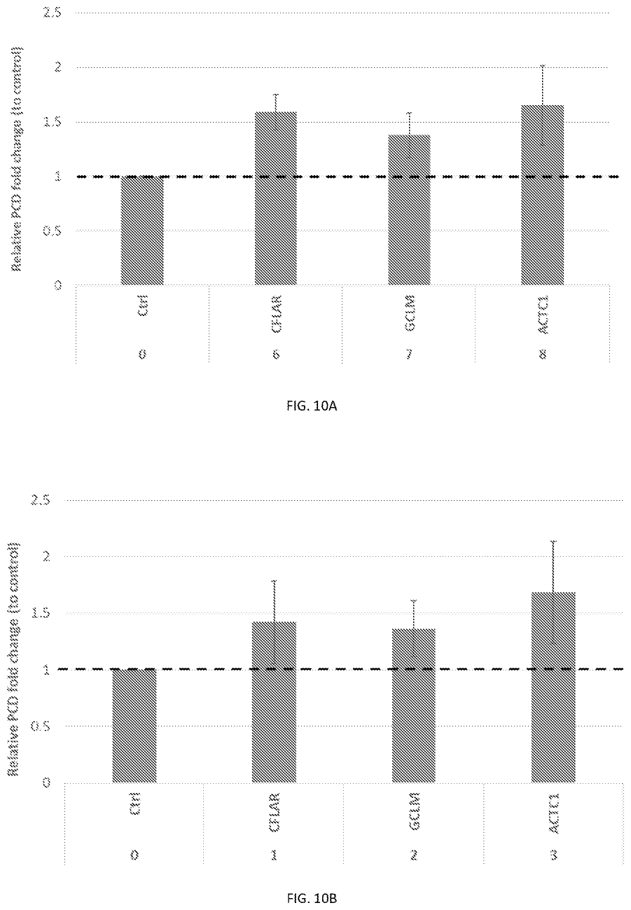

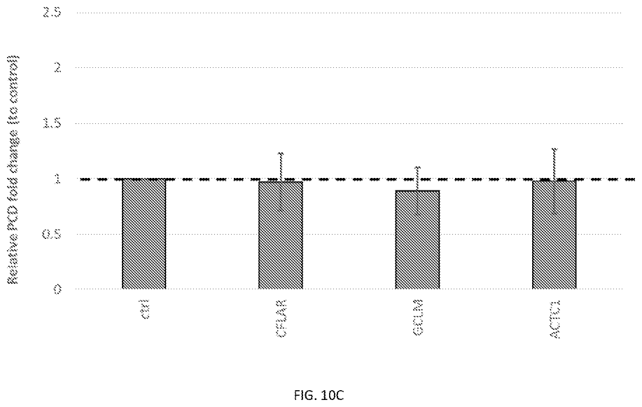

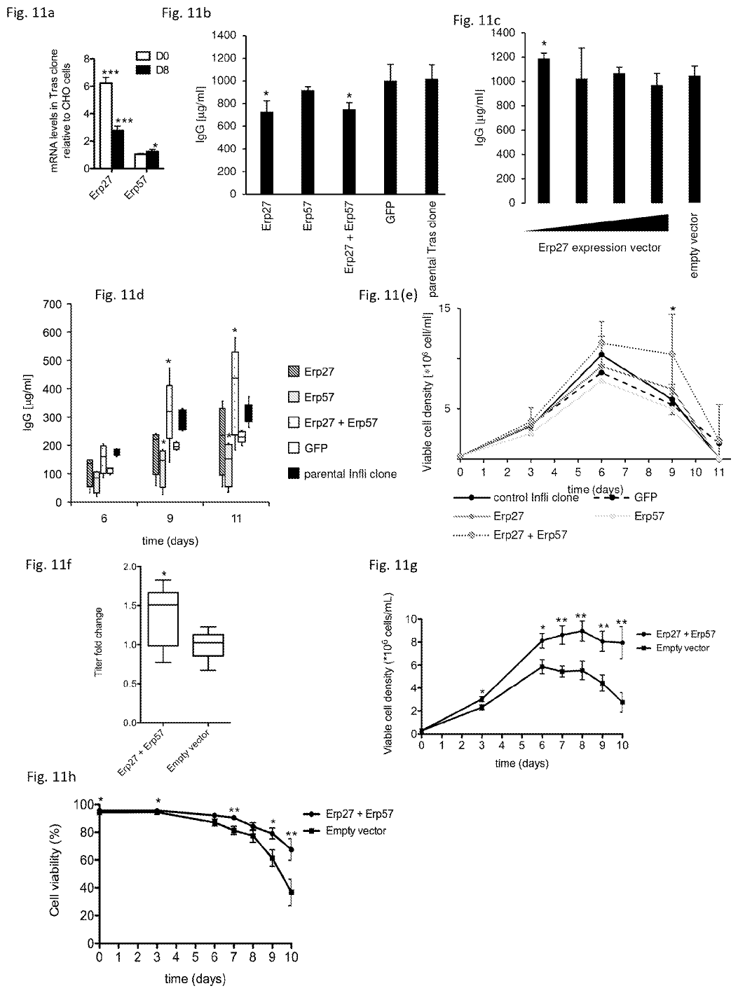

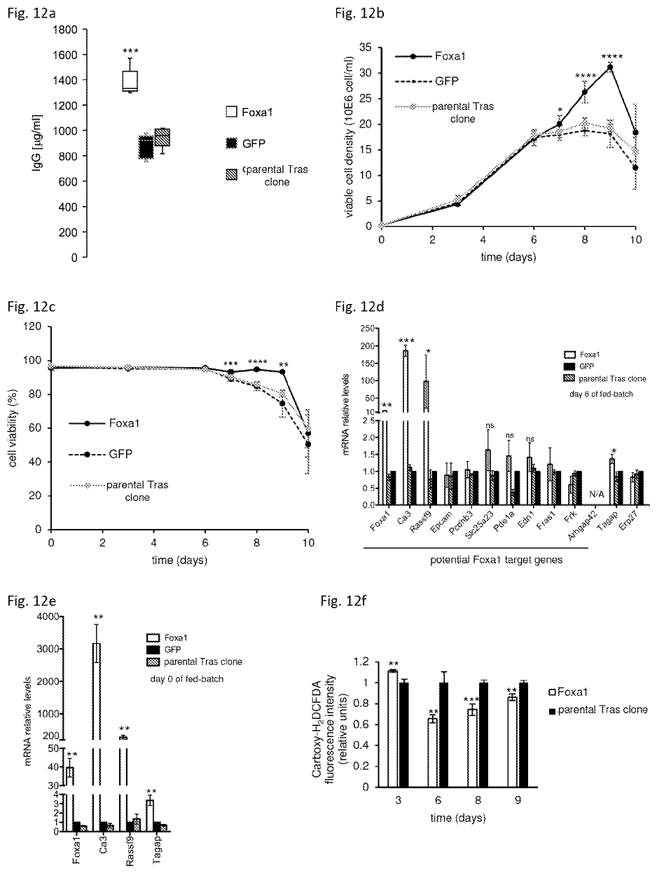

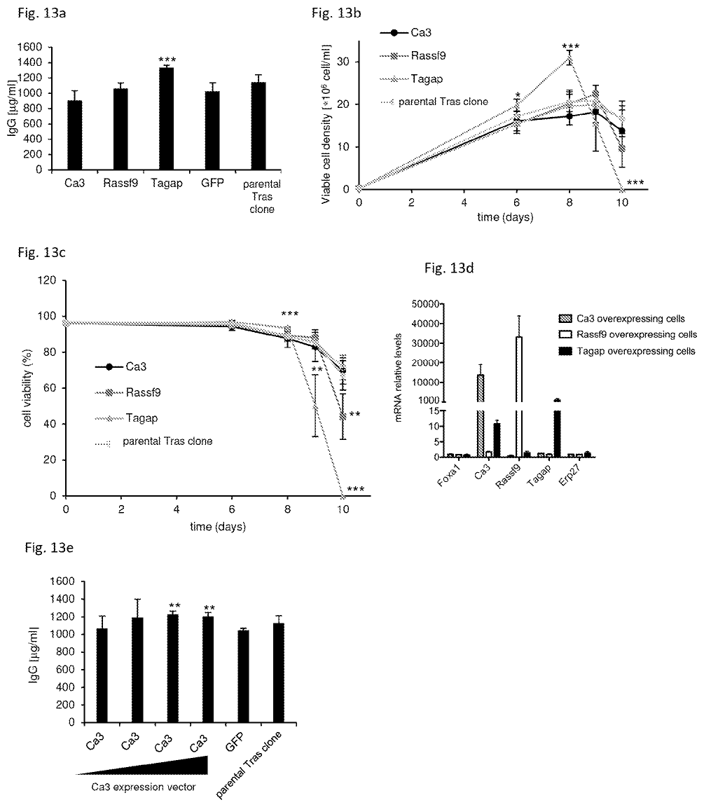

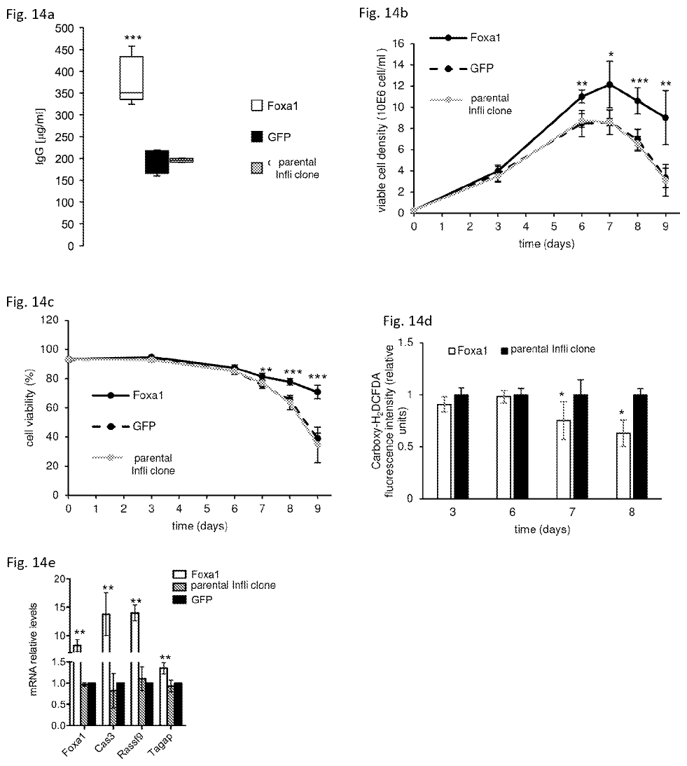

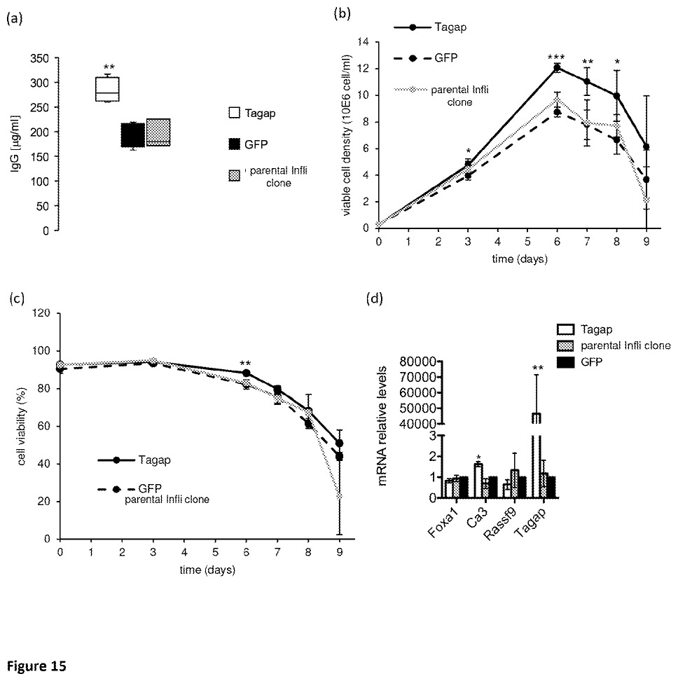

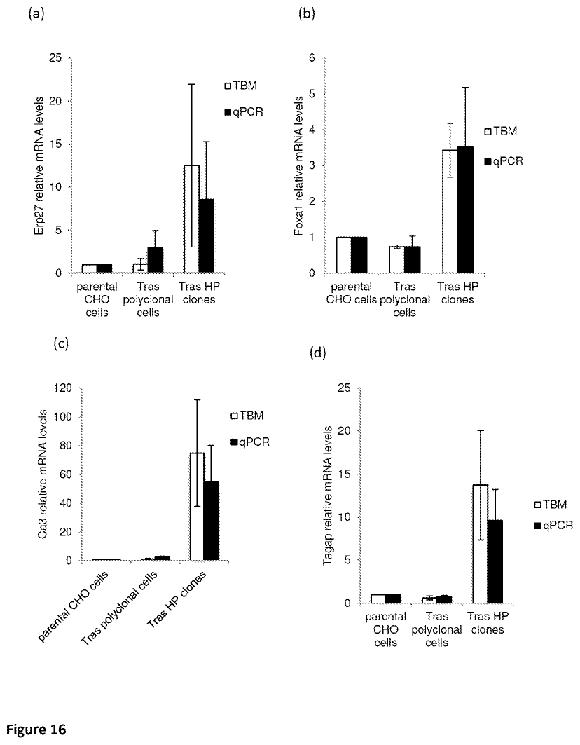

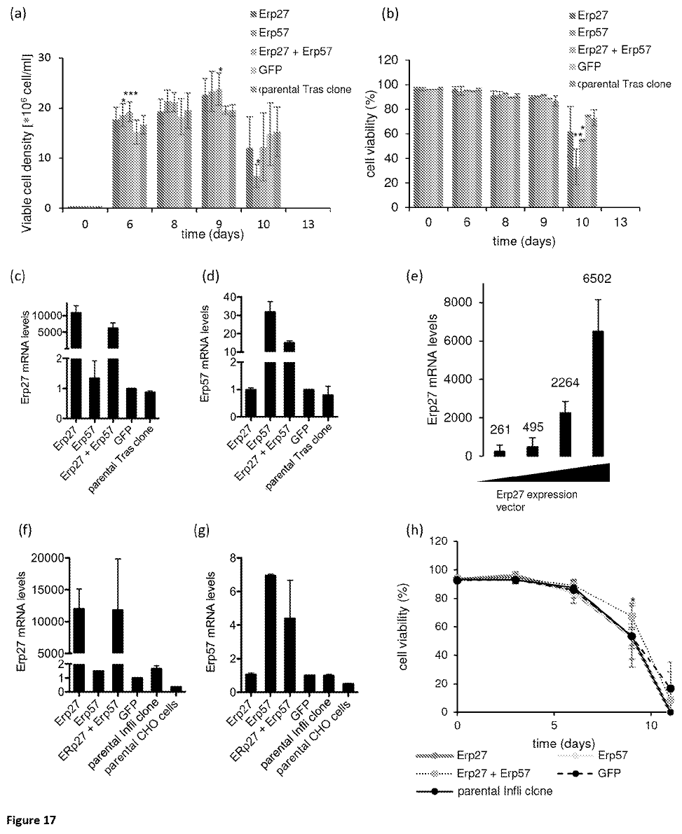

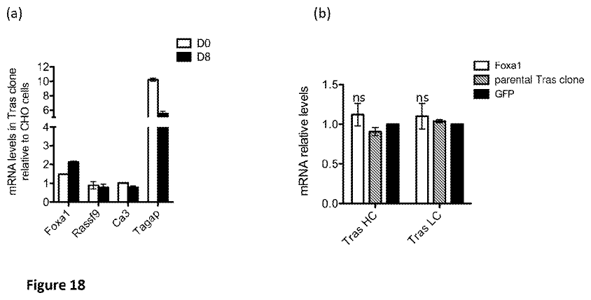

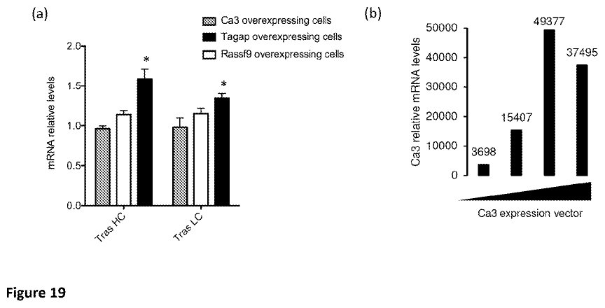

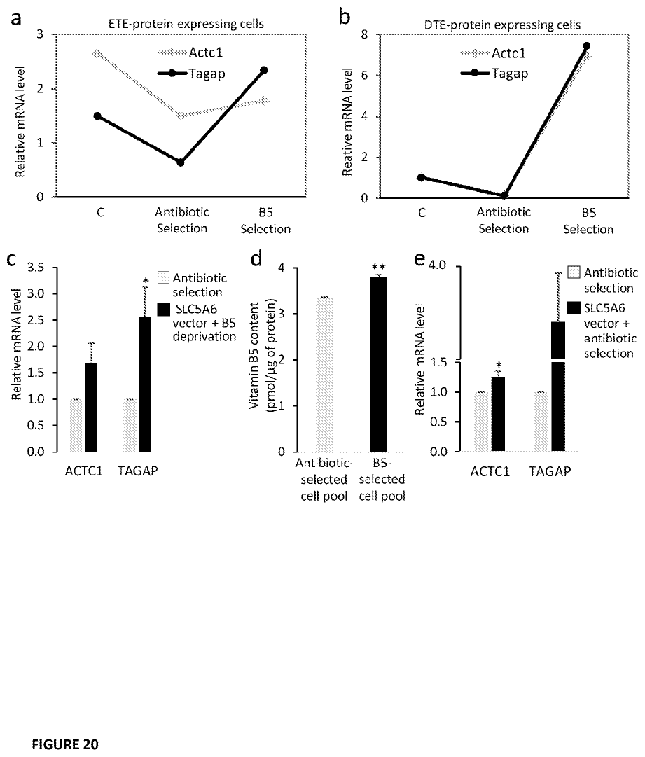

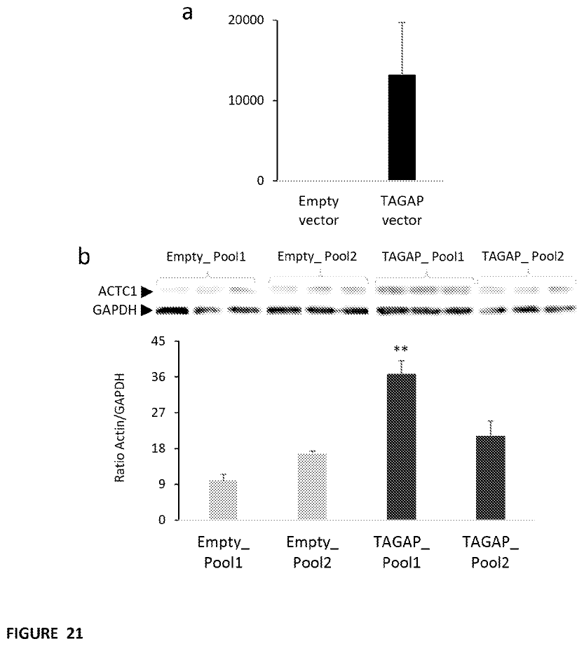

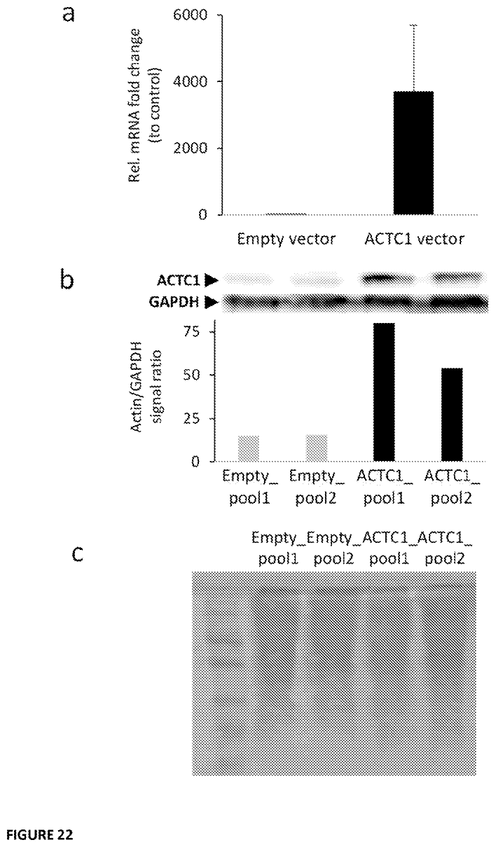

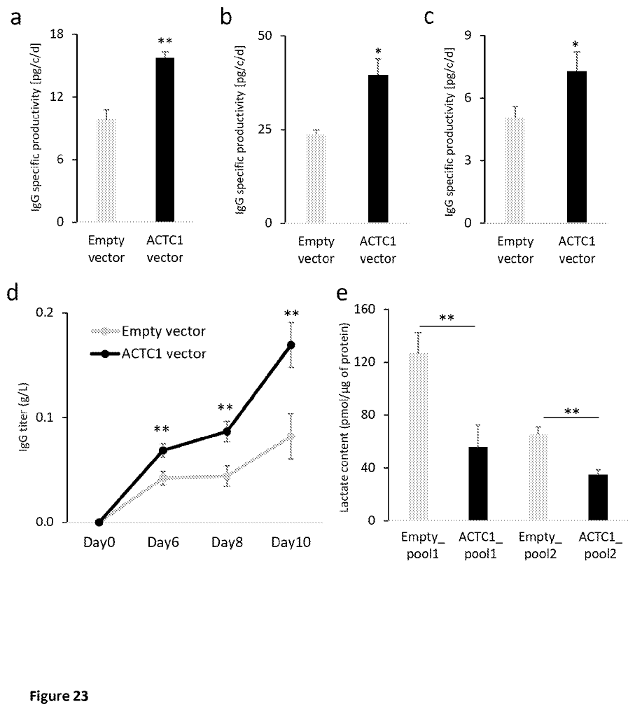

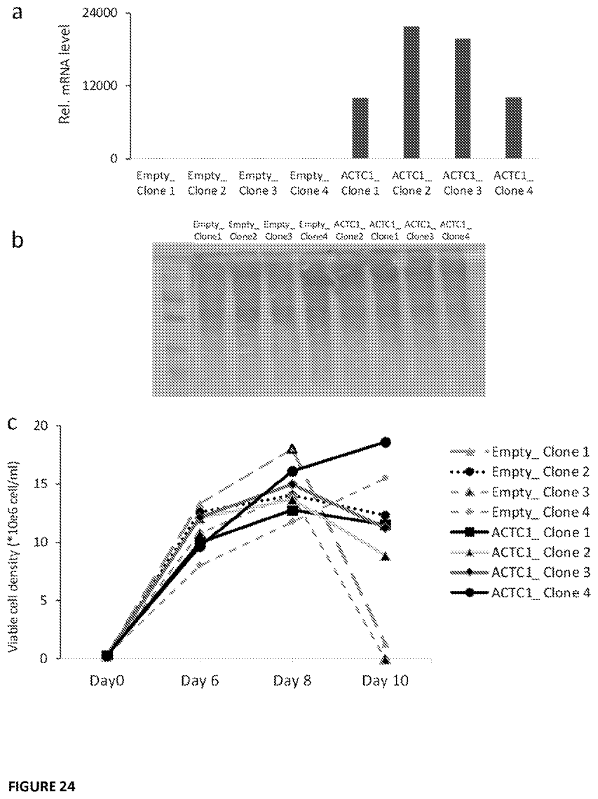

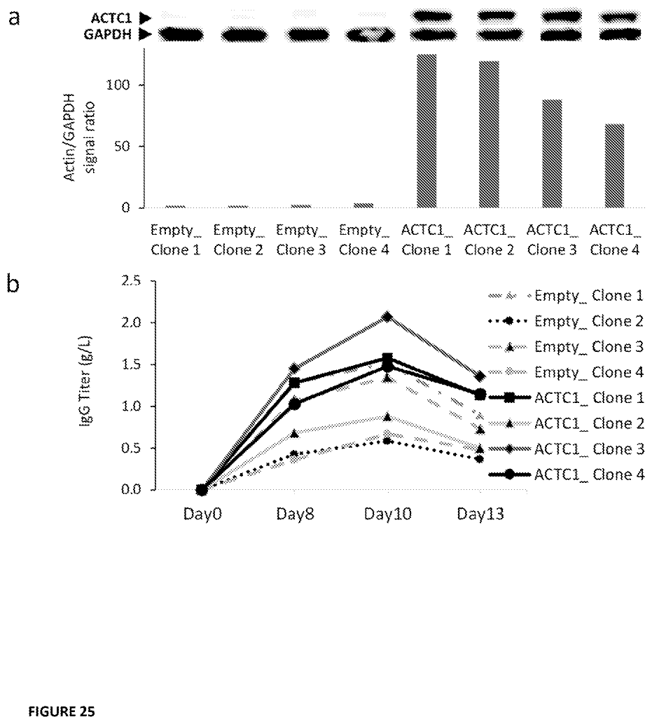

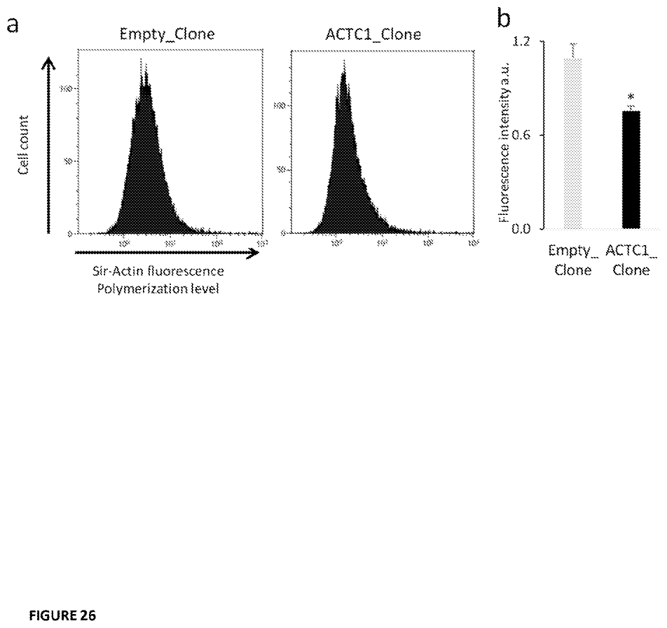

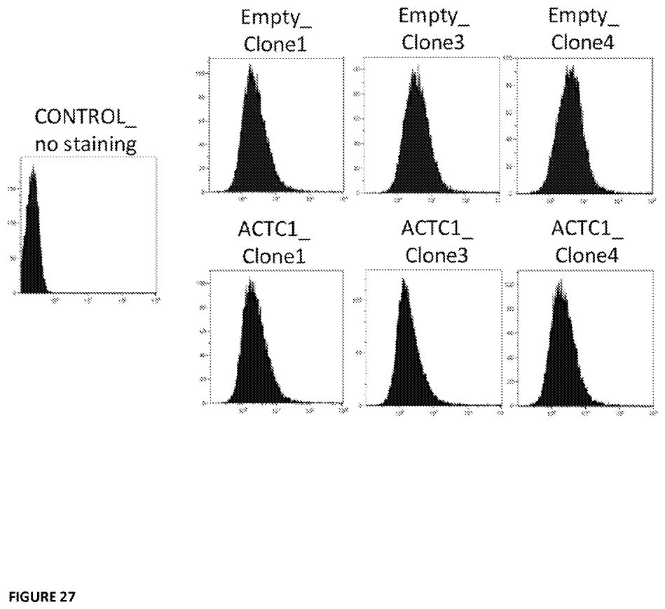

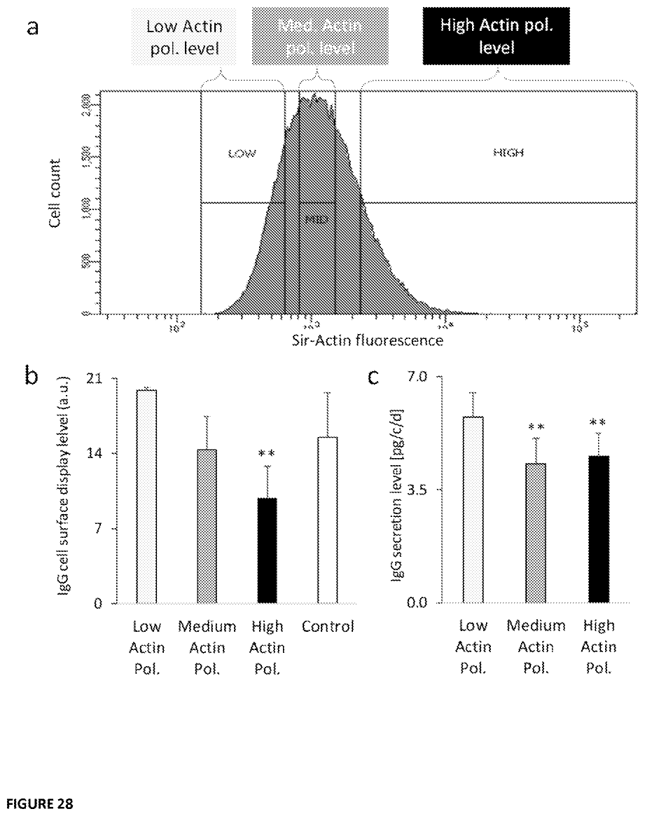

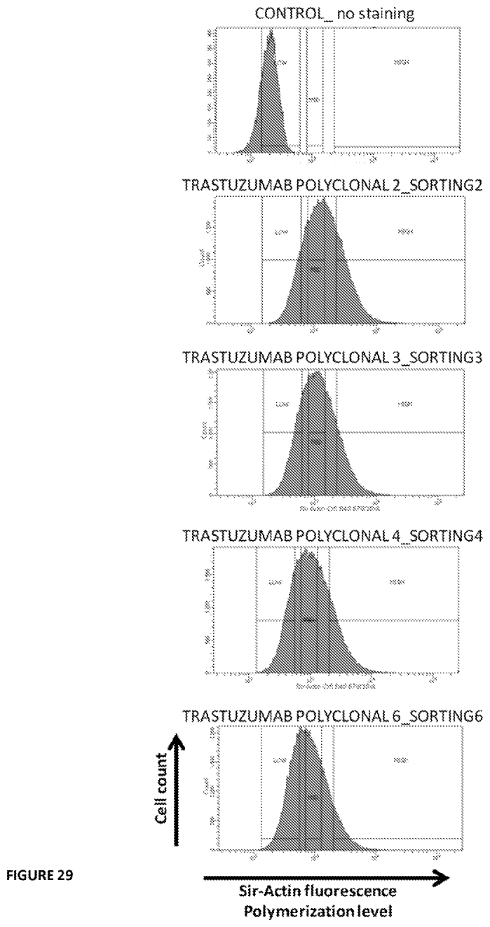

(A) Clones were isolated from the Trastuzumab polyclonal population used for the transcriptomic analysis (see FIG. 1C). Middle producing clones Tras6 and Tras14 that maintained a fast cell division rate in fed-batch cultures were used in the following experiments. (B) Production of the easy-to-express (ETE) Trastuzumab antibody at days 6 (white bars), 9 (grey bars) and 11 (black bars) of fed-batch cultures after stable overexpression of candidate MIPs in the Tras14 clones. SRP14 overexpression was used as positive control (LeFourn et al., 2013) and cells expressing GFP or transfected with an empty vector were used as negative control. (C) Production of Trastuzumab antibody at day 11 of fed-batch cultures. Stable cells expressing an increasing amount of the specified MIP were obtained by transfecting an increasing amount of MIP plasmid in Trastuzumab clone. (D) Production of Trastuzumab antibody at day 13 of fed-batch cultures after stable overexpression of Foxa1 in Trastuzumab clone. (E) Relative expression of Foxa1, Ca3 and Rassf9 in cells stably transfected with Foxa1 (white bars), GFP (grey bars) and in untransfected cells (black bars), to illustrate the increased expression of the Ca3 and Rassf9 secondary MIPs upon the overexpression of the Foxa1 primary MIP. RNA was extracted at day 8 of fed-batch cultures.

FIG. 3: Effect of candidate MIPs on difficult-to-express (DTE) proteins of interest; Infliximab expression.

MIPs were stably overexpressed in recombinant clones expressing the difficult-to-express (DTE) Infliximab antibody. (A) Production of Infliximab antibody at days 9 (grey bars) and 11 (black bars) of fed-batch cultures after overexpression of candidate genes. Cells transfected with an empty vector were used as negative control. (B) Viable cell density of cells at days 0 (white bars), 6 (light grey bars), 9 (black bars) and 11 (dark grey bars) of fed-batch culture.

FIG. 4 provides a schematic outline highlighting B5-target genes found to be regulated by PPAR.

FIG. 5: PPAR activation studies in easy-to-express (ETE) cells: endogenous PPAR agonists in B5-selected cells.

PPAR transient activation assay using a PPAR-reporter DsRed gene whose promoter contains PPAR-responsive elements (PPRE). (A) Antibiotic (AB)-selected ETE cells and B5-selected ETE (Trastuzumab) cells were transiently transfected with the peroxisome proliferator response element (PPRE)-DsRed reporter, or co-transfected with the PPRE reporter and mouse PPARα. DsRed activity was standardized relative to BFP2 marker (see Materials and Methods). (B) Negative control for PPAR transient assay corresponds to DsRed activity without PPRE reporter. Data from panels A and B represent mean fluorescence±SE of corrected DsRed activity from four independent experiments. Statistics: * P≤0.05 and ** P≤0.02 (1 sided t-test; paired sample). (C) Schematics explaining the transient PPARα activation data.

FIG. 6: PPAR activation studies in easy-to-express (ETE) clone: effect of exogenous pan PPAR agonist.

(A) ETE clone (Trastuzumab) non-treated (Control) or treated with 10 mM Bezafibrate PPARα ligand after 3 days of fed-batch culture (+Bezafibrate). Candidate gene expression were quantified at day 6 by RT-qPCR. (B) ETE cells treated with 10 mM Bezafibrate after 1-day fed-batch, and IgG titer measured after 10 days. Data are means±SE from four independent experiments. * P≤0.05 and ** P≤0.02 (t-test; 2 sided; unpaired sample, unequal variance).

FIG. 7: PPARα overexpression in difficult-to-express (DTE) cells.

Antibiotic-selected DTE (Infliximab) cells were stably transfected with mouse PPARα or with an empty vector. Analyses of gene expression, IgG titer and cell viability were performed comparing DTE clone with empty vector cell and PPARα overexpressing cells (PPARα_OE). (A) Gene expression of PPARα targets, PPARα and IgG (qRT-PCR) in none-treated cells or cells treated with Bezafibrate (BEZA). Bezafibrate was added at 10 mM at day 1 of the fed-batch culture. RNA was extracted at day 6 of the fed-batch culture. Infliximab IgG specific productivity (B) and cell viability (C) are illustrated in non-selective or B5-starving media. Cells were cultured in 12 well-plate at a starting amount of 2*105 cell/ml in non-selective or B5 starving medium for 5 days, then transferred in non-selective media. IgG specific productivity (PCD) was then measured over 3 days of culture in non-selective media. Each measurement is the result of three independent cultures. Statistics: * P≤0.05 and ** P≤0.02 (t-test; 1 sided; unpaired sample, unequal variance).

FIG. 8: Metabolic analysis of antibiotic- or B5-selected CHO cells overexpressing or not the PPARα MIP.

Vitamin B5 (FIG. 8A), lactate (FIG. 8B), acetylCoA (FIG. 8C), and ketone (3-Hydroxybutyrate) (FIG. 8D) were quantified by LC-HRMS (liquid chromatography coupled to high-resolution mass spectrometry) on puromycin or B5-selected polyclonal cell pools, as indicated. Data represent ±SE from four independent biological experiments. Statistics: * P≤0.05 and ** P≤0.02 (2 sided t-test; paired sample). The levels of these metabolites were also quantified on antibiotic-selected DTE (Infliximab) cells, on a DTE cell pool stably transfected with mouse PPARα expression vector (PPARα_OE) or with an empty vector (FIGS. 8E-8H). Data represent ±SE from three technical independent experiments. Statistics: * P≤0.05 and ** P≤0.02 (2 sided t-test; equal variance).

FIG. 9: ACTC1 overexpression in ETE and DTE CHO cells.

ETE (Tastuzumab, “TRAS”) and DTE (Fc-fusion protein) cells were stably transfected with Chinese hamster ACTC1 cDNA encoding actin or with an empty vector. Analyses of gene expression and IgG titer were performed comparing ETE clones (Control) with empty vector cells and ACTC1 overexpressing cells (ACTC1_OE). (A) ACTC1, IgG light chain (Lc) and heavy chain (Hc) gene expression (qRT-PCR). Each measurement is the result of two independent cultures. (B) IgG specific productivity (PCD), i.e. picogram of secreted IgG per cell and per day) is measured over 3 days of culture in non-selective media. (C) Production in DTE Fc-fusion protein (Ctrl=control, empty vector).

FIG. 10: effect of individual or combined expression of CFLAR, GCLM and ACTC1 on the secretion of an IgG1-bevacizumag-expressing CHO-M clone, an Fc-fusion-expressing CHO-M clone and a Fab-enzyme fusion-expressing clone.

A bevacizumag-expressing clone (FIG. 10A), an fc-fusion-expressing clone (FIG. 10B) and a fab-enzyme-fusion expressing clone (DTE) (FIG. 10C) were re-transfected with various individual or combination of transposable CFLAR- (CASP8- and FADD-like apoptosis regulator), GCLM-(Glutamate-cysteine ligase regulatory subunit), ACTC1-expression vectors. The specific productivity of the resulting cell pools was then evaluated through their subcultivation in batch conditioned every 3 or 4 days. Results were represented as a % of their respective bevacizumab- or Fc-fusion-control cells PCD values (pg/cell/day).

FIG. 11. Effect of Erp27 and/or Erp57 overexpression on the production of therapeutic proteins. Clones producing easy- or difficult-to-express therapeutic proteins were stably transfected with Erp27 or Erp57 expression vectors, or co-transfected with both Erp27 and Erp57 expression vectors. Gene expression, cell growth, cell viability and protein production were evaluated in fed-batch cultures in stable polyclonal populations (panels a-e) or in clones (panels f-h). (a) Quantification of Erp27 and Erp57 mRNA levels in the Tras-producing clone, represented as fold-change relative to their levels in the non-transfected parental CHO cells at day 0 and day 8 of fed-batch cultures, as assessed by qRT-PCR. Error bars are shown as SD, n=3, p values were determined using the unpaired one-tailed t-test. (b) The Tras-producing clone (parental Tras clone) was stably transfected with the Erp27 and/or Erp57 expression vectors, and the titers of the secreted trastuzumab antibody were determined from cell culture supernatants at the end of fed-batch cultures. Cells transfected with a GFP expression vector were used as control. Error bars are shown as SD, n=3, unpaired one-tailed t-test. (c) The parental Tras clone was stably transfected with decreasing amounts of the Erp27 expression vector together with an empty vector to keep the total amount of plasmid constant. Cells transfected with an empty vector plasmid were used as control. Trastuzumab titer were determined at the end of fed-batch cultures. Error bars are shown as SD, n=3, unpaired one-tailed t-test. (d) An infliximab producer clone was characterized in terms of the secreted monoclonal antibody titers obtained during fed-batch cultures using either the parental clone or derived cell populations obtained after transfection with the Erp27 and/or Erp57, or with the GFP expression vector, as indicated. Titers are illustrated as Tukey box-and-whisker diagram with median values (middle bar) and 25-50% and 50-75% quartiles (box). Whiskers extend to the lowest and highest values still within the 1.5-fold interquartile range. (e) Viable cell density of the fed-batch cultures analyzed in panel d. Error bars are shown as SD, unpaired one-tailed t-test (panels d and e, n≥4). (f) An etanercept producer clone was stably transfected with the Erp27 and Erp57 expression vectors, or with an empty vector as control. Cell colonies were isolated using a ClonePix® device, and the clones with the highest etanercept secretion halos were isolated and characterized for the etanercept titer at the end of fed-batch cultures. The titer fold-change relative to control cells is illustrated as Tukey box-and-whisker diagram as for panel d. (g, h) Viable cell density and cell viability of the fed-batch cultures analyzed in panel f. The error bars represent the SEM, panels f to h (n≥8).

FIG. 12. Effect of Foxa1 overexpression on Tras production.

The Tras producer clone was stably transfected with the Foxa1 or GFP expression vector. The trastuzumab titer (a), viable cell density (b) and cell viability (c) were evaluated during 10 days fed-batch cultures. n=5, unpaired one-tailed t-test. Titers are illustrated as a Tukey box-and-whisker diagram as described for FIG. 2, whereas error bars are shown as SD (panels b,c). (d) An RT-qPCR analysis of the mRNA levels of Foxa1 target genes and other relevant genes identified in FIG. 1c was performed on Foxa1 overexpressing cells, GFP expressing cells or the parental Tras clone at day 8 of the fed-batch culture. Error bars are shown as SD, n=3, paired one-tailed t-test. (e) RT-qPCR quantification of Foxa1, Ca3, Rassf9 and Tagap mRNA levels in Foxa1-overexpressing cells, GFP-expressing cells or in the parental Tras clone at day 0 of the fed-batch. Error bars are shown as SD, n=3, paired one-tailed t-test. (f) Evaluation of intracellular ROS levels using carboxy-H₂DCFDA in Foxa1 overexpressing cells and in parental Tras clone at day 0, 3, 6, 8 and 9 of the fed-batch cultures. Error bars are shown as SD, n=3, unpaired one-tailed t-test.

FIG. 13. Effect of Ca3, Rassf9 and Tagap overexpression on Tras production.

The Tras producer Tras6 clone was stably transfected with the Ca3, Rassf9, Tagap or GFP expression vector. The trastuzumab titer (a), viable cell density (b) and cell viability (c) were determined during 10-days fed-batch cultures. Error bars are shown as SD, n≥3, unpaired two-tailed t-test. (d) Quantification of the mRNA levels of candidate genes by RT-qPCR analyses in Ca3-, Rassf9- or Tagap-expressing stable populations. Data are presented relative to the mRNA levels in control GFP-expressing cells. Error bars are shown as SD, n=3, paired two-tailed t-test. (e) The Tras clone was stably transfected with various amounts of the Ca3 expression vector together with an empty vector to keep the total amount of plasmid constant. The trastuzumab titers obtained from these cells were assessed at the end of fed-batch cultures. Error bars are shown as SD, n=3, unpaired one-tailed t-test.

FIG. 14. Effect of Foxa1 overexpression on infliximab production.

The infliximab-producing clone was stably transfected with the Foxa1 or GFP expression vector, and the infliximab titer (a), viable cell density (b) and cell viability (c) were evaluated during 9-days fed-batch cultures. n=5, unpaired one-tailed t-test. Titers are depicted as described for FIG. 2 (panel a), whereas error bars are shown as SD (panels b,c). (d) Evaluation of intracellular ROS levels using carboxy-H₂DCFDA for Foxa1-overexpressing cells and for the parental infliximab-producing clone at days 3, 6, 7 and 8 of the fed-batch cultures. Error bars are shown as SD, n=3, unpaired one-tailed t-test. (e) RT-qPCR quantification of Foxa1, Ca3, Rassf9 and Tagap mRNA levels in Foxa1 overexpressing cells, GFP expressing cells or in the parental infliximab clone at day 6 of the fed-batch. Error bars are shown as SD, n=3, paired one-tailed t-test.

FIG. 15. Effect of Tagap overexpression on infliximab production.

The infliximab-producing clone was stably transfected with the Tagap or GFP expression vector, and the infliximab titer (a), viable cell density (b) and cell viability (c) were evaluated during 9-days fed-batch cultures. n=4, unpaired one-tailed t-test. Titers are illustrated as described for FIG. 2. Error bars are shown as SD for panels b,c. (d) RT-qPCR quantification of Foxa1, Ca3, Rassf9 and Tagap mRNA levels in Tagap-overexpressing cells, GFP-expressing cells or in the parental clone at day 6 of the fed-batch by RT-qPCR. Error bars are shown as SD, n=3, paired two-tailed t-test.

FIG. 16. mRNA levels of candidate genes obtained from the RNASeq analysis or using gPCR analysis. mRNA levels of Erp27 (a), Foxa1 (b), Ca3 (c) and Tagap (d) in parental CHO cells, Tras polyclonal cells and Tras high producer (HP) clones analyzed by RNASeq and shown in transcripts per kilobase million (TBM), or analyzed using RT-qPCR. Data are presented relative to parental CHO cells. Error bars are shown as SD. Three biological replicates were used for the Tras high producing clones, while three technical replicates were used for parental CHO cells and for the Tras-producing polyclonal cell population.

FIG. 17. Fed-batch culture analyses and mRNA levels of cells producing easy-to-express or difficult-to-express therapeutic proteins and overexpressing Erp27, Erp57 or both. Viable cell density (a) and cell viability (b) of the trastuzumab producing clone stably transfected with the expression vectors for Erp27 and/or Erp57 during fed-batch cultures. Error bars are shown as SD, n=3, unpaired one-tailed t-test. Quantification of Erp27 (c) and Erp57 (d) mRNA levels in the different cell populations by qRT-PCR. Data are presented relative to the mRNA levels of control GFP-expressing cells. Error bars are shown as SD, n=3. (e) Quantification by qRT-PCR of Erp27 mRNA levels in the Tras clone stably transfected with decreasing amounts of Erp27 expression vector and with an empty vector to keep the total amount of plasmid constant. Data are presented relative to Erp27 mRNA levels in control cells. Error bars are shown as SD, n=2. (f and g) qRT-PCR quantification of Erp27 and Erp57 mRNA levels in the infliximab-producing clone stably transfected with expression vectors for Erp27 and Erp57. Data are presented relative to mRNA levels in control cells. Error bars are shown as SD, n=2. (h) Cell viability of the different cell populations analyzed during fed-batch. Error bars are shown as SD, unpaired one-tailed t-test, n≥4.

FIG. 18. mRNA levels of candidate genes and trastuzumab HC and LC transgenes during fed-batch cultures. (a) RT-qPCR quantification of Foxa1, Rassf9, Ca3 and Tagap mRNA levels at day 0 and day 8 of fed-batch cultures in the trastuzumab (Tras) producing clone. Data are presented relative to the mRNA levels in CHO cells. Error bars are shown as SD, n=3. (b) RT-qPCR quantification of Tras heavy chain (HC) and light chain (LC) mRNA levels in Foxa1 overexpressing cells, GFP expressing cells or in the parental Tras clone at day 8 of fed-batch cultures. Data are presented relative to the mRNA levels in control GFP-expressing cells. Error bars are shown as SD, n=3, paired one-tailed t-test.

FIG. 19. Analyses of trastuzumab and Ca3 mRNA levels. (a) RT-qPCR quantification of Tras immunoglobulin heavy and light chain mRNA levels in Cas3, Rassf9 and Tagap-overexpressing cells. Data are presented relative to Tras heavy chain and light chain mRNA levels in control GFP-expressing cells. Error bars are shown as SD, n=3, paired one-tailed t-test. (b) RT-qPCR quantification of Ca3 mRNA levels in the Tras clone stably transfected with various amounts of Ca3 expression vector and with an empty vector to keep the total amount of plasmid constant. Data are presented relative to Ca3 mRNA levels in control GFP-expressing cells.

FIG. 20. Expression of the ACTC1 and TAGAP genes following vitamin B5 selection.

In (a) and (b) the Figure shows transcriptomic RNA sequencing (RNA-Seq) analyses of ACTC1 and TAGAP mRNA levels, comparing non-transfected non-selected parental control cells (C) with transfected cells submitted to antibiotic-selection or to B5-selection and expressing trastuzumab (ETE, panel a) and interferon-beta (DTE, panel b). After selection of transfected cells, cultures were grown in non-selective complete culture medium, and total mRNA was isolated and submitted to high-throughput sequencing to identify genes upregulated in cell populations submitted to the B5 selection process. The relative mRNA levels correspond to normalized read counts from RNA-Seq analyses. (c) Effect of SLC5A6 overexpression and selection by B5 deprivation on ACTC1 and TAGAP gene expression. Cells were co-transfected with the ACTC1 or TAGAP expression vector and the puromycin resistance gene, with or without the SLC5A6 expression vector, after which the cultures were selected either in B5-deficient medium (B5 Deprivation) or in the presence of puromycin (Antibiotic Selection), respectively. Selected cells were transferred to a non-selective culture medium followed by the quantification of ACTC1 and TAGAP mRNAs by RT-qPCR. mRNA levels of cells selected by B5 deprivation were normalized to those of antibiotic-selected cells. (d) The vitamin B5 content of cells transfected and selected as described for panel C was measured by LC-MS after 6 days of a batch culture. (e) Comparison of the ACTC1 and TAGAP mRNA levels of cells transfected with the antibiotic resistance gene without or with the SLC5A6 expression vector and submitted to antibiotic selection. Relative mRNA levels were determined by RT-qPCR and normalized to those of antibiotic resistant cells. Data are mean±SEM of 3 to 5 biological replicates. *P≤0.05; **P≤0.02 with respect to antibiotic selection (t-test; 1 tail).

FIG. 21. ACTC1 levels in ETE-producing cells overexpressing TAGAP

A puromycin-selected clone expressing the Trastuzumab antibody was stably re-transfected with CHO TAGAP expression vector, or with an empty vector and blasticidin resistance gene, and selected with blasticidin resistance. Resulting stable polyclonal cell pools were used to assess TAGAP relative mRNA levels by RT-qPCR (a); and the ACTC1 protein levels (b). Immunoblots of total protein extracts probed with ACTC1 or GAPDH mouse antibodies. The ratio of the ACTC1 signal was normalized to that of GAPDH, as quantified by ImageJ. Data represent the mean fluorescence±SEM of 3 replicates. **P≤0.02 with respect to cells transfected with the empty vector (t-test; 2 tails).

FIG. 22. Overexpression of ACTC1 in recombinant protein-producing cells

(a) A puromycin-selected clone expressing the infliximab antibody was stably re-transfected with CHO ACTC1 expression vector, or with an empty vector and blasticidin resistance gene, and selected with blasticidin resistance. The resulting stable cell pools were used to quantify the relative mRNA levels of ACTC1 by RT-qPCR. (b) Immunoblots of total protein extracts from the cell pools of panel B, probed with ACTC1 or GAPDH mouse antibodies. The ratio of the signal for ACTC1 relative to that of GAPDH was quantified using ImageJ. (c) Red Ponceau staining of total protein of the immunoblot membranes of panel C. Data represent the mean values±SEM of 3 replicates

FIG. 23. DTE recombinant protein production in cells overexpressing ACTC1

In (a), (b) and (c) the figure shows antibiotic-selected immunoglobulin gamma (IgG) expressing clones that were stably re-transfected with the ACTC1 or with an empty expression vector, and the IgG specific productivity of the resulting stable cell pools was measured following selection for resistance to another antibiotic. The specific productivities of the etanercept Fc-fusion (Enbrel©) (panel A), the Bevacizumab IgG1 (panel B), and the Infliximab IgG1 (panel C) are represented as picograms of secreted IgG per cell and per day, as average values±SEM of 3 replicates. (d) The levels of the Infliximab IgG of cells analyzed in panel C were assessed in fed-batch culture conditions over 3 days in non-selective medium, where the titers of the IgG released in the cell culture medium represent the average±SEM of 3 biological replicates. (e) The lactate content of pools of Infliximab-expressing cells transfected with the ACTC1 expression or with the empty vector was measured after 3 days of a batch culture from two independent cell pools using LC-MS assays. Lactate concentrations represent mean values±SEM from 3 technical replicates. *P≤0.05 and **P≤0.02 with respect to empty vector (t-test; 2 tails).

FIG. 24. Characterization of ACTC1-overexpressing cells

A Trastuzumab-expressing CHO cell clone was stably re-transfected with an antibiotic resistance plasmid, together with the CHO ACTC1 expression vector or with the empty expression vector. Stably transfected antibiotic-resistant cells were then selected, from which clones were isolated for further analysis. (a) quantification of ACTC1 relative mRNA levels, as determined by RT-qPCR. (b) Red Ponceau staining of total protein of the immunoblot membrane of FIG. 25a. (c) Viable cell density of the clones over 10 days of the fed-batch cultures performed in FIG. 25b.

FIG. 25. Characterization of the productivity of ACTC1-overexpressing cells

A Trastuzumab-expressing clone was stably re-transfected with the CHO ACTC1 or with an empty expression vector, and cell clones were isolated for further analysis. (a) Immunoblots of total protein extract labelled with ACTC1 or GAPDH mouse antibodies. The histogram shows the ratio of the ACTC1 signal relative to that of GAPDH, as assessed using Image J. Ponceau red-stained membranes are shown in FIG. 24a. (b) Secreted IgG titers in culture supernatants were assessed by double sandwich ELISA over 13 days of fed-batch cultures.

FIG. 26. Actin polymerization levels in ETE clones

(a) Representative Sir-actin fluorescent staining of F-actin on cells from a representative trastuzumab clone overexpressing ACTC1 (ACTC1_Clone2), or from a control clone transfected with the empty expression vector (Empty_Clone2). Unstained cells were used as negative controls. (b) Mean fluorescent signal of Sir-Actin staining from flow cytometry analyses. A total of 2×10⁴ cells were analysed per condition. Data illustrated on the graph represent the mean±SEM from the assay of 4 independent clones. *P<0.05 (t-test; 2 tails; unequal variance).

FIG. 27. Actin polymerization levels in ETE clones Sir-actin fluorescent histograms of F-actin on cells from all trastuzumab clones tested, overexpressing ACTC1 (ACTC1_Clones), or from control clones transfected with the empty expression vector (Empty_Clones), obtained from flow cytometry. Unstained cell were used as negative controls.

FIG. 28. Sorting of therapeutic protein-producing cell pools according to their F-actin polymerization level.

(a) Representative histograms of flow cytometry analyses of a trastuzumab-expressing polyclonal population treated by SiR-actin staining. Unstained control and other analysis are depicted in Fig S4. A total of 5*10⁵ cells were analysed per acquisition, among which 0.4 to 1.4*10 cells were sorted by cytofluorometry according to their low, medium or high actin polymerization (pol.) levels, as depicted.

(b) Selected cells were transferred to a antibiotic-containing culture medium followed by the analysis of IgG cell surface display by immunofluorescence staining cytofluorometry. (c) IgG secretion assays of the sorted cells of panel B. Histograms represent the average values±SEM from 6 cell pools. **P≤0.02 (t-test; 2 tails; paired) relative to the low actin polymerization category.

FIG. 29. Sorting of Trastuzumab-expressing cell pools according to their actin polymerization level

Representative histograms of flow cytometry analyses of a trastuzumab polyclonal population treated by Sir-actin staining, as described in the legend to FIG. 5. The histogram at the top corresponds to unstained control cells and the ones below represent cytometry analysis of independent Sir-actin stained cell pools.

### DESCRIPTION OF VARIOUS AND PREFERRED EMBODIMENTS OF THE INVENTION

The specific alternations in high producer cells, ergo cells that produce a protein of interest in, e.g., vitamin deprived cells at a level higher than the in corresponding cells growing in standard medium were identified. In particular, the metabolic factors that support the desired levels of protein of interest production. It was investigated what changes in the metabolism results from or are tied to the overexpression of specific CHO cell genes in a rare subpopulation of CHO cells. Thus, changes in CHO cell gene expression linked to increased CHO protein synthesis that were associated with high protein of interest production abilities were identified: The products, in particular proteins whose synthesis was associated with high recombinant protein production were called metabolism influencing products (MIPs), in particular RNA encoded proteins, but also noncoding RNAs. Thus, mRNA levels of highly producing CHO cells selected using either vitamin B5 selection or using conventional means were compared to identify mRNA levels changes that are specific hallmarks of high producer cells.

FIG. 1 illustrates the cell selection approaches and comparisons performed between various types of selected high producer cells and control cells.

Table 1 provides the list of candidate MIP-encoding genes identified by the various approaches. It should be noted that the metabolism-linked MIPs may be regulatory proteins such as transcription factors, like PPAR or Foxa1, whose increased mRNA and protein levels may activate in turn the expression of their target genes, as well as metabolic genes themselves, such as lipid and sugar catabolism genes, or anabolic genes encoding e.g. mRNA translation machinery components, structural proteins of the cell such as actin, or cell survival factors such as Ca3 or CDK15.

To identify genes and proteins that in fact cause improved cellular metabolic properties, candidate MIPs were expressed in CHO cells expressing, e.g., a therapeutic protein, to determine if their increased expression causes an improved protein of interest production (FIGS. 2 to 3). The effect of overexpressing regulatory MIPs (e.g. primary MIPs), such as Foxa1 or PPAR, on the expression of other MIPs (e.g. secondary MIPs), and whether they may collectively improve the cell metabolism, for instance the metabolism of lipids, lipid precursors such as acetyl CoA, and byproducts such as lactate, thereby possibly improving the cell's metabolic fitness, which in turn may further increase the production of the protein of interest such as therapeutic IgGs, were assessed. Alternatively, whether overexpressing structural MIPs such as actin leads to increased synthesis and thereby production of the protein of interest was also tested. These figures illustrate that some, but not all, MIP candidates, are capable to increase the production of the easy-to-express (ETE) Trastuzumab antibody, and/or the difficult-to-express (DTE) infliximab antibody. Certain MIPs could be demonstrated to improve the production of one, e.g., therapeutic protein but not the production of another therapeutic protein.

A eukaryotic, including a mammalian, cell, such as a recombinant mammalian cell, according to the present invention is capable of being maintained under cell culture conditions. Non-limiting example of this type of cells are HEK 293 (Human embryonic kidney), Chinese hamster ovary (CHOs) cells and mouse myeloma cells, including NS0 and Sp2/0 cells. Modified versions of CHO cell include CHO-K1 and CHO pro-3. In one preferred embodiment a SURE CHO-M Cell™ line (SELEXIS SA, Switzerland) is used. Cellular proteins of these eukaryotic cells support the expression of transgenes encoding proteins of interest with which the eukaryotic cells have been transfected. These cellular proteins are involved in, among others, lipid metabolism, signal transduction, protein transport, transcription and translation, cellular transport, protein repair, protein folding and cell adhesion, all of which are required for the expression of these transgenes encoding proteins of interest and are referred to herein, as are their counterpart in other species, such as humans, as metabolism influencing products (MIPs), in particular proteins, but also non-coding RNAs as the ones shown in Table 1. One or more transgenes expressing these MIPs (MIP transgenes) may be added to the cells via the MIP eukaryotic expression vectors described herein. Alternatively, or additionally, the endogenous MIP expression (i.e. expression of nucleic acids in the genome of a cell encoding one or more MIP) may be stimulated via the addition of one or more substances, that directly or indirectly influence the expression of an MIP, including an endogenous gene expressing an MIP, such as the PPAR agonist bezafibrate or via promoter swapping, in which such endogenous M IPs are put under the control of different exogenous promoters or endogenous promoters, wherein each of the promoters are associated with a specific expression level of such an MIP and thus can be used to alter the expression of such an endogenous MIPs. Preferably selected MIPs according to the present invention are MIPs whose expression results in a cell also harboring a transgene encoding a protein of interest (generally, but not necessarily on a separate vector, referred to herein as a carrier vector) to be expressed at a level that exceed the level of expression of the transgene when the cell has not been transfected with a vector comprising one or more of the selected MIPs. The nucleic acids encoding the MIPs generally comprise or consist of the coding sequences (CDS) of the cellular or human counterpart. Table 1 shows some MIPs.

Primary MIPs increase the expression of their target genes and of secondary MIPs and include regulatory proteins such as:

Foxa1 (Forkhead box protein A1) is a transcription factor that is involved in embryonic development, establishment of tissue-specific gene expression and regulation of gene expression in differentiated tissues. is thought to act as a ‘pioneer’ factor, ergo to open the compacted chromatin for other proteins, in the case of Foxa1, through interactions with nucleosomal core histones and thereby replacing linker histones at target enhancer and/or promoter sites.

PPARs (Peroxisome proliferator-activated receptors) are ligand-activated transcription factors. PPARs mainly exist in three subtypes; α, β/δ, and γ, each of which mediates the physiological actions of a large variety of fatty acids (FAs) and FA-derived molecules and are involved in FA metabolism. Activation of PPAR-β/δ enhances fatty acids metabolism. Thus, PPAR family plays a major regulatory role in energy homeostasis and metabolic function in a cell. All PPARs heterodimerize with the retinoid X receptor (RXR) and bind to specific regions on the DNA of target genes. These DNA sequences are called PPREs (peroxisome proliferator hormone response elements). The consensus sequence of the PPRE is composed of two AGGTCA-like sequences directionally aligned with a single nucleotide spacer. In general, this sequence occurs in the promoter region of a gene, and, when the PPAR binds its ligand, transcription of target genes is increased or decreased, depending on the gene. The promoter region with a PPRE, the TATA box, and the transcription start site may be located in a repressive chromatin structure. The binding of ligand to the PPAR/RXR/corepressor complex causes the release of the corepressor from the ligand-activated PPAR/RXR complex. The activated PPAR/RXR complex binds to the PPRE, inducing structural change in chromatin, with histone H1 released. The PPRE-bound PPAR/RXR targets a coactivator-acetyltransferase complex to the promoter. The coactivator-acetyltransferase complex acetylates the histone tails (Ac), thereby generating a transcriptionally active structure. Additional transcription factors (TF) and the RNA Pol II initiation complex are recruited to the accessible promoter and transcription is initiated. FIG. 4 highlights B5-target genes found to be regulated by PPAR, the majority of which eventually feed into the lipid metabolism.

Endogenous ligands (endogenous agonists) that activate PPARs include free fatty acids and eicosanoids. PPARs are also the molecular targets of a number of drugs (exogenous agonists). For instance fibrates, such as clofibrate, gemfibrozil, ciprofibrate, bezafibrate, and fenofibrate, activate PPARα. They are indicated for cholesterol disorders and disorders that feature high triglycerides. Bezafibrate also activates the other types of PPARs, that is PPARβ/δ and PPARγ and is thus considered a pan-PPAR activator. The antidiabetic thiazolidinediones (TZDs) activate PPARγ and are used for diseases that feature insulin resistance such as diabetes mellitus. GW501516 (also known as GW-501,516, GW1516, GSK-516) is a PPARS receptor agonist. The synthetic chemical perfluorooctanoic acid activates PPARα while the synthetic perfluorononanoic acid activates both PPARα and PPARγ.

Secondary MIPs are MIPs who are expressed as a result of the overexpression of primary MIP(s) such as PPARs and/or Foxa1. As described elsewhere herein, cells that expressed proteins of interest beyond a threshold level, not only expressed PPARs and unrelated MIPs at a level not observed in cells that did not express the protein of interest beyond the threshold level, but also MIPs whose expression was known or was likely to be influenced by PPARs such as Hmgcs2, Acot1 and Cyp4a14. Ca3 and Rassf9 are Foxa1 transcriptional target and thus might be secondary MIPs. The MIPs discussed below may or may not be secondary MIPs.

Structural MIPs

The cytoskeleton comprises of a network of actin microfilaments, microtubule and intermediate filaments required for multiple cellular processes, such as cell shape and resistance to mechanical deformation (Mays, Beck, & Nelson, 1994), protein synthesis (Hudder, Nathanson, & Deutscher, 2003), protein transport and secretion (Paavilainen, Bertling, Falck, & Lappalainen, 2004; Stamnes, 2002), association of cellular components (Knull & Walsh, 1992), and metabolic channeling (Aon & Cortassa, 2002). Moreover, an increase in monoclonal antibody production was correlated with a significant increase in cytoskeletal proteins such as actin, tubulin, or the actinin-binding cofilin (Dinnis et al., 2006). Recent studies have shown that suspension CHO cells have evolved from adherent cells by disruption of the extracellular attachment matrix accompanied with major changes in the cytoskeleton, such as increased actin filament expression, which is required for proper interaction with integrins, resistance to shear stress and cell proliferation in suspension (Walther, Whitfield, & James, 2016). Therefore, cytoskeleton organization and modulation of actin filament levels may impact suspension cell fitness and recombinant protein expression, from mRNA translation to protein secretion.

Structural MIPs directly contribute to the structure of a cell and include, e.g., Actin. actin monomers polymerize to form filaments that organize into dynamic networks with fundamental roles in multiple and diverse cellular processes. Turnover of actin networks drive multiple cellular processes, including cell movement, cell adhesion, changes in cell morphology, vesicle trafficking, and cytokinesis. ACTC1 is the major protein of the cardiac sarcomere thin filaments, which are responsible for the muscle contraction function of the heart. Consistently, ACTC1 deficiency has been mainly linked to heart diseases (Debold et al., 2010; Wang et al., 2016).

MIPs involved in signal transduction, vesicular trafficking activities and cell adhesion include, for example Tagap (T-cell activation GTPase-activating protein), Rassf9 (Ras Association Domain Family Member 9).

The protein encoded by the Rassf9 gene localizes to perinuclear endosomes. This protein associates with peptidylglycine alpha-amidating monooxygenase, and may be involved with the trafficking of this enzyme through secretory or endosomal pathways. Clstn3 (Calsyntenin 3) may modulate calcium-mediated postsynaptic signals.

TAGAP is not only a signaling protein, but is also involved in cytoskeleton organization (see ACTC1 above). As such TAGAP is involved in thymocyte loss of adhesion and thymocyte and T cells cytoskeleton reorganization (Connelly et al., 2014; Duke-Cohan et al., 2018). Alterations of the TAGAP gene has been associated with various autoimmune diseases (Eyre et al., 2010).

MIPs involved in the basic metabolism of a cell such as mRNA translation include, for example asparaginyl-t-RNA synthesase (see Table 1 for further examples).

Proteins involved in protein folding (also, protein folding proteins) include Erp27 (Endoplasmic Reticulum protein 27.7 kDa) which is thought to have chaperone activity, ERp57 is a lumenal protein of the endoplasmic reticulum (ER) and a member of the protein disulfide isomerase (PDI) family. ERP44 is also a protein disulfide isomerase, that is involved in protein quality control at the endoplasmic reticulum-Golgi interface.

Cell survival and/or proliferation proteins include CDK15 (Cyclin Dependent Kinase 15) which belongs to a large family of serine/threonine protein kinases that regulate cell proliferation, apoptosis, cell differentiation, and embryonic development. Ca3 (Carbonic Anhydrase 3) is involved in the reversible hydration of carbon dioxide.

Proteins involved in apoptosis include CFLAR (CASP8 And FADD Like Apoptosis Regulator) or SOD1 (Superoxide Dismutase 1).

Proteins involved in glutathione catabolism include GCLM (Glutamate-Cysteine Ligase Modifier Subunit) or GGCT (Gamma-glutamylcyclotransferase).

Eukaryotic cells (also referred to herein as eukaryotic host cells or just host cells) such as Chinese hamster ovary (CHO) cells are widely used in industrial processes for the production of recombinant therapeutic proteins. The viability of, e.g., CHO cells, NSO, BHK and human embryo kidney-293 (HEK-293) are dependent on vitamin uptake. Mammalian cells cannot synthesize them and mammals must therefore obtain them from their diet. The main function of vitamins is to act as cofactors or coenzymes in various enzymatic reactions such as Acetyl-CoA biosynthesis. It was shown that vitamin deprivation during fed-batch bioreactor production conditions can be used to improve the viability of cell clones and their productivity in terms of the titer of secreted recombinant therapeutic proteins. These effects were obtained by lowering the levels of e.g. the B5 or H vitamins. Vitamin metabolic protein may increase vitamin availability in a cell and in particular vitamin transport protein may serve as selectable marker. Thus, in its simplest form, in a medium that is deficient in one vitamin, recombinant eukaryotic cells expressing the respective vitamin transport protein as a selectable marker can grow better than cells not expressing the respective vitamin transport protein. The sodium-multivitamin transporter SLC5A6 has been characterized as a transport protein for both the B5 and H vitamins. Other examples of vitamin metabolic proteins include pantothenate kinases 1, 2 or 3. Pantothenate kinases are key regulatory enzyme in the biosynthesis of coenzyme A (CoA).

A transgene as used in the context of the present invention is an isolated deoxyribonucleotide (DNA) sequence coding for a given protein. In the case of MIP, transgenes the DNA sequence may also encode a non-coding RNA. The term transgene is used in the present context when referring to a DNA sequence that is introduced into a cell such as a eukaryotic host cell via transfection. Thus, a transgene is always exogenous, but might be heterologous or homologous.

Exogenous nucleic acid as it is used herein means that the referenced nucleic acid is introduced into the host cell. The source of the exogenous nucleic acid may be homologous or heterologous nucleic acid that expresses. Correspondingly, the term endogenous refers to a nucleic acid molecule that is present in the host cell prior to transfection. The term heterologous nucleic acid refers to a nucleic acid molecule derived from a source other than the species of the host cell, whereas the term homologous nucleic acid refers to a nucleic acid molecule derived from the same species as the host cell. Accordingly, an exogenous nucleic acid according to the invention can utilize either or both a heterologous or homologous nucleic acid. For example, a cDNA of a human interferon gene is a heterologous exogenous nucleic acid in a CHO cell, but a homologous exogenous nucleic acid in a HeLa cell. Similarly, the genes encoding MIPs indicated in Table 1, when introduced via a vector into CHO cells are exogenous nucleic acids, such exogenous nucleic acids being heterologous (e.g. human, mouse, E. coli) or homologous (e.g. Cricetulus griseus).

Apart from the MIP transgenes, some transgenes according to the present invention are transgenes encoding proteins of interest, such as therapeutic proteins, ergo proteins with therapeutic activity including immunoglobulins (Igs) and Fc-fusion proteins. Certain immunoglobulins such as Infliximab (Remicade) or coagulation factor VIII, are notably difficult to express, because of mostly uncharacterized cellular bottlenecks. With the help of the MIP expression vectors, recombinant eukaryotic cell and methods of the present invention these bottlenecks may be identified and/or opened.

The specific productivity such as the IgG productivity, of a clone expressing a transgene, such as a protein of interest, is determined as the slope of IgG concentration versus the integral number of viable cell (IVCD) calculated during the production phase, generally from day 3 to day 7, and is expressed as pg per cell and per day (pcd).

An easy-to-express (ETE) transgene, in particular a transgene encoding a protein of interest, such as a therapeutic protein is expressed in standard medium in a CHO at levels above 10 pcd. Examples of ETE transgenes are the Trastuzumab antibody.

An difficult-to-express (DTE) transgene, in particular a transgene encoding a protein, in particular a protein of interest, such as a therapeutic protein is expressed in standard medium in a CHO generally at levels below 10 pcd. Examples of DTE transgenes are the transgenes encoding infliximab IgG1 (Remicade), etanercept Fc-fusion (Enbrel©) or Bevacizumab, or other secreted proteins such as coagulation factor VIII as well as the interferon beta protein.

As used herein, the term transgene shall not include untranscribed flanking regions such as RNA transcription initiation signals, polyadenylation addition sites, promoters or enhancers.

A vector according to the present invention is a nucleic acid molecule capable of transporting another nucleic acid, such as nucleic acid encoding a MIP into a cell. For example, a plasmid is a type of vector, a retrovirus or lentivirus is another type of vector. In certain embodiments, the vector is linearized prior to transfection.

The MIP expression vector comprises regulatory sequences such as promoters, enhancers, locus control regions (LCRs), matrix attachment regions (MARs), scaffold attachment regions (SARs), insulator elements, and/or nuclear matrix-associating DNAs that lead to efficient transcription of a MIP integrated into the expression vector. These regulatory sequences are always exogenous and often heterologous (see below).

Promoters refer to DNA sequences capable of controlling the expression of a coding sequence. In some embodiments, the promoter sequence comprises proximal and more distal upstream elements, the latter elements are often referred to as enhancers. Accordingly, an “enhancer” is a DNA sequence that can stimulate promoter activity, and may be a homologous or heterologous.

The MIP expression vector, but also any other vector or recombinant cells disclosed herein may comprise one or more promoters selected from the group consisting of: CMV, EF1alpha, CMV/EF1alpha fusion promoter, SV40, RSV, PGK and combinations thereof, which may be used to, e.g., express any one or a combination of the MIPs at expression levels specific for the respective promoter. For example, the promoter CMV/EF1alpha (generally referred to as a very strong promoter), can be used to express the respective gene at a first expression level specific for the CMV/EF1alpha promoter (=CMV/EF1alpha promoter expression level+/−5% or 10%), while promoter CMV (generally referred to as a strong promoter when in full length (hereinafter “CMV promoter”) and as a weak promoter when provided as a modified full length CMV promoter for reduced expression (sometimes referred to as “minimal CMV promoter”), can be used to express the respective gene at a second expression level (=CMV promoter expression level+/−5% or 10%), wherein, the first expression level exceeds the second expression level specific for the CMV promoter. Depending on the type of protein of interest to be expressed, one or the other promoter can be used. The promoters are in certain embodiments inducible. Different promoters may be part of a promoter ladder comprising least two promoters.

Promoter swapping which includes introducing one or more promoters and/or generating variants of one or more promoters within a host cell (herein referred to as “recombinant promoters”), which exhibit more than one expression level (e.g. promoter ladders), or differing regulatory properties (e.g., tighter regulatory control for selected genes) can also be used to alter, e.g., the expression level of and MIP endogenous to a eukaryotic cell (host cell) such as a CHO cell.

A promoter ladder includes a plurality of promoters which differ in their level of promoter activity. A promoter ladder, which might include 2, 3, 4, 5 or more promoters each associated with an activity that provides for an expression level of a gene under the control of the promoter, e.g., a second expression level that exceeds a first expression level. The promoter ladder may be associated with a gene of an endogenous MIP, but also an exogenous counterpart. The ladder will allow switching the promoter dependent on the required MIP level for the expression of the transgene expressing a product of interest at a certain level. Such a ladder can also be used to optimize expression levels to be used in the context of different types of such transgenes.

A carrier vector according to the present invention is an expression vector that is adapted to transport a transgene expressing a protein of interest into the cell. It also includes regulatory sequences and generally has at least one restriction enzyme cleavage site adapted for insertion of a nucleic acid encoding a protein of interest and optionally an antibiotic resistance gene and/or a vitamin transport protein such as sodium-multivitamin transporter SLC5A6. An expression vector may also contain an origin of replication. As the person skilled in the art will readily understand the transgene expressing a protein of interest can also be integrated into the MIP vector.

A transposon is a mobile genetic element that efficiently transposes between vectors and chromosomes via a “cut and paste” or “copy and paste” mechanism. During transposition, the transposase of a transposon system (e.g., the PB transposase in the PiggyBac transposon system) recognizes transposon-specific inverted terminal repeat sequences (ITRs) located on both ends of the transposon (there is a 5′- and a 3′ ITR to any transposon system) and moves the contents from the original sites and integrates them into chromosomal sites, such as TTAA chromosomal sites. The powerful activity of, e.g., the PiggyBac transposon system enables genes of interest between the two ITRs to be easily mobilized into target genomes. The PiggyBac transposon system is described, e.g., in US patent publication 2010/0154070, which is incorporated herein by reference in its entirety (see also US patent publication 2015/0361451). Among non-viral vectors, transposons are attractive because of their ability to integrate single copies of DNA sequences with high frequency at multiple loci within the host genome. Unlike viral vectors, some transposons were reported not to integrate preferentially close to cellular genes, and they are thus less likely to introduce deleterious mutations. Moreover, transposons are readily produced and handled, comprising generally of a transposon donor vector/plasmid (or just “transposon vector” containing the cargo DNA flanked by inverted repeat sequences and of a transposase-expressing helper vector/plasmid (also referred to herein as “transposase expression vector”) or mRNA. Several transposon systems were developed to mobilize DNA in a variety of cell lines without interfering with endogenous transposon copies. For instance, the PiggyBac (PB) transposon originally isolated from the cabbage looper moth efficiently transposes cargo DNA into a variety of mammalian cells. In a transposon donor plasmid, epigenetic regulatory elements can be used to protect the cargo DNA from unwanted epigenetic effects when placed near the transgene on plasmid vectors. For example, MARs can increase cargo DNA genomic integration and transcription while preventing heterochromatin silencing, as exemplified by the potent human MAR 1-68 and MAR X-29 elements. They can also act as insulators and thereby prevent the activation of neighboring cellular genes. MAR elements have thus been used to mediate high and sustained expression in the context of plasmid or viral vectors (see US patent publication no. 2015/0361451, which is specifically incorporated herein by reference in its entirety).

MAR elements (also referred to as MAR sequences or MARs) belong to a wider group of epigenetic regulator elements which also include boundary or insulator elements such as cHS4, locus control regions (LCRs), stabilizing anti-repressor (STAR) elements, ubiquitously acting chromatin opening (UCOE) elements or histone modifiers such as histone deacetylase (HDAC).

MAR elements may be defined based on the identified MAR they are primarily based on: A MAR construct is, accordingly, a MAR element that whose majority of nucleotide (50% plus, preferably 60%, 70% or 80%) are based on MAR S4. Several simple sequence motifs such as high in A and T content have often been found within MARs Other motifs commonly found are the A-box, the T-box, DNA unwinding motifs, SATB1 binding sites (H-box, A/T/C25) and consensus topoisomerase II sites for vertebrates or Drosophila.

MARs are generally characterized as sequences in the DNA of eukaryotic chromosomes where the nuclear matrix attaches. The properties of MAR are only in part defined by their primary structure. For example, a typical primary structure found in MAR elements such as AT rich regions are known to result in tertiary structures, namely in certain curvatures that define the function of the MAR. Thus, MARs are often defined not only by their primary structure, but also by their secondary, tertiary structure, e.g. their degree of curvature and/or physical properties such as melting temperature.

An AT/TA-dinucleotide rich bent DNA region (hereinafter referred to as “AT-rich region”) as commonly found in MAR elements is a bent DNA region comprising a high number of A and Ts, in particular in form of the dinucleotides AT and TA. In a preferred embodiment, it contains at least 10% of dinucleotide TA, and/or at least 12% of dinucleotide AT on a stretch of 100 contiguous base pairs, preferably at least 33% of dinucleotide TA, and/or at least 33% of dinucleotide AT on a stretch of 100 contiguous base pairs (or on a respective shorter stretch when the AT-rich region is of shorter length), while having a bent secondary structure. However, the “AT-rich regions” may be as short as about 30 nucleotides or less, but is preferably about 50 nucleotides, about 75 nucleotides, about 100 nucleotides, about 150, about 200, about 250, about 300, about 350 or about 400 nucleotides long or longer.

Some binding sites are also often have relatively high A and T content such as the SATB1 binding sites (H-box, A/T/C25) and consensus Topoisomerase II sites for vertebrates (RNYNNCNNGYNGKTNYNY) (SEQ ID NO: 154) or Drosophila (GTNWAYATTNATNNR) (SEQ ID NO: 155). However, a binding site region (module), in particular a TFBS region, which comprises a cluster of binding sites, can be readily distinguished from AT and TA dinucleotides rich regions (“AT-rich regions”) from MAR elements high in A and T content by a comparison of the bending pattern of the regions. For example, for human MAR 1_68, the latter might have an average degree of curvature exceeding about 3.8 or about 4.0, while a TFBS region might have an average degree of curvature below about 3.5 or about 3.3. Regions of an identified MAR can also be ascertained by alternative means, such as, but not limited to, relative melting temperatures, as described elsewhere herein. However, such values are specie specific and thus may vary from specie to specie, and may, e.g., be lower. Thus, the respective AT and TA dinucleotides rich regions may have lower degrees of curvature such as from about 3.2 to about 3.4 or from about 3.4 to about 3.6 or from about 3.6 to about 3.8, and the TFBS regions may have proportionally lower degrees of curvatures, such a below about 2.7, below about 2.9, below about 3.1, below about 3.3. In SMAR Scan II, respectively lower window sizes will be selected by the skilled artisan.

Some preferred identified MAR elements include, but are not limited to, MAR 1_68, MAR X_29, MAR 1_6, MAR S4, MAR S46 including all their permutations as disclosed in WO2005040377 and US patent publication 20070178469, which are specifically incorporated by reference into the present application for the disclosure of the sequences of these and other MAR elements. The chicken lysozyme MAR is also a preferred embodiment (see, U.S. Pat. No. 7,129,062, which is also specifically incorporated herein for its disclosure of MAR elements).

If a vector is said to comprise a singular MAR this means that in this vector there is one MAR and there are no other MARs within the vector either of the same or a different type or structure. Such a singular MAR is in certain embodiments located downstream of the integration site of the transgene encoding, e.g., a protein of interest, e.g., between the transgene integration site and a 3′ ITR. In certain embodiments, such a transgene is a CDS encoding the MIP is situated between a 5′ ITR and a 3′ ITR. The MAR follows a polyadenylation signal at the 3′ end of the CDS encoding the MIP and is located between the polyadenylation site and the 3′ ITR. A promoter such as a CMV promoter and/or a CMV/EF1alpha fusion promoter is located 5′ ITR and the CDS encoding the MIP.

Transfection as used herein refers to the introduction of nucleic acids, including naked or purified nucleic acids or vectors carrying a specific nucleic acid into cells, in particular eukaryotic cells, including mammalian cells. Any know transfection method can be employed in the context of the present invention. Some of these methods include enhancing the permeability of a biological membrane to bring the nucleic acids into the cell. Prominent examples are electroporation or microporation. The methods may be used by themselves or can be supported by sonic, electromagnetic, and thermal energy, chemical permeation enhancers, pressure, and the like for selectively enhancing flux rate of nucleic acids into a host cell. Other transfection methods are also within the scope of the present invention, such as carrier-based transfection including lipofection or viruses (also referred to transduction) and chemical based transfection. However, any method that brings a nucleic acid inside a cell can be used. A transiently-transfected cell will carry/express transfected RNA/DNA for a short amount of time and not pass it on. A stably-transfected cell will continuously express transfected DNA and pass it on: the exogenous nucleic acid has integrated into the genome of a cell. A stably-transfected cell according to the present invention includes, e.g., a cell in which the MIP transgene has become part of the genome of the cell subsequent to transfection with a transposon vector.

A cell growing in a complete culture medium will have all vitamins available at standard concentrations. Standard concentrations are referred to herein as 1×. Standard concentrations for B1, B5 and H (1×) were set at 7.5 μM, 2.5 μM and 0.5 μM, respectively. B5 was determined to have for CHO cells a growth-limiting concentration range around 10⁻⁴× to 10⁻³× (0.25 to 2.5 nM), whereas 10⁻²× and higher concentrations allowed normal culture growth. The limiting concentrations of B1 was determined to be for CHO cells between 10⁻⁵× (15 μM) and 10⁻⁴× (150 μM), whereas it was lower than 10⁻⁵× (5 μM) for H.

In a medium having limiting concentration (limiting medium or depleted medium) of said vitamin the concentration is less than 1×, e.g. 10⁻¹×, 10⁻²×, 10⁻³×, 10⁻⁴×, 10⁻⁵×, relative to said standard concentration of the respective vitamin present in a complete medium (1×). The concentration of a vitamin is considered saturating if the concentration exceeds that in a standard reference medium (also referred to herein as a “saturated medium”) (e.g., 2×, 3×, 4×, 5×, or 10× the amount found in a complete medium).

The present invention takes, among others, advantage of the fact that in a limiting medium the growth and/or division of cells may be arrested, and the cell produces MIPs that cause a protein of interest to be produced at a maximum arrested/expression level (“MA/EL” in [g/1]). When the protein of interest is co-transfected into a cell with one or more of the genes encoding the MIPs produced during the cell arrest, the protein of interest may be produced at MA/EL which may exceed a maximum level (“ML” in [g/1]) of protein expressed by the same type of cells when the one or more MIPs are not present/when the cell growth and/or division is not arrested. The MA/EL may be more than 1.5× the ML, more than 2× the ML or even more than 2.5× or 3× the ML. For example, while a ML of a protein of interest, such as an antibody, that is expressed by recombinant cells, such as recombinant CHO cells that are not co-transfected with a MIP maybe about 1 g/l of the antibody, the MA/EL of the protein of interest, such as an antibody that is expressed by recombinant cells that also express one or more MIPs maybe about 1.5 g/l or 2 g/l of the antibody or more.

Expression systems/vectors generally contain a selectable marker gene which facilitates the selection of eukaryotic cells (host cells, also referred herein to recombinant eukaryotic cells) transformed with vectors containing the polynucleotide encoding the protein of interest. The selectable marker (or “selectable marker protein”) expressed by the gene are often based on antibiotic resistance. E.g. a puromycin resistance selection expression cassette can be used to identify, via the addition of puromycin, cells that has been successfully transformed with the cassette. However, selection without any resistance to antibiotics is also possible. In the context of the present invention, a vitamin metabolic protein, in particular a vitamin transport protein, may serve as selectable marker either alone or in combination with other selectable markers. Thus, in its simplest form, in a medium that is deficient in one vitamin such as B5 (Pantothenic acid), Vitamins B1 (thiamin), and/or H (B8 or biotin), recombinant eukaryotic cells expressing the respective vitamin transport protein as a selectable marker can grow better than cells not expressing the respective vitamin transport protein. However, as discussed herein, even in standard medium, the vitamin transport proteins provide a growth advantage and thus can be used as selectable marker. The expression systems of the present invention may contain, as selectable markers, vitamin metabolic protein(s), in particular, vitamin transport protein(s), such as sodium-multivitamin transporter SLC5A6, in addition to selectable marker genes based, e.g., on antibiotic resistance.

Nucleic acids and proteins having more than 50%, 60%, 70%, 75%, 80%, 85%, 90%, 95%, 96%, 97%, 98% or 99% sequence identity with the polynucleotides and proteins sequences disclosed herein, are also part of the present invention either alone or as part of any system (e.g. vectors and cells), cell, method and kit disclosed herein. Nucleic acids of the present invention may differ from any wild type sequence by at least one, two, three, four five, six, seven, eight, nine or more nucleotides. In many instances, nucleic acids made up of CDSs of the respective gene/cDNAs are preferred.

The term sequence identity refers to a measure of the identity of nucleotide sequences or amino acid sequences. In general, the sequences are aligned so that the highest order match is obtained. “Identity”, per se, has recognized meaning in the art and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity between two polynucleotide or polypeptide sequences, the term “identity” is well known to skilled artisans (Carillo, H. & Lipton, D., SIAM J Applied Math 48:1073 (1988)).

Whether any particular nucleic acid molecule is at least 50%, 60%, 70%, 75%, 80%, 85%, 90%, 95%, 96%, 97%, 98% or 99% identical to, for instance, a certain nucleic acid sequence encoding MIP, or a part thereof, can be determined conventionally using known computer programs such as DNAsis software (Hitachi Software, San Bruno, Calif.) for initial sequence alignment followed by ESEE version 3.0 DNA/protein sequence software for multiple sequence alignments.

Whether the amino acid sequence is at least 50%, 60%, 70%, 75%, 80%, 85%, 90%, 95%, 96%, 97%, 98% or 99% identical to, for instance a MIP in form of a protein, or a part thereof, can be determined conventionally using known computer programs such the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. Many of the MIPs are well studied and have one, but often more than one conserved region. As the person skilled in the art will appreciate a variation in a nucleic acid/protein sequence is preferably, if not exclusively, outside such conserved region(s) of the respective MIP.

When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleic acid or amino acid sequence and that gaps in homology of up to 5% of the total number of nucleotides in the reference sequence are allowed.

Effect of Selected MIPS on Transgene Expression

As discussed above, Foxa1 generally increases cell viability, viable cell density and the production of both easy-to-express and difficult-to-express therapeutic proteins when overexpressed. This effect may be allocated to the Foxa1-mediated Tagap upregulation. Indeed, when overexpressed, Tagap could temporarily increase viable cell density and an increase in the titer of easy-to-express and difficult-to-express therapeutic proteins was observed.

Tagap is a signaling protein member of the Rho GTPase-activating protein (GAP) family. In thymocytes, it was shown to regulate the abundance of active RhoA, thus promoting cytoskeleton reorganization and release of β1-integrin-mediated adhesion allowing thymocytes migration from the cortex to the medulla (Duke-Cohan et al., 2018). Moreover, Tagap and the cardiac muscle actin alpha (ACTC1) were found to be upregulated in vitamin B5 selected cells producing therapeutic proteins at very high levels, and Tagap overexpression was shown to increase the expression of ACTC1, which in turn increases the production of various therapeutic proteins. Thus, in CHO suspension cells, TAGAP could function as a mediator for intracellular cytoskeleton signal to cell surface integrins, hence improving cell proliferation, viability and adaptation to suspension.

Interestingly, spherical integrin clustering, as well as an increase in actin content and formation of a spherical actin sheath was observed in suspension-adapted CHO cells (Walther, Whitfield, & James, 2016). An increased expression of Tagap could therefore contribute to improve the actin-mediated adaptation of cells in a suspension environment. Tagap upregulation could also contribute to improve therapeutic protein secretion since the actin cytoskeleton is involved in the regulation of the secretory pathway (Stamnes, 2002). Notably, another candidate gene upregulated in Tras high producer clone, Arhgap42 (Rho GTPase Activating Protein 42), is a Rho GTPase-activating protein which was shown to localize to actin stress fiber and focal adhesions and to promote cell motility (Hu et al., 2018; Luo et al., 2017). Furthermore, Arhgap42 is also a Foxa1 target gene. Thus, Arhgap42 expression is also within the scope of the present invention, preferably to increase titer and viable cell density.

As noted above, the ACTC1 (Actin Alpha Cardiac Muscle 1) gene, is known to be involved in cardiac muscle alpha actin synthesis. It also acts to improve ETE and DTE therapeutic protein expression and secretion by recombinant eukaryotic cells such as CHO cells. It was observed that the increase of ACTC1 levels was accompanied with a decrease in overall actin polymerization, implying that the organization of the cytoskeleton controls or affects the expression or the secretion of the therapeutic proteins. To support this observation, it was shown that CHO cell pools with spontaneously decreased actin polymerization level secrete significantly higher levels of the recombinant protein. Since the augmented release of therapeutic proteins by actin-overexpressing cells was not accompanied by increased IgG light and heavy chain mRNA (data not shown), it was concluded that this actin effect is post-transcriptional.

The data supports that ACTC1 overexpression accumulates an excess of actin monomers, which may disturb intracellular balance with G/F-actin and thereby cause the observed decrease of the F-actin polymeric forms. An interplay of actin dynamics and gene expression has already been proposed in mammalian cells. For instance, it was found that the treatment of primary murine cell with chemical agents provoking F-actin disruption ellicited a global inhibition of translation and protein synthesis, and that this activated the cellular stress response (Silva, Sattlegger, & Castilho, 2016). Here, it could be shown that a decrease of actin polymerization, either spontaneous or elicited by ACTC1 overexpression, rather mediated an increase of recombinant protein expression by CHO cells, and that this did not impair cell division or viability. It can be inferred that F-actin depolymerization may provoke a turnover of actin assembly that may enhance vesicular and protein trafficking. For instance, colifin is an actin depolymerizing protein that induces actin reorganization, thereby promoting the exocytosis of small molecules and vesicular trafficking (Birkenfeld, Kartmann, Betz, & Roth, 2001). Similarly, CHO suspension cells selected for lower levels of polymerized actin may display higher cytoskeletal reorganization, which in turn may improve recombinant protein secretion. However, and another favorable effect of ACTC1 overexpression is the resulting decrease in the accumulation of the cell-toxic lactate by-product of early glycolysis. An interplay of the cytoskeleton with lactate accumulation was suggested by a report showing that cytoskeleton perturbation can inhibit the lactate transporter and import by oocytes (Tosco, Faelli, Gastaldi, Paulmichl, & Orsenigo, 2008), suggesting that CHO cell actin depolymerization might prevent the accumulation of toxic intracellular lactate concentrations.

Overall, several mechanisms may explain the positive effect of actin overexpression on protein production by eukaryotic cells such as CHO cells, which may pertain both to the basic metabolism of those cells and energy production by glycolysis, as well as by a potential activation of protein secretion. In sum, it could be shown that ACTC1 overexpression and/or the assay for spontaneous alterations in F-actin polymerization using SiR-Actin staining and cell sorting can both be used to facilitate the isolation of high expressor CHO cells from stable cell pools.

It could be shown that cytoskeletal protein and the modulation of cytoskeletal organization may be used to improve protein production for biotechnological purpose.

As noted above, Erp27 is a protein that selectively binds to unfolded proteins and interacts with the disulfide isomerase Erp57 in the ER (Alanen et al., 2006; Kober et al., 2013). As also noted above, Foxa1, is a pioneering transcription factor involved in the development of variety of organs (Zaret & Carroll, 2011). It could be shown that the expression of specific combinations of these MIPs yield increased cell density and viability in fed-batch cultures, higher production of easy-to-express as well as of difficult-to-express therapeutic proteins, and decreased reactive oxygen species, providing novel avenues towards highly efficient therapeutic protein production. Table 2 show genes upregulated in Tras high producer clones (HPC) versus parental CHO cells and versus Tras polyclonal cells (PC) (FIGS. 11-15).

The ER-located protein Erp27 was identified as being involved in the high-level production of both easy-to-express and difficult-to-express therapeutic proteins. Despite the fact that Erp27 is a redox-inactive member of the PDI family, it is likely to participate to protein folding, since it selectively binds to unfolded proteins and interacts with the disulfide isomerase Erp57 (Alanen et al., 2006; Kober et al., 2013). Notably difficult-to-express proteins are prone to misfolding, and the unfolded protein response (UPR) was shown to be activated upon expression of difficult-to-express proteins (reviewed in Hansen et al., 2017). Thus, Erp27 and Erp57 overexpression likely contribute directly to decrease the accumulation of misfolded difficult-to-express proteins, thereby preventing or delaying UPR-induced apoptosis. This explains well the increase in cell viability and viable cell density upon Erp27 and Erp57 co-overexpression in cells expressing difficult-to-express proteins. While Erp27 and Erp57 were shown to be upregulated upon ER stress (Bargsted, Hetz, & Matus, 2016; Kober et al., 2013), this upregulation might not be sufficient to deal with the large quantity of misfolded recombinant proteins. In addition to increasing the yield of therapeutic proteins, overexpression of Erp27 and Erp57 might also prevent quality issues of the product, as the antibody quality was found to decrease together with cell viability (Kaneko, Sato, & Aoyagi, 2010). In contrast, high production of the trastuzumab antibody did not trigger a full UPR response (Le Fourn et al., 2014), which is consistent with the finding that Erp27 overexpression, combined or not with Erp57, did not improve cell viability and had no or little effect on viable cell density in these conditions. Nevertheless, the folding capacity of CHO cells might still represent a bottleneck in these conditions, as indicated by the fact that Erp27 moderate overexpression increased the trastuzumab titer.

While protein folding in the ER was demonstrated to be a limiting step for the production of several therapeutic proteins, conflicting results were published concerning the effect of PDI and Erp57 overexpression on therapeutic protein production (reviewed in Hansen et al., 2017).

Ca3 upregulation was also observed in the easy- and difficult-to-express protein high producer clones as well as in Foxa1-overexpressing cells and to a lesser extent in Tagap-overexpressing cells. Notably, Ca3 was shown to inhibit H₂O₂-induced apoptosis and to reduce H₂O₂-induced ROS activity (Raisanen et al., 1999; Shi et al., 2018). It was also shown to protect cells against hypoxic stress (reviewed in Di Fiore et al., 2018). Importantly, accumulation of ROS was observed during fed-batch cultures, and oxidative stress was shown to affect the yield and galactosylation of antibodies (Ha, Hansen, Kol, Kildegaard, & Lee, 2018). Moreover, addition of the antioxidants baicalein or S-sulfocysteine in fed-batch cultures improved cell viability and antibody production in fed-batch cultures (Ha et al., 2018; Hecklau et al., 2016). Consistently, we found a decrease in ROS accumulation in Foxa1-overexpressing cells during the last days of the fed-batch cultures, and an increase in cell viability. In contrast, while Ca3 overexpression resulted in an increased Tras titer, we did not observe any positive effect on cell viability. A possible explanation is that Ca3 was not overexpressed at the correct level. It is also possible that the Foxa1-mediated increase in cell viability requires the activation of other genes. A possible candidate is CDK15 which is also upregulated in the Tras high producer clones and was shown to protect cells against apoptosis (Park, Kim, Kim, & Chung, 2014), however, whether CDK15 is a Foxa1 target gene remains to be tested.

Finally, Rassf9 upregulation was also observed in the easy- and difficult-to-express high producer clones as well as in Foxa1-overexpressing cells. Rassf9 was shown to associate with recycling endosomes and was proposed to regulate vesicular trafficking via its interaction with integral membrane proteins (Chen, Johnson, & Milgram, 1998). Although its overexpression did only result in an increase in therapeutic protein titer for Tras14, but not Tras6, it is possible that it is involved in the secretion of therapeutic proteins.

Overall, it could be observed that the upregulation of several CHO cell genes contributes to improving the production yields of various easy- and difficult-to-express therapeutic proteins. Interestingly, several of these CHO genes appear to be upregulated by the Foxa1 transcriptional activator. We therefore conclude that Foxa1 increased expression may elicit a transcriptional program that is favorable for high-level therapeutic protein production, and that this provides a convenient approach to improve the production of recombinant proteins of interest.

The invention in explained below by non-limiting examples.

### Examples

MIP Candidate Selection

RNA Seq Outline

Genetic and metabolic changes occurring in the cells during B5 selection were deciphered. To do so a transcriptomic analysis by RNASeq, comparing B5 to non-selected cells and antibiotic to non-selected cells (FIG. 1A) was performed. Genes which expression where significantly upregulated between AB-selection and B5-selection (at least a 1.5-fold increase with P>0.5) were identified, and detected in both ETE and DTE recombinant cell lines. Thirty-one (31) genes candidates as B5-selection targets were found (Table 1).

The expression pattern of these genes can be classified in two categories (FIG. 1B). The first category, which included most of the candidate genes, showed gene expression decreasing after transfection with the recombinant protein upon antibiotic (AB) selection (upper graph). However, gene expression was improved in B5-selected recombinant cells. The hypothesis for this expression pattern was that gene transcription is challenged due to the competition for the cellular machinery to produce the recombinant protein at high amount. On the contrary, B5 selection might improve general cell fitness and metabolism which could lead to improvement of target gene expression.

For the second expression pattern (FIG. 1B lower graph), target genes were induced in both AB and B5 selected cells as compared to non-transfected cells, with a higher expression in B5 selected cells. In this case, target gene could be induced in response to the recombinant protein, and be involved either in the different steps of the recombinant protein production and secretion from the cells, or being part of the detoxification process caused by the inflammation response.

B5 selection induced changes mainly in metabolic genes such as enzymes and transporters (9/31 target genes). As B5 selection is based on changes in primary metabolism due to B5 deprivation, it was anticipated that a substantial number of target genes would be part of diverse cellular metabolisms.

Surprisingly, five of these genes were involved in lipid metabolism. By looking deeper into the literature, we found that three of them, Hmgcs2, Acot1 and Cyp4a14 were targets of a common transcription factor, PPAR (Rakhshandehroo et al., 2010).

The hydroxymethylglutaryl CoA synthase2 (Hmgcs2) encodes a mitochondrial protein that catalyzes the first reaction of ketogenesis by condensing acetyl-CoA with acetoacetyl-CoA to form HMG-CoA. It determines the metabolic fate of fatty acids in the liver of starved animals (Vila-Brau et al, 2011).

Acot1 encodes an Acyl-CoA thioesterase which catalyzes the hydrolysis of acyl-CoAs to the free fatty acid and coenzyme A (CoASH). It is involved in long fatty-acid metabolism.

Cyp4a14, a cytochrome P450, have been showed to be involved in liver damage, inflammation, and fibrosis in mice (Zhang, 2017).

The peroxisome proliferator-activated receptors (PPARs) are ligand-activated transcription factors that belong to the superfamily of nuclear hormone receptors and play an important role in nutrient homeostasis (Kersten et al., 2000). Three different PPAR subtypes are known: PPARα, PPARβ/δ and PPARγ. All PPARs form a heterodimer with nuclear receptor RXR, followed by binding to PPAR response element (PPRE) sequence located in the promoter of its target genes. Activation of transcription by PPARs is dependent on a number of different steps including ligand binding to PPAR, binding of PPAR to the target gene, removal of corepressors and recruitment of coactivators, remodeling of the chromatin structure, and finally facilitation of gene transcription (Michalik et al., 2006). PPARs regulate the expression of genes that function in lipid and carbohydrate metabolism, vascular biology, tissue repair, cell proliferation and differentiation, and sexual dimorphism (Wahli et al., 2012). Thus, the study focused on PPAR and PPAR targets in order to check whether there is a link between B5 selection and PPAR activation leading to PPAR target activation.

Another target gene that was noted was the ACTC1 gene involved in actin synthesis. Cytoskeleton organization is important for many cellular components such as protein synthesis and secretion (Hudder et al, 2003) or stability of the metabolic network (Aon and Cortassa, 2002). Therefore, increase in recombinant protein production could be correlated with increase cytoskeleton together with increase secretory pathways (ER chaperone) and metabolic machinery (Dinnis et al, 2006). Recent studies have showed that suspension CHO cells have evolved from adherent cells by reorganization of their cytoskeleton in order to reinforce their subcortical actin sheath (Walther, 2016). Therefore, actin modulation could have an impact on suspension cell fitness and recombinant protein production.

Identification of Genes Associated with a High Productivity for Therapeutic Proteins

Genes displaying expression alterations that are associated with the production of therapeutic proteins at high levels by CHO cells were identified and tested as new cell engineering candidates for improving therapeutic protein production. For this purpose, a transcriptomic analysis was carried out to compare three different types of cells: CHO cell clones producing the easy-to-express trastuzumab (Tras) antibody at high levels while maintaining a high cell density, displaying an average specific productivity of 19.3 pg of Tras secreted per cell and per day (pg/cell/day) and an average maximum viable cell density (VCD) of 43.3 million cells per ml were analyzed. These cell lines were compared to a Tras polyclonal cell population obtained after antibiotic selection of cells expressing stably the transgenes (specific productivity of 7.4 pg/cell/day, maximum VCD of 36.3 million cells per ml), and to the parental untransfected CHO cells (FIG. 1c).

Candidate genes were selected according to two criteria: first 113 mRNAs were selected which were significantly upregulated in Tras high producer clones when compared to the parental CHO cells (FIG. 1c). Also selected were 1774 mRNAs that were upregulated in the high producer clones when compared to the polyclonal Tras-expressing cell pool. 51 mRNAs were found to match both criteria, corresponding to 32 genes whose upregulated expression may be associated to Tras high productivity (FIG. 1c, Table 2). Changes in the mRNA levels of the candidate genes were further confirmed on the different samples using RT-qPCR (FIG. 16, data not shown). Surprisingly, an ontology analysis indicated that candidate protein-coding genes were mostly associated to signaling and cell adhesion (Table 2, FIG. 1d). Also identified were genes involved in protein folding (Erp27), cell survival (Ca3, Cdk15, Vegfd), cell growth (Clstn3), vesicular trafficking (Rassf9, Clstn3) and cytoskeleton organization (Mybpc2, Tagap, Arhgap42), which are cellular functions that were previously proposed to influence therapeutic protein production (Table 2, FIG. 1d, Baek et al., 2015; Fischer et al., 2015; Hansen et al., 2017). Interestingly, most of these candidate genes were also upregulated in CHO cell clones producing at high level another easy-to-express antibody, bevacizumab, and the difficult-to-express interferon beta protein, when compared to their expression in the parental CHO cells (data not shown). This indicated that candidate gene upregulation is not solely linked to cells displaying a high trastuzumab antibody productivity, and thus suggested that such candidate genes could be involved in high-level production of various easy-to-express and difficult-to-express therapeutic proteins.

An overview of the positive effect of PPARs, ACTC1, and of other MIP candidates of various origins on the production of ETE and DTE CHO cell lines are illustrated in FIGS. 4 to 10.

FIGS. 1 and Table 2 show that:
Genes associated with Trastuzumab high productivity include genes involved in protein folding, cell survival, vesicular trafficking and cytoskeleton remodeling.
Foxa1, a pioneering transcription factor, is upregulated in Trastuzumab high producing clones and might activate a transcriptional response favorable for therapeutic protein production.
B5 selection induced changes in lipid metabolism genes.
PPAR transcription factor seems to be a regulator of some B5-target lipid genes.
Cytoskeleton regulation and morphology through actin production might play a role in cell fitness and recombinant protein production.

Comments FIG. 1 and Table 1:

A transcriptomic analysis was performed in order to identify genes associated with Trastuzumab high productivity. In this analysis, genes upregulated in Trastuzumab high producing clones compared to CHO-M WT cells were selected and compared to cells polyclonal for Trastuzumab production (FIG. 1C). 32 genes associated with high productivity were identified (candidate genes, Table 1). Importantly, expression of these genes can be causes or consequences of Trastuzumab high productivity. Further focus was put on potential candidate genes that could improve therapeutic protein productivity based on their functions (FIG. 1D).

An overview of the effect of these different MIPs Erp27, Erp57, Ca3, CDK15, Rassf9, Clstn3, Tagap and Foxa1) on Trastuzumab (ETE) and Infliximab (DTE) production is provided in FIGS. 2 and 3.

MIP Candidates Found in Therapeutic Protein High Producing Clones

Overexpression of Candidate Genes Increases Trastuzumab Production (FIG. 2):

FIG. 2A to FIG. 2E show the effect of candidate MIPs on Trastuzumab production, an easy-to-express (ETE) antibody. For this purpose, two Trastuzumab middle producing clones maintaining a fast cell division were isolated from the Trastuzumab polyclonal population used for the transcriptomic analysis. These clones were stably transfected with plasmids for the expression of MIPs (Tagap, Rassf9, Erp27, Erp57, Erp27+Erp57, Clstn3, CDK15, Ca3 and Foxa1). Trastuzumab production was evaluated in these stable populations at different time of fed-batch cultures. Overexpression of SRP14 was used as a positive control, cells expressing GFP or transfected with an empty vector were used as negative control. While overexpression of Rassf9, Foxa1 and Ca3 increased Trastuzumab production, Erp57, Clstn3 and CDK15 overexpression and Erp27 and Erp57 co-overexpression did not affect Trastuzumab production. Tagap overexpression had a variable but sometimes positive effect on Trastuzumab production. When strongly overexpressed, Erp27 decreased Trastuzumab production, when slightly overexpressed, it increased Trastuzumab production. According to databases, Ca3 and Rassf9 are Foxa1 transcriptional targets. An overexpression of Ca3 and Rassf9 was indeed found in Foxa1 overexpressing cells. These results strongly suggest that Foxa1 overexpression induces the transcription of genes which improve Trastuzumab production.

In sum it was found that:
Rassf9, Ca3 and Foxa1 overexpression improves Trastuzumab production.
When strongly overexpressed, Erp27 decreases Trastuzumab production, whereas when slightly overexpressed, it increases Trastuzumab production.
Overexpression of Erp57, Clstn3 and CDK15 and co-overexpression of Erp27 and Erp57 do not affect Trastuzumab production.
The Foxa1 transcriptional response might create a favorable environment for Trastuzumab production.

Overexpression of Specific Candidate MIPs Increase Infliximab Production (FIG. 3), a Difficult-to-Express (DTE) Antibody:

FIG. 3A and FIG. 3B show the effect of candidate MIPs on Infliximab production, a difficult-to-express (DTE) antibody. Infliximab producing clone was stably transfected with plasmids for the expression of MIPs. Production of Infliximab was evaluated in these stable populations at different time of fed-batch cultures. Cells transfected with an empty vector were used as negative control. While expression of Erp27 or of Erp57 did not increase Infliximab production, coexpression of Erp27 and Erp57 or expression of Tagap increased Infliximab production. Viable cell density was higher for cells overexpressing Tagap and Erp27+Erp57 at day 9 and 11 of fed-batch cultures.

In sum it was found that overexpression of Tagap and co-overexpression of Erp27 and Erp57 increases viable cell density at days 6 and 9 of fed-batch culture and improves Infliximab production.

Erp27 is a protein present in the endoplasmic reticulum which binds to unfolded protein (Kober et al., 2013). Although initially annotated as a protein disulfide isomerase (PDI), Erp27 does not have any redox activity. In particular, Erp27 contains the non-catalytic b and b′ domains of PDI, but it lacks the CXXC active site required to catalyze dithiol-disulfide exchange (Alanen et al., 2006). It is however known to interact with the PDI Erp57, which triggers disulfide bond formation (Alanen et al., 2006). An increased expression of Erp57 was notably found to increase thrombopoietin productivity in CHO cells (Hwang et al., 2003).

Erp27 Overexpression Alone or with Erp57 Improves Therapeutic Protein Production

As Erp27 was shown to bind in vitro and in vivo to the disulfide isomerase Erp57 (Alanen et al., 2006), it was hypothesized that the Erp27-Erp57 complex participates in therapeutic protein folding, providing a production advantage.

This hypothesis was evaluated by assessing the effect of Erp27 and Erp57 overexpression on trastuzumab secretion levels. For this purpose, clones were isolated from the trastuzumab polyclonal population previously used for the transcriptomic analysis, and the clone displaying a high productivity (1.8-fold that of the polyclonal population) while maintaining a fast cell division rate in fed-batch cultures was selected. Notably, the Erp27 mRNA levels of this clone were found to be upregulated by 3 to 6-fold when compared to those of parental CHO cells at day 0 or 8 of fed-batch cultures (FIG. 11a). In contrast, Erp57 mRNA levels were similar in the CHO parental cells and Tras producing clone at day 0, while a slight 1.2-fold upregulation in the clone was observed at day 8. This clone was stably transfected with the Erp27 and/or Erp57 expression vectors, or with a GFP expression vector as control, and the levels of secreted Tras were evaluated during fed-batch cultures of the polyclonal populations.

Upon Erp57 overexpression with or without Erp27 co-expression, the viable cell density was increased at day 6 of the fed batch culture, whereas cell viability was reduced at day 10 (FIG. 17a, b). Overall, the Tras titer levels of Erp57-overexpressing cells were similar to those of control cells (FIG. 11b). In contrast, growth and cell viability during fed-batch culture was not affected by Erp27 overexpression (FIG. 17 a, b). However, Erp27 overexpression led to a decrease of the Tras levels, which was also noted upon Erp57 co-expression (FIG. 11b). It was noticed that Erp27 overexpression led to a very substantial increase of the Erp27 mRNA levels when compared to the Tras producing clone (FIG. 17 c-d), suggesting that such overexpression levels might result in metabolic unbalance of these protein activities, thus rather reducing Tras expression. The amount of Erp27 expression vector in stably transfected cells was therefor titrated down. Indeed, a 14% increase of the Tras levels was observed upon Erp27 reduced overexpression (FIG. 11c and FIG. 17e). Overall, the result shown that moderate overexpression of Erp27 increased Tras production.

As the transcriptomic analysis indicated that Erp27 mRNA expression was also increased in clones expressing the difficult-to-express interferon beta at high level (data not shown), it was further assessed what the effects of Erp27 and Erp57 overexpression on the production of difficult-to-express therapeutic proteins were. The infliximab chimerical immunoglobulin (Infli) and the etanercept Fc-fusion were used as two additional examples of difficult-to-express therapeutic proteins. In contrast to the results obtained for Tras, the Infli titers were unaffected upon Erp27 overexpression and rather reduced upon Erp57 overexpression in an infliximab expressing clone (FIGS. 11d and 17f, g). However, co-overexpression of Erp27 and Erp57 resulted in a 61% and 72% increase in infliximab titers relative to GFP-expressing control cells at day 9 and 11 of the fed-batch cultures, respectively (FIG. 2d). Moreover, Erp27 and Erp57 co-overexpression yielded an increased viable cell density and cell viability at day 9 of the fed-batch (FIGS. 11e and 17h).

The effect of Erp27 and Erp57 co-overexpression in an etanercept producing clone was also assessed. Single subclones co-expressing Erp27 and Erp57 were isolated and their production was assessed using a ClonePix® cell colony imaging device. The cell colonies showing the widest etanercept secretion halo were isolated from the Erp27 and Erp57 overexpressing or control cell populations, and the derived cell clones were assessed for etanercept production in fed-batch cultures. The viable cell density and cell viability were enhanced upon Erp27 and Erp57 overexpression, together with an extended plateau phase of the viable cells and a 37% increase of the titer (FIG. 11f-h). Taken together, these results support that Erp27 moderate overexpression increases the production of an easy-to-express therapeutic protein, and that Erp27 and Erp57 combined overexpression could enhance viable cell density, cell viability and the titer of cells producing different difficult-to-express therapeutic proteins.

Foxa1 Overexpression Increases Trastuzumab Production and Reduces Oxidative Stress

Surprisingly it was found that among the 32 genes associated with Trastuzumab high productivity there is a pioneering transcription factor called Foxa1. Foxa1 might activate a transcriptional response favorable for therapeutic protein production. Foxa1 can bind to repressive heterochromatin structures, where it can release gene expression independently of other transcription factors (for a review, see Zaret & Carroll, 2011). It is involved in the development of different organs such as the liver, pancreas, lungs, and prostate (Friedman & Kaestner, 2006). Thus, we hypothesized that Foxa1 might activate a transcriptional program favorable for the production of therapeutic proteins such as Tras.

Consistently, Foxa1 mRNA expression was increased in the Tras clone compared to the parental CHO cells at day 0 and day 8 of fed-batch cultures, with an upregulation of 1.5 and 2.1-fold, respectively (FIG. 18a). A 3-fold upregulation was observed in the Tras high producer clone relative to the parental CHO cell controls in the transcriptomic analysis, thus indicating that Foxa1 expression may be further increased (see Table 2). The Tras-producing clone was therefore stably transfected with a Foxa1 expression vector. Stable expression of Foxa1 under the control of the strong CMV/EF1alpha promoter resulted in elevated cell death during the antibiotic-mediated selection (data not shown). However, the substitution of this strong promoter by a minimal CMV promoter abrogated this unwanted effect, and a 57% increase in the final Tras titer was obtained upon Foxa1 overexpression (FIG. 12a), whereas Foxa1 mRNA was upregulated by 40- and 14-fold at day 0 and 8 of fed-batch cultures, respectively (FIG. 12 d, e). While cell growth in fed-batch cultures was similar when comparing Foxa1-overexpressing to control cells up to day 6, Foxa1-overexpressing cells continued to divide up to day 9, reaching an average viable cell density of 31 million cells per ml, while control cells peaked at 19 million cells per ml at day 8 (FIG. 12b). Moreover, the viability of Foxa1-overexpressing cells remained above 90% until day 9, while control cell viability decreased from day 7 and was below 75% at day 9 (FIG. 12c).

Ca3, Rassf9 and Tagap are Upregulated Upon Foxa1 Overexpression in Tras Producing Clone

Several studies have demonstrated that productivity can be improved by extending cell survival in fed-batch cultures (for a review, see Kim et al., 2012). Among the 32 genes identified, Ca3 and CDK15 (see FIG. 2b) promote cell survival. Ca3 acts in protecting cells against oxidative stress (Di Fiore et al., 2018) whereas CDK15 protects cells against apoptosis (Park et al., 2014). Overexpression of these proteins might therefore extend the lifespan of cells in fed-batch culture, therefore improving productivity.

Another focus was on Rassf9 and Clstn3, two proteins found in transport vesicles (Chen et al., 1998; Rindler et al., 2007) that might possibly participate to therapeutic protein secretion.

Tagap is a signaling protein involved in thymocyte loss of adhesion and thymocyte and T cells cytoskeleton reorganization (Connelly et al., 2014; Duke-Cohan et al., 2018). Similarly, to actin, Tagap overexpression might improve cell adaptation to suspension and might trigger cytoskeleton reorganization thus improving secretion. Notably, Tagap was also overexpressed in B5 selected cells.

The fact that Foxa1 is a pioneering transcription factor suggested that it might directly increase the transcription of trastuzumab heavy chain (HC) and light chain (LC) transgenes. However, no significant change in the trastuzumab HC and LC mRNA levels upon Foxa1 overexpression was observed (FIG. 18b). To confirm that the Foxa1-mediated increase of trastuzumab titer results from the transcriptional activation of CHO cell genes that are also upregulated in the Tras high producer clones, (Table 2). The Harmonizome® web portal was used to identify potential Foxa1 target genes (Rouillard et al., 2016). Accordingly, 11 of the 25 protein-encoding genes identified to be upregulated in Tras high producer clones were predicted to be Foxa1 target genes, including the Foxa1 gene itself (Table 2). We therefore tested whether these genes were upregulated upon Foxa1 overexpression in the trastuzumab producing clone, revealing that Ca3 and Rassf9 were highly upregulated upon Foxa1 overexpression at day 8 of the fed-batch, whereas the expression of other Foxa1 potential target genes was not significantly altered (FIG. 12). Furthermore, while Erp27 was not upregulated in Foxa1 overexpressing cells, an upregulation of the Tagap candidate gene was observed, which was also found to be upregulated in vitamin B5 selected cells producing therapeutic proteins at very high levels. Notably, there was also a Rassf9, Ca3 and Tagap mRNA upregulation in Foxa1-overexpressing cells at day 0 of the fed-batch cultures, indicating that their upregulation was not a consequence of the high cell growth observed at day 8 in Foxa1-overexpressing cells (FIG. 12e). As Ca3 has been shown to protect cells from oxidative stress (reviewed in Di Fiore, Monti, Scaloni, De Simone, & Monti, 2018), the levels of intracellular reactive oxygen species (ROS) were evaluated using the reactive fluorescent dye carboxy-H₂DCFDA. Interestingly, while at day 3, there was a slight increase in ROS levels in Foxa1 overexpressing cells, ROS levels were reduced in Foxa1 overexpressing cells at day 6, 8 and 9 (FIG. 12f).

Ca3 and Tagap Overexpression Increase Trastuzumab Production

To test whether the Tras titer increase resulting from Foxa1 overexpression is the consequence of Ca3, Rassf9 and/or Tagap upregulation, the three candidate genes were stably overexpressed in the Tras-producing clone and the Tras titers obtained from fed-batch cultures were assessed. Consistently, a higher Tras titer was obtained upon Tagap overexpression in the Tras producing clone, whereas no effect was detected from the overexpression of Ca3 or Rassf9 (FIG. 13a). An increased viable cell density was observed at day 6 and day 8 of culture upon Tagap overexpression, with a maximum viable cell density of 31 million cells per ml at day 8 (FIG. 13b). However, cell viability strongly decreased starting from day 9 (FIG. 13c). The increase in Tras production upon Tagap overexpression was similar to the levels obtained upon Foxa1 overexpression, with a titer of 1331 μg/ml, despite the lower cell viability upon prolonged fed batch cultures observed from Tagap overexpression (FIGS. 12a-c and 13a-c). A slight increase in the expression of Tras HC and LC (1.6 and 1.3 respectively) was also observed upon Tagap overexpression (FIG. 19a).

Interestingly, a 10-fold increase in Ca3 expression was also observed upon Tagap overexpression (FIG. 13d). To assess if the lack of effect of Ca3 overexpression alone may result from unfavorable expression levels (FIG. 13a-c), the amount of the expression vector used to establish the stable cell lines was titrated. A slight increase in the trastuzumab titer was obtained upon the higher Ca3 overexpression levels, while viable cell density and viability were not affected (FIG. 13e and FIG. 19b and data not shown). In conclusion, while Tagap overexpression could recapitulate the Foxa1-mediated increase in trastuzumab titer and temporarily improve viable cell density, it rather decreased cell viability at the end of fed-batch cultures. Overall, it was therefore concluded that the positive effects of Foxa1 on cell viability and cell growth in culture, and on protein titer, may result from its effect on the expression of several target genes.

Foxa1 Also Improves the Production of Difficult-to-Express Therapeutic Proteins

The effect of Foxa1 overexpression on the secretion of the difficult-to-express infliximab was further assessed. Impressively, infliximab production was nearly doubled upon Foxa1 overexpression, where an average titer of 378 μg/ml was obtained, upon an 8.2-fold increase of Foxa1 mRNA levels (FIG. 14a, e). Notably, Foxa1-overexpressing cells showed a significantly increased viable cell density starting from day 6 until day 9, reaching a maximum viable cell density of 12.2 million cells/ml at day 7, while control cells only reached a viable cell density of 8.6 million cells/ml (FIG. 14b). Consistently, we observed that cell viability remained significantly higher in Foxa1 expressing cells, preventing the crash of cell viability observed from day 7 with the control cells (FIG. 14c). This was accompanied by a decrease in the accumulation of ROS at day 7 and 8 in the Foxa1 overexpressing cells (FIG. 14d). Similar to what was observed upon Foxa1 overexpression in the Tras-producing clone, Ca3, Rassf9 and Tagap mRNA levels were also upregulated upon Foxa1 overexpression (FIG. 14e). Consistently, we obtained a 45% increase in infliximab production upon Tagap overexpression, yielding an average titer of 283 μg/ml (FIG. 15a). Therefore, while Tagap overexpression could recapitulate the Foxa1-mediated increase of the Tras titer, it only partially mimicked the Foxa1-induced infliximab titer increase. As observed for the Tras-producing clone, Tagap overexpression resulted in a rapid increase in viable cell density for the infliximab clone, with a maximum viable cell density of 12 million cells/ml at day 6 (FIG. 15b). However, in contrast to Foxa1 overexpressing cells, cell viability remained mostly unchanged upon Tagap overexpression (FIG. 15c). Notably, Tagap overexpression in the infliximab producing clone also yielded an upregulation of Ca3 mRNA levels (FIG. 15d). Taken together, these results indicated that Foxa1 overexpression can be used to increase the production levels of difficult- as well as easy-to-express therapeutic proteins in CHO cells, and that this effect may result in part from the Foxa1-mediated increase in Tagap expression levels.

MIP Candidates Found in B5-Selection (PPAR)

FIG. 5A and FIG. 5B show significant increase in DsRed (Discosoma sp. Red) activity that was observed between AB and B5-selected cells with or without PPRE reporter sequence indicating that DsRed expression is induced independently from PPAR activation. This induction can be explained by the overall improved fitness of B5 over AB-selected cells.

However, when mPPARα was exogenously added, DsRed activity was significantly higher in B5-selected cells only when under control of PPRE reporter. The results indicate that B5 selection has generated stable cells constitutively producing an unknown PPAR agonist, probably during the B5 selection which could be sensed as a starvation stress by the cells. Therefore, exogenous PPARα was more activated in B5-selected cells as compared to AB-selected cells.

Summary
Unidentified PPAR agonists have accumulated during B5 starving-selection.
Better fitness in B5-selected cells leads to overall better genes expression compared to antibiotic-selected cells.
Activation of exogenous PPARα is higher in B5-selected cells leading to higher PPAR target gene expression compared to antibiotic-selected cells.

FIG. 6A and FIG. 6B show the activity of Bezafibrate (2-[4-[2-(4-chlorobenzamido)ethyl]phenoxy]-2-methylpropanoic acid). Bezafibrate has been reported to be a general PPAR pan-agonist (Wilson et al., 2000; Inoue et al., 2002).

It is already in clinical use as an antihyperlipidaemia drug. Addition of bezafibrate on ETE after 3 days fed-batch induces PPAR target genes HmgCs2 and Acot1 found in the RNA-Seq screening as well as known PPAR targets DBI1, AscI1 (Rakhshandehroo et al., 2010) and RXR nuclear receptor (FIG. 6A). Cyp4a14, was not induced upon bezafibrate addition, which could mean that other regulations through other agonist or PPAR could be controlling the expression of this gene. Interestingly, the B5-target Slc22a14, was activated in response to bezafibrate. Slc22a14 gene have been showed to be involved in mouse male fertility (Maruyama, 2016), but no function associated with PPAR response have been described.

Addition of bezafibrate at day 3 of the fed-batch significantly improved cell survival when subjected to starvation stress during the fed-batch process (data not showed), however no improvement of IgG production was observed in normal fed-batch conditions of feeding (see M&M for fed-batch culture). Day 3 of fed-batch corresponded to the exponential phase of cell division. Addition of bezafibrate earlier during fed-batch process (day 1), showed to be beneficial for recombinant protein at the end of the fed-batch (FIG. 6B).

Bezafibrate was also tested in DTE cells. However, although the same target genes are induced, the cell production and fitness weren't improved. Therefore, PPAR activation and target genes induction through bezafibrate appears not sufficient to overcome the bottlenecks of cells synthesizing difficult-to-express proteins.

As bezafibrate can alter cell division and metabolism at high concentration as it as a strong wide effect on all PPAR activation, increasing bezafibrate in both ETE and DTE cells doesn't show any beneficial effect on both cell division, viability and recombinant protein production (data not showed).

In sum it was found (see FIG. 6) that:
PPAR targets identified by B5-selection can be chemically induced in CHO cells.
Slc22a14 transporter highlighted as a B5-target, is showed to be a new PPAR target.
This induction lead to better IgG production in ETE cells.
Recombinant cells expressing a DTE protein are not affected by bezafibrate induction.

When grown in complete non-stressful medium, PPARα overexpression (e.g. PPARα_OE) didn't show any difference in PPAR-target gene expression and IgG production when comparing to wild-type and empty vector cells. However, when bezafibrate was added, exogenous PPARα present in PPARα_OE was activated and subsequently induced the transcription of PPAR-target genes as well as RXR nuclear factor and IgG light and heave chains (FIG. 7A). This increase led to higher IgG productivity of PPARα_OE cells (FIG. 7B).

In sum it was found (FIGS. 7A and B) that:
Activation of exogenous PPAR might generate DTE cells with improved cell fitness which leads to improved production of DTE therapeutic proteins.

In sum it was found (FIGS. 8A-D) that:
Lactate is decreased in B5-selected cells.
PPAR overexpression lead to decrease in lactate content in CHO cells.

MIP Candidates Found in B5-Selection (Actin)

FIG. 9 already shows that the overexpression of the Actin gene generated ETE cells with improved production of the therapeutic protein. An Fc-fusion-expressing clone was re-transfected with a transposable ACTC1-expression vectors. The specific productivity of the resulting cell pools was then evaluated through their subcultivation in batch condition every 3 or 4 days. Results were represented as a fold change of PCD to Fc-fusion-control cells PCD value. The results suggest that actin overexpression in suspension CHO cells may improve therapeutic protein production and secretion by modulating cytoskeleton organization and polymerization.

In further experiments, CHO cells were co-transfected with expression vectors encoding an “easy-to-express” (ETE) Trastuzumab or a “difficult-to-express” (DTE) Infliximab or etanercept (Enbrel®) therapeutic protein, together with the vitamin B5 transporter SLC5A6 or with an antibiotic resistance gene as a control. Cells were then selected for their aptitude to survive in a B5-deficient culture medium or for antibiotic resistance, respectively, and differentially expressed cellular genes were identified by RNA sequencing. After antibiotic selection, the expression of both ACTC1 and TAGAP was lower than in non-transfected cells, while they were increased after B5 selection (FIGS. 20a and 20 b). The increase of TAGAP expression following SLC5A6 expression and vitamin B5 starvation was validated using four independent Trastuzumab-expressing CHO cell lines isolated using either antibiotic or B5 selections (FIG. 20c).

Gene induction after B5 selection may be caused either by B5 starvation occurring during the selective process, as found in a previous study (Pourcel et al., 2019), by the overexpression of SLC5A6 itself, as it mediates higher vitamin B5 intake into the cell (FIG. 20d), or by a combination of both effects. B5 is an essential cofactor for Acetyl CoA, a key element in central metabolism and energy metabolism, which could be linked to cytoskeleton regulation. To distinguish between these possibilities, cell lines overexpressing SLC5A6 transporter were generated without any B5 deprivation, which indicated that increased SLC5A6 expression suffices to upregulate significantly the ACTC1 gene, whereas a non-significant increase of TAGAP expression was noted (FIG. 20e). Therefore, the B5 selection process might activate the ACTC1 gene expression by the increased B5 intracellular import mediated by SLC5A6 overexpression, whereas a significant increase of TAGAP expression required a combination of both SLC5A6 overexpression and B5 starvation. It was also observed that TAGAP overexpression increased ACTC1 mRNA and protein accumulation (FIG. 21), suggesting that the increased ACTC1 expression resulting from the B5 selection process may result in part from the upregulation of TAGAP.

The effect of ACTC1 overexpression on recombinant protein expression was assessed on antibiotic-selected cell clones expressing several DTE proteins, such as the etanercept (Enbrel©) Fc-fusion or the Bevacizumab or Infliximab IgG1, as well as on a clone expressing the ETE Trastuzumab immunoglobulin. These cell clones were re-transfected with the ACTC1 coding sequences together with another antibiotic selection gene, followed by the selection of antibiotic resistant cells overexpressing ACTC1 (FIGS. 22a-c). The specific productivity of the resulting cell pools was then evaluated in batch culture conditions after 3 to 4 days, indicating a positive effect of ACTC1 high level expression on the production of the DTE proteins by CHO cells (FIGS. 23a-c). Further analysis on Infliximab-producing ACTC1-overexpressing cell pools showed a significantly increased IgG titer after 10 days of fed batch culture (FIG. 23d).

Next individual clones overexpressing ACTC1 were analyzed. To do so, a Trastuzumab-producing clone was re-transfected with the ACTC1 or empty expression vector, and single colonies were picked using a Clonepix® device. Eight clones transfected with the empty vector and 24 ACTC1-expressing clones were validated for ACTC1 transcript accumulation, among which 4 control clones and 4 ACTC1 high expressor clones were randomly picked for further analysis (FIG. 24a). ACTC1 protein overexpression was validated by western blot (FIGS. 25a and 24b). Among the four ACTC1-overexpressing clones, 3 showed the highest IgG titers after 13 days of fed batch culture as compared to the empty vector clones (FIGS. 25b and 24c).

To determine if the increased therapeutic protein secretion elicited by ACTC1 overexpression may result from cellular metabolic alterations, we measured primary metabolism markers by mass spectrometry analysis of pools of ACTC1-overexpressing cells. Notably, we assessed the accumulation of lactate, a toxic by-product of the early steps of glycolysis, which has been well documented as a bottleneck for therapeutic protein production (Lao & Toth, 1997). This revealed a strong reduction of lactate accumulation by ACTC1-overexpressing cells after 3 days in batch culture, when compared to control cells (FIG. 2e). Overall, we therefore concluded that ACTC1 gene overexpression significantly improved the secretion of various therapeutic proteins, and that this effect may be linked to a decrease in the accumulation of the toxic lactate metabolic by-product.

Implication of Actin Polymerization Level in the Secretion of Recombinant Proteins

We next assessed whether the actin polymerization status may be affected by ACTC1-overexpression. To do so, we relied on SiR-Actin staining, which specifically binds to F-Actin (Lukinavicius et al., 2014), yielding fluorescence level of stained cells that are proportional to actin polymerization. Comparison of SiR-Actin-staining of the ACTC1 and trastuzumab-expressing clones relative to control clones revealed higher fluorescence in the control clones than in the ACTC1-overexpressing ones, indicating that actin polymerization level was significantly reduced by ACTC1 overexpression (FIGS. 26a, b and 27).

To further assess whether actin polymerization may affect recombinant protein expression, two independent CHO cell polyclonal populations expressing the Trastuzumab protein, but not submitted to an ACTC1 vector transfection, were stained with SiR-actin. Stained cells were then sorted in three independent cell batches according to their low, medium or high fluorescence level (FIGS. 28a and 29), to obtain 6 cell pools for each fluorescence level. The IgG secretion level and IgG specific productivity of cells displaying low, medium and high levels of polymerized actin were then assessed (FIGS. 28 b, c). High SiR-Actin staining cells showed a significantly lower IgG expression levels than cells displaying low SiR-Actin staining, thus supporting the conclusion that cells with lower actin polymerization levels mediate higher recombinant protein secretion, even without ACTC1 overexpression.

Effect of Individual or Combined Expression of MIP(s) on the Secretion of Different Therapeutic Protein-Expressing Cho Clones

Comments FIG. 10:

An bevacizumag-expressing clone (FIG. 10A), an fc-fusion-expressing clone (FIG. 10B) and an fab-enzyme-fusion expressing clone (FIG. 10C) were re-transfected with various individual or combination of transposable CFLAR-, GCLM-, ACTC1-expression vectors. The specific productivity of the resulting cell pools was then evaluated through their subcultivation in batch conditioned every 3 or 4 days. Results were represented as a % of their respective bevacizumab- or Fc-fusion-control cells PCD values (pg-1. cell-1.day-1).

Summary FIG. 10:
Secretion of therapeutic proteins by CHO cells was increased after transfection of vectors expressing MIP such as CFLAR-, GCLM-, ACTC1-expression vectors.

TABLE 1
Candidate genes identified through transcriptomic analyses (the fourth and fifth columns) and literature
screening (the sixth column). The third column describes the functional classes of the candidate genes.

| | | | Found | Found | Found |
|---|---|---|---|---|---|
| | | | through B5- | through high | through |
| | | | selection | recombinant | literature |
| Gene symbols | PROTEIN/RNA MIP | Functional classes | screening | cell screening | screening |
| KEAP1 | Kelch Like ECH Associated | UPR response/protein | | | ✓ |
| | Protein 1 | ubiquitination | | | |
| IRE1alpha | Inositol-Requiring Enzyme 1 | UPR response/protein | | | ✓ |
| | | ubiquitination | | | |
| HRD1 | E3 ubiquitin-protein ligase | UPR response/protein | | | ✓ |
| | | ubiquitination | | | |
| XTP3-B-long | Endoplasmic Reticulum Lectin 1 | UPR response/protein | | | ✓ |
| | | ubiquitination | | | |
| SPOP | Speckle Type BTB/POZ Protein | Apoptosis, ubiquitination | | | ✓ |
| | | and proteasomal degradation | | | |
| GGCX_iso1 | Gamma-Glutamyl Carboxylase | posttranslational | | | ✓ |
| | | protein modification | | | |
| GGCX_iso2 | Gamma-Glutamyl Carboxylase | posttranslational | | | ✓ |
| | | protein modification | | | |
| VKORC1L1_iso1 | Vitamin K Epoxide Reductase | posttranslational | | | ✓ |
| | Complex Subunit 1 Like 1 | protein modification | | | |
| SUMF1 | Sulfatase Modifying Factor 1 | posttranslational | | | ✓ |
| | | protein modification | | | |
| GnTI_MGAT | alpha-1,3-mannosyl-glycoprotein | posttranslational | | | ✓ |
| | beta-1, 2-N- | protein modification | | | |
| | acetylglucosaminyltransferase | | | | |
| GlcNAcTII_MGAT2 | Mannosyl (Alpha-1,6-)- | posttranslational | | | ✓ |
| | Glycoprotein Beta-1,2-N- | protein modification | | | |
| | Acetylglucosaminyltransferase | | | | |
| GT1 | Galactosyltransferase 1 | posttranslational | | | ✓ |
| | | protein modification | | | |
| Lypd4 | LY6/PLAUR Domain Containing 4 | Metabolism of proteins, | | ✓ | |
| | | Post-translational | | | |
| | | modification | | | |
| TIG | Trigger factor | protein export | | | ✓ |
| CAV3 | Caveolin 3 | protein scaffolding | | | ✓ |
| STIM2.2 | Stromal Interaction Molecule 2 | calcium homeostasis | | | ✓ |
| STIM2.1 | Stromal Interaction Molecule 2 | calcium homeostasis | | | ✓ |
| STIM1 | Stromal Interaction Molecule 1 | calcium homeostasis | | | ✓ |
| Orai1 | ORAI Calcium Release-Activated | calcium homeostasis | | | ✓ |
| | Calcium Modulator 1 | | | | |
| Orai2 | ORAI Calcium Release-Activated | calcium homeostasis | | | ✓ |
| | Calcium Modulator 2 | | | | |
| Orai3 | ORAI Calcium Release-Activated | calcium homeostasis | | | ✓ |
| | Calcium Modulator 3 | | | | |
| GPX7 | Glutathione Peroxidase 7 | redox homeostasis | | | ✓ |
| GGCT | Gamma- glutamylcyclotransferase | glutathione catabolism, | | | ✓ |
| | | glutathione homeostasis | | | |
| TOP1 | DNA Topoisomerase I | DNA modeling | | | ✓ |
| MSL1 | Male Specific Lethal 1 Homolog | DNA modeling | | | ✓ |
| MOF | Ortholog Of Drosophila Males | DNA recombination/repair | | | ✓ |
| | Absent On The First | | | | |
| MutL | DNA mismatch repair protein MutL | DNA recombination/repair | | | ✓ |
| APEX1_var1 | Apurinic/Apyrimidinic | DNA recombination/repair | | | ✓ |
| | Endodeoxyribonuclease 1 | | | | |
| ATL | DNA base-flipping protein | DNA recombination/repair | | | ✓ |
| SRS2 | zinc finger SWIM domain- | DNA recombination/repair | | | ✓ |
| | containing protein 7 | | | | |
| Samhd1 | SAM And HD Domain Containing | DNA recombination/repair | | ✓ | |
| | Deoxynucleoside Triphosphate | | | | |
| | Triphosphohydrolase 1 | | | | |
| RSF1 | Remodeling And Spacing Factor 1 | Mitotic and chromosome | | | ✓ |
| | | maintenance | | | |
| Cyp4a14 | cytochrome P450 | transcription | ✓ | | |
| Acot1 | acyl-coenzyme A thioesterase 1 | Lipid Metabolism | ✓ | | |
| Hmgcs2 | 3-hydroxy-3-methylglutaryl-CoA | Lipid Metabolism | ✓ | | |
| | synthase 2 | | | | |
| Tbxas1 | thromboxane A synthase 1 | Lipid Metabolism | ✓ | | |
| | (platelet) | | | | |
| Adh7 | alcohol dehydrogenase 7 | Lipid Metabolism | ✓ | | |
| LSS_iso1 | Lanosterol Synthase | lipid metabolism | | | ✓ |
| PORCN | Porcupine O-Acyltransferase | lipid metabolism | | | ✓ |
| DMP4 | Dentin Matrix Protein 4 | glucose metabolism | | | ✓ |
| Gmpr | guanosine monophosphate reductase | Nucleotide Metabolism | ✓ | | |
| Slc28a3 | solute carrier | Nucleotide Metabolism | ✓ | | |
| Pde1a | phosphodiesterase 1A, calmodulin- | Nucleotide Metabolism | | ✓ | |
| | dependent | | | | |
| MTH1d | MutT Human Homolog 1 | Nucleotide/nucleoside | | | ✓ |
| | | diphosphate metabolism | | | |
| MTH2_iso1 | MutT Homolog 2 | Nucleotide/nucleoside | | | ✓ |
| | | diphosphate metabolism | | | |
| MTH3 | MutT Homolog 3 | Nucleotide/nucleoside | | | ✓ |
| | | diphosphate metabolism | | | |
| Nat8 | N-acetyltransferase 8 | Amino Acid Metabolism | ✓ | | |
| CTH | Cystathionase | amino acid biosynthesis | | | ✓ |
| CBS | Cystathionine-Beta-Synthase | amino acid biosynthesis | | | ✓ |
| GS | glutamine synthetase (glutamate- | amino acid biosynthesis | | | ✓ |
| | ammonia ligase) | | | | |
| GCLM | Glutamate-Cysteine Ligase Modifier | glutathione synthesis | | | ✓ |
| | Subunit | | | | |
| Lacc1 | laccase (multicopper oxidoreductase) | Secondary Metabolism | ✓ | | |
| Slc22a14 | solute carrier | Transport | ✓ | | |
| Slc5a3 | solute carrier | Transport | ✓ | | |
| Abca9 | ATP-binding cassette, sub-family A, | Transport | | ✓ | |
| | member 9 | | | | |
| Slc25a23 | solute carrier family 25 member 23 | Transport | | ✓ | |
| SLC7A11 | Solute Carrier Family 7 Member 11 | Transport | | | ✓ |
| SLC35A1 | Solute Carrier Family 35 Member A1 | Transport | | | ✓ |
| MCT1 | Monocarboxylate Transporter 1 | Transport | | | ✓ |
| RAMP1 | Receptor Activity Modifying | Transport | | | ✓ |
| | Protein 1 | | | | |
| RAMP2 | Receptor Activity Modifying | Transport | | | ✓ |
| | Protein 2 | | | | |
| RAMP3 | Receptor Activity Modifying | Transport | | | ✓ |
| | Protein 3 | | | | |
| YidC | Membrane protein insertase YidC | Transport | | | ✓ |
| ASIC1 | Acid Sensing Ion Channel Subunit 1 | Transport | | | ✓ |
| TMEM20 | Transmembrane Protein 20 | Transport | | | ✓ |
| Nars | asparaginyl-tRNA synthetase | Translation | ✓ | | |
| Trmt61a | tRNA methyltransferase 61 | Translation | ✓ | | |
| | homolog A | | | | |
| Paip1 | poly(A) binding protein interacting | Translation | | | ✓ |
| | protein 1 | | | | |
| Npm1 | nucleophosmin, (nucleolar | Translation | | | ✓ |
| | phosphoprotein B23, numatrin) | | | | |
| Casc3 | cancer susceptibility candidate 3 | Translation | | | ✓ |
| GADD34 | Growth Arrest And DNA-Damage- | translation | | | ✓ |
| | Inducible 34 | | | | |
| Actc1 | actin, alpha, cardiac muscle 1 | Cytoskeleton | ✓ | | |
| Fn3krp | fructosamine 3 kinase related | Protein Repair | ✓ | | |
| | protein | | | | |
| Tagap | T-cell activation RhoGTPase | Signal Transduction | ✓ | ✓ | |
| | activating protein | | | | |
| Mt1 | metallothionein 1 | Signal Transduction | ✓ | | |
| Rxfp1 | relaxin/insulin-like family peptide | Signal Transduction | | ✓ | |
| | receptor 1 | | | | |
| Edn1 | endothelin 1 | Signal Transduction | | ✓ | |
| Erp27 | endoplasmic reticulum protein 27 | protein folding | | ✓ | |
| Erp57 | Endoplasmic Reticulum Resident | protein folding | | ✓ | ✓ |
| | Protein 57 | | | | |
| Dsbc | Thiol:disulfide interchange protein | protein folding, | | | ✓ |
| | | oxidative stress | | | |
| FkpA | FKBP-type peptidyl-prolyl cis-trans | protein folding | | | ✓ |
| | isomerase | | | | |
| Skp | chaperone protein Skp | protein folding | | | ✓ |
| ERp44 | Endoplasmic reticulum resident | Protein folding, | | | ✓ |
| | protein 44 | protein quality control | | | |
| Foxa1 | forkhead box A1 | Transcription | | ✓ | |
| PPARA | peroxisome proliferator activated | Transcription | | | ✓ |
| | receptor alpha | | | | |
| PPAR gamma_iso1 | Peroxisome Proliferator Activated | Transcription | | | ✓ |
| | Receptor Gamma | | | | |
| PPAR gamma_iso2 | Peroxisome Proliferator Activated | Transcription | | | ✓ |
| | Receptor Gamma | | | | |
| PCBD1 | Pterin-4 Alpha-Carbinolamine | Transcription regulation | | | ✓ |
| | Dehydratase 1 | | | | |
| XBP1s | X-box-binding protein-1 | Transcription regulation | | | ✓ |
| CEBPalpha | CCAAT Enhancer Binding Protein | Transcription regulation | | | ✓ |
| | Alpha | | | | |
| Cdk15 | cyclin-dependent kinase 15 | cell survival/proliferation | | ✓ | |
| Vegfd | vascular endothelial growth factor D | cell survival/proliferation | | ✓ | |
| Frk | fyn-related kinase | cell survival/proliferation | | ✓ | |
| Ca3 | carbonic anhydrase III, muscle | cell survival/proliferation | | ✓ | |
| | specific | | | | |
| FGF4 | Fibroblast Growth Factor 4 | cell survival/proliferation | | | ✓ |
| Bag3 | BCL2-Associated Athanogene 3 | cell survival/proliferation | | | ✓ |
| Mapk7 | Mitogen-Activated Protein Kinase 7 | cell survival/proliferation | | | ✓ |
| CFLAR | CASP8 And FADD Like Apoptosis | Apoptosis cell survival/ | | | |
| | Regulator | cell death pathways | | | |
| SOD1 | Superoxide Dismutase 1 | Apoptosis, oxidative stress | | | ✓ |
| Beclin-1 | Coiled-Coil, Moesin-Like BCL2 | Autophagy, cell | | | ✓ |
| | Interacting Protein | death/apoptosis | | | |
| Wip1 | Wild-Type P53-Induced | cell stress response, | | | ✓ |
| | Phosphatase 1 | apoptosis | | | |
| HSP27 | Endoplasmic Reticulum Protein 27 | Protein folding, apoptosis, | | | ✓ |
| | | cell development and | | | |
| | | differentiation | | | |
| SIRT1 | Sirtuin 1 | metobolism, apoptosis, | | | ✓ |
| | | autophagy, epigenetic | | | |
| | | gene silencing | | | |
| Rassf9 | Ras association domain family | vesicular trafficking | | ✓ | |
| | member 9 | | | | |
| Arhgap42 | Rho GTPase activating protein 42 | cell adhesion; | | ✓ | |
| | | vesicular trafficking | | | |
| Clstn3 | calsyntenin 3 | cell adhesion; | | ✓ | |
| | | vesicular trafficking | | | |
| Dpt | Dermatopontin | cell adhesion, | | ✓ | |
| | | extracellular matrix | | | |
| Epcam | epithelial cell adhesion molecule | cell adhesion, | | ✓ | |
| | | extracellular matrix | | | |
| Fras1 | Fraser syndrome 1 | cell adhesion, | | ✓ | |
| | | extracellular matrix | | | |
| Igdcc4 | immunoglobulin superfamily, DCC | cell adhesion, | | ✓ | |
| | subclass, member 4 | extracellular matrix | | | |
| Nid2 | nidogen 2 | cell adhesion, | | ✓ | |
| | | extracellular matrix | | | |
| Pcdhb3 | protocadherin beta 3 | cell adhesion, | | ✓ | |
| | | extracellular matrix | | | |
| Egfr | epidermal growth factor receptor | “Response to recombinant | ✓ | | |
| | | protein/Linked to | | | |
| | | inflammation” | | | |
| Fcer2 | Fc fragment of IgE, low affinity II | | ✓ | | |
| Cxcl10 | chemokine (C-X-C motif) ligand 10 | | ✓ | | |
| Lum | Lumican | | ✓ | | |
| Vcam1 | vascular cell adhesion molecule 1 | | ✓ | | |
| Mpp7 | membrane protein, palmitoylated 7 | | ✓ | | |
| Nckap5 | NCK-associated protein 5 | | ✓ | | |
| Mybpc2 | myosin-binding protein C, fast-type | | ✓ | ✓ | |
| Plekha6 | pleckstrin homology domain containing | | ✓ | ✓ | |
| Tnfsf15 | tumor necrosis factor superfamily | | ✓ | | |
| Xlr3a | X-linked lymphocyte-regulated | | ✓ | | |
| | protein 3A | | | | |
| Sirpa | signal-regulatory protein alpha | | ✓ | | |
| Cc12 | C-C motif chemokine 2 | | ✓ | | |
| Gbp5 | guanylate binding protein 5 | | ✓ | | |
| Klra2 | killer cell lectin-like receptor 2 | | | ✓ | |
| Klrb1f | killer cell lectin-like receptor | | | ✓ | |
| | subfamily B member 1F | | | | |
| SPINK6 | Serine Peptidase Inhibitor, Kazal | response to recombinant | | ✓ | |
| | Type 6 | protein/linked to | | | |
| | | inflammation | | | |
| A20 | Tumor Necrosis Factor Inducible | response to recombinant | | | ✓ |
| | Protein A20 | protein/linked to | | | |
| | | inflammation | | | |
| HGF | Hepatocyte Growth Factor | response to recombinant | | | ✓ |
| | | protein/linked to | | | |
| | | inflammation | | | |
| NaN | uncharacterized LOC103159978 | non-coding RNAs | | ✓ | |
| | (LOC103159978), ncRNA | | | | |
| NaN | uncharacterized LOC103162358 | | | ✓ | |
| | (LOC103162358), transcript variant | | | | |
| | X1, ncRNA | | | | |
| NaN | uncharacterized LOC103160835 | | | ✓ | |
| | (LOC103160835), ncRNA | | | | |
| NaN | uncharacterized LOC103159713 | | | ✓ | |
| | (LOC103159713), ncRNA | | | | |
| NaN | uncharacterized LOC103164404 | | | ✓ | |
| | (LOC103164404), ncRNA | | | | |
| NaN | uncharacterized LOC103163127 | | | ✓ | |
| | (LOC103163127), transcript variant | | | | |
| | X6, ncRNA | | | | |
| NaN | uncharacterized LOC103159176 | | | ✓ | |
| | (LOC103159176), ncRNA | | | | |

TABLE 2
Genes upregulated in Tras high producer clones (HPC) versus parental CHO cells and versus Tras polyclonal cells (PC).

| Gene | | fold change | fold change | | Foxa1 |
|---|---|---|---|---|---|
| Symbol¹ | Detail | (HPC/CHO cells) | (HPC/PC) | functional classes | target gene² |
| Abca9 | ATP-binding cassette, sub-family A, | 3.45 | 5.08 | Transporters | |
| | member 9 | | | | |
| Slc25a23 | solute carrier family 25 (mitochondrial | 2.54 | 2.00 | Transporters | ✓ |
| | carrier; phosphate carrier), member 23 | | | | |
| Erp27 | endoplasmic reticulum protein 27 | 4.20 | 3.65 | protein folding | |
| Ca3 | carbonic anhydrase III, muscle specific | 19.86 | 15.68 | cell survival | ✓ |
| Cdk15 | cyclin-dependent kinase 15 | 2.93 | 2.54 | cell survival | |
| Vegfd | c-fos induced growth factor (vascular | 4.35 | 3.90 | cell proliferation regulation/ | |
| | endothelial growth factor D) | | | cell survival/signaling/cell | |
| | | | | differentiation | |
| Frk | fyn-related kinase | 5.40 | 10.91 | cell proliferation regulation/ | ✓ |
| | | | | signaling/cell differentiation | |
| Foxa1 | forkhead box A1 | 2.95 | 3.03 | chromatin remodeling | ✓ |
| Pde1a | phosphodiesterase 1A, calmodulin- | 2.79 | 4.93 | Signaling | ✓ |
| | dependent | | | | |
| Plekha6 | pleckstrin homology domain containing, | 3.51 | 3.56 | Signaling | |
| | family A member 6 | | | | |
| Rxfp1 | relaxin/insulin-like family peptide | 5.78 | 5.18 | Signaling | |
| | receptor 1 | | | | |
| Edn1 | endothelin 1 | 17.91 | 10.27 | Signaling | ✓ |
| Tagap | T-cell activation RhoGTPase activating | 4.94 | 8.42 | signaling/cytoskeleton | |
| | protein | | | organization | |
| Arhgap42 | Rho GTPase activating protein 42 | 12.00 | 10.79 | signaling/cytoskeleton | ✓ |
| | | | | organization | |
| Rassf9 | Ras association (RalGDS/AF-6) domain | 12.22 | 14.76 | vesicular trafficking | ✓ |
| | family (N-terminal) member 9 | | | | |
| Clstn3 | calsyntenin 3 | 3.45 | 2.69 | cell adhesion/vesicular | |
| | | | | trafficking/cell growth | |
| Dpt | Dermatopontin | 5.01 | 5.17 | cell adhesion/cell proliferation | |
| | | | | regulation | |
| Epcam | epithelial cell adhesion molecule | 4.56 | 5.56 | cell adhesion/signaling/cell | ✓ |
| | | | | proliferation regulation | |
| Fras1 | Fraser syndrome 1 | 5.99 | 5.28 | cell adhesion/signaling | ✓ |
| Igdcc4 | immunoglobulin superfamily, DCC subclass, | 7.11 | 2.53 | N/A | |
| | member 4 | | | | |
| Mybpc2 | myosin-binding protein C, fast-type | 29.47 | 3.32 | cytoskeleton organization/cell | |
| | (LOC100774229) | | | adhesion | |
| Nid2 | nidogen 2 (osteonidogen) | 5.88 | 4.37 | cell adhesion | |
| Pcdhb3 | protocadherin beta 3 | 2.83 | 2.43 | cell adhesion | |
| Klra2 | killer cell lectin-like receptor 2 | 8.13 | 1.86 | immune response | |
| | (LOC100762405) | | | | |
| Klrb1f | killer cell lectin-like receptor subfamily B | 4.56 | 6.67 | immune response | |
| | member 1F (LOC100757275) | | | | |
| NaN | uncharacterized LOC103159978 | 5.51 | 5.20 | non-coding RNAs | |
| | (LOC103159978), hereafter called | | | | |
| | ncRNA978 | | | | |
| NaN | uncharacterized LOC103162358 | 2.36 | 2.53 | non-coding RNAs | |
| | (LOC103162358), ncRNA358 | | | | |
| NaN | uncharacterized LOC103160835 | 10.23 | 6.75 | non-coding RNAs | |
| | (LOC103160835), ncRNA835 | | | | |
| NaN | uncharacterized LOC103159713 | 3.45 | 4.90 | non-coding RNAs | |
| | (LOC103159713), ncRNA713 | | | | |
| NaN | uncharacterized LOC103164404 | 2.83 | 2.47 | non-coding RNAs | |
| | (LOC103164404), ncRNA404 | | | | |
| NaN | uncharacterized LOC103163127 | 4.53 | 5.03 | non-coding RNAs | |
| | (LOC103163127), ncRNA127 | | | | |
| NaN | uncharacterized LOC103159176 | 5.09 | 9.06 | non-coding RNAs | |
| | (LOC103159176), ncRNA176 | | | | |

¹Genes upregulated in Tras high producer cell clones versus parental CHO cells and versus Tras polyclonal cells were selected according to the following criteria: a log2 fold change > 0.5 and a p-value < 0.05.
²Genes listed as Foxa1 target genes according to ChIP-seq datasets (ENCODE Transcription Factor Targets dataset) and to low or high-throughput transcription factor functional studies (TRANSFAC Curated Transcription Factor Targets Dataset) obtained using the Harmonizome web portal (Rouillard et al., 2016).

TABLE 3
Sequences encoding selected tested and possible MIPs

| SEQ | | | Coding |
|---|---|---|---|
| ID | | | sequence |
| NO | Gene symbol | Organism | (CDS) = ✓ |
| 1 | Cyp4a14 | Cricetulus griseus | ✓ |
| 2 | Acot1 | Cricetulus griseus | ✓ |
| 3 | Hmgcs2 | Cricetulus griseus | ✓ |
| 4 | Tbxas1 | Cricetulus griseus | ✓ |
| 5 | Adh7 | Cricetulus griseus | ✓ |
| 6 | guanosine monophosphate | Cricetulus griseus | |
| | reductase (Gmpr), transcript | | |
| | variant X1 (mRNA) | | |
| 7 | guanosine monophosphate | Cricetulus griseus | |
| | reductase (Gmpr), transcript | | |
| | variant X1 (mRNA) | | |
| 8 | Slc28a3 | Cricetulus griseus | ✓ |
| 9 | Nat8 | Cricetulus griseus | ✓ |
| 10 | Lacc1 | Cricetulus griseus | ✓ |
| 11 | Slc22a14 | Cricetulus griseus | ✓ |
| 12 | Slc5a3 | Cricetulus griseus | ✓ |
| 13 | Nars | Cricetulus griseus | ✓ |
| 14 | Trmt61a | Cricetulus griseus | ✓ |
| 15 | Actc1 | Cricetulus griseus | ✓ |
| 16 | Fn3kpr1 | Cricetulus griseus | ✓ |
| 17 | Tagap | Cricetulus griseus | ✓ |
| 18 | Mt1 | Cricetulus griseus | ✓ |
| 19 | PPARalpha | Mus musculus | ✓ |
| 20 | Rassf9 | Cricetulus griseus | ✓ |
| 21 | Erp27 | Cricetulus griseus | ✓ |
| 22 | Erp57 | Cricetulus griseus | ✓ |
| 23 | Clstn3 | Cricetulus griseus | ✓ |
| 24 | CDK15 | Cricetulus griseus | ✓ |
| 25 | Ca3 | Cricetulus griseus | ✓ |
| 26 | Foxa1 | Cricetulus griseus | ✓ |
| 27 | NPM1 | Cricetulus griseus | ✓ |
| 28 | Casc3 | Cricetulus griseus | ✓ |
| 29 | Paip1, isoform p45 | Cricetulus griseus | ✓ |
| 30 | Paip1, isoform p65 | Cricetulus griseus | ✓ |
| 31 | CFLAR | Homo sapiens | ✓ |
| 32 | GCLM | Homo sapiens | ✓ |
| 33 | ACTC1 | Homo sapiens | ✓ |
| 34 | GGCT | Homo sapiens | ✓ |
| 35 | SOD1 | Homo sapiens | ✓ |
| 36 | ERp27 | Cricetulus griseus | ✓ |
| 37 | ERp57 | Cricetulus griseus | ✓ |
| 38 | HSP27 | Homo sapiens | ✓ |
| 39 | Dsbc | E. coli | ✓ |
| 40 | FkpA | E. coli | ✓ |
| 41 | Skp | E. coli | ✓ |
| 42 | Beclin-1 | Homo sapiens | ✓ |
| 43 | Wip-1 | Homo sapiens | ✓ |
| 44 | MTH1d | Homo sapiens | ✓ |
| 45 | MTH2, isoform 1 | Homo sapiens | ✓ |
| 46 | MTH3 | Homo sapiens | ✓ |
| 47 | PPAR alpha | Homo sapiens | ✓ |
| 48 | PPAR gamma, isoform 1 | Homo sapiens | ✓ |
| 49 | PPAR gamma, isoform 1 | Homo sapiens | ✓ |
| 50 | SIRT1 | Homo sapiens | ✓ |
| 51 | RSF1 | Homo sapiens | ✓ |
| 52 | SPOP | Homo sapiens | ✓ |
| 53 | Lypd4 | Homo sapiens | ✓ |
| 54 | SLC7A11 | Homo sapiens | ✓ |
| 55 | ERp44 | Homo sapiens | ✓ |
| 56 | ERp57 | Homo sapiens | ✓ |
| 57 | ERP27 | Homo sapiens | ✓ |
| 58 | KEAP1 | Homo sapiens | ✓ |
| 59 | GGCX, isoform 1 | Homo sapiens | ✓ |
| 60 | GGCX, isoform 2 | Homo sapiens | ✓ |
| 61 | VKORC1L1, isoform 1 | Homo sapiens | ✓ |
| 62 | SPINK6 | Homo sapiens | ✓ |
| 63 | TIG | E. coli | ✓ |
| 64 | CAV3 | Homo sapiens | ✓ |
| 65 | STIM2.2 | Homo sapiens | ✓ |
| 66 | STIM2.1 | Homo sapiens | ✓ |
| 67 | STIM1 | Homo sapiens | ✓ |
| 68 | Orai1 | Homo sapiens | ✓ |
| 69 | Orai2 | Homo sapiens | ✓ |
| 70 | Orai3 | Homo sapiens | ✓ |
| 71 | DMP4 | Homo sapiens | ✓ |
| 72 | TMEM20 | Homo sapiens | ✓ |
| 73 | TOP1 | Homo sapiens | ✓ |
| 74 | MOF | Homo sapiens | ✓ |
| 75 | MSL1 | Homo sapiens | ✓ |
| 76 | SUMF1 | Homo sapiens | ✓ |
| 77 | PORCN | Homo sapiens | ✓ |
| 78 | MutL | E. coli | ✓ |
| 79 | GADD34 | Homo sapiens | ✓ |
| 80 | A20 | Homo sapiens | ✓ |
| 81 | IRE1 alpha | Homo sapiens | ✓ |
| 82 | LSS, isoform 1 | Homo sapiens | ✓ |
| 83 | CEBP alpha | Homo sapiens | ✓ |
| 84 | XBP1s | Homo sapiens | ✓ |
| 85 | GPX7 | Homo sapiens | ✓ |
| 86 | CTH | Homo sapiens | ✓ |
| 87 | CBS | Homo sapiens | ✓ |
| 88 | FGF4 | Homo sapiens | ✓ |
| 89 | GnTI_MGAT | Homo sapiens | ✓ |
| 90 | GlcNAcTII_MGAT2 | Homo sapiens | ✓ |
| 91 | APEX1, variant 1 | Homo sapiens | ✓ |
| 92 | ATL | E. coli | ✓ |
| 93 | HRD1 | Homo sapiens | ✓ |
| 94 | XTP3-B-long | Homo sapiens | ✓ |
| 95 | HGF | Homo sapiens | ✓ |
| 96 | PCBD1 | Homo sapiens | ✓ |
| 97 | RAMP1 | Homo sapiens | ✓ |
| 98 | RAMP2 | Homo sapiens | ✓ |
| 99 | RAMP3 | Homo sapiens | ✓ |
| 100 | YidC | E. coli | ✓ |
| 101 | GT1 | Homo sapiens | ✓ |
| 102 | MCT1 | Homo sapiens | ✓ |
| 103 | GS | Homo sapiens | ✓ |
| 104 | Bag3 | Homo sapiens | ✓ |
| 105 | SRS2 | Homo sapiens | ✓ |
| 106 | SLC35A1 | Homo sapiens | ✓ |
| 107 | MapK7 | Cricetulus griseus | ✓ |
| 108 | ASIC1 | Cricetulus griseus | ✓ |
| 109 | SamHD1 | Cricetulus griseus | ✓ |
| 110 | Abca9 | Cricetulus griseus | ✓ |
| 111 | Slc25a23 | Cricetulus griseus | ✓ |
| 112 | Vegfd | Cricetulus griseus | ✓ |
| 113 | Frk | Cricetulus griseus | ✓ |
| 114 | Edn1 | Cricetulus griseus | ✓ |
| 115 | Arhgap42 | Cricetulus griseus | ✓ |
| 116 | Epcam | Cricetulus griseus | ✓ |
| 117 | Fras1 | Cricetulus griseus | ✓ |
| 118 | Pcdhb3 | Cricetulus griseus | ✓ |
| 119 | Pde1a | Cricetulus griseus | ✓ |

TABLE 4
List of primers used for RT-qPCR analysis of FIGS. 16-19

| | | SEQ ID | | SEQ ID |
|---|---|---|---|---|
| Gene | Forward primer (FP) | NO (FP) | Reverse primer (RP) | NO (RP) |
| Erp27 | TGCAGCTGGCTTATTTAACACC | 120 | CCTGGAAGAGCTTAGCTGCC | 137 |
| Erp57 | TGGAACTCACGGACGAAAACT | 121 | AGGGGCGAAGAACTCGACTA | 138 |
| SDHA | TGGCGTGGATGTCACTAAGG | 122 | CAGCACCTGCCCTTTGTAGT | 139 |
| Foxa1 | AAAGGGGACCCCCACTACTC | 123 | TGCCTTGAAGTCCAGCTTGT | 140 |
| Ca3 | GGAATCGCTGTTGTTGGCAT | 124 | GAGCCTCCTTGCCCTTAGTC | 141 |
| Rassf9 | TGGCACAGCTAGAAGAACGG | 125 | TCTTCACTTCCGTCGATGCC | 142 |
| Epcam | TGTTTGGTGATGAAGGCGGA | 126 | TCGTTGTTCTGTATGGCCCC | 143 |
| Pcdhb3 | CTGGGTCTAGGCGCTATTCTG | 127 | CTACCCTGAGCCCCAAATCC | 144 |
| Slc25a23 | TCCGAGATTCAGCAGAGCTTC | 128 | CGCCATTCCTGCCAATCAATG | 145 |
| Pde1a | TGGGTGTTTCTTGGGGTAGG | 129 | ACCCACCAAGAGTCACGTTG | 146 |
| Edn1 | AGAAGGTTGGAGGCCATCAC | 130 | TGCTCGGTTGTGTGTCAACT | 147 |
| Fras1 | CACACCCACCTGGAAAGTCA | 131 | GTTAGGCCATCTTCCCGAGC | 148 |
| Frk | GCCCAGTCCCCTCTTGATTT | 132 | GCAGAGCTGAGAGAGTTCCC | 149 |
| Arhgap42 | CAGTTCAACTTGCAGAATACAAGG | 133 | TGGCTGGGTGGTCTGTAATC | 150 |
| Tagap | GCCCACCATCCTACGAAGAG | 134 | GAGCCGTGTTCCATTTGAGC | 151 |
| Tras HC | GACTCCGATGGGTCGTTCTT | 135 | CATGACGGAGCAGGAGAACA | 152 |
| Tras LC | GCGGACTACGAGAAGCACAA | 136 | CGGTTGAACGACTTGGTCAC | 153 |

Material and Methods

MIP Candidate Selection and Discovery

Candidate Gene Sequences and DNA Vector Constructs

Genomic and cDNA sequences of RNAseq MIP candidates were determined after alignment of the homologous genes in mice using NCBI BLAST software. Transcript sequence and accumulation of the corresponding genes was determined using SELEXIS CHO-M gene expression database.

CHO-M (SURE CHO-M Cell Line™ (SELEXIS Inc., San Francisco, USA)), cDNA library was amplified by reverse transcription from 1 ug total RNA isolated from 106 CHO-M cells (NucleoSpin™ RNA kit; Macherey-Nagel) using the GoScript Reverse transcription System (Promega).

MIP coding sequences (CDS) were cloned into the pBSK_ITR_BT+_EGFP_X29_ITR vector (SELEXIS Inc., San Francisco, USA), by cutting out the green fluorescent protein (GFP) gene and replacing it with the MIP CDS.

Vectors were constructed as follow: The CDS were amplified from CHO-M cDNA library by PCR (PHUSION High-Fidelity DNA Polymerase; Finnzymes, THERMO FISHER SCIENTIFIC) from ATG to Stop using primers carrying restriction site. Then, the cDNA products and pBSK_ITR_BT+_EGFP_X29_ITR vectors were double-digested by the corresponding restriction enzymes. Finally, the cDNAs were ligated into the pBSK_ITR_BT vector where the GFP sequence was cut out after digestions with the same restriction enzymes.

The pBSK_ITR_BT+_EGFP_X29_ITR vector includes an expression cassette composed of the CMV/EF1alpha promoter and the BGH polyadenylation signal followed by the hMAR X-29. The expression cassette is flanked by the inverted terminal sequences of the piggyBac transposon.

The GFP protein was expressed using a eukaryotic expression cassette composed of a human cytomegalovirus (CMV) enhancer and human glyceraldehydes 3-phosphate dehydrogenase (GAPDH) promoter upstream of the coding sequence followed by a simian virus 40 (SV40) polyadenylation signal, the human gastrin terminator and a SV40 enhancer (Le Fourn et al., 2013). The pSG5_PPARα vector was obtained from Issemann and Green, 1990.

The BLASTICIDIN vector (pBlast) contains the blasticidin resistance gene under the control of the SV40 promoter originated from pRc/RSVplasmid (INVITROGEN/LIFE TECHNOLOGIES).

RNASeq Analysis

Cells used for the RNASeq analysis are the following:
CHO-M WT cells
Polyclonal cell population expressing the Etanercept (ENBREL) Fc-fusion (difficult-to express) selected with puromycin and B5, or with puromycin only.
Polyclonal cell population expressing the Trastuzumab IgG (easy to express) selected with puromycin and B5, or with puromycin only.
Clones expressing the Trastuzumab IgG (easy to express) selected with puromycin.
Clone expressing the Bevacizumab IgG (easy to express) selected with puromycin.
Clone expressing the Interferon beta (difficult to express) selected with puromycin.

These cells were grown for 4 days in spintubes without antibiotic selection. Total RNA was isolated from cells using the NucleoSpin RNA kit (Macherey-Nagel). RNA quality was evaluated using the Fragment Analyzer (Advanced Analytical). RNA-seq library preparation was achieved using 0.5 μg to 1 μg of total converted to cDNA using the Illumina TruSeq® stranded mRNA-seq reagents (ILLUMINA). The RNA-seq library 100 nt paired end was sequenced on the Illumina HiSeq 2500©. Reads were mapped to the CHO-K1 transcriptome (RefSeq, 2014).

Cell Culture, Stable Transformation and Stable Polyclonal Line Analyses

Suspension Chinese hamster ovary cells (CHO-M) were maintained in suspension culture in SFM4CHO Hyclone serum-free medium (SFM, THERMO SCIENTIFIC) supplemented with L-glutamine (PAA, Austria) and HT supplement (GIBCO, INVITROGEN LIFE SCIENCES) at 37° C., 5% CO2 in humidified air. Other cell media used for these experiments is the Deficient BalanCD CHO-M Growth A (B-CDmin; Irvine Scientific), supplemented with vitamin B1 (thiamine Hydrochloride; SIGMA ALDRICH), vitamin B5 (Calcium DL-Pantothenate; TCI) and vitamin H (Biotin, SIGMA ALDRICH).

CHO-M cells were transfected with pBSK-MIP, pBlast, and pCS2-U5-PBU3 IgG1-Hc or IgG1-Lc expression vectors by electroporation according to the manufacturer's recommendations (NEONDEVICES, INVITROGEN). Production of stable cell lines was achieved using SFM4CHO media complemented with 7.5 μg/ml of blasticidin for 3 weeks.

GFP and IgG1-producing cell polyclonal lines expressing the GFP or IgG were selected for further experiments as follow: For blasticidin selection, cells were seeded in SFM media supplemented with 10 mg/ml blasticidin for 2 weeks, then transferred into well with SFM media for 5 days, then into 50 ml spin tubes with SFM media.

For double selection of the cells with puromycin then B5, polyclonal stable cell lines were first selected with puromycin, then cells were seeded at 20 000 cells/ml in 24-well plate in B5 selective media for 7 days (B-CDfull media was used as negative control), then transferred in SFM full media wells for 7 days, then seeded into pin tube with SFM media.

The percentage of fluorescent cells and the fluorescence intensity of GFP positive cells were determined by flow cytometry analysis using a CyAn ADP flow cytometer (BECKMAN COULTER). Immunoglobulin concentrations in cell culture supernatants were measured by sandwich ELISA. GFP, IgG1Lc, IgG1Hc and MIP transcript accumulation was confirmed by RT-quantitative PCR assays before analyses.

Surface IgG display was assessed by FACS analysis using a flow cytometery (Beckman Coulter™). Stable clones expressing IgG were obtained by cell sorting on FACS Aria III (BD), expanded and analyzed for IgG production levels (sandwich ELISA).

Transient Assay for Measurement of Peroxisome Proliferator-Activated Receptor Response Element (PPRE) and PPAR Activation

Transient transfection assay was performed as follows: CHO cells were transfected with PPRE-TK-DsRed (provided by Michalik lab., University of Lausanne) or TK-DsRed (PPRE sequence was cut out of the previous vector) without or with pSG5_PPARα vector. pE-BFP2-Nuc(2×NLS) was used as internal transfection control. It contains eBFP2 (enhanced blue fluorescent protein 2) coding sequences under the control of minimal CMV promoter and nuclear localization sequence NLS. Cell were observed 48h after transfection by flow cytometry using a Beckman Coulter Gallios Cell Counter© and signal analyzed by Kaluza Acquisition® software. DsRed activity (detection: 638 nm) was standardized relative to BFP2 marker (detection 488 nm).

Fed-Batch Performance Evaluation

Growth and IgG secretion performances in fed-batch culture were performed according to Le Fourn et al., 2013, with the following changes: IgG producing clone stably transfected for the expression of MIPs were seeded at 300′000 cells/ml in 5 mL culture medium in falcon of 50 mL. Viable cell density and IgG titer (g/L) were evaluated after 3, 6, 8, 9, 10 and 13 days.

Quantitative PCR Analysis

For quantitative PCR (qPCR) analysis, total RNA was extracted from 106 cells and reverse transcribed into cDNA using the GoScript Reverse transcription System (Promega). Transcripts accumulation was quantified by qPCR using the LightCycler© 480 SYBR Green I Master and the LightCycler 480 II instrument (Roche). Transcript levels were normalized to that of SDHA housekeeping gene.

Metabolite Analyzes (Metabolite Extraction, Sample Amount Normalization)

Metabolite Extraction For metabolite quantification, cell pellets were extracted with 1000 μL pre-cooled MeOH:H₂O (4:1, v/v) solvent mixture as a best compromise to efficiently precipitate proteins, quench the metabolism and extract a broad range of polar metabolites. The samples were then probe-sonicated (4 pulses×5 sec) to lyse the cells completely and improve the metabolite extraction. To promote the protein precipitation the samples were incubated for 1 hour at −20° C., followed by 15 min centrifugation at 13,000 rpm at 4° C. The resulting supernatant was collected and evaporated to dryness in a vacuum concentrator (LABCONCO, Missouri, US). Then sample extracts were reconstituted in 100 μL MeOH:Water (4:1) and injected into the LC-MS system.

Protein Quantification

The protein pellets were evaporated and lysed in 20 mM Tris-HCl (pH 7.5), 4M guanidine hydrochloride, 150 mM NaCl, 1 mM Na₂ EDTA, 1 mM EGTA, 1% Triton, 2.5 mM sodium pyrophosphate, 1 mM beta-glycerophosphate, 1 mM Na₃CO₄, 1 μg/ml leupeptin using brief probe-sonication (5 pulses×5 sec). BCA Protein Assay Kit (THERMO SCIENTIFIC, Masschusetts, US) was used to measure (A562 nm) total protein concentration (HIDEX, Turku, Finland).

Data Acquisition—LC-HRMS

Extracted samples were analyzed by Hydrophilic Interaction Liquid Chromatography coupled to high resolution mass spectrometry (HILIC-HRMS) in negative ionization modes using a Q-Exactive© instrument (Quadrupole Orbitrap© mass spectrometer) (THERMO FISHER SCIENTIFIC) operating at mass resolving power of 70,000 full width half maximum (FWHM). Metabolites were chromatographic separated using a ZIC pHILIC (100 mm, 2.1 mm I.D. and 5 μm particle size) column. The mobile phase was composed of A=20 mM ammonium acetate and 20 mM NH₄OH in water at pH 9.3 and B=100% ACN. The linear gradient elution from 90% B (0-1.5 min) down to 50% B was applied (1.5 min-8 min), followed by an isocratic step (8 min-11 min) and linear gradient down to 45% B (11 min-12 min). These conditions were held 3 min. Finally, the initial chromatographic conditions were established as a post-run during 9 min for column re-equilibration. The flow rate was 300 μL/min, column temperature 30° C. and sample injection volume 2.5 pfl. ESI source conditions were set as follows: probe heater temperature 200° C., sheath gas 60 a.u., auxiliary gas 15 a.u., capillary temperature 280° C. and ESI spray voltage −3600 V. Full scan mode was used as acquisition mode to quantify lactate, pyruvate, 3-hydroxybutyrate and pantothenic acid, while acetyl-CoA was quantified using parallel reaction monitoring (PRM) acquisition mode using 30 eV as collision energy.

Data Processing

Raw LC-HRMS data was processed using the Thermo Fisher Scientific software (Xcalibur 4.0 QuanBrowser®, THERMO FISHER SCIENTIFIC). Metabolite quantification was performed using external calibration curve.

Statistical Analysis

The results are expressed as means±standard error of the mean (SEM) or means±standard deviation (SD). Statistical analysis was performed using the one or two-tailed Student's t-test. Asterisks in the figure panels refer to statistical probabilities. Statistical probability values of less than 0.05 were considered significant.

Material and Methods for:

Evaluating the Effect of Individual or Combined Expression of MIP(s) on the Secretion of Different Therapeutic Protein-Expressing Cho Clones

CFLAR, GCLM, ACTC1:

DNA Vector Constructs

The PB transposase expression vector pCS2+U5V5PBU3 contains the PB transposase coding sequence surrounded by the 5′ and 3′ untranslated terminal regions (UTR) of the Xenopus laevis beta-globin gene. This plasmid was constructed as follows: the 3′ UTR 317 bp fragment from pBSSK/SB10 (kindly provided by Dr S. Ivies) was inserted into pCS2+U5 (INVITROGEN/LIFE Technologies, Paisley, UK) to yield pCS2+U5U3. The PB transposase coding sequence (2067 bp, GenBank accession number: EF587698) was synthesized by ATG:biosynthetic (Merzhausen, Germany) and cloned in the pCS2+U5U3 backbone between the two UTRs. The PB control vector corresponds to the unmodified pCS2+U5 plasmid (FIG. 10, left panel). The different transposons vectors were generated by introducing the PB 235 bp 3′ and 310 bp 5′ inverted terminal repeats (ITRs), synthesized by ATG:biosynthetic (Merzhausen, Germany), into the pBluescript SK-plasmid (pBSK ITR3′-ITR5′, FIG. 1, right panel). The puromycin resistance gene (PuroR), under the control of the SV40 promoter from pRc/RSV plasmid (INVITROGEN/LIFE Technologies), was then inserted between the two ITRs. The MAR 1-68 and MAR X-29 elements, the puromycin resistance and GFP genes used in this study were as previously described.

Cell Culture, Stable Transfection and Subcloning of CHO Cell Lines

Suspension Chinese hamster ovary cells (CHO-K1) were maintained in SFM4CHO Hyclone serum-free medium (THERMO SCIENTIFIC) supplemented with L-glutamine (PAA, Austria) and HT supplement (GIBCO, INVITROGEN life sciences) at 37° C., 5% C02 in humidified air. CHO-K1 cells were transfected with recombinant protein of interest expression vector bearing-puromycin resistance gene by electroporation according to the manufacturer's recommendations (Neon devices, Invitrogen). Two days later, the cells were transferred in T75 plates in medium containing 10 ug/ml of puromycin and the cells were further cultivated under selection for two weeks. Stable individual cell clones expressing bevacizumab IgG, Fc-fusion or circulating hormone were then generated by limiting dilution, expanded and analyzed for growth performance and production levels. Bevacizumab IgG-, Fc-fusion-producing cell clones expressing the highest protein levels were selected for further biochemical experiments.

Circulating hormone expressing CHOM clones were analyzed by SDS-PAGE and immunoblotting.

Some of these clones were then co-transfected with the various metabolic-improving proteins (MIPs) expressing vector and a plasmid bearing the blasticidin resistance gene by electroporation as described below. Cells were then cultivated in medium containing 10 ug/ml of blasticidin for two weeks as described above. Stable clones were isolated by limited dilution and clones isolated using clonepix device before to be analyzed for growth and production.

Cell Culture and Transfection Analysis

CHO-M cells were maintained in suspension culture in SFM4CHO Hyclone serum-free medium (THERMO SCIENTIFIC) supplemented with L-glutamine (PAA, Austria) and HT supplement (GIBCO, INVITROGEN life sciences) at 37° C., 5% CO₂ in humidified air. Transposon donor plasmids were transferred in these cells by electroporation according to the manufacturer's recommendations (Neon devices, INVITROGEN). Quantification of recombinant protein secretion level was performed from batch cultures as described previously (see Le Fourn et al., 2013). Briefly, cell populations expressing immunoglobulins were evaluated in batch cultivation into 50 ml minibioreactor tubes (TPP, Switzerland) at 37° C. in 5% CO₂ humidified incubator for 7 days. Immunoglobulin concentrations in cell culture supernatants were measured by sandwich ELISA.

Alternatively, two clones were isolated from non-sorted and non-selected populations expressing each of the three IgGs using a ClonePix© device. Briefly, semi-solid media was used to immobilize single cells, and colonies secreting high amounts of IgG were picked ten days post-embedding. These cell lines were passaged every 3-4 days in spin tube bioreactors at a density of 3×1 05 cells/ml in a peptone-containing growth medium (Hyclone SFM4CHO supplemented with 8 mM glutamine) in a humidified incubator maintained at 37° C. and 5% CO₂, with orbital shaking at 180 rpm.

IgG titers were determined from cells seeded at a cell density of 1×105 cells per ml and grown for 6 days in 5 ml of Complete Medium in 50 ml Spin tube bioreactors when assessing polyclonal cell populations. Alternatively, shake flask cultures of clonal populations were inoculated at a density of 3×05 cells/ml into SFM4CHO media to initiate the fed batch production process. Fed batch production assays were performed with 25 ml of culture volume in 125 ml shake flasks or 5 ml in 50 ml TPP culture tubes in humidified incubators maintained at 37° C. and 5% C02 with shaking at 0 rpm (25 ml shake flask and spin tubes). The production was carried out for ten days by feeding 16%, of the initial culture volume of chemically defined concentrated feed (HYCLONE, Cell Boost 5, 52 g/l) on days zero, three and six to eight. No glutamine and glucose feeding were applied during the culture run. The viability and viable cell density (VCD) of the culture was measured daily using a GUAVA® machine (MILLIPORE). A double sandwich ELISA assay was used to determine MAb concentrations secreted into the culture media.

Batch and Fed-Batch Cultivation

Growth and production performances of individual clones expressing Bevacizumab IgG-, Fc-fusion and a circulating hormone were evaluated in batch cultivation into 50-ml minibioreactor (TPP, Switzerland) at 37° C. in 5% CO₂ humidified incubator for 7 days. At day 3, day 4 and day 7 of the cell cultivation, cell density and viability were determined using the Guava EasyCyte® flow cytometry system (MILLIPORE). IgG titer in cell culture supernatants was measured by sandwich ELISA. Cell density (Cv.ml 1) and IgG titer values (pg.ml) were plotted at the indicated process time sampling day.

The specific IgG productivity of the recombinant-protein expressing clones was determined as the slope of MIPs concentration versus integral number of viable cell (IVCD) calculated from day 3 to day 7 (production phase), and expressed as pg per cell and per day (pcd). For fed-batch production cultures, cells were seeded at 0.3×106 cells/ml into 125 ml shake flasks in 25 ml of SFM4CHO Hyclone serum-free medium. Cultures were maintained at 37° C. and 5% CO₂ under agitation. Cultures were fed in a daily based with a commercial Hyclone Feed (THERMO SCIENTIFIC). Cell densities and IgG production were daily evaluated.

Erp27 and Erp57:

DNA Vector Constructs

To obtain candidate gene coding sequences (CDS), total RNA was isolated from CHO-M cells (SURE CHO-M Cell Line™, Selexis SA, Switzerland) using the NucleoSpin™ RNA kit (MACHEREY-NAGEL). Reverse transcription was performed using the GoScript Reverse transcription System (Promega). Candidate gene CDS were inserted into the pBSK_ITR_BT+_X29_ITR (pBSK_ITR) or the pBSK_ITR_Blast vectors. The pBSK_ITR vector includes an expression cassette composed of the CMV/EF1alpha promoter and the BGH polyadenylation signal followed by the hMAR X-29 (Le Fourn, Girod, Buceta, Regamey, & Mermod, 2014). The expression cassette is flanked by the inverted terminal sequences of the piggyBac transposon. In the pBSK_ITR_Blast vector, a blasticidin resistance gene under the control of the SV40 promoter was inserted after the hMAR X-29. In experiments where Erp27 and Erp57 were overexpressed in difficult-to-express protein-expressing cells or upon titration of Erp27 or Ca3 overexpression, the pBSK_ITR plasmid was used and cells were co-transfected with a plasmid carrying the blasticidin resistance under the control of the SV40 promoter. In other experiments the pBSK_ITR_Blast vector was used. In experiments were Foxa1 was overexpressed, the CMV/EF1alpha promoter was replaced by a minimal CMV promoter for both Foxa1 and GFP expressions. The piggyBac transposase expression vector (pCS2+U5V5PBU3) was previously described (Ley et al., 2013).

Reactive Oxygen Species Analysis

The intracellular reactive oxygen species (ROS) level was detected by using 6-carboxy-2′,7′-dichlorodihydrofluorescein diacetate (carboxy-H₂DCFDA, THERMOFISHER SCIENTIFIC). At different days of the fed-batch cultures, 2 million cells were incubated in PBS containing 50 μM carboxy-H₂DCFDA for 30 minutes. Cells were then centrifuged, resuspended in 1 ml PBS and stained with DAPI to exclude dead cells. Carboxy-H₂DCFDA fluorescence was analyzed by flow cytometry in the DAPI negative cell populations (Gallios®, BECKMAN COULTER).

Cell Culture, Stable Transformation and Stable Polyclonal Line Analyses

Cells were maintained in suspension culture in SFM4CHO Hyclone serum-free medium (GE Healthcare) supplemented with 5% HyClone Cell Boost 5 supplement (GE HEALTHCARE), 8 mM L-glutamine (PAA, Austria) and 1×HT supplement (GIBCO) at 37° C. in a humidified incubator with 5% C02. Polyclonal CHO-M cells producing the trastuzumab or infliximab antibody were generated and characterized as previously described (Le Fourn et al., 2014). IgG expressing stable clones were obtained by cell sorting on FACSAria II (BD), expanded and analyzed for IgG production levels by sandwich ELISA. Stable cell lines overexpressing the candidate genes were obtained by re-transfecting trastuzumab or infliximab-producing clones with pBSK_ITR_CDS, pBlast and pCS2+U5V5PBU3 or with pBSK_ITR_Blast_CDS and pCS2+U5V5PBU3 using electroporation following the manufacturer's protocol (Neon® transfection system 100 uL Kit, INVITROGEN). Cells with stable insertions were selected using 3 or 7.5 μg/ml of blasticidin (INVIVOGEN). For etanercept producing clones, single subclones co-expressing Erp27 and Erp57 were isolated and their production was assessed using a ClonePix cell colony imaging device. The cell colonies showing the widest etanercept secretion halo were isolated from the Erp27 and Erp57 overexpressing or control cell populations.

ACTC1 and TAGAP:

DNA Vector Constructs

Genomic and cDNA sequences of the ACTC1 and TAGAP genes were determined after alignment to the homologous genes in mice using NCBI BLAST software. Transcript sequence RNAseq analysis were performed on Selexis SA CHO K1 cells (CHO-M). The cDNA libraries were generated by reverse transcription from 1 ug total RNA isolated from 10⁶ CHO-M cells (NucleoSpin™ RNA kit; MACHEREY-NAGEL) using the GoScript® Reverse transcription System (PROMEGA). The ACTC1 and TAGAP coding sequences (CDS) were cloned into the pBSK_ITR_BT+_EGFP_X29_ITR transposable expression vector (Le Fourn, Girod, Buceta, Regamey, & Mermod, 2014), yielding the pBSK-ACTC1 and pBSK-TAGAP expression vectors. The pBSK_ITR_BT+_EGFP_X29_ITR vector comprises an expression cassette composed of the CMV/EF1alpha fusion promoter and the BGH polyadenylation signal followed by the hMAR X-29. The expression cassette is flanked by the inverted terminal sequences of the piggyBac transposon. The blasticidin vector (pBlast) contains the blasticidin resistance gene under the control of the SV40 promoter originated from pRc/RSVplasmid (Invitrogen/Life Technologies).

Cell Culture and Stable Transfections

CHO K1 cells were maintained in suspension culture in SFM4CHO Hyclone® serum-free medium (SFM, ThermoScientific™) supplemented with L-glutamine (PAA, Austria) and HT supplement (GIBCO, INVITROGEN LIFE SCIENCES) at 37° C., 5% CO₂ in humidified air. Other cell media used for these experiments is the Deficient BalanCD CHO Growth A (B-CDmin©; IRVINE SCIENTIFIC), supplemented with vitamin B1 (thiamine Hydrochloride; SIGMA ALDRICH), vitamin B5 (Calcium DL-Pantothenate; TCI) and vitamin H (Biotin, SIGMA ALDRICH). CHO cells were transfected with pBSK-ACTC1 or TAGAP, pBlast, and pCS2-U5-PBU3 IgG1-Hc or IgG1-Lc expression vectors by electroporation according to the manufacturer's recommendations (NEONDEVICES, INVITROGEN). Production of stable cell lines was achieved by culturing transfected cells in the SFM4CHO media complemented with 7.5 μg/ml of blasticidin for 3 weeks. Polyclonal cell populations expressing the IgG were selected for further experiments as follow: for blasticidin selection, cells were seeded in SFM4CHO media supplemented with 10 μg/ml blasticidin for 2 weeks, then cultured into wells containing non-supplemented culture medium for 5 days, and then transferred into 50 ml spin tubes.

Analyses of Stable Polyclonal and Monoclonal Lines

Fed-Batch Performance Evaluation, IgG cell surface staining, IgG cell secretion assay and vitamin B5 metabolite quantification, were performed as previously described (Pourcel et al., 2019). Briefly, IgG secretion performances in fed-batch culture were performed as previously reported (Le Fourn et al., 2014). The assay of cell surface IgG was as reported previously (Brezinsky et al., 2003), and cell pools expressing recombinant IgG protein were subcloned using ClonePix™ FL Imager from Molecular Devices®. For vitamin B5 metabolite quantification, cell pellets were extracted with 1 mL of cold MeOH:H₂O (4:1, v/v) solvent mixture, then probe-sonicated. Supernatant obtained after 1 hour incubation at −20° C., followed by 15 min centrifugation at 13,000 rpm at 4° C. were collected and evaporated to dryness then reconstituted in 100 μL MeOH:Water (4:1) and injected into the LC-MS system. The protein pellets were evaporated and lysed in 20 mM Tris-HCl (pH 7.5), 4 M guanidine hydrochloride, 150 mM NaCl, 1 mM Na2EDTA, 1 mM EGTA, 1% Triton, 2.5 mM sodium pyrophosphate, 1 mM beta-glycerophosphate, 1 mM Na3VO4, 1 μg/ml leupeptin using brief probe-sonication. Extracted samples were analysed by HILIC-HRMS in negative ionization modes using a Q-Exactive® instrument (Thermo Fisher Scientific©) operating at mass resolving power of 70,000 full width half maximum. Raw LC-HRMS data was processed using the Thermo Fisher Scientific® software (Xcalibur® 4.0 QuanBrowser©, THERMO FISHER SCIENTIFIC). Metabolite quantification was performed using external calibration curves.

RNA RT-PCR and Sequencing RNA-Seq Analysis

For RNA reverse transcription and real time quantitative PCR (RT-qPCR) analysis, total RNA was extracted from 106 cells and reverse transcribed into cDNA using polyT primers. Transcripts accumulation was quantified by qPCR using the SYBR Green-Taq polymerase kit from EUROGENTEC Inc. and ABI Prism 7700 PCR machine (APPLIED BIOSYSTEMS). Transcript levels were normalized to that of the GAPDH housekeeping gene. RNASeq analysis of the B5- and puromycin-selected CHO cell was as previously described (Pourcel et al., 2019).

Briefly, total RNA was extracted from i) parental CHO cells, ii) CHO cell clones expressing the Interferon beta and the B5 transporter SLC5A6 expression vectors subjected to B5 deprivation/puromycin selection or puromycin selection only, iii) CHO cell pools expressing the Trastuzumab and SLC5A6 expression vectors selected as previously with B5 deprivation/puromycin selection or puromycin selection only. cDNA was obtained from 0.5 μg to 1 μg of total RNA using the Illumina TruSeq® stranded mRNA-seq reagents (ILLUMINA). The RNA-seq library 100 nt paired end was sequenced on the Illumina HiSeq 2500®. Reads were mapped to the CHO-K1 transcriptome (RefSeq, 2014).

Protein Sample Preparation and Immunoblotting

Total actin content was evaluated as follow. Protein extraction was performed from 10⁷ cells washed in PBS, after which the cell pellet was resuspended in RIPA lysis buffer (150 Mm NaCl, 50 mM Tris-HCl pH 8.0, 1% NP-40, 0.1% sodium deoxycholate, 0.1% SDS) and agitated for 30 min. The cell debris were pelleted by centrifugation (5 min, 15.000 g) and the supernatant collected. Equal volumes of proteins samples were processed for denaturing gel electrophoresis and immunoblotting, using 6-14% SDS/Page gels, Mini-Protean Tetra Gel (Bio-Rad) and Mini trans Blot Cell (Bio-Rad), and proteins were blotted onto nitrocellulose membranes. Membranes were blocked in TBST (Tris Base 20 mM, NaCl 135 mM, Tween-20 0.1%, pH 7.6) with 5% skim milk powder for 1 h at room temperature. The membranes were then incubated overnight with anti-alpha-cardiac Actin Polyclonal Antibody (PA5-21396, Invitrogen, dilution 1:500) or anti-GAPDH (sc-32233, SANTA CRUZ BIOTECHNOLOGY, dilution 1:500), then incubated for 1 h with HRP-conjugated secondary antibody, anti-mouse (G21040, Invitrogen, dilution 1:1000). Protein bands were visualized by using SuperSignal West Pico PLUS® (34580, THERMO SCIENTIFIC) and ChemiDoc® Imaging System (BIO-RAD). Resulting protein bands intensities were quantified with FIJI distribution of ImageJ® (NIH, Bethesda, Md.).

Analysis of Actin Polymerization by Fluorescence-Activated Cell Sorting

The level of polymerized actin (F-actin) was assessed on cell cultures initiated by seeding 2×10⁵ cell/ml in SFM and culturing for 3 days at 37° C. with 5% CO₂. Three aliquots of 10⁶ cells were collected from each culture, and the cells were resuspended in fresh media supplemented by 200 nM of SiR-Actin (CY-SC001©, SPIROCHROME) and incubated for 4 h at 37° C., 5% CO₂. Cells were then sorted by FACS (BD FACS Aria II, BD BIOSCIENCES, San Jose, Calif.), sorting cells depending on their level of fluorescence (Abs 652 nm, Em 674 nm; low, medium and high fluorescence). These cell populations were expanded and maintained at 37° C., 5% CO₂ until further analysis.

It will be appreciated that the systems (vectors/cells etc.), methods and kits of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as limiting.

### BIBLIOGRAPHY

Alanen, H. I., Williamson, R. A., Howard, M. J., Hatahet, F. S., Salo, K. E., Kauppila, A., . . . Ruddock, L. W. (2006). ERp27, a new non-catalytic endoplasmic reticulum-located human protein disulfide isomerase family member, interacts with ERp57. J Biol Chem, 281(44), 33727-33738. doi:10.1074/jbc.M604314200
Aon M A, Cortassa S. Coherent and robust modulation of a metabolic network by cytoskeletal organization and dynamics. Biophys Chem. 2002 Jun. 19; 97(2-3):213-31.

Baek, E., Kim, C. L., Park, J. H., & Lee, G. M. (2015). Cell Engineering for Therapeutic Protein Production. In M. Al-Rubeai (Ed.), Cell Engineering, vol 9. Animal Cell Culture (pp. 565-590). doi:10.1007/978-3-319-10320-4_18
Bargsted, L., Hetz, C., & Matus, S. (2016). ERp57 in neurodegeneration and regeneration. Neural Regen Res, 11(2), 232-233. doi:10.4103/1673-5374.177722
Becker, E., Florin, L., Pfizenmaier, K., & Kaufmann, H. (2008). An XBP-1 dependent bottle-neck in production of IgG subtype antibodies in chemically defined serum-free Chinese hamster ovary (CHO) fed-batch processes. J Biotechnol, 135(2), 217-223. doi:10.1016/j.jbiotec.2008.03.008
Birkenfeld, J., Kartmann, B., Betz, H., & Roth, D. (2001). Cofilin activation during Ca(2+)-triggered secretion from adrenal chromaffin cells. Biochemical and biophysical research communications, 286(3), 493-498. doi:10.1006/bbrc.2001.5435
Borth, N., Mattanovich, D., Kunert, R., & Katinger, H. (2005). Effect of increased expression of protein disulfide isomerase and heavy chain binding protein on antibody secretion in a recombinant CHO cell line. Biotechnol Prog, 21(1), 106-111. doi:10.1021/bp0498241
Brezinsky, S. C. G., Chiang, G. G., Szilvasi, A., Mohan, S., Shapiro, R. I., MacLean, A., Thill, G. (2003). A simple method for enriching populations of transfected CHO cells for cells of higher specific productivity. Journal of Immunological Methods, 277(1-2), 141-155. doi:10.1016/s0022-1759(03)00108-x
Cain, K., Peters, S., Hailu, H., Sweeney, B., Stephens, P., Heads, J., . . . Dickson, A. (2013). A CHO cell line engineered to express XBP1 and ERO1-Lalpha has increased levels of transient protein expression. Biotechnol Prog, 29(3), 697-706. doi:10.1002/btpr.1693
Chen, L., Johnson, R. C., & Milgram, S. L. (1998). P-CIP1, a novel protein that interacts with the cytosolic domain of peptidylglycine alpha-amidating monooxygenase, is associated with endosomes. J Biol Chem, 273(50), 33524-33532. doi:10.1074/jbc.273.50.33524
Chung, J. Y., Lim, S. W., Hong, Y. J., Hwang, S. O., & Lee, G. M. (2004). Effect of doxycycline-regulated calnexin and calreticulin expression on specific thrombopoietin productivity of recombinant Chinese hamster ovary cells. Biotechnol Bioeng, 85(5), 539-546. doi:10.1002/bit.10919
Connelly T M, Berg A S, Harris L R 3rd, Hegarty J P, Ruggiero F M, Deiling S M, Brinton D L, Koltun W A. T-cell activation Rho GTPase-activating protein expression varies with inflammation location and severity in Crohn's disease. J Surg Res. 2014 August; 190(2):457-64.
Davis, R., Schooley, K., Rasmussen, B., Thomas, J., & Reddy, P. (2000). Effect of PDI overexpression on recombinant protein secretion in CHO cells. Biotechnol Prog, 16(5), 736-743. doi:10.1021/bp000107q
Debold, E. P., Saber, W., Cheema, Y., Bookwalter, C. S., Trybus, K. M., Warshaw, D. M., & Vanburen, P. (2010). Human actin mutations associated with hypertrophic and dilated cardiomyopathies demonstrate distinct thin filament regulatory properties in vitro. Journal of molecular and cellular cardiology, 48(2), 286-292. doi:10.1016/j.yjmcc.2009.09.014
Di Fiore, A., Monti, D. M., Scaloni, A., De Simone, G., & Monti, S. M. (2018). Protective Role of Carbonic Anhydrases III and VII in Cellular Defense Mechanisms upon Redox Unbalance. Oxid Med Cell Longev, 2018, 2018306. doi:10.1155/2018/2018306
Dinnis D M, Stansfield S H, Schlatter S, Smales C M, Alete D, Birch J R, Racher A J, Marshall C T, Nielsen L K, James D C. Functional proteomic analysis of GS-NS0 murine myeloma cell lines with varying recombinant monoclonal antibody production rate. Biotechnol Bioeng. 2006 Aug. 5; 94(5):830-41.
Duke-Cohan, J. S., Ishikawa, Y., Yoshizawa, A., Choi, Y. I., Lee, C. N., Acuto, O., . . . Reinherz, E. L. (2018). Regulation of thymocyte trafficking by Tagap, a GAP domain protein linked to human autoimmunity. Sci Signal, 11(534). doi:10.1126/scisignal.aan8799
Eyre, S., Hinks, A., Bowes, J., Flynn, E., Martin, P., Wilson, A. G., Barton, A. (2010). Overlapping genetic susceptibility variants between three autoimmune disorders: rheumatoid arthritis, type 1 diabetes and coeliac disease. Arthritis research & therapy, 12(5), R175. doi:10.1186/ar3139
Farrell, A., McLoughlin, N., Milne, J. J., Marison, I. W., & Bones, J. (2014). Application of multi-omics techniques for bioprocess design and optimization in chinese hamster ovary cells. J Proteome Res, 13(7), 3144-3159. doi:10.1021/pr500219b
Fischer, S., Handrick, R., & Otte, K. (2015). The art of CHO cell engineering: A comprehensive retrospect and future perspectives. Biotechnol Adv, 33(8), 1878-1896. doi:10.1016/j.biotechadv.2015.10.015
Friedman, J. R., & Kaestner, K. H. (2006). The Foxa family of transcription factors in development and metabolism. Cell Mol Life Sci, 63(19-20), 2317-2328. doi:10.1007/s00018-006-6095-6
Gulis, G., Simi, K. C., de Toledo, R. R., Maranhao, A. Q., & Brigido, M. M. (2014). Optimization of heterologous protein production in Chinese hamster ovary cells under overexpression of spliced form of human X-box binding protein. BMC Biotechnol, 14, 26. doi:10.1186/1472-6750-14-26
Gutierrez-Gonzalez, M., Latorre, Y., Zuniga, R., Aguillon, J. C., Molina, M. C., & Altamirano, C. (2019). Transcription factor engineering in CHO cells for recombinant protein production. Crit Rev Biotechnol, 39(5), 665-679. doi:10.1080/07388551.2019.1605496
Ha, T. K., Hansen, A. H., Kol, S., Kildegaard, H. F., & Lee, G. M. (2018). Baicalein Reduces Oxidative Stress in CHO Cell Cultures and Improves Recombinant Antibody Productivity. Biotechnol J, 13(3), e1700425. doi:10.1002/biot.201700425
Hansen, H. G., Nilsson, C. N., Lund, A. M., Kol, S., Grav, L. M., Lundqvist, M., . . . Kildegaard, H. F. (2015). Versatile microscale screening platform for improving recombinant protein productivity in Chinese hamster ovary cells. Sci Rep, 5, 18016. doi:10.1038/srep18016
Hansen, H. G., Pristovsek, N., Kildegaard, H. F., & Lee, G. M. (2017). Improving the secretory capacity of Chinese hamster ovary cells by ectopic expression of effector genes: Lessons learned and future directions. Biotechnol Adv, 35(1), 64-76. doi:10.1016/j.biotechadv.2016.11.008
Haredy, A. M., Nishizawa, A., Honda, K., Ohya, T., Ohtake, H., & Omasa, T. (2013). Improved antibody production in Chinese hamster ovary cells by ATF4 overexpression. Cytotechnology, 65(6), 993-1002. doi:10.1007/s10616-013-9631-x
Hayes, N. V., Smales, C. M., & Klappa, P. (2010). Protein disulfide isomerase does not control recombinant IgG4 productivity in mammalian cell lines. Biotechnol Bioeng, 105(4), 770-779. doi:10.1002/bit.22587
Hecklau, C., Pering, S., Seibel, R., Schnellbaecher, A., Wehsling, M., Eichhorn, T., . . . Zimmer, A. (2016). S-Sulfocysteine simplifies fed-batch processes and increases the CHO specific productivity via anti-oxidant activity. J Biotechnol, 218, 53-63. doi:10.1016/j.jbiotec.2015.11.022
Hu, Q., Lin, X., Ding, L., Zeng, Y., Pang, D., Ouyang, N., . . . Yao, H. (2018). ARHGAP42 promotes cell migration and invasion involving PI3K/Akt signaling pathway in nasopharyngeal carcinoma. Cancer Med, 7(8), 3862-3874. doi:10.1002/cam4.1552
Hudder A, Nathanson L, Deutscher M P. Organization of mammalian cytoplasm. Mol Cell Biol. 2003 December; 23(24):9318-26.
Hwang, S. O., Chung, J. Y., & Lee, G. M. (2003). Effect of doxycycline-regulated ERp57 expression on specific thrombopoietin productivity of recombinant CHO cells. Biotechnol Prog, 19(1), 179-184. doi:10.1021/bp025578m
Inoue I, Itoh F, Aoyagi S, Tazawa S, Kusama H, Akahane M, Mastunaga T, Hayashi K, Awata T, Komoda T, Katayama S. Fibrate and statin synergistically increase the transcriptional activities of PPARalpha/RXRalpha and decrease the transactivation of NFkappaB. Biochem Biophys Res Commun. 2002 Jan. 11; 290(1):131-9.
Issemann I, Green S. Activation of a member of the steroid hormone receptor superfamily by peroxisome proliferators. Nature. 1990 Oct. 18; 347(6294):645-50.
Johari, Y. B., Estes, S. D., Alves, C. S., Sinacore, M. S., & James, D. C. (2015). Integrated cell and process engineering for improved transient production of a “difficult-to-express” fusion protein by CHO cells. Biotechnol Bioeng, 112(12), 2527-2542. doi:10.1002/bit.25687
Kaneko, Y., Sato, R., & Aoyagi, H. (2010). Changes in the quality of antibodies produced by Chinese hamster ovary cells during the death phase of cell culture. J Biosci Bioeng, 109(3), 281-287. doi:10.1016/j.jbiosc.2009.09.043
Kersten S, Desvergne B, Wahli W. Roles of PPARs in health and disease. Nature. 2000 May 25; 405(6785):421-4.
Kim, J. Y., Kim, Y. G., & Lee, G. M. (2012). CHO cells in biotechnology for production of recombinant proteins: current state and further potential. Appl Microbiol Biotechnol, 93(3), 917-930. doi:10.1007/s00253-011-3758-5
Kober, F. X., Koelmel, W., Kuper, J., Drechsler, J., Mais, C., Hermanns, H. M., & Schindelin, H. (2013). The crystal structure of the protein-disulfide isomerase family member ERp27 provides insights into its substrate binding capabilities. J Biol Chem, 288(3), 2029-2039. doi:10.1074/jbc.M112.410522
Ku, S. C., Ng, D. T., Yap, M. G., & Chao, S. H. (2008). Effects of overexpression of X-box binding protein 1 on recombinant protein production in Chinese hamster ovary and NS0 myeloma cells. Biotechnol Bioeng, 99(1), 155-164. doi:10.1002/bit.21562
Knull, H. R., & Walsh, J. L. (1992). Association of glycolytic enzymes with the cytoskeleton. Current topics in cellular regulation, 33, 15-30.
Lao, M. S., & Toth, D. (1997). Effects of ammonium and lactate on growth and metabolism of a recombinant Chinese hamster ovary cell culture. Biotechnology progress, 13(5), 688-691. doi:10.1021/bp9602360
Le Fourn, V., Girod, P. A., Buceta, M., Regamey, A., & Mermod, N. (2014). CHO cell engineering to prevent polypeptide aggregation and improve therapeutic protein secretion. Metab Eng, 21, 91-102. doi:10.1016/j.ymben.2012.12.003
Ley, D., Harraghy, N., Le Fourn, V., Bire, S., Girod, P. A., Regamey, A., . . . Mermod, N. (2013). MAR elements and transposons for improved transgene integration and expression. PLoS One, 8(4), e62784. doi:10.1371/journal.pone.0062784
Lukinavičius, G., Reymond, L., D'Este, E., Masharina, A., Gottfert, F., Ta, H., Johnsson, K. (2014). Fluorogenic probes for live-cell imaging of the cytoskeleton. Nature Methods, 11, 731. doi:10.1038/nmeth.2972
Luo, W., Janostiak, R., Tolde, O., Ryzhova, L. M., Koudelkova, L., Dibus, M., . . . Rosel, D. (2017). ARHGAP42 is activated by Src-mediated tyrosine phosphorylation to promote cell motility. J Cell Sci, 130(14), 2382-2393. doi:10.1242/jcs.197434
Maruyama S Y, Ito M, Ikami Y, Okitsu Y, Ito C, Toshimori K, Fujii W, Yogo K. A critical role of solute carrier 22a14 in sperm motility and male fertility in mice. Sci Rep. 2016 Nov. 4; 6:36468.
Mays, R. W., Beck, K. A., & Nelson, W. J. (1994). Organization and function of the cytoskeleton in polarized epithelial cells: a component of the protein sorting machinery. Current Opinion in Cell Biology, 6(1), 16-24.
Michalik L, Auwerx J, Berger J P, Chatterjee V K, Glass C K, Gonzalez F J, Grimaldi P A, Kadowaki T, Lazar M A, O'Rahilly S, Palmer C N, Plutzky J, Reddy J K, Spiegelman B M, Staels B, Wahli W. International Union of Pharmacology. LXI. Peroxisome proliferator-activated receptors. Pharmacol Rev. 2006 December; 58(4):726-41.
Mohan, C., Park, S. H., Chung, J. Y., & Lee, G. M. (2007). Effect of doxycycline-regulated protein disulfide isomerase expression on the specific productivity of recombinant CHO cells: thrombopoietin and antibody. Biotechnol Bioeng, 98(3), 611-615. doi:10.1002/bit.21453
Nishimiya, D., Mano, T., Miyadai, K., Yoshida, H., & Takahashi, T. (2013). Overexpression of CHOP alone and in combination with chaperones is effective in improving antibody production in mammalian cells. Appl Microbiol Biotechnol, 97(6), 2531-2539. doi:10.1007/s00253-012-4365-9
Ohya, T., Hayashi, T., Kiyama, E., Nishii, H., Miki, H., Kobayashi, K., . . . Ohtake, H. (2008). Improved production of recombinant human antithrombin III in Chinese hamster ovary cells by ATF4 overexpression. Biotechnol Bioeng, 100(2), 317-324. doi:10.1002/bit.21758
Paavilainen, V. O., Bertling, E., Falck, S., & Lappalainen, P. (2004). Regulation of cytoskeletal dynamics by actin-monomer-binding proteins. Trends in Cell Biology, 14(7), 386-394. doi:10.1016/j.tcb.2004.05.002
Park, M. H., Kim, S. Y., Kim, Y. J., & Chung, Y. H. (2014). ALS2CR7 (CDK15) attenuates TRAIL induced apoptosis by inducing phosphorylation of survivin Thr34. Biochem Biophys Res Commun, 450(1), 129-134. doi:10.1016/j.bbrc.2014.05.070
Pourcel, L., Buron, F., Garcia, F., Delaloix, M. S., Le Fourn, V., Girod, P. A., & Mermod, N. (2019). Transient vitamin B5 starving improves mammalian cell homeostasis and protein production. Metabolic Engineering, under review.
Pybus, L. P., Dean, G., West, N. R., Smith, A., Daramola, O., Field, R., . . . James, D. C. (2014). Model-directed engineering of “difficult-to-express” monoclonal antibody production by Chinese hamster ovary cells. Biotechnol Bioeng, 111(2), 372-385. doi:10.1002/bit.25116
Rahimpour, A., Vaziri, B., Moazzami, R., Nematollahi, L., Barkhordari, F., Kokabee, L., . . . Mahboudi, F. (2013). Engineering the cellular protein secretory pathway for enhancement of recombinant tissue plasminogen activator expression in Chinese hamster ovary cells: effects of CERT and XBP1s genes. J Microbiol Biotechnol, 23(8), 1116-1122. doi:10.4014/jmb.1302.02035
Raisanen, S. R., Lehenkari, P., Tasanen, M., Rahkila, P., Harkonen, P. L., & Vaananen, H. K. (1999). Carbonic anhydrase III protects cells from hydrogen peroxide-induced apoptosis. FASEB J, 13(3), 513-522. doi:10.1096/fasebj.13.3.513
Rakhshandehroo M, Knoch B, Müller M, Kersten S. Peroxisome proliferator-activated receptor alpha target genes. PPAR Res. 2010; 2010: 612089.
Rindler M J, Xu C F, Gumper I, Cen C, Sonderegger P, Neubert T A. Calsyntenins are secretory granule proteins in anterior pituitary gland and pancreatic islet alpha cells. J Histochem Cytochem. 2008 April; 56(4):381-8.
Rouillard, A. D., Gundersen, G. W., Fernandez, N. F., Wang, Z., Monteiro, C. D., McDermott, M. G., & Ma'ayan, A. (2016). The harmonizome: a collection of processed datasets gathered to serve and mine knowledge about genes and proteins. Database (Oxford), 2016. doi:10.1093/database/baw100
Shi, C., Uda, Y., Dedic, C., Azab, E., Sun, N., Hussein, A. I., . . . Pajevic, P. D. (2018). Carbonic anhydrase III protects osteocytes from oxidative stress. FASEB J, 32(1), 440-452. doi:10.1096/fj.201700485RR
Silva, R. C., Sattlegger, E., & Castilho, B. A. (2016). Perturbations in actin dynamics reconfigure protein complexes that modulate GCN2 activity and promote an eIF2 response. Journal of cell science, 129(24), 4521-4533. doi:10.1242/jcs.194738
Stamnes, M. (2002). Regulating the actin cytoskeleton during vesicular transport. Curr Opin Cell Biol, 14(4), 428-433. doi:10.1016/S0955-0674(02)00349-6
Tannous, A., Pisoni, G. B., Hebert, D. N., & Molinari, M. (2015). N-linked sugar-regulated protein folding and quality control in the ER. Semin Cell Dev Biol, 41, 79-89. doi:10.1016/j.semcdb.2014.12.001
Tastanova, A., Schulz, A., Folcher, M., Tolstrup, A., Puklowski, A., Kaufmann, H., & Fussenegger, M. (2016). Overexpression of YY1 increases the protein production in mammalian cells. J Biotechnol, 219, 72-85. doi:10.1016/j.jbiotec.2015.12.005
Tigges, M., & Fussenegger, M. (2006). Xbp1-based engineering of secretory capacity enhances the productivity of Chinese hamster ovary cells. Metab Eng, 8(3), 264-272. doi:10.1016/j.ymben.2006.01.006
Tosco, M., Faelli, A., Gastaldi, G., Paulmichl, M., & Orsenigo, M. N. (2008). Endogenous lactate transport in Xenopus laevis oocyte: dependence on cytoskeleton and regulation by protein kinases. Journal of comparative physiology. B, Biochemical, systemic, and environmental physiology, 178(4), 457-463. doi:10.1007/s00360-007-0238-4
Vilá-Brau A, De Sousa-Coelho A L, Mayordomo C, Haro D, Marrero P F. Human HMGCS2 regulates mitochondrial fatty acid oxidation and FGF21 expression in HepG2 cell line. J Biol Chem. 2011 Jun. 10; 286(23):20423-30.
Wahli W, Michalik L. PPARs at the crossroads of lipid signaling and inflammation. Trends Endocrinol Metab. 2012 July; 23(7):351-63.
Walther, C. G., Whitfield, R., & James, D. C. (2016). Importance of Interaction between Integrin and Actin Cytoskeleton in Suspension Adaptation of CHO cells. Appl Biochem Biotechnol, 178(7), 1286-1302. doi:10.1007/s12010-015-1945-z
Wang, L., Wang, X., & Wang, C. C. (2015). Protein disulfide-isomerase, a folding catalyst and a redox-regulated chaperone. Free Radic Biol Med, 83, 305-313. doi:10.1016/j.freeradbiomed.2015.02.007
Wurm, F. M. (2004). Production of recombinant protein therapeutics in cultivated mammalian cells. Nat Biotechnol, 22(11), 1393-1398. doi:10.1038/nbt1026
Zaret, K. S., & Carroll, J. S. (2011). Pioneer transcription factors: establishing competence for gene expression. Genes Dev, 25(21), 2227-2241. doi:10.1101/gad.176826.111
Zhang X, Li S, Zhou Y, Su W, Ruan X, Wang B, Zheng F, Warner M, Gustafsson J Å, Guan Y. Ablation of cytochrome P450 omega-hydroxylase 4A14 gene attenuates hepatic steatosis and fibrosis. Proc Natl Acad Sci USA. 2017 Mar. 21; 114(12):3181-3185.

1. A eukaryotic expression system comprising:
at least one metabolism influencing product (MIP) expression vector comprising at least one nucleic acid encoding the at least one MIP under the control of at least one regulatory sequence.

2. The eukaryotic expression system of claim 1, wherein the MIP encoded is:
at least one transcription factor,
at least one factor that regulates RNA translation,
at least one structural protein and/or protein folding proteins or a protein interacting with the respective protein folding protein,
at least one protein involved in signal transduction, vesicular trafficking and or cell adhesion activities,
at least one protein involved in cell survival and/or proliferation,
at least one protein involved in apoptosis, and/or
at least one protein involved in glutathione catabolism.

3. The eukaryotic expression system of claim 1, wherein the at least one MIP comprises at least one PPAR selected from the group consisting of PPARα, PPARβ/δ and PPARγ.

4. The eukaryotic expression system of claim 1, wherein the at least one MIP comprises Foxa1 (Forkhead box protein A1) and/or a secondary MIPs of Foxa1.

5. The eukaryotic expression system of claim 1, wherein the at least one MIP is actin.

6. The eukaryotic expression system of claim 1, wherein the at least one MIP is a protein folding protein comprising Erp27 and optionally Erp57.

7. The eukaryotic expression system of claim 1, wherein the at least one regulatory sequence is a promoter selected from the group consisting of CMV, EF alpha, CMV/EF1 alpha, SV40, RSV, PGK, a promoter having an expression level of CMV, EF1alpha, CMV/EF1 alpha, SV40, RSV, PGK and combinations thereof.

8. The eukaryotic expression system of claim 1, wherein the at least one MIP comprises at least one primary MIP and at least one, or two or three further MIPs which is/are neither a primary nor a secondary MIP.

9. The eukaryotic expression system of claim 1 comprising at least 2, 3, 4, 5 or more MIPs.

10. The eukaryotic expression system of claim 1, wherein the MIP expression vector further comprises a first ITR (inverted terminal repeat) upstream and a second ITR downstream of the nucleic acid encoding the MIP.

11. The eukaryotic expression system of claim 1, wherein the at least one regulatory sequence comprises a MAR element or MAR construct, such as MAR 1-68 and/or MAR X-29, including a singular MAR element or MAR construct, optionally between the first and second ITR.

12. The eukaryotic expression system of claim 1, wherein the MIP expression vector is a transposon donor vector and wherein the expression system further comprises a transposase-expressing helper vector or mRNA.

13. The eukaryotic expression system of claim 1 comprising a carrier vector comprising at least one restriction enzyme cleavage site adapted for insertion of a nucleic acid encoding a protein of interest and optionally further comprising an antibiotic resistance gene and/or a vitamin transport protein such as sodium-multivitamin transporter SLC5A6.

14. The eukaryotic expression system of claim 12, wherein the transposase expressing helper vector comprises a sequence encoding a PB transposase, optionally flanked, upstream and downstream by untranslated Preliminary Amendment terminal regions (UTR).

15. A method comprising:
(a) transfecting a cell with the expression vector of the expression system of claim 1 and/or
adding to the eukaryotic cell at least one activator of a protein product of a gene expressing a MIP, and
(b) transfecting the cells with a carrier vector comprising a protein of interest.

16. The method of claim 15, wherein the at least one activator is added to the eukaryotic cell and is an activator of at least one, two or all PPARs in including PPARα, PPARβ/δ and/or PPARγ, such as bezafibrate.

17. The method of claim 15, wherein a MA/EL (maximum arrested/expression level) of the protein of interest is more than 1.5×the ML (maximum level), more than 2×the ML or even more than 2.5× or 3×the ML.

18. A kit comprising in one container, said eukaryotic expression system of claim 1 and, in a second container, instructions of how to use said system.

19. The kit of claim 18, further comprising at least one activator of the at least one MIP, wherein the MIP is preferably at least one PPAR, in particular PPARα, PPARβ/δ or PPARγ, and the activator is an activator of at least one, two or all PPARs such as bezafibrate.

20. A recombinant eukaryotic cell comprising the eukaryotic expression system of claim 1.

21. The recombinant eukaryotic cell according to claim 20, wherein the cell is at least stably transfected with the MIP expression vector or a part thereof comprising the at least one MIP and is optionally a Chinese Hamster Ovary (CHO) cell.

22. A recombinant eukaryotic cell comprising at least one endogenous MIP under the control of at least one exogenous promoter selected from the group of CMV, EF1alpha, CMV/EF1 alpha, SV40, RSV, PGK, a exogenous or recombinant endogenous promoter having an expression level of CMV, EF1alpha, CMV/EF1alpha, SV40, RSV, PGK and combinations thereof.

23. The recombinant eukaryotic cell according to claim 22, wherein the at least one MIP is under the control of a combination of promoters of a promoter ladder.

24. (canceled)

25. The eukaryotic expression system of claim 2, wherein the
at least one transcription factor is Foxa1 (Forkhead box protein A1) or PPAR (Peroxisome proliferator-activated receptor),
at least one factor that regulates RNA translation is Casc3 (Cancer susceptibility candidate gene 3),
at least one structural protein is actin, the protein folding protein is Erp27 (Endoplasmic Reticulum Protein 27), or the protein interacting with the respective protein folding protein is Erp57 (Endoplasmic Reticulum Protein 57),
at least one protein involved in signal transduction, vesicular trafficking and/or cell adhesion activities is Tagap (T cell activation GTPase activating protein), Rassf9 (Ras Association Domain Family Member 9), and/or Clstn3 (Calsyntenin 3),
at least one protein involved in cell survival and/or proliferation is CDK15 (Cyclin Dependent Kinase 15) or Ca3 (Carbonic Anhydrase 3),
at least one protein involved in apoptosis is CFLAR (CASP8 And FADD Like Apoptosis Regulator) or SOD1 (Superoxide Dismutase 1), and
at least one protein involved in glutathione catabolism is GCLM (Glutamate-Cysteine Ligase Modifier Subunit) or GGCT (Gamma-glutamylcyclotransferase).

26. The eukaryotic expression system of claim 4, wherein the secondary MIP of Foxa1 is Ca3 (Carbonic Anhydrase 3), Rassf9 (Ras Association Domain Family Member 9), Tagap (T cell activation GTPase activating protein) or a combination thereof.
